SEQUENCE LISTING (195 sequences)

SEQ ID NO: 1 - RNA, 3770 nt, Homo sapiens
  Gata6-Em
gacccacagccuggcacccuucggcgagcgcuguuuguuuagggcucggugaguccaaucaggagcccaggcugcaguuuuccggcagagcaguaagaggcgccuccucucuccuuuuuauucaccagcagcgcggcgcagaccccggacucgcgcucgcccgcuggcgcccucggcuucucuccgcgccugggagcacccuccgccgcggccguucuccaugcgcagcgcccgcccgaggagcuagacgucagcuuggagcggcgccggaccguggauggccuugacugacggcggcuggugcuugccgaagcgcuucggggccgcgggugcggacgccagcgacuccagagccuuuccagcgcgggagcccuccacgccgccuucccccaucucuuccucguccuccuccugcucccggggcggagagcggggccccggcggcgccagcaacugcgggacgccucagcucgacacggaggcggcggccggacccccggcccgcucgcugcugcucaguuccuacgcuucgcaucccuucggggcuccccacggaccuucggcgccuggggucgcgggccccgggggcaaccugucgagcugggaggacuugcugcuguucacugaccucgaccaagccgcgaccgccagcaagcugcugugguccagccgcggcgccaagcugagccccuucgcacccgagcagccggaggagauguaccagacccucgccgcucucuccagccaggguccggccgccuacgacggcgcgcccggcggcuucgugcacucugcggccgcggcggcagcagccgcggcggcggccagcuccccggucuacgugcccaccacccgcguggguuccaugcugcccggccuaccguaccaccugcaggggucgggcagugggccagccaaccacgcgggcggcgcgggcgcgcaccccggcuggccucaggccucggccgacagcccuccauacggcagcggaggcggcgcggcuggcggcggggccgcggggccuggcggcgcuggcucagccgcggcgcacgucucggcgcgcuuccccuacucucccagcccgcccauggccaacggcgccgcgcgggagccgggaggcuacgcggcggcgggcagugggggcgcgggaggcgugagcggcggcggcaguagccuggcggccaugggcggccgcgagccccaguacagcucgcugucggccgcgcggccgcugaacgggacguaccaccaccaccaccaccaccaccaccaccauccgagccccuacucgcccuacgugggggcgccacugacgccugccuggcccgccggacccuucgagaccccggugcugcacagccugcagagccgcgccggagccccgcucccggugccccggggucccagugcagaccugcuggaggaccuguccgagagccgcgagugcgugaacugcggcuccauccagacgccgcuguggcggcgggacggcaccggccacuaccugugcaacgccugcgggcucuacagcaagaugaacggccucagccggccccucaucaagccgcagaagcgcgugccuucaucacggcggcuuggauuguccugugccaacugucacaccacaacuaccaccuuauggcgcagaaacgccgagggugaacccgugugcaaugcuuguggacucuacaugaaacuccauggggugcccagaccacuugcuaugaaaaaagagggaauucaaaccaggaaacgaaaaccuaagaacauaaauaaaucaaagacuugcucugguaauagcaauaauuccauucccaugacuccaacuuccaccucuucuaacucagaugauugcagcaaaaauacuucccccacaacacaaccuacagccucaggggcgggugccccggugaugacuggugcgggagagagcaccaaucccgagaacagcgagcucaaguauucgggucaagaugggcucuacauaggcgucagucucgccucgccggccgaagucacguccuccgugcgaccggauuccuggugcgcccuggcccuggccugagcccacgccgccaggaggcagggagggcuccgccgcgggccucacuccacucgugucugcuuuugugcagcgguccagacaguggcgacugcgcugacagaacgugauucucgugccuuuauuuugaaagagauguuuuucccaagaggcuugcugaaagagugagagaagauggaagggaagggccagugcaacugggcgcuugggccacuccagccagcccgccuccggggcggacccugcuccacuuccagaagccaggacuaggaccugggccuugccugcuauggaauauugagagagauuuuuuaaaaaagauuuugcauuuuguccaaaaucaugugcuucuucugaucaauuuugguuguuccagaauuucuucauaccuuuuccacauccagauuucaugugcguucauggagaagaucacuugaggccauuugguacacaucucuggaggcugagucgguucaugaggucucuuaucaaaaauauuacucaguuugcaagacugcauuguaacuuuaacauacacugugacugacguuucucaaaguucauauuguguggcugaucugaagucagucggaauuuguaaacaggguagcaaacaagauauuuuucuuccauguauacaauaauuuuuuuaaaaagugcaauuugcguugcagcaaucaguguuaaaucauuugcauaagauuuaacagcauuuuuuauaaugaauguaaacauuuuaacuuaaugguacuuaaaauaauuuaaaagaaaaauguuaacuuagacauucuuaugcuucuuuuacaacuacaucccauuuuauauuuccaauuguuaaagaaaaauauuucaagaacaaaucuucucucaggaaaauugccuuucucuauuuguuaagaauuuuuauacaagaacaccaauauacccccuuuauuuuacuguggaauaugugcuggaaaaauugcaacaacacuuuacuaccuaacggauagcauuuguaaauacucuagguaucuguaaacacucugaugaagucuguauagugugacuaacccacaggcagguugguuuacauuaauuuuuuuuuuugaaugggauguccuauggaaaccuauuucaccagaguuuuaaaaauaaaaaggguauuguuuugucuucuguacagugaguuccuucccuuuucaaagcuuucuuuuuaugcuguaugugacuauagauauucauauaaaacaagugcacgugaaguuugcaaaaugcuuuaaggccuuccuuucaaagcauaguccuuuuggagccguuuuguaccuuuuauaccuuggcuuauuugaaguugacacaugggguuaguuacuacucuccaugugcauuggggacaguuuuuauaagugggaaggacucaguauuauuauauuugagaugauaagcauuuuguuugggaacaaugcuuaaaaauauuccagaaaguucagauuuuuuuucuuugugaaugaaauauauucuggcccacgaacagggcgauuuccuuucaguuuuuuccuuuugcaacgugccuugaagucucaaagcucaccugagguugcagacguuacccccaacagaagauagguagaaaugauuccaguggccucuuuguauuuucuucauuguugaguagauuucaggaaaucaggagguguuucacaauacagaaugauggccuuuaacugug

SEQ ID NO: 2 - RNA, 2352 nt, Homo sapiens
  Nkx2-1-Em
gaaacuuaaagguguuuaccuugucaucagcauguaagcuaauuaucucgggcaagauguaggcuucuauugucuuguugcuuuagcgcuuacgccccgccucugguggcugccuaaaaccuggcgccgggcuaaaacaaacgcgaggcagcccccgagccuccacucaagccaauuaaggaggacucgguccacuccguuacguguacauccaacaagaucggcguuaagguaacaccagaauauuuggcaaagggagaaaaaaaaagcagcgaggcuucgccuucccccucucccuuuuuuuuccuccucuuccuuccuccuccagccgccgccgaaucaugucgaugaguccaaagcacacgacuccguucucagugucugacaucuugaguccccuggaggaaagcuacaagaaagugggcauggagggcggcggccucggggcuccgcuggcggcguacaggcagggccaggcggcaccgccaacagcggccaugcagcagcacgccguggggcaccacggcgccgucaccgccgccuaccacaugacggcggcgggggugccccagcucucgcacuccgccguggggggcuacugcaacggcaaccugggcaacaugagcgagcugccgccguaccaggacaccaugaggaacagcgccucuggccccggaugguacggcgccaacccagacccgcgcuuccccgccaucucccgcuucaugggcccggcgagcggcaugaacaugagcggcaugggcggccugggcucgcugggggacgugagcaagaacauggccccgcugccaagcgcgccgcgcaggaagcgccgggugcucuucucgcaggcgcagguguacgagcuggagcgacgcuucaagcaacagaaguaccugucggcgccggagcgcgagcaccuggccagcaugauccaccugacgcccacgcaggucaagaucugguuccagaaccaccgcuacaaaaugaagcgccaggccaaggacaaggcggcgcagcagcaacugcagcaggacagcggcggcggcgggggcggcgggggcaccgggugcccgcagcagcaacaggcucagcagcagucgccgcgacgcguggcggugccgguccuggugaaagacggcaaaccgugccaggcgggugcccccgcgccgggcgccgccagccuacaaggccacgcgcagcagcaggcgcagcaccaggcgcaggccgcgcaggcggcggcagcggccaucuccgugggcagcgguggcgccggccuuggcgcacacccgggccaccagccaggcagcgcaggccagucuccggaccuggcgcaccacgccgccagccccgcggcgcugcagggccagguauccagccugucccaccugaacuccucgggcucggacuacggcaccauguccugcuccaccuugcuauacggucggaccuggugagaggacgccgggccggcccuagcccagcgcucugccucaccgcuucccuccugcccgccacacagaccaccauccaccgcugcuccacgcgcuucgacuuuucuuaacaaccuggccgcguuuagaccaaggaacaaaaaaaccacaaaggccaaacugcuggacgucuuucuuuuuuucccccccuaaaauuuguggguuuuuuuuuuuaaaaaaagaaaaugaaaaacaaccaagcgcauccaaucucaaggaaucuuuaagcagagaagggcauaaaacagcuuuggggugucuuuuuuuggugauucaaauggguuuuccacgcuagggcggggcacagauuggagagggcucugugcugacauggcucuggacucuaaagaccaaacuucacucugggcacacucugccagcaaagaggacucgcuuguaaauaccaggauuuuuuuuuuuuuuugaagggaggacgggagcuggggagaggaaagagucuucaacauaacccacuugucacugacacaaaggaagugcccccuccccggcacccucuggccgccuaggcucagcggcgaccgcccuccgcgaaaauaguuuguuuaaugugaacuuguagcuguaaaacgcugucaaaaguuggacuaaaugccuaguuuuuaguaaucuguacauuuuguuguaaaaagaaaaaccacucccaguccccagcccuucacauuuuuuaugggcauugacaaaucuguguauauuauuuggcaguuugguauuugcggcgucagucuuuuucuguuguaacuuauguagauauuuggcuuaaauauaguuccuaagaagcuucuaauaaauuauacaaauuaaaaagauucuuuuucugauuaaaaaaaaaaaaaaaaaa

SEQ ID NO: 3 - RNA, 2428 nt, Homo sapiens
  Foxa2-Em
cccgcccacuuccaacuaccgccuccggccugcccagggagagagagggaguggagcccagggagagggagcgcgagagagggagggaggaggggacggugcuuuggcugacuuuuuuuuaaaagaggguggggguggggggugauugcuggucguuuguuguggcuguuaaauuuuaaacugccaugcacucggcuuccaguaugcugggagcggugaagauggaagggcacgagccguccgacuggagcagcuacuaugcagagcccgagggcuacuccuccgugagcaacaugaacgccggccuggggaugaacggcaugaacacguacaugagcaugucggcggccgccaugggcagcggcucgggcaacaugagcgcgggcuccaugaacaugucgucguacgugggcgcuggcaugagcccgucccuggcggggaugucccccggcgcgggcgccauggcgggcaugggcggcucggccggggcggccggcguggcgggcauggggccgcacuugagucccagccugagcccgcucggggggcaggcggccggggccaugggcggccuggcccccuacgccaacaugaacuccaugagccccauguacgggcaggcgggccugagccgcgcccgcgaccccaagaccuacaggcgcagcuacacgcacgcaaagccgcccuacucguacaucucgcucaucaccauggccauccagcagagccccaacaagaugcugacgcugagcgagaucuaccaguggaucauggaccucuuccccuucuaccggcagaaccagcagcgcuggcagaacuccauccgccacucgcucuccuucaacgacuguuuccugaaggugccccgcucgcccgacaagcccggcaagggcuccuucuggacccugcacccugacucgggcaacauguucgagaacggcugcuaccugcgccgccagaagcgcuucaagugcgagaagcagcuggcgcugaaggaggccgcaggcgccgccggcagcggcaagaaggcggccgccggagcccaggccucacaggcucaacucggggaggccgccgggccggccuccgagacuccggcgggcaccgagucgccucacucgagcgccuccccgugccaggagcacaagcgagggggccugggagagcugaaggggacgccggcugcggcgcugagccccccagagccggcgcccucucccgggcagcagcagcaggccgcggcccaccugcugggcccgccccaccacccgggccugccgccugaggcccaccugaagccggaacaccacuacgccuucaaccacccguucuccaucaacaaccucauguccucggagcagcagcaccaccacagccaccaccaccaccaaccccacaaaauggaccucaaggccuacgaacaggugaugcacuaccccggcuacgguucccccaugccuggcagcuuggccaugggcccggucacgaacaaaacgggccuggacgccucgccccuggccgcagauaccuccuacuaccaggggguguacucccggcccauuaugaacuccucuuaagaagacgacggcuucaggcccggcuaacucuggcaccccggaucgaggacaagugagagagcaagugggggucgagacuuuggggagacgguguugcagagacgcaagggagaagaaauccauaacacccccaccccaacacccccaagacagcagucuucuucacccgcugcagccguuccgucccaaacagagggccacacagauaccccacguucuauauaaggaggaaaacgggaaagaauauaaaguuaaaaaaaagccuccgguuuccacuacuguguagacuccugcuucuucaagcaccugcagauucugauuuuuuuguuguuguuguucuccuccauugcuguuguugcagggaagucuuacuuaaaaaaaaaaaaaaauuuugugagugacucgguguaaaaccauguaguuuuaacagaaccagaggguuguacuauuguuuaaaaacaggaaaaaaaauaauguaagggucuguuguaaaugaccaagaaaaagaaaaaaaaagcauucccaaucuugacacggugaaauccaggucucggguccgauuaauuuaugguuucugcgugcuuuauuuauggcuuauaaauguguauucuggcugcaagggccagaguuccacaaaucuauauuaaaguguuauacccgguuuuaucccuugaaucuuuucuuccagauuuuucuuuucuuuacuuggcuuacaaaauauacaggcuuggaaauuauuucaagaaggagggagggauacccugucugguugcagguuguauuuuauuuuggcccagggaguguugcuguuuucccaacauuuuauuaauaaaauuuucagacauaaaaaa

SEQ ID NO: 4 - RNA, 1402 nt, Homo sapiens
  Id2-Em
ggggacgaagggaagcuccagcguguggccccggcgagugcggauaaaagccgccccgccgggcucgggcuucauucugagccgagcccggugccaagcgcagcuagcucagcaggcggcagcggcggccugagcuucagggcagccagcucccucccggucucgccuucccucgcggucagcaugaaagccuucagucccgugagguccguuaggaaaaacagccugucggaccacagccugggcaucucccggagcaaaaccccuguggacgacccgaugagccugcuauacaacaugaacgacugcuacuccaagcucaaggagcuggugcccagcaucccccagaacaagaaggugagcaagauggaaauccugcagcacgucaucgacuacaucuuggaccugcagaucgcccuggacucgcaucccacuauugucagccugcaucaccagagacccgggcagaaccaggcguccaggacgccgcugaccacccucaacacggauaucagcauccuguccuugcaggcuucugaauucccuucugaguuaaugucaaaugacagcaaagcacuguguggcugaauaagcgguguucaugauuucuuuuauucuuugcacaacaacaacaacaacaaauucacggaaucuuuuaagugcugaacuuauuuuucaaccauuucacaaggaggacaaguugaauggaccuuuuuaaaaagaaaaaaaaaauggaaggaaaacuaagaaugaucaucuucccaggguguucucuuacuuggacugugauauucguuauuuaugaaaaagacuuuuaaaugcccuuucugcaguuggaagguuuucuuuauauacuauucccaccauggggagcgaaaacguuaaaaucacaaggaauugcccaaucuaagcagacuuugccuuuuuucaaagguggagcgugaauaccagaaggauccaguauucagucacuuaaaugaagucuuuuggucagaaauuaccuuuuugacacaagccuacugaaugcuguguauauauuuauauauaaauauaucuauuugagugaaaccuugugaacucuuuaauuagaguuuucuuguauaguggcagagaugucuauuucugcauucaaaaguguaaugauguacuuauucaugcuaaacuuuuuauaaaaguuuaguuguaaacuuaacccuuuuauacaaaauaaaucaaguguguuuauugaauggugauugccugcuuuauuucagaggaccagugcuuugauuuuuauuaugcuauguuauaacugaacccaaauaaauacaaguucaaauuuauguagacuguauaagauuauaauaaaacaugucugaagucaaaaaaaaaaaaaaaaaaaaaaaaaaaaaaaaaaaaaaaa

SEQ ID NO: 5 - RNA, 3158 nt, Homo sapiens
  Gata6-Ad
auugaucuccacgcccggggcagaaauaggaucuuugagaagucucaaaugggaucuuugagaagucagaucccauuugaacuagaaaaaggaguggaggcgagguagcgugcagccuacgcucuuguuaacccgucgaucuccuaccauacccgucucccccaccccaccucaggagcuagacgucagcuuggagcggcgccggaccguggauggccuugacugacggcggcuggugcuugccgaagcgcuucggggccgcgggugcggacgccagcgacuccagagccuuuccagcgcgggagcccuccacgccgccuucccccaucucuuccucguccuccuccugcucccggggcggagagcggggccccggcggcgccagcaacugcgggacgccucagcucgacacggaggcggcggccggacccccggcccgcucgcugcugcucaguuccuacgcuucgcaucccuucggggcuccccacggaccuucggcgccuggggucgcgggccccgggggcaaccugucgagcugggaggacuugcugcuguucacugaccucgaccaagccgcgaccgccagcaagcugcugugguccagccgcggcgccaagcugagccccuucgcacccgagcagccggaggagauguaccagacccucgccgcucucuccagccaggguccggccgccuacgacggcgcgcccggcggcuucgugcacucugcggccgcggcggcagcagccgcggcggcggccagcuccccggucuacgugcccaccacccgcguggguuccaugcugcccggccuaccguaccaccugcaggggucgggcagugggccagccaaccacgcgggcggcgcgggcgcgcaccccggcuggccucaggccucggccgacagcccuccauacggcagcggaggcggcgcggcuggcggcggggccgcggggccuggcggcgcuggcucagccgcggcgcacgucucggcgcgcuuccccuacucucccagcccgcccauggccaacggcgccgcgcgggagccgggaggcuacgcggcggcgggcagugggggcgcgggaggcgugagcggcggcggcaguagccuggcggccaugggcggccgcgagccccaguacagcucgcugucggccgcgcggccgcugaacgggacguaccaccaccaccaccaccaccaccaccaccauccgagccccuacucgcccuacgugggggcgccacugacgccugccuggcccgccggacccuucgagaccccggugcugcacagccugcagagccgcgccggagccccgcucccggugccccggggucccagugcagaccugcuggaggaccuguccgagagccgcgagugcgugaacugcggcuccauccagacgccgcuguggcggcgggacggcaccggccacuaccugugcaacgccugcgggcucuacagcaagaugaacggccucagccggccccucaucaagccgcagaagcgcgugccuucaucacggcggcuuggauuguccugugccaacugucacaccacaacuaccaccuuauggcgcagaaacgccgagggugaacccgugugcaaugcuuguggacucuacaugaaacuccauggggugcccagaccacuugcuaugaaaaaagagggaauucaaaccaggaaacgaaaaccuaagaacauaaauaaaucaaagacuugcucugguaauagcaauaauuccauucccaugacuccaacuuccaccucuucuaacucagaugauugcagcaaaaauacuucccccacaacacaaccuacagccucaggggcgggugccccggugaugacuggugcgggagagagcaccaaucccgagaacagcgagcucaaguauucgggucaagaugggcucuacauaggcgucagucucgccucgccggccgaagucacguccuccgugcgaccggauuccuggugcgcccuggcccuggccugagcccacgccgccaggaggcagggagggcuccgccgcgggccucacuccacucgugucugcuuuugugcagcgguccagacaguggcgacugcgcugacagaacgugauucucgugccuuuauuuugaaagagauguuuuucccaagaggcuugcugaaagagugagagaagauggaagggaagggccagugcaacugggcgcuugggccacuccagccagcccgccuccggggcggacccugcuccacuuccagaagccaggacuaggaccugggccuugccugcuauggaauauugagagagauuuuuuaaaaaagauuuugcauuuuguccaaaaucaugugcuucuucugaucaauuuugguuguuccagaauuucuucauaccuuuuccacauccagauuucaugugcguucauggagaagaucacuugaggccauuugguacacaucucuggaggcugagucgguucaugaggucucuuaucaaaaauauuacucaguuugcaagacugcauuguaacuuuaacauacacugugacugacguuucucaaaguucauauuguguggcugaucugaagucagucggaauuuguaaacaggguagcaaacaagauauuuuucuuccauguauacaauaauuuuuuuaaaaagugcaauuugcguugcagcaaucaguguuaaaucauuugcauaagauuuaacagcauuuuuuauaaugaauguaaacauuuuaacuuaaugguacuuaaaauaauuuaaaagaaaaauguuaacuuagacauucuuaugcuucuuuuacaacuacaucccauuuuauauuuccaauuguuaaagaaaaauauuucaagaacaaaucuucucucaggaaaauugccuuucucuauuuguuaagaauuuuuauacaagaacaccaauauacccccuuuauuuuacuguggaauaugugcuggaaaaauugcaacaacacuuuacuaccuaacggauagcauuuguaaauacucuagguaucuguaaacacucugaugaagucuguauagugugacuaacccacaggcagguugguuuacauuaauuuuuuuuuuugaaugggauguccuauggaaaccuauuucaccagaguuuuaaaaauaaaaaggguauuguuuugucuucugu

SEQ ID NO: 6 - RNA, 2197 nt, Homo sapiens
  Nkx2-1-Ad
cugacagacacguagaccaacagugcggccccaggguucguccccagacucgcucgcucauuuguuggcgacuggggcucagcgcagcgaagcccgaugugguccggaggcagugggaaggcgcggggcugggaggccgcggcgggagggaggagcagccccggcaggcucagccgccgccgaaucaugucgaugaguccaaagcacacgacuccguucucagugucugacaucuugaguccccuggaggaaagcuacaagaaagugggcauggagggcggcggccucggggcuccgcuggcggcguacaggcagggccaggcggcaccgccaacagcggccaugcagcagcacgccguggggcaccacggcgccgucaccgccgccuaccacaugacggcggcgggggugccccagcucucgcacuccgccguggggggcuacugcaacggcaaccugggcaacaugagcgagcugccgccguaccaggacaccaugaggaacagcgccucuggccccggaugguacggcgccaacccagacccgcgcuuccccgccaucucccgcuucaugggcccggcgagcggcaugaacaugagcggcaugggcggccugggcucgcugggggacgugagcaagaacauggccccgcugccaagcgcgccgcgcaggaagcgccgggugcucuucucgcaggcgcagguguacgagcuggagcgacgcuucaagcaacagaaguaccugucggcgccggagcgcgagcaccuggccagcaugauccaccugacgcccacgcaggucaagaucugguuccagaaccaccgcuacaaaaugaagcgccaggccaaggacaaggcggcgcagcagcaacugcagcaggacagcggcggcggcgggggcggcgggggcaccgggugcccgcagcagcaacaggcucagcagcagucgccgcgacgcguggcggugccgguccuggugaaagacggcaaaccgugccaggcgggugcccccgcgccgggcgccgccagccuacaaggccacgcgcagcagcaggcgcagcaccaggcgcaggccgcgcaggcggcggcagcggccaucuccgugggcagcgguggcgccggccuuggcgcacacccgggccaccagccaggcagcgcaggccagucuccggaccuggcgcaccacgccgccagccccgcggcgcugcagggccagguauccagccugucccaccugaacuccucgggcucggacuacggcaccauguccugcuccaccuugcuauacggucggaccuggugagaggacgccgggccggcccuagcccagcgcucugccucaccgcuucccuccugcccgccacacagaccaccauccaccgcugcuccacgcgcuucgacuuuucuuaacaaccuggccgcguuuagaccaaggaacaaaaaaaccacaaaggccaaacugcuggacgucuuucuuuuuuucccccccuaaaauuuguggguuuuuuuuuuuaaaaaaagaaaaugaaaaacaaccaagcgcauccaaucucaaggaaucuuuaagcagagaagggcauaaaacagcuuuggggugucuuuuuuuggugauucaaauggguuuuccacgcuagggcggggcacagauuggagagggcucugugcugacauggcucuggacucuaaagaccaaacuucacucugggcacacucugccagcaaagaggacucgcuuguaaauaccaggauuuuuuuuuuuuuuugaagggaggacgggagcuggggagaggaaagagucuucaacauaacccacuugucacugacacaaaggaagugcccccuccccggcacccucuggccgccuaggcucagcggcgaccgcccuccgcgaaaauaguuuguuuaaugugaacuuguagcuguaaaacgcugucaaaaguuggacuaaaugccuaguuuuuaguaaucuguacauuuuguuguaaaaagaaaaaccacucccaguccccagcccuucacauuuuuuaugggcauugacaaaucuguguauauuauuuggcaguuugguauuugcggcgucagucuuuuucuguuguaacuuauguagauauuuggcuuaaauauaguuccuaagaagcuucuaauaaauuauacaaauuaaaaagauucuuuuucugauuaaaaaaaaaaaaaaaaaa

SEQ ID NO: 7 - RNA, 2415 nt, Homo sapiens
  Foxa2-Ad
cggccgcugcuagaggggcugcuugcgccaggcgccggccgccccacugcgggucccuggcggccggugucugaggagucggagagccgaggcggccagaccgugcgccccgcgcuucucccgaggccguuccgggucugaacuguaacagggaggggccucgcaggagcagcagcgggcgaguuaaaguaugcugggagcggugaagauggaagggcacgagccguccgacuggagcagcuacuaugcagagcccgagggcuacuccuccgugagcaacaugaacgccggccuggggaugaacggcaugaacacguacaugagcaugucggcggccgccaugggcagcggcucgggcaacaugagcgcgggcuccaugaacaugucgucguacgugggcgcuggcaugagcccgucccuggcggggaugucccccggcgcgggcgccauggcgggcaugggcggcucggccggggcggccggcguggcgggcauggggccgcacuugagucccagccugagcccgcucggggggcaggcggccggggccaugggcggccuggcccccuacgccaacaugaacuccaugagccccauguacgggcaggcgggccugagccgcgcccgcgaccccaagaccuacaggcgcagcuacacgcacgcaaagccgcccuacucguacaucucgcucaucaccauggccauccagcagagccccaacaagaugcugacgcugagcgagaucuaccaguggaucauggaccucuuccccuucuaccggcagaaccagcagcgcuggcagaacuccauccgccacucgcucuccuucaacgacuguuuccugaaggugccccgcucgcccgacaagcccggcaagggcuccuucuggacccugcacccugacucgggcaacauguucgagaacggcugcuaccugcgccgccagaagcgcuucaagugcgagaagcagcuggcgcugaaggaggccgcaggcgccgccggcagcggcaagaaggcggccgccggagcccaggccucacaggcucaacucggggaggccgccgggccggccuccgagacuccggcgggcaccgagucgccucacucgagcgccuccccgugccaggagcacaagcgagggggccugggagagcugaaggggacgccggcugcggcgcugagccccccagagccggcgcccucucccgggcagcagcagcaggccgcggcccaccugcugggcccgccccaccacccgggccugccgccugaggcccaccugaagccggaacaccacuacgccuucaaccacccguucuccaucaacaaccucauguccucggagcagcagcaccaccacagccaccaccaccaccaaccccacaaaauggaccucaaggccuacgaacaggugaugcacuaccccggcuacgguucccccaugccuggcagcuuggccaugggcccggucacgaacaaaacgggccuggacgccucgccccuggccgcagauaccuccuacuaccaggggguguacucccggcccauuaugaacuccucuuaagaagacgacggcuucaggcccggcuaacucuggcaccccggaucgaggacaagugagagagcaagugggggucgagacuuuggggagacgguguugcagagacgcaagggagaagaaauccauaacacccccaccccaacacccccaagacagcagucuucuucacccgcugcagccguuccgucccaaacagagggccacacagauaccccacguucuauauaaggaggaaaacgggaaagaauauaaaguuaaaaaaaagccuccgguuuccacuacuguguagacuccugcuucuucaagcaccugcagauucugauuuuuuuguuguuguuguucuccuccauugcuguuguugcagggaagucuuacuuaaaaaaaaaaaaaaauuuugugagugacucgguguaaaaccauguaguuuuaacagaaccagaggguuguacuauuguuuaaaaacaggaaaaaaaauaauguaagggucuguuguaaaugaccaagaaaaagaaaaaaaaagcauucccaaucuugacacggugaaauccaggucucggguccgauuaauuuaugguuucugcgugcuuuauuuauggcuuauaaauguguauucuggcugcaagggccagaguuccacaaaucuauauuaaaguguuauacccgguuuuaucccuugaaucuuuucuuccagauuuuucuuuucuuuacuuggcuuacaaaauauacaggcuuggaaauuauuucaagaaggagggagggauacccugucugguugcagguuguauuuuauuuuggcccagggaguguugcuguuuucccaacauuuuauuaauaaaauuuucagacauaaaaaa

SEQ ID NO: 8 - RNA, 1681 nt, Homo sapiens
  Id2-Ad
caaaggcggccuggccagcgcggagcucccggcccggagcugcuucugauuaccgcgaggggcccggacgcgagagccgccgcggggccugcccuagaggcggagugaugaacuguggcuuccccccugcggugcugaacucgcccguguagcugugauuuuagagcugccgacagcucuaagcugggcucgcgccccgcccaccccgcggggauuggcugcgaacgcggaagaaccaagcccacgccccgcgcccgcgcccaccaauggaagcgcccgcucgucuugauagacgugccaccuuccgccaauggggacgaagggaagcuccagcguguggccccggcgagugcggauaaaagccgccccgccgggcucgggcuucauucugagccgagcccggugccaagcgcagcuagcucagcaggcggcagcggcggccugagcuucagggcagccagcucccucccggucucgccuucccucgcggucagcaugaaagccuucagucccgugagguccguuaggaaaaacagccugucggaccacagccugggcaucucccggagcaaaaccccuguggacgacccgaugagccugcuauacaacaugaacgacugcuacuccaagcucaaggagcuggugcccagcaucccccagaacaagaaggugagcaagauggaaauccugcagcacgucaucgacuacaucuuggaccugcagaucgcccuggacucgcaucccacuauugucagccugcaucaccagagacccgggcagaaccaggcguccaggacgccgcugaccacccucaacacggauaucagcauccuguccuugcaggcuucugaauucccuucugaguuaaugucaaaugacagcaaagcacuguguggcugaauaagcgguguucaugauuucuuuuauucuuugcacaacaacaacaacaacaaauucacggaaucuuuuaagugcugaacuuauuuuucaaccauuucacaaggaggacaaguugaauggaccuuuuuaaaaagaaaaaaaaaauggaaggaaaacuaagaaugaucaucuucccaggguguucucuuacuuggacugugauauucguuauuuaugaaaaagacuuuuaaaugcccuuucugcaguuggaagguuuucuuuauauacuauucccaccauggggagcgaaaacguuaaaaucacaaggaauugcccaaucuaagcagacuuugccuuuuuucaaagguggagcgugaauaccagaaggauccaguauucagucacuuaaaugaagucuuuuggucagaaauuaccuuuuugacacaagccuacugaaugcuguguauauauuuauauauaaauauaucuauuugagugaaaccuugugaacucuuuaauuagaguuuucuuguauaguggcagagaugucuauuucugcauucaaaaguguaaugauguacuuauucaugcuaaacuuuuuauaaaaguuuaguuguaaacuuaacccuuuuauacaaaauaaaucaaguguguuuauugaauggugauugccugcuuuauuucagaggaccagugcuuugauuuuuauuaugcuauguuauaacugaacccaaauaaauacaaguucaaauuuauguagacuguauaagauuauaauaaaacaugucugaagucaauaccu

SEQ ID NO: 9 - DNA, 21 bp, Artificial Sequence
  Gata6-Em Fwd
ctcggcttctctccgcgcctg

SEQ ID NO: 10 - DNA, 20 bp, Artificial Sequence
  Gata6-Em Fwd
ttgactgacggcggctggtg

SEQ ID NO: 11 - DNA, 21 bp, Artificial Sequence
  Gata6-Em Rev
agctgaggcgtcccgcagttg

SEQ ID NO: 12 - DNA, 20 bp, Artificial Sequence
  Gata6-Em Rev
ctcccgcgctggaaaggctc

SEQ ID NO: 13 - DNA, 20 bp, Artificial Sequence
  Gata6-Ad Fwd
gcggtttcgttttcggggac

SEQ ID NO: 14 - DNA, 20 bp, Artificial Sequence
  Gata6-Ad Fwd
aggacccagactgctgcccc

SEQ ID NO: 15 - DNA, 20 bp, Artificial Sequence
  Gata6-Ad Rev
aagggatgcgaagcgtagga

SEQ ID NO: 16 - DNA, 20 bp, Artificial Sequence
  Gata6-Ad Rev
ctgaccagcccgaacgcgag

SEQ ID NO: 17 - DNA, 20 bp, Artificial Sequence
  Nkx2-1-Em Fwd
aaacctggcgccgggctaaa

SEQ ID NO: 18 - DNA, 20 bp, Artificial Sequence
  Nkx2-1-Em Fwd
cagcgaggcttcgccttccc

SEQ ID NO: 19 - DNA, 21 bp, Artificial Sequence
  Nkx2-1-Em Rev
ggagagggggaaggcgaagcc

SEQ ID NO: 20 - DNA, 20 bp, Artificial Sequence
  Nkx2-1-Em Rev
tcgacatgattcggcggcgg

SEQ ID NO: 21 - DNA, 20 bp, Artificial Sequence
  Nkx2-1-Ad Fwd
agcgaagcccgatgtggtcc

SEQ ID NO: 22 - DNA, 20 bp, Artificial Sequence
  Nkx2-1-Ad Fwd
tccggaggcagtgggaaggc

SEQ ID NO: 23 - DNA, 21 bp, Artificial Sequence
  Nk2-1-Ad Rev
ccgccctccatgcccactttc

SEQ ID NO: 24 - DNA, 20 bp, Artificial Sequence
  Nk2-1-Ad Rev
gacatgattcggcggcggct

SEQ ID NO: 25 - DNA, 21 bp, Artificial Sequence
  Foxa2-Em Fwd
tgccatgcactcggcttccag

SEQ ID NO: 26 - DNA, 20 bp, Artificial Sequence
  Foxa2-Em Fwd
cagggagagggagggcgaga

SEQ ID NO: 27 - DNA, 20 bp, Artificial Sequence
  Foxa2-Em Rev
tcatgttgcccgagccgctg

SEQ ID NO: 28 - DNA, 20 bp, Artificial Sequence
  Foxa2-Em Rev
cccccacccccaccctcttt

SEQ ID NO: 29 - DNA, 21 bp, Artificial Sequence
  Foxa2-Ad Fwd
ctgctagaggggctgcttgcg

SEQ ID NO: 30 - DNA, 20 bp, Artificial Sequence
  Foxa2-Ad Fwd
cgcttctcccgaggccgttc

SEQ ID NO: 31 - DNA, 20 bp, Artificial Sequence
  Foxa2-Ad Rev
acggctcgtgcccttccatc

SEQ ID NO: 32 - DNA, 20 bp, Artificial Sequence
  Foxa2-Ad Rev
taactcgcccgctgctgctc

SEQ ID NO: 33 - DNA, 20 bp, Artificial Sequence
  Id2-Em Fwd
aacccctgtggacgacccga

SEQ ID NO: 34 - DNA, 20 bp, Artificial Sequence
  Id2-Em Fwd
tgcggataaaagccgccccg

SEQ ID NO: 35 - DNA, 20 bp, Artificial Sequence
  Id2-Em Rev
gcccgggtctctggtgatgc

SEQ ID NO: 36 - DNA, 20 bp, Artificial Sequence
  Id2-Em Rev
agctagctgcgcttggcacc

SEQ ID NO: 37 - DNA, 20 bp, Artificial Sequence
  Id2-Ad Fwd
ctgcggtgctgaactcgccc

SEQ ID NO: 38 - DNA, 20 bp, Artificial Sequence
  Id2-Ad Fwd
ccccctgcggtgctgaactc

SEQ ID NO: 39 - DNA, 20 bp, Artificial Sequence
  Id2-Ad Rev
gacgagcgggcgcttccatt

SEQ ID NO: 40 - DNA, 20 bp, Artificial Sequence
  Id2-Ad Rev
taactcgcccgctgctgctc

SEQ ID NO: 41 - RNA, 21 nt, Artificial Sequence
  Gata6-Em sense siRNA
ucaggagcgcaggcugcagtt

SEQ ID NO: 42 - RNA, 21 nt, Artificial Sequence
  Gata6-Em sense siRNA
gaggcgccuccucucuccutt

SEQ ID NO: 43 - RNA, 21 nt, Artificial Sequence
  Gata6-Em antisense siRNA
cugcagccugcgcuccugatt

SEQ ID NO: 44 - RNA, 21 nt, Artificial Sequence
  Gata6-Em antisense siRNA
aggagagaggaggcgccuctt

SEQ ID NO: 45 - RNA, 21 nt, Artificial Sequence
  FoxA2-Em sense siRNA
accgccaugcacucggcuutt

SEQ ID NO: 46 - RNA, 21 nt, Artificial Sequence
  FoxA2-Em antisense siRNA
aagccgagugcauggcggutt

SEQ ID NO: 47 - RNA, 58 nt, Artificial Sequence
  Nkx2-1-Em shRNA
ccggcccatgaagaagaaagcaattctcgagaattgctttcttcttcatgggtttttg

SEQ ID NO: 48 - RNA, 62 nt, Artificial Sequence
  Nkx2-1-Em shRNA
gtaccgggggatcatccttgtagataaactcgagtttatctacaaggatgatcccttttttg

SEQ ID NO: 49 - RNA, 58 nt, Artificial Sequence
  Nkx2-1-Em shRNA
ccggattcggaatcagctagcaattctcgagaattgctagctgattccgaattttttg

SEQ ID NO: 50 - protein, 595 aa, Homo sapiens
  Gata6-Em
MALTDGGWCLPKRFGAAGADASDSRAFPAREPSTPPSPISSSSSSCSRGGERGPGGASNCGTPQLDTEAAAGPPARSLLLSSYASHPFGAPHGPSAPGVAGPGGNLSSWEDLLLFTDLDQAATASKLLWSSRGAKLSPFAPEQPEEMYQTLAALSSQGPAAYDGAPGGFVHSAAAAAAAAAAASSPVYVPTTRVGSMLPGLPYHLQGSGSGPANHAGGAGAHPGWPQASADSPPYGSGGGAAGGGAAGPGGAGSAAAHVSARFPYSPSPPMANGAAREPGGYAAAGSGGAGGVSGGGSSLAAMGGREPQYSSLSAARPLNGTYHHHHHHHHHHPSPYSPYVGAPLTPAWPAGPFETPVLHSLQSRAGAPLPVPRGPSADLLEDLSESRECVNCGSIQTPLWRRDGTGHYLCNACGLYSKMNGLSRPLIKPQKRVPSSRRLGLSCANCHTTTTTLWRRNAEGEPVCNACGLYMKLHGVPRPLAMKKEGIQTRKRKPKNINKSKTCSGNSNNSIPMTPTSTSSNSDDCSKNTSPTTQPTASGAGAPVMTGAGESTNPENSELKYSGQDGLYIGVSLASPAEVTSSVRPDSWCALALA

SEQ ID NO: 51 - protein, 371 aa, Homo sapiens
  Nkx2-1-Em
MSMSPKHTTPFSVSDILSPLEESYKKVGMEGGGLGAPLAAYRQGQAAPPTAAMQQHAVGHHGAVTAAYHMTAAGVPQLSHSAVGGYCNGNLGNMSELPPYQDTMRNSASGPGWYGANPDPRFPAISRFMGPASGMNMSGMGGLGSLGDVSKNMAPLPSAPRRKRRVLFSQAQVYELERRFKQQKYLSAPEREHLASMIHLTPTQVKIWFQNHRYKMKRQAKDKAAQQQLQQDSGGGGGGGGTGCPQQQQAQQQSPRRVAVPVLVKDGKPCQAGAPAPGAASLQGHAQQQAQHQAQAAQAAAAAISVGSGGAGLGAHPGHQPGSAGQSPDLAHHAASPAALQGQVSSLSHLNSSGSDYGTMSCSTLLYGRTW

SEQ ID NO: 52 - protein, 463 aa, Homo sapiens
  Foxa2-Em
MHSASSMLGAVKMEGHEPSDWSSYYAEPEGYSSVSNMNAGLGMNGMNTYMSMSAAAMGSGSGNMSAGSMNMSSYVGAGMSPSLAGMSPGAGAMAGMGGSAGAAGVAGMGPHLSPSLSPLGGQAAGAMGGLAPYANMNSMSPMYGQAGLSRARDPKTYRRSYTHAKPPYSYISLITMAIQQSPNKMLTLSEIYQWIMDLFPFYRQNQQRWQNSIRHSLSFNDCFLKVPRSPDKPGKGSFWTLHPDSGNMFENGCYLRRQKRFKCEKQLALKEAAGAAGSGKKAAAGAQASQAQLGEAAGPASETPAGTESPHSSASPCQEHKRGGLGELKGTPAAALSPPEPAPSPGQQQQAAAHLLGPPHHPGLPPEAHLKPEHHYAFNHPFSINNLMSSEQQHHHSHHHHQPHKMDLKAYEQVMHYPGYGSPMPGSLAMGPVTNKTGLDASPLAADTSYYQGVYSRPIMNSS

SEQ ID NO: 53 - protein, 134 aa, Homo sapiens
  Id2-Em
MKAFSPVRSVRKNSLSDHSLGISRSKTPVDDPMSLLYNMNDCYSKLKELVPSIPQNKKVSKMEILQHVIDYILDLQIALDSHPTIVSLHHQRPGQNQASRTPLTTLNTDISILSLQASEFPSELMSNDSKALCG

SEQ ID NO: 54 - protein, 449 aa, Homo sapiens
  Gata6-Ad
MYQTLAALSSQGPAAYDGAPGGFVHSAAAAAAAAAAASSPVYVPTTRVGSMLPGLPYHLQGSGSGPANHAGGAGAHPGWPQASADSPPYGSGGGAAGGGAAGPGGAGSAAAHVSARFPYSPSPPMANGAAREPGGYAAAGSGGAGGVSGGGSSLAAMGGREPQYSSLSAARPLNGTYHHHHHHHHHHPSPYSPYVGAPLTPAWPAGPFETPVLHSLQSRAGAPLPVPRGPSADLLEDLSESRECVNCGSIQTPLWRRDGTGHYLCNACGLYSKMNGLSRPLIKPQKRVPSSRRLGLSCANCHTTTTTLWRRNAEGEPVCNACGLYMKLHGVPRPLAMKKEGIQTRKRKPKNINKSKTCSGNSNNSIPMTPTSTSSNSDDCSKNTSPTTQPTASGAGAPVMTGAGESTNPENSELKYSGQDGLYIGVSLASPAEVTSSVRPDSWCALALA

SEQ ID NO: 55 - protein, 401 aa, Homo sapiens
  Nkx2-1-Ad
MWSGGSGKARGWEAAAGGRSSPGRLSRRRIMSMSPKHTTPFSVSDILSPLEESYKKVGMEGGGLGAPLAAYRQGQAAPPTAAMQQHAVGHHGAVTAAYHMTAAGVPQLSHSAVGGYCNGNLGNMSELPPYQDTMRNSASGPGWYGANPDPRFPAISRFMGPASGMNMSGMGGLGSLGDVSKNMAPLPSAPRRKRRVLFSQAQVYELERRFKQQKYLSAPEREHLASMIHLTPTQVKIWFQNHRYKMKRQAKDKAAQQQLQQDSGGGGGGGGTGCPQQQQAQQQSPRRVAVPVLVKDGKPCQAGAPAPGAASLQGHAQQQAQHQAQAAQAAAAAISVGSGGAGLGAHPGHQPGSAGQSPDLAHHAASPAALQGQVSSLSHLNSSGSDYGTMSCSTLLYGRTW

SEQ ID NO: 56 - protein, 457 aa, Homo sapiens
  Foxa2-Ad
MLGAVKMEGHEPSDWSSYYAEPEGYSSVSNMNAGLGMNGMNTYMSMSAAAMGSGSGNMSAGSMNMSSYVGAGMSPSLAGMSPGAGAMAGMGGSAGAAGVAGMGPHLSPSLSPLGGQAAGAMGGLAPYANMNSMSPMYGQAGLSRARDPKTYRRSYTHAKPPYSYISLITMAIQQSPNKMLTLSEIYQWIMDLFPFYRQNQQRWQNSIRHSLSFNDCFLKVPRSPDKPGKGSFWTLHPDSGNMFENGCYLRRQKRFKCEKQLALKEAAGAAGSGKKAAAGAQASQAQLGEAAGPASETPAGTESPHSSASPCQEHKRGGLGELKGTPAAALSPPEPAPSPGQQQQAAAHLLGPPHHPGLPPEAHLKPEHHYAFNHPFSINNLMSSEQQHHHSHHHHQPHKMDLKAYEQVMHYPGYGSPMPGSLAMGPVTNKTGLDASPLAADTSYYQGVYSRPIMNSS

SEQ ID NO: 57 - protein, 141 aa, Homo sapiens [sequence omitted]

SEQ ID NO: 58 - DNA, 21 bp, Artificial Sequence
  Target sequence of Gata6-Em
aatcaggagcgcaggctgcag

SEQ ID NO: 59 - DNA, 21 bp, Artificial Sequence
  Target sequence of Gata6-Em
aagaggcgcctcctctctcct

SEQ ID NO: 60 - DNA, 21 bp, Artificial Sequence
  Target sequence of Foxa2-Em
aaaccgccatgcactcggctt

SEQ ID NO: 61 - DNA, 24 bp, Artificial Sequence
  Primers for HPRT Fwd
tgaccttgatttattttgcatacc

SEQ ID NO: 62 - DNA, 23 bp, Artificial Sequence
  Primers for HPRT Fwd
tttgctttccttggtcaggcagt

SEQ ID NO: 63 - DNA, 20 bp, Artificial Sequence
  Primers for HPRT Rev
cgagcaagacgttcagtcct

SEQ ID NO: 64 - DNA, 22 bp, Artificial Sequence
  Primers for HPRT Rev
cgtggggtccttttcaccagca

SEQ ID NO: 65 - RNA, 2219 nt, Homo sapiens
  Surfactant protein A
gacuuggaggcagagacccaagcagcuggaggcucuguguguggccuggagaccccacaaccuccagccggaggccugaagcaugaggccaugccaggugccaggagcagcgacuggacccagagccauguggcugugcccucuggcccucaaccucaucuugauggcagccucuggugcugugugcgaagugaaggacguuuguguuggaagcccugguauccccggcacuccuggaucccacggccugccaggcagggacgggagagauggucucaaaggagacccuggcccuccaggccccauggguccaccuggagaaaugccauguccuccuggaaaugaugggcugccuggagccccugguaucccuggagaguguggagagaagggggagccuggcgagaggggcccuccagggcuuccagcucaucuagaugaggagcuccaagccacacuccacgacuuuagacaucaaauccugcagacaaggggagcccucagucugcagggcuccauaaugacaguaggagagaaggucuucuccagcaaugggcaguccaucacuuuugaugccauucaggaggcaugugccagagcaggcggccgcauugcugucccaaggaauccagaggaaaaugaggccauugcaagcuucgugaagaaguacaacacauaugccuauguaggccugacugagggucccagcccuggagacuuccgcuacucagacgggaccccuguaaacuacaccaacugguaccgaggggagcccgcaggucggggaaaagagcaguguguggagauguacacagaugggcaguggaaugacaggaacugccuguacucccgacugaccaucugugaguucugagaggcauuuaggccaugggacagggaggacgcucucuggccuucggccuccauccugaggcuccacuuggucugugagaugcuagaacucccuuucaacagaauucacuuguggcuauugggacuggaggcacccuuagccacuucauuccucugaugggcccugacucuuccccauaaucacugaccagccuugacacuccccuugcaaacucucccagcacugcaccccaggcagccacucuuagccuuggccuucgacaugagauggagcccuccuuauuccccaucugguccaguuccuucacuuacagauggcagcagugaggucuugggguagaaggacccuccaaagucacacaaagugccugccuccugguccccucagcucucucucugcaacccagugccaucaggaugagcaauccuggccaagcauaaugacagagagaggcagacuucggggaagcccugacugugcagagcuaaggacacaguggagauucucuggcacucugaggucucuguggcaggccuggucaggcucuccaugagguuagaaggccagguaguguuccagcagggugguggccaagccaaccccaugauugauguguacgauucacuccuuugagucuuugaauggcaacucagcccccugaccugaagacagccagccuaggccucuagggugaccuagagccgccuucagaugugacccgaguaacuuucaacugaugaacaaaucugcacccuacuucagauuucagugggcauucacaccaccccccacaccacuggcucugcuuucuccuuucauuaauccauucacccagauauuucauuaaaauuaucacgugccaggucuuaggauaugucguggggugggcaagguaaucagugacaguugaagauuuuuuuuucccagagcuuaugucuucaucugugaaaugggaauaagauacuuguugcugucacaguuauuaccauccccccagcuaccaaaauuacuaccagaacuguuacuauacacagaggcuauugacugagcaccuaucauuugccaagaaccuugacaagcacuucuaauacagcauauuauguacuauucaaucuuuacacaaugucacgggaccaguauuguuuccucauuuuuuauaaggacacugaagcuuggaggaguuaaauguuuugaguauuauuccagagagcaaguggcagaggcuggauccaaacccaucuuccuggaccugaagcuuaugcuuccagccaccccacuccugagcugaauaaagaugauuuaagcuuaauaaaucgugaauguguucacaaaaaaaaaaaaaaaa

SEQ ID NO: 66 - protein, 263 aa, Homo sapiens
  Surfactant protein A
MRPCQVPGAATGPRAMWLCPLALNLILMAASGAVCEVKDVCVGSPGIPGTPGSHGLPGRDGRDGLKGDPGPPGPMGPPGEMPCPPGNDGLPGAPGIPGECGEKGEPGERGPPGLPAHLDEELQATLHDFRHQILQTRGALSLQGSIMTVGEKVFSSNGQSITFDAIQEACARAGGRIAVPRNPEENEAIASFVKKYNTYAYVGLTEGPSPGDFRYSDGTPVNYTNWYRGEPAGRGKEQCVEMYTDGQWNDRNCLYSRLTICEF

SEQ ID NO: 67 - RNA, 2189 nt, Homo sapiens
  surfactant protein A1 (SFTPA1), transcript variant 3
gacuuggaggcagagacccaagcagcuggaggcucugugugugggucgcugauuucuuggagccugaaaagaaagagcagcgacuggacccagagccauguggcugugcccucuggcccucaaccucaucuugauggcagccucuggugcugugugcgaagugaaggacguuuguguuggaagcccugguauccccggcacuccuggaucccacggccugccaggcagggacgggagagauggucucaaaggagacccuggcccuccaggccccauggguccaccuggagaaaugccauguccuccuggaaaugaugggcugccuggagccccugguaucccuggagaguguggagagaagggggagccuggcgagaggggcccuccagggcuuccagcucaucuagaugaggagcuccaagccacacuccacgacuuuagacaucaaauccugcagacaaggggagcccucagucugcagggcuccauaaugacaguaggagagaaggucuucuccagcaaugggcaguccaucacuuuugaugccauucaggaggcaugugccagagcaggcggccgcauugcugucccaaggaauccagaggaaaaugaggccauugcaagcuucgugaagaaguacaacacauaugccuauguaggccugacugagggucccagcccuggagacuuccgcuacucagacgggaccccuguaaacuacaccaacugguaccgaggggagcccgcaggucggggaaaagagcaguguguggagauguacacagaugggcaguggaaugacaggaacugccuguacucccgacugaccaucugugaguucugagaggcauuuaggccaugggacagggaggacgcucucuggccuucggccuccauccugaggcuccacuuggucugugagaugcuagaacucccuuucaacagaauucacuuguggcuauugggacuggaggcacccuuagccacuucauuccucugaugggcccugacucuuccccauaaucacugaccagccuugacacuccccuugcaaacucucccagcacugcaccccaggcagccacucuuagccuuggccuucgacaugagauggagcccuccuuauuccccaucugguccaguuccuucacuuacagauggcagcagugaggucuugggguagaaggacccuccaaagucacacaaagugccugccuccugguccccucagcucucucucugcaacccagugccaucaggaugagcaauccuggccaagcauaaugacagagagaggcagacuucggggaagcccugacugugcagagcuaaggacacaguggagauucucuggcacucugaggucucuguggcaggccuggucaggcucuccaugagguuagaaggccagguaguguuccagcagggugguggccaagccaaccccaugauugauguguacgauucacuccuuugagucuuugaauggcaacucagcccccugaccugaagacagccagccuaggccucuagggugaccuagagccgccuucagaugugacccgaguaacuuucaacugaugaacaaaucugcacccuacuucagauuucagugggcauucacaccaccccccacaccacuggcucugcuuucuccuuucauuaauccauucacccagauauuucauuaaaauuaucacgugccaggucuuaggauaugucguggggugggcaagguaaucagugacaguugaagauuuuuuuuucccagagcuuaugucuucaucugugaaaugggaauaagauacuuguugcugucacaguuauuaccauccccccagcuaccaaaauuacuaccagaacuguuacuauacacagaggcuauugacugagcaccuaucauuugccaagaaccuugacaagcacuucuaauacagcauauuauguacuauucaaucuuuacacaaugucacgggaccaguauuguuuccucauuuuuuauaaggacacugaagcuuggaggaguuaaauguuuugaguauuauuccagagagcaaguggcagaggcuggauccaaacccaucuuccuggaccugaagcuuaugcuuccagccaccccacuccugagcugaauaaagaugauuuaagcuuaauaaaucgugaauguguucacaaaaaaaaaaaaaaaa

SEQ ID NO: 68 - protein, 248 aa, Homo sapiens
  surfactant protein A1 (SFTPA1), transcript variant 3
MWLCPLALNLILMAASGAVCEVKDVCVGSPGIPGTPGSHGLPGRDGRDGLKGDPGPPGPMGPPGEMPCPPGNDGLPGAPGIPGECGEKGEPGERGPPGLPAHLDEELQATLHDFRHQILQTRGALSLQGSIMTVGEKVFSSNGQSITFDAIQEACARAGGRIAVPRNPEENEAIASFVKKYNTYAYVGLTEGPSPGDFRYSDGTPVNYTNWYRGEPAGRGKEQCVEMYTDGQWNDRNCLYSRLTICEF

SEQ ID NO: 69 - RNA, 2075 nt, Homo sapiens
  surfactant protein A1 (SFTPA1), transcript variant 5
gacuuggaggcagagacccaagcagcuggaggcucuguguguggcagccuggagaccccacaaccuccagccggaggccugaagcaugaggccaugccaggugccaggagcagcgacuggacccagagccauguggcugugcccucuggcccucaaccucaucuugauggcagccucuggugcugugugcgaagugaaggacguuuguguuggaaccccugguaucccuggagaguguggagagaagggggagccuggcgagaggggcccuccagggcuuccagcucaucuagaugaggagcuccaagccacacuccacgacuuuagacaucaaauccugcagacaaggggagcccucagucugcagggcuccauaaugacaguaggagagaaggucuucuccagcaaugggcaguccaucacuuuugaugccauucaggaggcaugugccagagcaggcggccgcauugcugucccaaggaauccagaggaaaaugaggccauugcaagcuucgugaagaaguacaacacauaugccuauguaggccugacugagggucccagcccuggagacuuccgcuacucagacgggaccccuguaaacuacaccaacugguaccgaggggagcccgcaggucggggaaaagagcaguguguggagauguacacagaugggcaguggaaugacaggaacugccuguacucccgacugaccaucugugaguucugagaggcauuuaggccaugggacagggaggacgcucucuggccuucggccuccauccugaggcuccacuuggucugugagaugcuagaacucccuuucaacagaauucacuuguggcuauugggacuggaggcacccuuagccacuucauuccucugaugggcccugacucuuccccauaaucacugaccagccuugacacuccccuugcaaacucucccagcacugcaccccaggcagccacucuuagccuuggccuucgacaugagauggagcccuccuuauuccccaucugguccaguuccuucacuuacagauggcagcagugaggucuugggguagaaggacccuccaaagucacacaaagugccugccuccugguccccucagcucucucucugcaacccagugccaucaggaugagcaauccuggccaagcauaaugacagagagaggcagacuucggggaagcccugacugugcagagcuaaggacacaguggagauucucuggcacucugaggucucuguggcaggccuggucaggcucuccaugagguuagaaggccagguaguguuccagcagggugguggccaagccaaccccaugauugauguguacgauucacuccuuugagucuuugaauggcaacucagcccccugaccugaagacagccagccuaggccucuagggugaccuagagccgccuucagaugugacccgaguaacuuucaacugaugaacaaaucugcacccuacuucagauuucagugggcauucacaccaccccccacaccacuggcucugcuuucuccuuucauuaauccauucacccagauauuucauuaaaauuaucacgugccaggucuuaggauaugucguggggugggcaagguaaucagugacaguugaagauuuuuuuuucccagagcuuaugucuucaucugugaaaugggaauaagauacuuguugcugucacaguuauuaccauccccccagcuaccaaaauuacuaccagaacuguuacuauacacagaggcuauugacugagcaccuaucauuugccaagaaccuugacaagcacuucuaauacagcauauuauguacuauucaaucuuuacacaaugucacgggaccaguauuguuuccucauuuuuuauaaggacacugaagcuuggaggaguuaaauguuuugaguauuauuccagagagcaaguggcagaggcuggauccaaacccaucuuccuggaccugaagcuuaugcuuccagccaccccacuccugagcugaauaaagaugauuuaagcuuaauaaaucgugaauguguucacaaaaaaaaaaaaaaaa

SEQ ID NO: 70 - protein, 214 aa, Homo sapiens
  surfactant protein A1 (SFTPA1), transcript variant 5
MRPCQVPGAATGPRAMWLCPLALNLILMAASGAVCEVKDVCVGTPGIPGECGEKGEPGERGPPGLPAHLDEELQATLHDFRHQILQTRGALSLQGSIMTVGEKVFSSNGQSITFDAIQEACARAGGRIAVPRNPEENEAIASFVKKYNTYAYVGLTEGPSPGDFRYSDGTPVNYTNWYRGEPAGRGKEQCVEMYTDGQWNDRNCLYSRLTICEF

SEQ ID NO: 71 - RNA, 2083 nt, Homo sapiens
  surfactant protein A1 (SFTPA1), transcript variant 6
gacuuggaggcagagacccaagcagcuggaggcucugugugugggucgcugauuucuuggagccugaaaagaaaguaacacagcagggaugaggacagauggugugagucagugagagcagcgacuggacccagagccauguggcugugcccucuggcccucaaccucaucuugauggcagccucuggugcugugugcgaagugaaggacguuuguguuggaaccccugguaucccuggagaguguggagagaagggggagccuggcgagaggggcccuccagggcuuccagcucaucuagaugaggagcuccaagccacacuccacgacuuuagacaucaaauccugcagacaaggggagcccucagucugcagggcuccauaaugacaguaggagagaaggucuucuccagcaaugggcaguccaucacuuuugaugccauucaggaggcaugugccagagcaggcggccgcauugcugucccaaggaauccagaggaaaaugaggccauugcaagcuucgugaagaaguacaacacauaugccuauguaggccugacugagggucccagcccuggagacuuccgcuacucagacgggaccccuguaaacuacaccaacugguaccgaggggagcccgcaggucggggaaaagagcaguguguggagauguacacagaugggcaguggaaugacaggaacugccuguacucccgacugaccaucugugaguucugagaggcauuuaggccaugggacagggaggacgcucucuggccuucggccuccauccugaggcuccacuuggucugugagaugcuagaacucccuuucaacagaauucacuuguggcuauugggacuggaggcacccuuagccacuucauuccucugaugggcccugacucuuccccauaaucacugaccagccuugacacuccccuugcaaacucucccagcacugcaccccaggcagccacucuuagccuuggccuucgacaugagauggagcccuccuuauuccccaucugguccaguuccuucacuuacagauggcagcagugaggucuugggguagaaggacccuccaaagucacacaaagugccugccuccugguccccucagcucucucucugcaacccagugccaucaggaugagcaauccuggccaagcauaaugacagagagaggcagacuucggggaagcccugacugugcagagcuaaggacacaguggagauucucuggcacucugaggucucuguggcaggccuggucaggcucuccaugagguuagaaggccagguaguguuccagcagggugguggccaagccaaccccaugauugauguguacgauucacuccuuugagucuuugaauggcaacucagcccccugaccugaagacagccagccuaggccucuagggugaccuagagccgccuucagaugugacccgaguaacuuucaacugaugaacaaaucugcacccuacuucagauuucagugggcauucacaccaccccccacaccacuggcucugcuuucuccuuucauuaauccauucacccagauauuucauuaaaauuaucacgugccaggucuuaggauaugucguggggugggcaagguaaucagugacaguugaagauuuuuuuuucccagagcuuaugucuucaucugugaaaugggaauaagauacuuguugcugucacaguuauuaccauccccccagcuaccaaaauuacuaccagaacuguuacuauacacagaggcuauugacugagcaccuaucauuugccaagaaccuugacaagcacuucuaauacagcauauuauguacuauucaaucuuuacacaaugucacgggaccaguauuguuuccucauuuuuuauaaggacacugaagcuuggaggaguuaaauguuuugaguauuauuccagagagcaaguggcagaggcuggauccaaacccaucuuccuggaccugaagcuuaugcuuccagccaccccacuccugagcugaauaaagaugauuuaagcuuaauaaaucgugaauguguucacaaaaaaaaaaaaaaaa

SEQ ID NO: 72 - protein, 199 aa, Homo sapiens
  surfactant protein A1 (SFTPA1), transcript variant 6
MWLCPLALNLILMAASGAVCEVKDVCVGTPGIPGECGEKGEPGERGPPGLPAHLDEELQATLHDFRHQILQTRGALSLQGSIMTVGEKVFSSNGQSITFDAIQEACARAGGRIAVPRNPEENEAIASFVKKYNTYAYVGLTEGPSPGDFRYSDGTPVNYTNWYRGEPAGRGKEQCVEMYTDGQWNDRNCLYSRLTICEF

SEQ ID NO: 73 - RNA, 2159 nt, Homo sapiens
  surfactant protein A1 (SFTPA1), transcript variant 4
gacuuggaggcagagacccaagcagcuggaggcucugugugugggagcagcgacuggacccagagccauguggcugugcccucuggcccucaaccucaucuugauggcagccucuggugcugugugcgaagugaaggacguuuguguuggaagcccugguauccccggcacuccuggaucccacggccugccaggcagggacgggagagauggucucaaaggagacccuggcccuccaggccccauggguccaccuggagaaaugccauguccuccuggaaaugaugggcugccuggagccccugguaucccuggagaguguggagagaagggggagccuggcgagaggggcccuccagggcuuccagcucaucuagaugaggagcuccaagccacacuccacgacuuuagacaucaaauccugcagacaaggggagcccucagucugcagggcuccauaaugacaguaggagagaaggucuucuccagcaaugggcaguccaucacuuuugaugccauucaggaggcaugugccagagcaggcggccgcauugcugucccaaggaauccagaggaaaaugaggccauugcaagcuucgugaagaaguacaacacauaugccuauguaggccugacugagggucccagcccuggagacuuccgcuacucagacgggaccccuguaaacuacaccaacugguaccgaggggagcccgcaggucggggaaaagagcaguguguggagauguacacagaugggcaguggaaugacaggaacugccuguacucccgacugaccaucugugaguucugagaggcauuuaggccaugggacagggaggacgcucucuggccuucggccuccauccugaggcuccacuuggucugugagaugcuagaacucccuuucaacagaauucacuuguggcuauugggacuggaggcacccuuagccacuucauuccucugaugggcccugacucuuccccauaaucacugaccagccuugacacuccccuugcaaacucucccagcacugcaccccaggcagccacucuuagccuuggccuucgacaugagauggagcccuccuuauuccccaucugguccaguuccuucacuuacagauggcagcagugaggucuugggguagaaggacccuccaaagucacacaaagugccugccuccugguccccucagcucucucucugcaacccagugccaucaggaugagcaauccuggccaagcauaaugacagagagaggcagacuucggggaagcccugacugugcagagcuaaggacacaguggagauucucuggcacucugaggucucuguggcaggccuggucaggcucuccaugagguuagaaggccagguaguguuccagcagggugguggccaagccaaccccaugauugauguguacgauucacuccuuugagucuuugaauggcaacucagcccccugaccugaagacagccagccuaggccucuagggugaccuagagccgccuucagaugugacccgaguaacuuucaacugaugaacaaaucugcacccuacuucagauuucagugggcauucacaccaccccccacaccacuggcucugcuuucuccuuucauuaauccauucacccagauauuucauuaaaauuaucacgugccaggucuuaggauaugucguggggugggcaagguaaucagugacaguugaagauuuuuuuuucccagagcuuaugucuucaucugugaaaugggaauaagauacuuguugcugucacaguuauuaccauccccccagcuaccaaaauuacuaccagaacuguuacuauacacagaggcuauugacugagcaccuaucauuugccaagaaccuugacaagcacuucuaauacagcauauuauguacuauucaaucuuuacacaaugucacgggaccaguauuguuuccucauuuuuuauaaggacacugaagcuuggaggaguuaaauguuuugaguauuauuccagagagcaaguggcagaggcuggauccaaacccaucuuccuggaccugaagcuuaugcuuccagccaccccacuccugagcugaauaaagaugauuuaagcuuaauaaaucgugaauguguucacaaaaaaaaaaaaaaaa

SEQ ID NO: 74 - protein, 248 aa, Homo sapiens; identical to SEQ ID NO: 68
  surfactant protein A1 (SFTPA1), transcript variant 4

SEQ ID NO: 75 - RNA, 2228 nt, Homo sapiens
  surfactant protein A1 (SFTPA1), transcript variant 1
gacuuggaggcagagacccaagcagcuggaggcucugugugugggucgcugauuucuuggagccugaaaagaaaguaacacagcagggaugaggacagauggugugagucagugagagcagcgacuggacccagagccauguggcugugcccucuggcccucaaccucaucuugauggcagccucuggugcugugugcgaagugaaggacguuuguguuggaagcccugguauccccggcacuccuggaucccacggccugccaggcagggacgggagagauggucucaaaggagacccuggcccuccaggccccauggguccaccuggagaaaugccauguccuccuggaaaugaugggcugccuggagccccugguaucccuggagaguguggagagaagggggagccuggcgagagggcccuccagggcuuccagcucaucuagaugaggagcuccaagccacacuccacgacuuuagacaucaaauccugcagacaaggggagcccucagucugcagggcuccauaaugacaguaggagagaaggucuucuccagcaaugggcaguccaucacuuuugaugccauucaggaggcaugugccagagcaggcggccgcauugcuguccaaggaauccagaggaaaaugaggccauugcaagcuucgugaagaaguacaacacauaugccuauguaggccugacugagggucccagcccuggagacuuccgcuacucagacgggaccccuguaaacuacaccaacugguaccgaggggagcccgcaggucggggaaaagagcaguguguggagauguacacagaugggcaguggaaugacaggaacugccuguacucccgacugaccaucugugaguucugagaggcauuuaggccaugggacagggaggacgcucucuggccuucggccuccauccugaggcuccacuuggucugugagaugcuagaacucccuuucaacagaauucacuuguggcuauugggacuggaggcacccuuagccacuucauuccucugaugggcccugacucuuccccauaaucacugaccagccuugacacuccccuugcaaacucucccagcacugcaccccaggcagccacucuuagccuuggccuucgacaugagauggagcccuccuuauuccccaucugguccaguuccuucacuuacagauggcagcagugaggucuugggguagaaggacccuccaaagucacacaaagugccugccuccugguccccucagcucucucucugcaacccagugccaucaggaugagcaauccuggccaagcauaaugacagagagaggcagacuucggggaagcccugacugugcagagcuaaggacacaguggagauucucuggcacucugaggucucuguggcaggccuggucaggcucuccaugagguuagaaggccagguaguguuccagcagggugguggccaagccaaccccaugauugauguguacgauucacuccuuugagucuuugaauggcaacucagcccccugaccugaagacagccagccuaggccucuagggugaccuagagccgccuucagaugugacccgaguaacuuucaacugaugaacaaaucugcacccuacuucagauuucagugggcauucacaccaccccccacaccacuggcucugcuuucuccuuucauuaauccauucacccagauauuucauuaaaauuaucacgugccaggucuuaggauaugucguggggugggcaagguaaucagugacaguugaagauuuuuuuuucccagagcuuaugucuucaucugugaaaugggaauaagauacuuguugcugucacaguuauuaccauccccccagcuaccaaaauuacuaccagaacuguuacuauacacagaggcuauugacugagcaccuaucauuugccaagaaccuugacaagcacuucuaauacagcauauuauguacuauucaaucuuuacacaaugucacgggaccaguauuguuuccucauuuuuuauaaggacacugaagcuuggaggaguuaaauguuuugaguauuauuccagagagcaaguggcagaggcuggauccaaacccaucuuccuggaccugaagcuuaugcuuccagccaccccacuccugagcugaauaaagaugauuuaagcuuaauaaaucgugaauguguucacaaaaaaaaaaaaaaaa

SEQ ID NO: 76 - protein, 248 aa, Homo sapiens; identical to SEQ ID NO: 68
  surfactant protein A1 (SFTPA1), transcript variant 1

SEQ ID NO: 77 - RNA, 3681 nt, Homo sapiens
  Transcript variant pulmonary surfactant-associated protein B precursor
uguaaaugcucuucugacuaaugcaaaccauguguccauagaaccagaagauuuuuccaggggaaaagagcccccacgccccgcccagcuauaaggggccaugcaccaagcaggguacccaggcugcagaggugccauggcugagucacaccugcugcaguggcugcugcugcugcugcccacgcucuguggcccaggcacugcugccuggaccaccucauccuuggccugugcccagggcccugaguucuggugccaaagccuggagcaagcauugcagugcagagcccuagggcauugccuacaggaagucuggggacaugugggagccgaugaccuaugccaagagugugaggacaucguccacauccuuaacaagauggccaaggaggccauuuuccaggacacgaugaggaaguuccuggagcaggagugcaacguccuccccuugaagcugcucaugccccagugcaaccaagugcuugacgacuacuucccccuggucaucgacuacuuccagaaccagacugacucaaacggcaucuguaugcaccugggccugugcaaaucccggcagccagagccagagcaggagccagggaugucagacccccugcccaaaccucugcgggacccucugccagacccucugcuggacaagcucguccucccugugcugcccggggcccuccaggcgaggccugggccucacacacaggaucucuccgagcagcaauuccccauuccucuccccuauugcuggcucugcagggcucugaucaagcggauccaagccaugauucccaagggugcgcuagcuguggcaguggcccaggugugccgcgugguaccucugguggcgggcggcaucugccagugccuggcugagcgcuacuccgucauccugcucgacacgcugcugggccgcaugcugccccagcuggucugccgccucguccuccggugcuccauggaugacagcgcuggcccaaggucgccgacaggagaauggcugccgcgagacucugagugccaccucugcauguccgugaccacccaggccgggaacagcagcgagcaggccauaccacaggcaaugcuccaggccuguguuggcuccuggcuggacagggaaaagugcaagcaauuuguggagcagcacacgccccagcugcugacccuggugcccaggggcugggaugcccacaccaccugccaggcccucggggugugugggaccauguccagcccucuccaguguauccacagccccgaccuuugaugagaacucagcuguccagcugcaaaggaaaagccaagugagacgggcucugggaccauggugaccaggcucuuccccugcucccuggcccucgccagcugccaggcugaaaagaagccucagcucccacaccgcccuccucaccgcccuuccucggcagucacuuccacugguggaccacgggcccccagcccugugucggccuugucugucucagcucaaccacagucugacaccagagcccacuuccauccucucuggugugaggcacagcgagggcagcaucuggaggagcucugcagccuccacaccuaccacgaccucccagggcugggcucaggaaaaaccagccacugcuuuacaggacaggggguugaagcugagccccgccucacacccacccccaugcacucaaagauuggauuuuacagcuacuugcaauucaaaauucagaagaauaaaaaaugggaacauacagaacucuaaaagauagacaucagaaauuguuaaguuaagcuuuuucaaaaaaucagcaauuccccagcguagucaaggguggacacugcacgcucuggcaugaugggauggcgaccgggcaagcuuucuuccucgagaugcucugcugcuugagagcuauugcuuuguuaagauauaaaaagggguuucuuuuugucuuucuguaagguggacuuccagcuuuugauugaaaguccuagggugauucuauuucugcugugauuuaucugcugaaagcucagcugggguugugcaagcuagggacccauuccuguguaauacaaugucugcaccaaugcuaauaaaguccuauucucuuuuaugagaaagaaaaagacaccguccuuuaaagugcugcaguauggccagacgugguggcucacaccugcaaucccagcaccuuaggaggccgaggcaggaggauccuugaggucaggaguucgagaccagccucgccaacauggugaaaccccauuucuacuaaaaauacaaaaaauuagccaagugugguggcauaugccuguaaucccaacuacucagaaggccgaggcaggagaauuacuugaacgcaggagaaucacugcagcccaggaggcagagguugcagugagccgagauugcaccacugcacuccagccugggugacagagcaagacuccaucucaguaaauaaauaaauaaauaaaaagcgcugcaguagcuguggccucacccugaagucagcgggcccaggccuaccucacucucucccuuggcagagaagcagacguccauagcuccucucccucacaagcgcucccagccugcccuccagcugcugcucuccccucccagucucuacucacugggaugagguuaggucaugaggacaccaaaaaccuaaaaauaaacaaaaagccaaacaagccuuagcuuuucuuaaagacugaaaugccuggaagugucccuuuauuuauaaaauaacuuuugucauauuucuuauacauguuucuuguaagaaauucagaaacuacagacaaagagaguggaaauuacccacugucaggccucugagcccaagcuaagccaucauauccccugugcccugcacguauacacccagauggccugaagcaacugaagauccacaaaagaagugaaaauagccaguuccugccuuaacugaugacauuccaccauugugauuuguuccugccccacccuaacugaucaauugaccuugugacaauacaccuuccccacccuugagaaggugcuuuguaauauucuccccacccaccccacgcccgcacccccgcacccuuaagaagguauuuuguaauauucucuccgccauugagaaugugcuuuguaagauccacccccugcccacaaaaaauugcuccuaacuccaccgccuaucccaaaccuacaagaacuaaugauaaucccaccacccuuugcugacucuuuuuggacucagcccaccugcacccaggugauuaaaaagcuuuauuguucacacaaagccuguuugguagucucuucacagggaagcaugugacacccacaaucccaccuagcccaggagagagcuacggcaggguguguguuuugacacugagcuuggggcuuuuuccaucuucuccccacagccucuggcuccacaccuccaccguucaagcgccagaaagagcugucuaugcagccugcucuugggccuggggaugagacacacaauucauuggcuccuggauuuuaaguagacauuuguaaaucuauagcuaacuacuguccuuaaagccauuguuuccauuacaaaauccaacucucugagagaaaaggguguuuuaaauuuaaaaaaauaaaaacaaaaaaguuugauugagaaaaaaaaaaaaaaa

SEQ ID NO: 78 - protein, 393 aa, Homo sapiens
  pulmonary surfactant-associated protien B precursor
MHQAGYPGCRGAMAESHLLQWLLLLLPTLCGPGTAAWTTSSLACAQGPEFWCQSLEQALQCRALGHCLQEVWGHVGADDLCQECEDIVHILNKMAKEAIFQDTMRKFLEQECNVLPLKLLMPQCNQVLDDYFPLVIDYFQNQTDSNGICMHLGLCKSRQPEPEQEPGMSDPLPKPLRDPLPDPLLDKLVLPVLPGALQARPGPHTQDLSEQQFPIPLPYCWLCRALIKRIQAMIPKGALAVAVAQVCRVVPLVAGGICQCLAERYSVILLDTLLGRMLPQLVCRLVLRCSMDDSAGPRSPTGEWLPRDSECHLCMSVTTQAGNSSEQAIPQAMLQACVGSWLDREKCKQFVEQHTPQLLTLVPRGWDAHTTCQALGVCGTMSSPLQCIHSPDL

SEQ ID NO: 79 - RNA, 2854 nt, Homo sapiens
  pulmonary surfactant-associated protein B precursor, transcript variant 2
uguaaaugcucuucugacuaaugcaaaccauguguccauagaaccagaagauuuuuccaggggaaaagagcccccacgccccgcccagcuauaaggggccaugcaccaagcaggguacccaggcugcagaggugccauggcugagucacaccugcugcaguggcugcugcugcugcugcccacgcucuguggcccaggcacugcugccuggaccaccucauccuuggccugugcccagggcccugaguucuggugccaaagccuggagcaagcauugcagugcagagcccuagggcauugccuacaggaagucuggggacaugugggagccgaugaccuaugccaagagugugaggacaucguccacauccuuaacaagauggccaaggaggccauuuuccaggacacgaugaggaaguuccuggagcaggagugcaacguccuccccuugaagcugcucaugccccagugcaaccaagugcuugacgacuacuucccccuggucaucgacuacuuccagaaccagacugacucaaacggcaucuguaugcaccugggccugugcaaaucccggcagccagagccagagcaggagccagggaugucagacccccugcccaaaccucugcgggacccucugccagacccucugcuggacaagcucguccucccugugcugcccggggcccuccaggcgaggccugggccucacacacaggaucucuccgagcagcaauuccccauuccucuccccuauugcuggcucugcagggcucugaucaagcggauccaagccaugauucccaagggugcgcuagcuguggcaguggcccaggugugccgcgugguaccucugguggcgggcggcaucugccagugccuggcugagcgcuacuccgucauccugcucgacacgcugcugggccgcaugcugccccagcuggucugccgccucguccuccggugcuccauggaugacagcgcuggcccaaggucgccgacaggagaauggcugccgcgagacucugagugccaccucugcauguccgugaccacccaggccgggaacagcagcgagcaggccauaccacaggcaaugcuccaggccuguguuggcuccuggcuggacagggaaaagugcaagcaauuuguggagcagcacacgccccagcugcugacccuggugcccaggggcugggaugcccacaccaccugccaggcccucggggugugugggaccauguccagcccucuccaguguauccacagccccgaccuuugaugagaacucagcuguccagaaaaagacaccguccuuuaaagugcugcaguauggccagacgugguggcucacaccugcaaucccagcaccuuaggaggccgaggcaggaggauccuugaggucaggaguucgagaccagccucgccaacauggugaaaccccauuucuacuaaaaauacaaaaaauuagccaagugugguggcauaugccuguaaucccaacuacucagaaggccgaggcaggagaauuacuugaacgcaggagaaucacugcagcccaggaggcagagguugcagugagccgagauugcaccacugcacuccagccugggugacagagcaagacuccaucucaguaaauaaauaaauaaauaaaaagcgcugcaguagcuguggccucacccugaagucagcgggcccaggccuaccucacucucucccuuggcagagaagcagacguccauagcuccucucccucacaagcgcucccagccugcccuccagcugcugcucuccccucccagucucuacucacugggaugagguuaggucaugaggacaccaaaaaccuaaaaauaaacaaaaagccaaacaagccuuagcuuuucuuaaagacugaaaugccuggaagugucccuuuauuuauaaaauaacuuuugucauauuucuuauacauguuucuuguaagaaauucagaaacuacagacaaagagaguggaaauuacccacugucaggccucugagcccaagcuaagccaucauauccccugugcccugcacguauacacccagauggccugaagcaacugaagauccacaaaagaagugaaaauagccaguuccugccuuaacugaugacauuccaccauugugauuuguuccugccccacccuaacugaucaauugaccuugugacaauacaccuuccccacccuugagaaggugcuuuguaauauucuccccacccaccccacgcccgcacccccgcacccuuaagaagguauuuuguaauauucucuccgccauugagaaugugcuuuguaagauccacccccugcccacaaaaaauugcuccuaacuccaccgccuaucccaaaccuacaagaacuaaugauaaucccaccacccuuugcugacucuuuuuggacucagcccaccugcacccaggugauuaaaaagcuuuauuguucacacaaagccuguuugguagucucuucacagggaagcaugugacacccacaaucccaccuagcccaggagagagcuacggcaggguguguguuuugacacugagcuuggggcuuuuuccaucuucuccccacagccucuggcuccacaccuccaccguucaagcgccagaaagagcugucuaugcagccugcucuugggccuggggaugagacacacaauucauuggcuccuggauuuuaaguagacauuuguaaaucuauagcuaacuacuguccuuaaagccauuguuuccauuacaaaauccaacucucugagagaaaaggguguuuuaaauuuaaaaaaauaaaaacaaaaaaguuugauugagaaaaaaaaaaaaaaa

SEQ ID NO: 80 - RNA, 1437 nt, Homo sapiens
  napsin A aspartic peptidase
ggaaagaaaaugaggccccaggacaccuggguucacacccagguccccagcgaugucuccaccaccgcugcugcaaccccugcugcugcugcugccucugcugaauguggagccuuccggggccacacugauccgcaucccucuucaucgaguccaaccuggacgcaggauccugaaccuacugaggggauggagagaaccagcagagcuccccaaguugggggccccauccccuggggacaagcccaucuucguaccucucucgaacuacagggaugugcaguauuuuggggaaauugggcugggaacgccuccacaaaacuucacuguugccuuugacacuggcuccuccaaucucugggucccguccaggagaugccacuucuucagugugcccugcugguuacaccaccgauuugaucccaaagccucuagcuccuuccaggccaaugggaccaaguuugccauucaauauggaacugggcggguagauggaauccugagcgaggacaagcugacuauugguggaaucaagggugcaucagugauuuucggggaggcucucugggagcccagccuggucuucgcuuuugcccauuuugaugggauauugggccucgguuuucccauucugucuguggaaggaguucggcccccgauggauguacugguggagcaggggcuauuggauaagccugucuucuccuuuuaccucaacagggacccugaagagccugauggaggagagcugguccuggggggcucggacccggcacacuacaucccaccccucaccuucgugccagucacggucccugccuacuggcagauccacauggagcgugugaaggugggcccagggcugacucucugugccaagggcugugcugccauccuggauacgggcacgucccucaucacaggacccacugaggagauccgggcccugcaugcagccauugggggaauccccuugcuggcuggggaguacaucauccugugcucggaaaucccaaagcuccccgcagucuccuuccuucuugggggggucugguuuaaccucacggcccaugauuacgucauccagacuacucgaaauggcguccgccucugcuuguccgguuuccaggcccuggaugucccuccgccugcagggcccuucuggauccucggugacgucuucuuggggacguauguggccgucuucgaccgcggggacaugaagagcagcgcccgggugggccuggcgcgcgcucgcacucgcggagcggaccucggauggggagagacugcgcaggcgcaguuccccgggugacgcccaagugaagcgcaugcgcagcggguggucgcggagguccugcuacccaguaaaaauccacuauuuccauugaaaaaaaaaaaaaaaaaaaaaaaaaaaaaaaaaaaaaaaaaaaaaaa

SEQ ID NO: 81 - protein, 420 aa, Homo sapiens
  napsin A aspartic peptidase
MSPPPLLQPLLLLLPLLNVEPSGATLIRIPLHRVQPGRRILNLLRGWREPAELPKLGAPSPGDKPIFVPLSNYRDVQYFGEIGLGTPPQNFTVAFDTGSSNLWVPSRRCHFFSVPCWLHHRFDPKASSSFQANGTKFAIQYGTGRVDGILSEDKLTIGGIKGASVIFGEALWEPSLVFAFAHFDGILGLGFPILSVEGVRPPMDVLVEQGLLDKPVFSFYLNRDPEEPDGGELVLGGSDPAHYIPPLTFVPVTVPAYWQIHMERVKVGPGLTLCAKGCAAILDTGTSLITGPTEEIRALHAAIGGIPLLAGEYIILCSEIPKLPAVSFLLGGVWFNLTAHDYVIQTTRNGVRLCLSGFQALDVPPPAGPFWILGDVFLGTYVAVFDRGDMKSSARVGLARARTRGADLGWGETAQAQFPG

SEQ ID NO: 82 - RNA, 4832 nt, Homo sapiens
  tumor protein p63 (TP63), transcript variant 2
cccggcuuuauaucuauauauacacagguauauguguauauuuuauauaauuguucuccguucguugauaucaaagacaguugaaggaaaugaauuuugaaacuucacggugugccacccuacaguacugcccugacccuuacauccagcguuucguagaaaccccagcucauuucucuuggaaagaaaguuauuaccgauccaccaugucccagagcacacagacaaaugaauuccucaguccagagguuuuccagcauaucugggauuuucuggaacagccuauauguucaguucagcccauugacuugaacuuuguggaugaaccaucagaagauggugcgacaaacaagauugagauuagcauggacuguauccgcaugcaggacucggaccugagugaccccauguggccacaguacacgaaccuggggcuccugaacagcauggaccagcagauucagaacggcuccucguccaccagucccuauaacacagaccacgcgcagaacagcgucacggcgcccucgcccuacgcacagcccagcuccaccuucgaugcucucucuccaucacccgccauccccuccaacaccgacuacccaggcccgcacaguuucgacguguccuuccagcagucgagcaccgccaagucggccaccuggacguauuccacugaacugaagaaacucuacugccaaauugcaaagacaugccccauccagaucaaggugaugaccccaccuccucagggagcuguuauccgcgccaugccugucuacaaaaaagcugagcacgucacggagguggugaagcggugccccaaccaugagcugagccgugaauucaacgagggacagauugccccuccuagucauuugauucgaguagaggggaacagccaugcccaguauguagaagaucccaucacaggaagacagagugugcugguaccuuaugagccaccccagguuggcacugaauucacgacagucuuguacaauuucauguguaacagcaguuguguuggagggaugaaccgccguccaauuuuaaucauuguuacucuggaaaccagagaugggcaaguccugggccgacgcugcuuugaggcccggaucugugcuugcccaggaagagacaggaaggcggaugaagauagcaucagaaagcagcaaguuucggacauacaaagaacggugaugguacgaagcgcccguuucgucagaacacacaugguauccagaugacauccaucaagaaacgaagauccccagaugaugaacuguuauacuuaccagugaggggccgugagacuuaugaaaugcuguugaagaucaaagagucccuggaacucaugcaguaccuuccucagcacacaauugaaacguacaggcaacagcaacagcagcagcaccagcacuuacuucagaaacagaccucaauacagucuccaucuucauaugguaacagcuccccaccucugaacaaaaugaacagcaugaacaagcugccuucugugagccagcuuaucaacccucagcagcgcaacgcccucacuccuacaaccauuccugauggcaugggagccaacauucccaugaugggcacccacaugccaauggcuggagacaugaauggacucagccccacccaggcacucccucccccacucuccaugccauccaccucccacugcacacccccaccuccguaucccacagauugcagcauugucaggaucuggcaagucugaaaaucccugagcaauuucgacaugcgaucuggaagggcauccuggaccaccggcagcuccacgaauucuccuccccuucucaucuccugcggaccccaagcagugccucuacagucagugugggcuccagugagacccggggugagcguguuauugaugcugugcgauucacccuccgccagaccaucucuuucccaccccgagaugaguggaaugacuucaacuuugacauggaugcucgccgcaauaagcaacagcgcaucaaagaggagggggagugagccucaccaugugagcucuuccuaucccucuccuaacugccagcccccuaaaagcacuccugcuuaaucuucaaagccuucucccuagcuccuccccuuccucuugucugauuucuuaggggaaggagaaguaagaggcuaccucuuaccuaacaucugaccuggcaucuaauucugauucuggcuuuaagccuucaaaacuauagcuugcagaacuguagcugccauggcuagguagaagugagcaaaaaagaguugggugucuccuuaagcugcagagauuucucauugacuuuuauaaagcauguucacccuuauagucuaagacuauauauauaaauguauaaauauacaguauagauuuuuggguggggggcauugaguauuguuuaaaauguaauuuaaaugaaagaaaauugaguugcacuuauugaccauuuuuuaauuuacuuguuuuggauggcuugucuauacuccuucccuuaagggguaucauguauggugauagguaucuagagcuuaaugcuacaugugagugacgaugauguacagauucuuucaguucuuuggauucuaaauacaugccacaucaaaccuuugaguagauccauuuccauugcuuauuauguagguaagacuguagauauguauucuuuucucaguguugguauauuuuauauuacugacauuucuucuagugaugaugguucacguuggggugauuuaauccaguuauaagaagaaguucauguccaaacguccucuuuaguuuuugguugggaaugaggaaaauucuuaaaaggcccauagcagccaguucaaaaacacccgacgucauguauuugagcauaucaguaacccccuuaaauuuaauaccagauaccuuaucuuacaauauugauugggaaaacauuugcugccauuacagagguauuaaaacuaaauuucacuacuagauugacuaacucaaauacacauuugcuacuguuguaagaauucugauugauuugauugggaugaaugccaucuaucuaguucuaacagugaaguuuuacugucuauuaauauucaggguaaauaggaaucauucagaaauguugagucuguacuaaacaguaagauaucucaaugaaccauaaauucaacuuuguaaaaaucuuuugaagcauagauaauauuguuugguaaauguuucuuuuguuugguaaauguuucuuuuaaagacccuccuauucuauaaaacucugcauguagaggcuuguuuaccuuucucucucuaagguuuacaauaggaguggugauuugaaaaauauaaaauuaugagauugguuuuccuguggcauaaauugcaucacuguaucauuuucuuuuuuaaccgguaagaguuucaguuuguuggaaaguaacugugagaacccaguuucccguccaucucccuuagggacuacccauagacaugaaagguccccacagagcaagagauaagucuuucauggcugcuguugcuuaaaccacuuaaacgaagaguucccuugaaacuuugggaaaacauguuaaugacaauauuccagaucuuucagaaauauaacacauuuuuuugcaugcaugcaaaugagcucugaaaucuucccaugcauucuggucaagggcugucauugcacauaagcuuccauuuuaauuuuaaagugcaaaagggccagcguggcucuaaaagguaauguguggauugccucugaaaaguguguauauauuuugugugaaauugcauacuuuguauuuugauuauuuuuuuuuucuucuugggauagugggauuuccagaaccacacuugaaaccuuuuuuuaucguuuuuguauuuucaugaaaauaccauuuaguaagaauaccacaucaaauaagaaauaaugcuacaauuuuaagaggggagggaagggaaaguuuuuuuuuauuauuuuuuuaaaauuuuguauguuaaagagaaugaguccuugauuucaaaguuuuguuguacuuaaaugguaauaagcacuguaaacuucugcaacaagcaugcagcuuugcaaacccauuaaggggaagaaugaaagcuguuccuugguccuaguaagaagacaaacugcuucccuuacuuugcugaggguuugaauaaaccuaggacuuccgagcuaugucaguacuauucagguaacacuagggccuuggaaauuccuguacugugucucauggauuuggcacuagccaaagcgaggcacccuuacuggcuuaccuccucauggcagccuacucuccuugaguguaugaguagccaggguaagggguaaaaggauaguaagcauagaaaccacuagaaagugggcuuaauggaguucuuguggccucagcucaaugcaguuagcugaagaauugaaaaguuuuuguuuggagacguuuauaaacagaaauggaaagcagaguuuucauuaaauccuuuuaccuuuuuuuuuucuugguaauccccuaaaauaacaguaugugggauauugaauguuaaagggauauuuuuuucuauuauuuuuauaauuguacaaaauuaagcaaauguuaaaaguuuuauaugcuuuauuaauguuuucaaaagguauuauacaugugauacauuuuuuaagcuucaguugcuugucuucugguacuuucuguuaugggcuuuuggggagccagaagccaaucuacaaucucuuuuuguuugccaggacaugcaauaaaauuuaaaaaauaaauaaaaacuaauuaagaaauugaaaaaaaaaaaaaaaaaa

SEQ ID NO: 83 - protein, 555 aa, Homo sapiens
  tumor protein p63 (TP63), transcript variant 2
MNFETSRCATLQYCPDPYIQRFVETPAHFSWKESYYRSTMSQSTQTNEFLSPEVFQHIWDFLEQPICSVQPIDLNFVDEPSEDGATNKIEISMDCIRMQDSDLSDPMWPQYTNLGLLNSMDQQIQNGSSSTSPYNTDHAQNSVTAPSPYAQPSSTFDALSPSPAIPSNTDYPGPHSFDVSFQQSSTAKSATWTYSTELKKLYCQIAKTCPIQIKVMTPPPQGAVIRAMPVYKKAEHVTEVVKRCPNHELSREFNEGQIAPPSHLIRVEGNSHAQYVEDPITGRQSVLVPYEPPQVGTEFTTVLYNFMCNSSCVGGMNRRPILIIVTLETRDGQVLGRRCFEARICACPGRDRKADEDSIRKQQVSDSTKNGDGTKRPFRQNTHGIQMTSIKKRRSPDDELLYLPVRGRETYEMLLKIKESLELMQYLPQHTIETYRQQQQQQHQHLLQKQTSIQSPSSYGNSSPPLNKMNSMNKLPSVSQLINPQQRNALTPTTIPDGMGANIPMMGTHMPMAGDMNGLSPTQALPPPLSMPSTSHCTPPPPYPTDCSIVRIWQV

SEQ ID NO: 84 - RNA, 2870 nt, Homo sapiens
  tumor protein p63 (TP63), transcript variant 3
cccggcuuuauaucuauauauacacagguauauguguauauuuuauauaauuguucuccguucguugauaucaaagacaguugaaggaaaugaauuuugaaacuucacggugugccacccuacaguacugcccugacccuuacauccagcguuucguagaaaccccagcucauuucucuuggaaagaaaguuauuaccgauccaccaugucccagagcacacagacaaaugaauuccucaguccagagguuuuccagcauaucugggauuuucuggaacagccuauauguucaguucagcccauugacuugaacuuuguggaugaaccaucagaagauggugcgacaaacaagauugagauuagcauggacuguauccgcaugcaggacucggaccugagugaccccauguggccacaguacacgaaccuggggcuccugaacagcauggaccagcagauucagaacggcuccucguccaccagucccuauaacacagaccacgcgcagaacagcgucacggcgcccucgcccuacgcacagcccagcuccaccuucgaugcucucucuccaucacccgccauccccuccaacaccgacuacccaggcccgcacaguuucgacguguccuuccagcagucgagcaccgccaagucggccaccuggacguauuccacugaacugaagaaacucuacugccaaauugcaaagacaugccccauccagaucaaggugaugaccccaccuccucagggagcuguuauccgcgccaugccugucuacaaaaaagcugagcacgucacggagguggugaagcggugccccaaccaugagcugagccgugaauucaacgagggacagauugccccuccuagucauuugauucgaguagaggggaacagccaugcccaguauguagaagaucccaucacaggaagacagagugugcugguaccuuaugagccaccccagguuggcacugaauucacgacagucuuguacaauuucauguguaacagcaguuguguuggagggaugaaccgccguccaauuuuaaucauuguuacucuggaaaccagagaugggcaaguccugggccgacgcugcuuugaggcccggaucugugcuugcccaggaagagacaggaaggcggaugaagauagcaucagaaagcagcaaguuucggacaguacaaagaacggugaugguacgaagcgcccguuucgucagaacacacaugguauccagaugacauccaucaagaaacgaagauccccagaugaugaacuguuauacuuaccagugaggggccgugagacuuaugaaaugcuguugaagaucaaagagucccuggaacucaugcaguaccuuccucagcacacaauugaaacguacaggcaacagcaacagcagcagcaccagcacuuacuucagaaacaucuccuuucagccugcuucaggaaugagcuuguggagccccggagagaaacuccaaaacaaucugacgucuucuuuagacauuccaagcccccaaaccgaucaguguacccauagagcccuaucucuauauuuuaaguguguguguuguauuuccauguguauaugugaguguguguguguguaugugugugcguguguaucuagcccucauaaacaggacuugaagacacuuuggcucagagacccaacugcucaaaggcacaaagccacuagugagagaaucuuuugaagggacucaaaccuuuacaagaaaggauguuuucugcagauuuuguauccuuagaccggccauuggugggugaggaaccacuguguuugucugugagcuuucuguuguuuccugggagggaggggucagguggggaaaggggcauuaagauguuuauuggaacccuuuucugucuucuucuguuguuuuucuaaaauucacagggaagcuuuugagcaggucucaaacuuaagaugucuuuuuaagaaaaggagaaaaaaguuguuauugucugugcauaaguaaguuguaggugacugagagacucagucagacccuuuuaaugcuggucauguaauaauauugcaaguaguaagaaacgaaggugucaaguguacugcugggcagcgaggugaucauuaccaaaaguaaucaacuuuguggguggagaguucuuugugagaacuugcauuauuuguguccuccccucauguguagguagaacauuucuuaaugcuguguaccugccucugccacuguauguuggcaucuguuaugcuaaaguuuuucuuguacaugaaacccuggaagaccuacuacaaaaaaacuguuguuuggcccccauagcaggugaacucauuuugugcuuuuaauagaaagacaaauccaccccaguaauauugcccuuacguaguuguuuaccauuauucaaagcucaaaauagaauuugaagcccucucacaaaaucugugauuaauuugcuuaauuagagcuucuaucccucaagccuaccuaccauaaaaccagccauauuacugauacuguucagugcauuuagccaggagacuuacguuuugaguaagugagauccaagcagacguguuaaaaucagcacuccuggacuggaaauuaaagauugaaaggguagacuacuuuucuuuuuuuuacucaaaaguuuagagaaucucuguuucuuuccauuuuaaaaacauauuuuaagauaauagcauaaagacuuuaaaaauguuccuccccuccaucuucccacacccagucaccagcacuguauuuucugucaccaagacaaugauuucuuguuauugaggcuguugcuuuuguggaugugugauuuuaauuuucaauaaacuuuugcaucuugguuu

SEQ ID NO: 85 - protein, 487 aa, Homo sapiens
  tumor protein p63 (TP63), transcript variant 3
MNFETSRCATLQYCPDPYIQRFVETPAHFSWKESYYRSTMSQSTQTNEFLSPEVFQHIWDFLEQPICSVQPIDLNFVDEPSEDGATNKIEISMDCIRMQDSDLSDPMWPQYTNLGLLNSMDQQIQNGSSSTSPYNTDHAQNSVTAPSPYAQPSSTFDALSPSPAIPSNTDYPGPHSFDVSFQQSSTAKSATWTYSTELKKLYCQIAKTCPIQIKVMTPPPQGAVIRAMPVYKKAEHVTEVVKRCPNHELSREFNEGQIAPPSHLIRVEGNSHAQYVEDPITGRQSVLVPYEPPQVGTEFTTVLYNFMCNSSCVGGMNRRPILIIVTLETRDGQVLGRRCFEARICACPGRDRKADEDSIRKQQVSDSTKNGDGTKRPFRQNTHGIQMTSIKKRRSPDDELLYLPVRGRETYEMLLKIKESLELMQYLPQHTIETYRQQQQQQHQHLLQKHLLSACFRNELVEPRRETPKQSDVFFRHSKPPNRSVYP

SEQ ID NO: 86 - RNA, 4696 nt, Homo sapiens
  tumor protein p63 (TP63), transcript variant 4
agagagagaaagagagagagggacuugaguucuguuaucuucuuaaguagauucauauuguaagggucucgggguggggggguuggcaaaauccuggagccagaagaaaggacagcagcauugaucaaucuuacagcuaacauguuguaccuggaaaacaaugcccagacucaauuuagugagccacaguacacgaaccuggggcuccugaacagcauggaccagcagauucagaacggcuccucguccaccagucccuauaacacagaccacgcgcagaacagcgucacggcgcccucgcccuacgcacagccagcuccaccuucgaugcucucucuccaucacccgccauccccuccaacaccgacuacccaggcccgcacaguuucgacguguccuuccagcagucgagcaccgccaagucggccaccuggacguauuccacugaacugaagaaacucuacugccaaauugcaaagacaugccccauccagaucaaggugaugaccccaccuccucagggagcuguuauccgcgccaugccugucuacaaaaaagcugagcacgucacggagguggugaagcggugccccaaccaugagcugagccgugaauucaacgagggacagauugccccuccuagucauuugauucgaguagaggggaacagccaugcccaguauguagaagaucccaucacaggaagacagagugugcugguaccuuaugagccaccccagguuggcacugaauucacgacagucuuguacaauuucauguguaacagcaguuguguuggagggaugaaccgccguccaauuuuaaucauuguuacucuggaaaccagagaugggcaaguccugggccgacgcugcuuugaggcccggaucugugcuugcccaggaagagacaggaaggcggaugaagauagcaucagaaagcagcaaguuucggacaguacaaagaacggugaugguacgaagcgcccguuucgucagaacacacaugguauccagaugacauccaucaagaaacgaagauccccagaugaugaacuguuauacuuaccagugaggggccgugagacuuaugaaaugcuguugaagaucaaagagucccuggaacucaugcaguaccuuccucagcacacaauugaaacguacaggcaacagcaacagcagcagcaccagcacuuacuucagaaacagaccucaauacagucuccaucuucauaugguaacagcuccccaccucugaacaaaaugaacagcaugaacaagcugccuucugugagccagcuuaucaacccucagcagcgcaacgcccucacuccuacaaccauuccugauggcaugggagccaacauucccaugaugggcacccacaugccaauggcuggagacaugaauggacucagccccacccaggcacucccucccccacucuccaugccauccaccucccacugcacacccccaccuccguaucccacagauugcagcauugucaguuucuuagcgagguugggcuguucaucaugucuggacuauuucacgacccaggggcugaccaccaucuaucagauugagcauuacuccauggaugaucuggcaagucugaaaaucccugagcaauuucgacaugcgaucuggaagggcauccuggaccaccggcagcuccacgaauucuccuccccuucucaucuccugcggaccccaagcagugccucuacagucagugugggcuccagugagacccggggugagcguguuauugaugcugugcgauucacccuccgccagaccaucucuuucccaccccgagaugaguggaaugacuucaacuuugacauggaugcucgccgcaauaagcaacagcgcaucaaagaggagggggagugagccucaccaugugagcucuuccuaucccucuccuaacugccagcccccuaaaagcacuccugcuuaaucuucaaagccuucucccuagcuccuccccuuccucuugucugauuucuuaggggaaggagaaguaagaggcuaccucuuaccuaacaucugaccuggcaucuaauucugauucuggcuuuaagccuucaaaacuauagcuugcagaacuguagcugccauggcuagguagaagugagcaaaaaagaguugggugucuccuuaagcugcagagauuucucauugacuuuuauaaagcauguucacccuuauagucuaagacuauauauauaaauguauaaauauacaguauagauuuuuggguggggggcauugaguauuguuuaaaauguaauuuaaaugaaagaaaauugaguugcacuuauugaccauuuuuuaauuuacuuguuuuggauggcuugucuauacuccuucccuuaagggguaucauguauggugauagguaucuagagcuuaaugcuacaugugagugacgaugauguacagauucuuucaguucuuuggauucuaaauacaugccacaucaaaccuuugaguagauccauuuccauugcuuauuauguagguaagacuguagauauguauucuuuucucaguguugguauauuuuauauuacugacauuucuucuagugaugaugguucacguuggggugauuuaauccaguuauaagaagaaguucauguccaaacguccucuuuaguuuuugguugggaaugaggaaaauucuuaaaaggcccauagcagccaguucaaaaacacccgacgucauguauuugagcauaucaguaacccccuuaaauuuaauaccagauaccuuaucuuacaauauugauugggaaaacauuugcugccauuacagagguauuaaaacuaaauuucacuacuagauugacuaacucaaauacacauuugcuacuguuguaagaauucugauugauuugauugggaugaaugccaucuaucuaguucuaacagugaaguuuuacugucuauuaauauucaggguaaauaggaaucauucagaaauguugagucuguacuaaacaguaagauaucucaaugaaccauaaauucaacuuuguaaaaaucuuuugaagcauagauaauauuguuugguaaauguuucuuuuguuugguaaauguuucuuuuaaagacccuccuauucuauaaaacucugcauguagaggcuuguuuaccuuucucucucuaagguuuacaauaggaguggugauuugaaaaauauaaaauuaugagauugguuuuccuguggcauaaauugcaucacuguaucauuuucuuuuuuaaccgguaagaguuucaguuuguuggaaaguaacugugagaacccaguuucccguccaucucccuuagggacuacccauagacaugaaagguccccacagagcaagagauaagucuuucauggcugcuguugcuuaaaccacuuaaacgaagaguucccuugaaacuuugggaaaacauguuaaugacaauauuccagaucuuucagaaauauaacacauuuuuuugcaugcaugcaaaugagcucugaaaucuucccaugcauucuggucaagggcugucauugcacauaagcuuccauuuuaauuuuaaagugcaaaagggccagcguggcucuaaaagguaauguguggauugccucugaaaaguguguauauauuuugugugaaauugcauacuuuguauuuugauuauuuuuuuuuucuucuugggauagugggauuuccagaaccacacuugaaaccuuuuuuuaucguuuuuguauuuucaugaaaauaccauuuaguaagaauaccacaucaaauaagaaauaaugcuacaauuuuaagaggggagggaagggaaaguuuuuuuuuauuauuuuuuuaaaauuuuguauguuaaagagaaugaguccuugauuucaaaguuuuguuguacuuaaaugguaauaagcacuguaaacuucugcaacaagcaugcagcuuugcaaacccauuaaggggaagaaugaaagcuguuccuugguccuaguaagaagacaaacugcuucccuuacuuugcugaggguuugaauaaaccuaggacuuccgagcuaugucaguacuauucagguaacacuagggccuuggaaauuccuguacugugucucauggauuuggcacuagccaaagcgaggcacccuuacuggcuuaccuccucauggcagccuacucuccuugaguguaugaguagccaggguaagggguaaaaggauaguaagcauagaaaccacuagaaagugggcuuaauggaguucuuguggccucagcucaaugcaguuagcugaagaauugaaaaguuuuuguuuggagacguuuauaaacagaaauggaaagcagaguuuucauuaaauccuuuuaccuuuuuuuuuucuugguaauccccuaaaauaacaguaugugggauauugaauguuaaagggauauuuuuuucuauuauuuuuauaauuguacaaaauuaagcaaauguuaaaaguuuuauaugcuuuauuaauguuuucaaaagguauuauacaugugauacauuuuuuaagcuucaguugcuugucuucugguacuuucuguuaugggcuuuuggggagccagaagccaaucuacaaucucuuuuuguuugccaggacaugcaauaaaauuuaaaaaauaaauaaaaacuaauuaagaaauugaaaaaaaaaaaaaaaaaa

SEQ ID NO: 87 - protein, 586 aa, Homo sapiens
  tumor portein p63 (TP63), transcript variant 4
MLYLENNAQTQFSEPQYTNLGLLNSMDQQIQNGSSSTSPYNTDHAQNSVTAPSPYAQPSSTFDALSPSPAIPSNTDYPGPHSFDVSFQQSSTAKSATWTYSTELKKLYCQIAKTCPIQIKVMTPPPQGAVIRAMPVYKKAEHVTEVVKRCPNHELSREFNEGQIAPPSHLIRVEGNSHAQYVEDPITGRQSVLVPYEPPQVGTEFTTVLYNFMCNSSCVGGMNRRPILIIVTLETRDGQVLGRRCFEARICACPGRDRKADEDSIRKQQVSDSTKNGDGTKRPFRQNTHGIQMTSIKKRRSPDDELLYLPVRGRETYEMLLKIKESLELMQYLPQHTIETYRQQQQQQHQHLLQKQTSIQSPSSYGNSSPPLNKMNSMNKLPSVSQLINPQQRNALTPTTIPDGMGANIPMMGTHMPMAGDMNGLSPTQALPPPLSMPSTSHCTPPPPYPTDCSIVSFLARLGCSSCLDYFTTQGLTTIYQIEHYSMDDLASLKIPEQFRHAIWKGILDHRQLHEFSSPSHLLRTPSSASTVSVGSSETRGERVIDAVRFTLRQTISFPPRDEWNDFNFDMDARRNKQQRIKEEGE

SEQ ID NO: 88 - RNA, 4602 nt, Homo sapiens
  tumor protein p63 (TP63), transcript variant 5
agagagagaaagagagagagggacuugaguucuguuaucuucuuaaguagauucauauuguaagggucucgggguggggggguuggcaaaauccuggagccagaagaaaggacagcagcauugaucaaucuuacagcuaacauguuguaccuggaaaacaaugcccagacucaauuuagugagccacaguacacgaaccuggggcuccugaacagcauggaccagcagauucagaacggcuccucguccaccagucccuauaacacagaccacgcgcagaacagcgucacggcgcccucgcccuacgcacagcccagcuccaccuucgaugcucucucuccaucacccgccauccccuccaacaccgacuacccaggcccgcacaguuucgacguguccuuccagcagucgagcaccgccaagucggccaccuggacguauuccacugacugaagaaacucuacugccaaauugcaaagacaugccccauccagaucaaggugaugaccccaccuccucagggagcuguuauccgcgccaugccugucuacaaaaaagcugagcacgucacggagguggugaagcggugccccaaccaugagcugagccgugaauucaacgagggacagauugccccuccuagucauuugauucgaguagaggggaacagccaugcccaguauguagaagaucccaucacaggaagacagagugugcugguaccuuaugagccaccccagguuggcacugaauucacgacagucuuguacaauuucauguguaacagcaguuguguuggagggaugaaccgccguccaauuuuaaucauuguuacucuggaaaccagagaugggcaaguccugggccgacgcugcuuugaggcccggaucugugcuugcccaggaagagacaggaaggcggaugaagauagcaucagaaagcagcaaguuucggacaguacaaagaacggugaugguacgaagcgcccguuucgucagaacacacaugguauccagaugacauccaucaagaaacgaagauccccagaugaugaacuguuauacuuaccagugaggggccgugagacuuaugaaaugcuguugaagaucaaagagucccuggaacucaugcaguaccuuccucagcacacaauugaaacguacaggcaacagcaacagcagcagcaccagcacuuacuucagaaacagaccucaauacagucuccaucuucauaugguaacagcuccccaccucugaacaaaaugaacagcaugaacaagcugccuucugugagccagcuuaucaacccucagcagcgcaacgcccucacuccuacaaccauuccugauggcaugggagccaacauucccaugaugggcacccacaugccaauggcuggagacaugaauggacucagccccacccaggcacucccucccccacucuccaugccauccaccucccacugcacacccccaccuccguaucccacagauugcagcauugucaggaucuggcaagucugaaaaucccugagcaauuucgacaugcgaucuggaagggcauccuggaccaccggcagcuccacgaauucuccuccccuucucaucuccugcggaccccaagcagugccucuacagucagugugggcuccagugagacccggggugagcguguuauugaugcugugcgauucacccuccgccagaccaucucuuucccaccccgagaugaguggaaugacuucaacuuugacauggaugcucgccgcaauaagcaacagcgcaucaaagaggagggggagugagccucaccaugugagcucuuccuaucccucuccuaacugccagcccccuaaaagcacuccugcuuaaucuucaaagccuucucccuagcuccuccccuuccucuugucugauuucuuaggggaaggagaaguaagaggcuaccucuuaccuaacaucugaccuggcaucuaauucugauucuggcuuuaagccuucaaaacuauagcuugcagaacuguagcugccauggcuagguagaagugagcaaaaaagaguugggugucuccuuaagcugcagagauuucucauugacuuuuauaaagcauguucacccuuauagucuaagacuauauauauaaauguauaaauauacaguauagauuuuuggguggggggcauugaguauuguuuaaaauguaauuuaaaugaaagaaaauugaguugcacuuauugaccauuuuuuaauuuacuuguuuuggauggcuugucuauacuccuucccuuaagggguaucauguauggugauagguaucuagagcuuaaugcuacaugugagugacgaugauguacagauucuuucaguucuuuggauucuaaauacaugccacaucaaaccuuugaguagauccauuuccauugcuuauuauguagguaagacuguagauauguauucuuuucucaguguugguauauuuuauauuacugacauuucuucuagugaugaugguucacguuggggugauuuaauccaguuauaagaagaaguucauguccaaacguccucuuuaguuuuugguugggaaugaggaaaauucuuaaaaggcccauagcagccaguucaaaaacacccgacgucauguauuugagcauaucaguaacccccuuaaauuuaauaccagauaccuuaucuuacaauauugauugggaaaacauuugcugccauuacagagguauuaaaacuaaauuucacuacuagauugacuaacucaaauacacauuugcuacuguuguaagaauucugauugauuugauugggaugaaugccaucuaucuaguucuaacagugaaguuuuacugucuauuaauauucaggguaaauaggaaucauucagaaauguugagucuguacuaaacaguaagauaucucaaugaaccauaaauucaacuuuguaaaaaucuuuugaagcauagauaauauuguuugguaaauguuucuuuuguuugguaaauguuucuuuuaaagacccuccuauucuauaaaacucugcauguagaggcuuguuuaccuuucucucucuaagguuuacaauaggaguggugauuugaaaaauauaaaauuaugagauugguuuuccuguggcauaaauugcaucacuguaucauuuucuuuuuuaaccgguaagaguuucaguuuguuggaaaguaacugugagaacccaguuucccguccaucucccuuagggacuacccauagacaugaaagguccccacagagcaagagauaagucuuucauggcugcuguugcuuaaaccacuuaaacgaagaguucccuugaaacuuugggaaaacauguuaaugacaauauuccagaucuuucagaaauauaacacauuuuuuugcaugcaugcaaaugagcucugaaaucuucccaugcauucuggucaagggcugucauugcacauaagcuuccauuuuaauuuuaaagugcaaaagggccagcguggcucuaaaagguaauguguggauugccucugaaaaguguguauauauuuugugugaaauugcauacuuuguauuuugauuauuuuuuuuuucuucuugggauagugggauuuccagaaccacacuugaaaccuuuuuuuaucguuuuuguauuuucaugaaaauaccauuuaguaagaauaccacaucaaauaagaaauaaugcuacaauuuuaagaggggagggaagggaaaguuuuuuuuuauuauuuuuuuaaaauuuuguauguuaaagagaaugaguccuugauuucaaaguuuuguuguacuuaaaugguaauaagcacuguaaacuucugcaacaagcaugcagcuuugcaaacccauuaaggggaagaaugaaagcuguuccuugguccuaguaagaagacaaacugcuucccuuacuuugcugaggguuugaauaaaccuaggacuuccgagcuaugucaguacuauucagguaacacuagggccuuggaaauuccuguacugugucucauggauuuggcacuagccaaagcgaggcacccuuacuggcuuaccuccucauggcagccuacucuccuugaguguaugaguagccaggguaagggguaaaaggauaguaagcauagaaaccacuagaaagugggcuuaauggaguucuuguggccucagcucaaugcaguuagcugaagaauugaaaaguuuuuguuuggagacguuuauaaacagaaauggaaagcagaguuuucauuaaauccuuuuaccuuuuuuuuuucuugguaauccccuaaaauaacaguaugugggauauugaauguuaaagggauauuuuuuucuauuauuuuuauaauuguacaaaauuaagcaaauguuaaaaguuuuauaugcuuuauuaauguuuucaaaagguauuauacaugugauacauuuuuuaagcuucaguugcuugucuucugguacuuucuguuaugggcuuuuggggagccagaagccaaucuacaaucucuuuuuguuugccaggacaugcaauaaaauuuaaaaaauaaauaaaaacuaauuaagaaauugaaaaaaaaaaaaaaaaaa

SEQ ID NO: 89 - protein, 461 aa, Homo sapiens
  tumor protein p63 (TP63), transcript variant 5
MLYLENNAQTQFSEPQYTNLGLLNSMDQQIQNGSSSTSPYNTDHAQNSVTAPSPYAQPSSTFDALSPSPAIPSNTDYPGPHSFDVSFQQSSTAKSATWTYSTELKKLYCQIAKTCPIQIKVMTPPPQGAVIRAMPVYKKAEHVTEVVKRCPNHELSREFNEGQIAPPSHLIRVEGNSHAQYVEDPITGRQSVLVPYEPPQVGTEFTTVLYNFMCNSSCVGGMNRRPILIIVTLETRDGQVLGRRCFEARICACPGRDRKADEDSIRKQQVSDSTKNGDGTKRPFRQNTHGIQMTSIKKRRSPDDELLYLPVRGRETYEMLLKIKESLELMQYLPQHTIETYRQQQQQQHQHLLQKQTSIQSPSSYGNSSPPLNKMNSMNKLPSVSQLINPQQRNALTPTTIPDGMGANIPMMGTHMPMAGDMNGLSPTQALPPPLSMPSTSHCTPPPPYPTDCSIVRIWQV

SEQ ID NO: 90 - RNA, 2640 nt, Homo sapiens
  tumor protein p63 (TP63), transcript variant 6
agagagagaaagagagagagggacuugaguucuguuaucuucuuaaguagauucauauuguaagggucucgggguggggggguuggcaaaauccuggagccagaagaaaggacagcagcauugaucaaucuuacagcuaacauguuguaccuggaaaacaaugcccagacucaauuuagugagccacaguacacgaaccuggggcuccugaacagcauggaccagcagauucagaacggcuccucguccaccagucccuauaacacagaccacgcgcagaacagcgucacggcgcccucgcccuacgcacagcccagcuccaccuucgaugcucucucuccaucacccgccauccccuccaacaccgacuacccaggcccgcacaguuucgacguguccuuccagcagucgagcaccgccaagucggccaccuggacguauuccacugaacugaagaaacucuacugccaaauugcaaagacaugccccauccagaucaaggugaugaccccaccuccucagggagcuguuauccgcgccaugccugucuacaaaaaagcugagcacgucacggagguggugaagcggugccccaaccaugagcugagccgugaauucaacgagggacagauugccccuccuagucauuugauucgaguagaggggaacagccaugcccaguauguagaagaucccaucacaggaagacagagugugcugguaccuuaugagccaccccagguuggcacugaauucacgacagucuuguacaauuucauguguaacagcaguuguguuggagggaugaaccgccguccaauuuuaaucauuguuacucuggaaaccagagaugggcaaguccugggccgacgcugcuuugaggcccggaucugugcuugcccaggaagagacaggaaggcggaugaagauagcaucagaaagcagcaaguuucggacaguacaaagaacggugaugguacgaagcgcccguuucgucagaacacacaugguauccagaugacauccaucaagaaacgaagauccccagaugaugaacuguuauacuuaccagugaggggccgugagacuuaugaaaugcuguugaagaucaaagagucccuggaacucaugcaguaccuuccucagcacacaauugaaacguacaggcaacagcaacagcagcagcaccagcacuuacuucagaaacaucuccuuucagccugcuucaggaaugagcuuguggagccccggagagaaacuccaaaacaaucugacgucuucuuuagacauuccaagcccccaaaccgaucaguguacccauagagcccuaucucuauauuuuaaguguguguguuguauuuccauguguauaugugaguguguguguguguaugugugugcguguguaucuagcccucauaaacaggacuugaagacacuuuggcucagagacccaacugcucaaaggcacaaagccacuagugagagaaucuuuugaagggacucaaaccuuuacaagaaaggauguuuucugcagauuuuguauccuuagaccggccauuggugggugaggaaccacuguguuugucugugagcuuucuguuguuuccugggagggaggggucagguggggaaaggggcauuaagauguuuauuggaacccuuuucugucuucuucuguuguuuuucuaaaauucacagggaagcuuuugagcaggucucaaacuuaagaugucuuuuuaagaaaaggagaaaaaaguuguuauugucugugcauaaguaaguuguaggugacugagagacucagucagacccuuuuaaugcuggucauguaauaauauugcaaguaguaagaaacgaaggugucaaguguacugcugggcagcgaggugaucauuaccaaaaguaaucaacuuuguggguggagaguucuuugugagaacuugcauuauuuguguccuccccucauguguagguagaacauuucuuaaugcuguguaccugccucugccacuguauguuggcaucuguuaugcuaaaguuuuucuuguacaugaaacccuggaagaccuacuacaaaaaaacuguuguuuggcccccauagcaggugaacucauuuugugcuuuuaauagaaagacaaauccaccccaguaauauugcccuuacguaguuguuuaccauuauucaaagcucaaaauagaauuugaagcccucucacaaaaucugugauuaauuugcuuaauuagagcuucuaucccucaagccuaccuaccauaaaaccagccauauuacugauacuguucagugcauuuagccaggagacuuacguuuugaguaagugagauccaagcagacguguuaaaaucagcacuccuggacuggaaauuaaagauugaaaggguagacuacuuuucuuuuuuuuacucaaaaguuuagagaaucucuguuucuuuccauuuuaaaaacauauuuuaagauaauagcauaaagacuuuaaaaauguuccuccccuccaucuucccacacccagucaccagcacuguauuuucugucaccaagacaaugauuucuuguuauugaggcuguugcuuuuguggaugugugauuuuaauuuucaauaaacuuuugcaucuugguuu

SEQ ID NO: 91 - protein, 393 aa, Homo sapiens
  tumor protein p63 (TP63), transcript variant 6
MLYLENNAQTQFSEPQYTNLGLLNSMDQQIQNGSSSTSPYNTDHAQNSVTAPSPYAQPSSTFDALSPSPAIPSNTDYPGPHSFDVSFQQSSTAKSATWTYSTELKKLYCQIAKTCPIQIKVMTPPPQGAVIRAMPVYKKAEHVTEVVKRCPNHELSREFNEGQIAPPSHLIRVEGNSHAQYVEDPITGRQSVLVPYEPPQVGTEFTTVLYNFMCNSSCVGGMNRRPILIIVTLETRDGQVLGRRCFEARICACPGRDRKADEDSIRKQQVSDSTKNGDGTKRPFRQNTHGIQMTSIKKRRSPDDELLYLPVRGRETYEMLLKIKESLELMQYLPQHTIETYRQQQQQQHQHLLQKHLLSACFRNELVEPRRETPKQSDVFFRHSKPPNRSVYP

SEQ ID NO: 92 - RNA, 4927 nt, Homo sapiens
  tumor protein p63 (TP63), transcript variant 1
cccggcuuuauaucuauauauacacagguauauguguauauuuuauauaauuguucuccguucguugauaucaaagacaguugaaggaaaugaauuuugaaacuucacggugugccacccuacaguacugcccugacccuuacauccagcguuucguagaaaccccagcucauuucucuuggaaagaaaguuauuaccgauccaccaugucccagagcacacagacaaaugaauuccucaguccagagguuuuccagcauaucugggauuuucuggaacagccuauauguucaguucagcccauugacuugaacuuuguggaugaaccaucagaagauggugcgacaaacaagauugagauuagcauggacuguauccgcaugcaggacucggaccugagugaccccauguggccacaguacacgaaccuggggcuccugaacagcauggaccagcagauucagaacggcuccucguccaccagucccuauaacacagaccacgcgcagaacagcgucacggcgcccucgcccuacgcacagcccagcuccaccuucgaugcucucucuccaucacccgccauccccuccaacaccgacuacccaggcccgcacaguuucgacguguccuuccagcagucgagcaccgccaagucggccaccuggacguauuccacugaacugaagaaacucuacugccaaauugcaaagacaugccccauccagaucaaggugaugaccccaccuccucagggagcuguuauccgcgccaugccugucuacaaaaaagcugagcacgucacggagguggugaagcggugccccaaccaugagcugagccgugaauucaacgagggacagauugccccuccuagucauuugauucgaguagaggggaacagccaugcccaguauguagaagaucccaucacaggaagacagagugugcugguaccuuaugagccaccccagguuggcacugaauucacgacagucuuguacaauuucauguguaacagcaguuguguuggagggaugaaccgccguccaauuuuaaucauuguuacucuggaaaccagagaugggcaaguccugggccgacgcugcuuugaggcccggaucugugcuugcccaggaagagacaggaaggcggaugaagauagcaucagaaagcagcaaguuucggacaguacaaagaacggugaugguacgaagcgcccguuucgucagaacacacaugguauccagaugacauccaucaagaaacgaagauccccagaugaugaacuguuauacuuaccagugaggggccgugagacuuaugaaaugcuguugaagaucaaagagucccuggaacucaugcaguaccuuccucagcacacaauugaaacguacaggcaacagcaacagcagcagcaccagcacuuacuucagaaacagaccucaauacagucuccaucuucauaugguaacagcuccccaccucugaacaaaaugaacagcaugaacaagcugccuucugugagccagcuuaucaacccucagcagcgcaacgcccucacuccuacaaccauuccugauggcaugggagccaacauucccaugaugggcacccacaugccaauggcuggagacaugaauggacucagccccacccaggcacucccucccccacucuccaugccauccaccucccacugcacacccccaccuccguaucccacagauugcagcauugucaguuucuuagcgagguugggcuguucaucaugucuggacuauuucacgacccaggggcugaccaccaucuaucagauugagcauuacuccauggaugaucuggcaagucugaaaaucccugagcaauuucgacaugcgaucuggaagggcauccuggaccaccggcagcuccacgaauucuccuccccuucucaucuccugcggaccccaagcagugccucuacagucagugugggcuccagugagacccggggugagcguguuauugaugcugugcgauucacccuccgccagaccaucucuuucccaccccgagaugaguggaaugacuucaacuuugacauggaugcucgccgcaauaagcaacagcgcaucaaagaggagggggagugagccucaccaugugagcucuuccuaucccucuccuaacugccagcccccuaaaagcacuccugcuuaaucuucaaagccuucucccuagcuccuccccuuccucuugucugauuucuuaggggaaggagaaguaagaggcuaccucuuaccuaacaucugaccuggcaucuaauucugauucuggcuuuaagccuucaaaacuauagcuugcagaacuguagcugccauggcuagguagaagugagcaaaaaagaguugggugucuccuuaagcugcagagauuucucauugacuuuuauaaagcauguucacccuuauagucuaagacuauauauauaaauguauaaauauacaguauagauuuuuggguggggggcauugaguauuguuuaaaauguaauuuaaaugaaagaaaauugaguugcacuuauugaccauuuuuuaauuuacuuguuuuggauggcuugucuauacuccuucccuuaagggguaucauguauggugauagguaucuagagcuuaaugcuacaugugagugacgaugauguacagauucuuucaguucuuuggauucuaaauacaugccacaucaaaccuuugaguagauccauuuccauugcuuauuauguagguaagacuguagauauguauucuuuucucaguguugguauauuuuauauuacugacauuucuucuagugaugaugguucacguuggggugauuuaauccaguuauaagaagaaguucauguccaaacguccucuuuaguuuuugguugggaaugaggaaaauucuuaaaaggcccauagcagccaguucaaaaacacccgacgucauguauuugagcauaucaguaacccccuuaaauuuaauaccagauaccuuaucuuacaauauugauugggaaaacauuugcugccauuacagagguauuaaaacuaaauuucacuacuagauugacuaacucaaauacacauuugcuacuguuguaagaauucugauugauuugauugggaugaaugccaucuaucuaguucuaacagugaaguuuuacugucuauuaauauucaggguaaauaggaaucauucagaaauguugagucuguacuaaacaguaagauaucucaaugaaccauaaauucaacuuuguaaaaaucuuuugaagcauagauaauauuguuugguaaauguuucuuuuguuugguaaauguuucuuuuaaagacccuccuauucuauaaaacucugcauguagaggcuuguuuaccuuucucucucuaagguuuacaauaggaguggugauuugaaaaauauaaaauuaugagauugguuuuccuguggcauaaauugcaucacuguaucauuuucuuuuuuaaccgguaagaguuucaguuuguuggaaaguaacugugagaacccaguuucccguccaucucccuuagggacuacccauagacaugaaagguccccacagagcaagagauaagucuuucauggcugcuguugcuuaaaccacuuaaacgaagaguucccuugaaacuuugggaaaacauguuaaugacaauauuccagaucuuucagaaauauaacacauuuuuuugcaugcaugcaaaugagcucugaaaucuucccaugcauucuggucaagggcugucauugcacauaagcuuccauuuuaauuuuaaagugcaaaagggccagcguggcucuaaaagguaauguguggauugccucugaaaaguguguauauauuuugugugaaauugcauacuuuguauuuugauuauuuuuuuuuucuucuugggauagugggauuuccagaaccacacuugaaaccuuuuuuuaucguuuuuguauuuucaugaaaauaccauuuaguaagaauaccacaucaaauaagaaauaaugcuacaauuuuaagaggggagggaagggaaaguuuuuuuuuauuauuuuuuuaaaauuuuguauguuaaagagaaugaguccuugauuucaaaguuuuguuguacuuaaaugguaauaagcacuguaaacuucugcaacaagcaugcagcuuugcaaacccauuaaggggaagaaugaaagcuguuccuugguccuaguaagaagacaaacugcuucccuuacuuugcugaggguuugaauaaaccuaggacuuccgagcuaugucaguacuauucagguaacacuagggccuuggaaauuccuguacugugucucauggauuuggcacuagccaaagcgaggcacccuuacuggcuuaccuccucauggcagccuacucuccuugaguguaugaguagccaggguaagggguaaaaggauaguaagcauagaaaccacuagaaagugggcuuaauggaguucuuguggccucagcucaaugcaguuagcugaagaauugaaaaguuuuuguuuggagacguuuauaaacagaaauggaaagcagaguuuucauuaaauccuuuuaccuuuuuuuuuucuugguaauccccuaaaauaacaguaugugggauauugaauguuaaagggauauuuuuuucuauuauuuuuauaauuguacaaaauuaagcaaauguuaaaaguuuuauaugcuuuauuaauguuuucaaaagguauuauacaugugauacauuuuuuaagcuucaguugcuugucuucugguacuuucuguuaugggcuuuuggggagccagaagccaaucuacaaucucuuuuuguuugccaggacaugcaauaaaauuuaaaaaauaaauaaaaacuaauuaagaaauugaaaaaaaaaaaaaaaaaa

SEQ ID NO: 93 - protein, 680 aa, Homo sapiens
  tumor protein p63 (TP63), transcript variant 1
MNFETSRCATLQYCPDPYIQRFVETPAHFSWKESYYRSTMSQSTQTNEFLSPEVFQHIWDFLEQPICSVQPIDLNFVDEPSEDGATNKIEISMDCIRMQDSDLSDPMWPQYTNLGLLNSMDQQIQNGSSSTSPYNTDHAQNSVTAPSPYAQPSSTFDALSPSPAIPSNTDYPGPHSFDVSFQQSSTAKSATWTYSTELKKLYCQIAKTCPIQIKVMTPPPQGAVIRAMPVYKKAEHVTEVVKRCPNHELSREFNEGQIAPPSHLIRVEGNSHAQYVEDPITGRQSVLVPYEPPQVGTEFTTVLYNFMCNSSCVGGMNRRPILIIVTLETRDGQVLGRRCFEARICACPGRDRKADEDSIRKQQVSDSTKNGDGTKRPFRQNTHGIQMTSIKKRRSPDDELLYLPVRGRETYEMLLKIKESLELMQYLPQHTIETYRQQQQQQHQHLLQKQTSIQSPSSYGNSSPPLNKMNSMNKLPSVSQLINPQQRNALTPTTIPDGMGANIPMMGTHMPMAGDMNGLSPTQALPPPLSMPSTSHCTPPPPYPTDCSIVSFLARLGCSSCLDYFTTQGLTTIYQIEHYSMDDLASLKIPEQFRHAIWKGILDHRQLHEFSSPSHLLRTPSSASTVSVGSSETRGERVIDAVRFTLRQTISFPPRDEWNDFNFDMDARRNKQQRIKEEGE

SEQ ID NO: 94 - RNA, 2320 nt, Homo sapiens
  keratin 5
ucgacagcucucucgcccagcccaguucuggaagggauaaaaagggggcaucaccguuccuggguaacagagccaccuucugcguccugcugagcucuguucucuccagcaccucccaacccacuagugccugguucucuugcuccaccaggaacaagccaccaugucucgccagucaaguguguccuuccggagcgggggcagucguagcuucagcaccgccucugccaucaccccgucugucucccgcaccagcuucaccuccgugucccgguccggggguggcgguggugguggcuucggcagggucagccuugcgggugcuuguggaguggguggcuauggcagccggagccucuacaaccuggggggcuccaagaggauauccaucagcacuagugguggcagcuucaggaaccgguuuggugcuggugcuggaggcggcuauggcuuuggagguggugccgguaguggauuugguuucggcgguggagcuggugguggcuuugggcucgguggcggagcuggcuuuggagguggcuucgguggcccuggcuuuccugucugcccuccuggagguauccaagaggucacugucaaccagagucuccugacuccccucaaccugcaaaucgaccccagcauccagagggugaggaccgaggagcgcgagcagaucaagacccucaacaauaaguuugccuccuucaucgacaaggugcgguuccuggagcagcagaacaagguucuggacaccaaguggacccugcugcaggagcagggcaccaagacugugaggcagaaccuggagccguuguucgagcaguacaucaacaaccucaggaggcagcuggacagcaucgugggggaacggggccgccuggacucagagcugagaaacaugcaggaccugguggaagacuucaagaacaaguaugaggaugaaaucaacaagcguaccacugcugagaaugaguuugugaugcugaagaaggauguagaugcugccuacaugaacaagguggagcuggaggccaagguugaugcacugauggaugagauuaacuucaugaagauguucuuugaugcggagcugucccagaugcagacgcaugucucugacaccucagugguccucuccauggacaacaaccgcaaccuggaccuggauagcaucaucgcugaggucaaggcccaguaugaggagauugccaaccgcagccggacagaagccgaguccugguaucagaccaaguaugaggagcugcagcagacagcuggccggcauggcgaugaccuccgcaacaccaagcaugagaucucugagaugaaccggaugauccagaggcugagagccgagauugacaaugucaagaaacagugcgccaaucugcagaacgccauugcggaugccgagcagcguggggagcuggcccucaaggaugccaggaacaagcuggccgagcuggaggaggcccugcagaaggccaagcaggacauggcccggcugcugcgugaguaccaggagcucaugaacaccaagcuggcccuggacguggagaucgccacuuaccgcaagcugcuggagggcgaggaaugcagacucaguggagaaggaguuggaccagucaacaucucuguugucacaagcaguguuuccucuggauauggcaguggcaguggcuauggcgguggccucgguggaggucuuggcggcggccucgguggaggucuugccggagguagcaguggaagcuacuacuccagcagcagugggggugucggccuagguggugggcucaguguggggggcucuggcuucagugcaagcaguggccgagggcugggggugggcuuuggcaguggcggggguagcagcuccagcgucaaauuugucuccaccaccuccuccucccggaagagcuucaagagcuaagaaccugcugcaagucacugccuuccaagugcagcaacccagcccauggagauugccucuucuaggcaguugcucaagccauguuuuauccuuuucuggagaguagucuagaccaagccaauugcagaaccacauucuuugguucccaggagagccccauucccagccccuggucucccgugccgcaguucuauauucugcuucaaaucagccuucagguuucccacagcauggccccugcugacacgagaacccaaaguuuucccaaaucuaaaucaucaaaacagaauccccaccccaaucccaaauuuuguuuugguucuaacuaccuccagaauguguucaauaaaaugcuuuuauaauauaaaaaaaaaaaaaaaaaaa

SEQ ID NO: 95 - protein, 590 aa, Homo sapiens
  keratin 5
MSRQSSVSFRSGGSRSFSTASAITPSVSRTSFTSVSRSGGGGGGGFGRVSLAGACGVGGYGSRSLYNLGGSKRISISTSGGSFRNRFGAGAGGGYGFGGGAGSGFGFGGGAGGGFGLGGGAGFGGGFGGPGFPVCPPGGIQEVTVNQSLLTPLNLQIDPSIQRVRTEEREQIKTLNNKFASFIDKVRFLEQQNKVLDTKWTLLQEQGTKTVRQNLEPLFEQYINNLRRQLDSIVGERGRLDSELRNMQDLVEDFKNKYEDEINKRTTAENEFVMLKKDVDAAYMNKVELEAKVDALMDEINFMKMFFDAELSQMQTHVSDTSVVLSMDNNRNLDLDSIIAEVKAQYEEIANRSRTEAESWYQTKYEELQQTAGRHGDDLRNTKHEISEMNRMIQRLRAEIDNVKKQCANLQNAIADAEQRGELALKDARNKLAELEEALQKAKQDMARLLREYQELMNTKLALDVEIATYRKLLEGEECRLSGEGVGPVNISVVTSSVSSGYGSGSGYGGGLGGGLGGGLGGGLAGGSSGSYYSSSSGGVGLGGGLSVGGSGFSASSGRGLGVGFGSGGGSSSSVKFVSTTSSSRKSFKS

SEQ ID NO: 96 - RNA, 2450 nt, Homo sapiens
  keratin 6
auauuucauaccuuucuagaaacugggugugaucucacuguugguaaagcccagcccuucccaaccugcaagcucaccuuccaggacugggcccagcccaugcucuccauauauaagcugcugccccgagccugauuccuaguccugcuucucuucccucucuccuccagccucucacacucuccucagcucucucaucuccuggaaccauggccagcacauccaccaccaucaggagccacagcagcagccgccgggguuucagugccaacucagccaggcucccuggggucagccgcucuggcuucagcagcgucuccgugucccgcuccaggggcagugguggccuggguggugcauguggaggagcuggcuuuggcagccgcagucuguauggccuggggggcuccaagaggaucuccauuggagggggcagcugugccaucaguggcggcuauggcagcagagccggaggcagcuauggcuuugguggcgccgggaguggauuugguuucggugguggagccggcauuggcuuuggucugggugguggagccggccuugcugguggcuuugggggcccuggcuucccugugugccccccuggaggcauccaagaggucaccgucaaccagagucuccugacuccccucaaccugcaaaucgaucccaccauccagcgggugcgggcugaggagcgugaacagaucaagacccucaacaacaaguuugccuccuucaucgacaaggugcgguuccuggagcagcagaacaagguucuggaaacaaaguggacccugcugcaggagcagggcaccaagacugugaggcagaaccuggagccguuguucgagcaguacaucaacaaccucaggaggcagcuggacagcauugucggggaacggggccgccuggacucagagcucagaggcaugcaggaccugguggaggacuucaagaacaaauaugaggaugaaaucaacaagcgcacagcagcagagaaugaauuugugacucugaagaaggauguggaugcugccuacaugaacaagguugaacugcaagccaaggcagacacucucacagacgagaucaacuuccugagagccuuguaugaugcagagcugucccagaugcagacccacaucucagacacaucuguggugcuguccauggacaacaaccgcaaccuggaccuggacagcaucaucgcugaggucaaggcccaauaugaggagauugcucagagaagccgggcugaggcugaguccugguaccagaccaaguacgaggagcugcaggucacagcaggcagacauggggacgaccugcgcaacaccaagcaggagauugcugagaucaaccgcaugauccagaggcugagaucugagaucgaccacgucaagaagcagugcgccaaccugcaggccgccauugcugaugcugagcagcguggggagauggcccucaaggaugccaagaacaagcuggaagggcuggaggaugcccugcagaaggccaagcaggaccuggcccggcugcugaaggaguaccaggagcugaugaaugucaagcuggcccuggacguggagaucgccaccuaccgcaagcugcuggagggugaggagugcaggcugaauggcgaaggcguuggacaagucaacaucucuguggugcaguccaccgucuccaguggcuauggcggugccaguggugucggcaguggcuuaggccuggguggaggaagcagcuacuccuauggcaguggucuuggcguuggagguggcuucaguuccagcaguggcagagccauuggggguggccucagcucuguuggaggcggcaguuccaccaucaaguacaccaccaccuccuccuccagcaggaagagcuauaagcacuaaagugcgucugcuagcucucggucccacaguccucaggccccucucuggcugcagagcccucuccucagguugccuuuccucuccuggccuccagucuccccugcugucccagguagagcuggguauggaugcuuagugcccucacuucuucucucucucucuauaccaucugagcacccauugcucaccaucagaucaaccucugauuuuacaucaugauguaaucaccacuggagcuucacuguuacuaaauuauuaauuucuugccuccaguguucuaucucugaggcugagcauuauaagaaaaugaccucugcuccuuuucauugcagaaaauugccaggggcuuauuucagaacaacuuccacuuacuuuccacuggcucucaaacucucuaacuuauaaguguugugaacccccacccaggcaguauccaugaaagcacaagugacuaguccuaugauguacaaagccuguaucucugugaugauuucugugcucuucgcuguuugcaauugcuaaauaaagcagauuuauaauacaaua

SEQ ID NO: 97 - protein, 564 aa, Homo sapiens
  keratin 6
MASTSTTIRSHSSSRRGFSANSARLPGVSRSGFSSVSVSRSRGSGGLGGACGGAGFGSRSLYGLGGSKRISIGGGSCAISGGYGSRAGGSYGFGGAGSGFGFGGGAGIGFGLGGGAGLAGGFGGPGFPVCPPGGIQEVTVNQSLLTPLNLQIDPTIQRVRAEEREQIKTLNNKFASFIDKVRFLEQQNKVLETKWTLLQEQGTKTVRQNLEPLFEQYINNLRRQLDSIVGERGRLDSELRGMQDLVEDFKNKYEDEINKRTAAENEFVTLKKDVDAAYMNKVELQAKADTLTDEINFLRALYDAELSQMQTHISDTSVVLSMDNNRNLDLDSIIAEVKAQYEEIAQRSRAEAESWYQTKYEELQVTAGRHGDDLRNTKQEIAEINRMIQRLRSEIDHVKKQCANLQAAIADAEQRGEMALKDAKNKLEGLEDALQKAKQDLARLLKEYQELMNVKLALDVEIATYRKLLEGEECRLNGEGVGQVNISVVQSTVSSGYGGASGVGSGLGLGGGSSYSYGSGLGVGGGFSSSSGRAIGGGLSSVGGGSSTIKYTTTSSSSRKSYKH

SEQ ID NO: 98 - RNA, 1753 nt, Homo sapiens
  keratin 7
cagccccgccccuaccuguggaagcccagccgcccgcucccgcggauaaaaggcgcggaguguccccgaggucagcgagugcgcgcuccuccucgcccgccgcuagguccaucccggcccagccaccauguccauccacuucagcuccccgguauucaccucgcgcucagccgccuucucgggccgcggcgcccaggugcgccugagcuccgcucgccccggcggccuuggcagcagcagccucuacggccucggcgccucacggccgcgcguggccgugcgcucugccuaugggggcccggugggcgccggcauccgcgaggucaccauuaaccagagccugcuggccccgcugcggcuggacgccgaccccucccuccagcgggugcgccaggaggagagcgagcagaucaagacccucaacaacaaguuugccuccuucaucgacaaggugcgguuucuggagcagcagaacaagcugcuggagaccaaguggacgcugcugcaggagcagaagucggccaagagcagccgccucccagacaucuuugaggcccagauugcuggccuucggggucagcuugaggcacugcagguggaugggggccgccuggaggcggagcugcggagcaugcaggaugugguggaggacuucaagaauaaguacgaagaugaaauuaaccaccgcacagcugcugagaaugaguuuguggugcugaagaaggauguggaugcugccuacaugagcaagguggagcuggaggccaagguggaugcccugaaugaugagaucaacuuccucaggacccucaaugagacggaguugacagagcugcagucccagaucuccgacacaucuguggugcuguccauggacaacagucgcucccuggaccuggacggcaucaucgcugaggucaaggcgcaguaugaggagauggccaaaugcagccgggcugaggcugaagccugguaccagaccaaguuugagacccuccaggcccaggcugggaagcauggggacgaccuccggaauacccggaaugagauuucagagaugaaccgggccauccagaggcugcaggcugagaucgacaacaucaagaaccagcgugccaaguuggaggccgccauugccgaggcugaggagcguggggagcuggcgcucaaggaugcucgugccaagcaggaggagcuggaagccgcccugcagcggggcaagcaggauauggcacggcagcugcgugaguaccaggaacucaugagcgugaagcuggcccuggacaucgagaucgccaccuaccgcaagcugcuggagggcgaggagagccgguuggcuggagauggagugggagccgugaauaucucugugaugaauuccacugguggcaguagcaguggcgguggcauugggcugacccucgggggaaccaugggcagcaaugcccugagcuucuccagcagugcggguccugggcuccugaaggcuuauuccauccggaccgcauccgccagucgcaggagugcccgcgacugagccgccucccaccacuccacuccuccagccaccacccacaaucacaagaagauucccaccccugccucccaugccuggucccaagacagugagacagucuggaaagugaugucagaauagcuuccaauaaagcagccucauucugaggccugagugauccacgugaaaaaaaaaaaaaaaaaaaaaaaaaaaaaaaaaaaaaaaaaaaaaaaaaaa

SEQ ID NO: 99 - protein, 469 aa, Homo sapiens
  keratin 7
MSIHFSSPVFTSRSAAFSGRGAQVRLSSARPGGLGSSSLYGLGASRPRVAVRSAYGGPVGAGIREVTINQSLLAPLRLDADPSLQRVRQEESEQIKTLNNKFASFIDKVRFLEQQNKLLETKWTLLQEQKSAKSSRLPDIFEAQIAGLRGQLEALQVDGGRLEAELRSMQDVVEDFKNKYEDEINHRTAAENEFVVLKKDVDAAYMSKVELEAKVDALNDEINFLRTLNETELTELQSQISDTSVVLSMDNSRSLDLDGIIAEVKAQYEEMAKCSRAEAEAWYQTKFETLQAQAGKHGDDLRNTRNEISEMNRAIQRLQAEIDNIKNQRAKLEAAIAEAEERGELALKDARAKQEELEAALQRGKQDMARQLREYQELMSVKLALDIEIATYRKLLEGEESRLAGDGVGAVNISVMNSTGGSSSGGGIGLTLGGTMGSNALSFSSSAGPGLLKAYSIRTASASRRSARD

SEQ ID NO: 100 - RNA, 89 nt, Homo sapiens
  microRNA 9-1 (MIR9-1)
cggggttggttgttatctttggttatctagctgtatgagtggtgtggagtcttcataaagctagataaccgaaagtaaaaataacccca

SEQ ID NO: 101 - RNA, 87 nt, Homo sapiens
  microRNA 9-2 (MIR9-2)
ggaagcgagttgttatctttggttatctagctgtatgagtgtattggtcttcataaagctagataaccgaaagtaaaaactccttca

SEQ ID NO: 102 - RNA, 90 nt, Homo sapiens
  microRNA 9-3 (MIR9-3)
ggaggcccgtttctctctttggttatctagctgtatgagtgccacagagccgtcataaagctagataaccgaaagtagaaatgattctca

SEQ ID NO: 103 - RNA, 87 nt, Homo sapiens
  microRNA let-7d (MIRLET7D)
cctaggaagaggtagtaggttgcatagttttagggcagggattttgcccacaaggaggtaactatacgacctgctgcctttcttagg

SEQ ID NO: 104 - RNA, 3677 nt, Homo sapiens
  VEGFA
ucgcggaggcuuggggcagccggguagcucggaggucguggcgcugggggcuagcaccagcgcucugucgggaggcgcagcgguuagguggaccggucagcggacucaccggccagggcgcucggugcuggaauuugauauucauugauccggguuuuaucccucuucuuuuuucuuaaacauuuuuuuuuaaaacuguauuguuucucguuuuaauuuauuuuugcuugccauuccccacuugaaucgggccgacggcuuggggagauugcucuacuuccccaaaucacuguggauuuuggaaaccagcagaaagaggaaagagguagcaagagcuccagagagaagucgaggaagagagagacggggucagagagagcgcgcgggcgugcgagcagcgaaagcgacaggggcaaagugagugaccugcuuuugggggugaccgccggagcgcggcgugagcccucccccuugggaucccgcagcugaccagucgcgcugacggacagacagacagacaccgcccccagccccagcuaccaccuccuccccggccggcggcggacaguggacgcggcggcgagccgcgggcaggggccggagcccgcgcccggaggcgggguggagggggucggggcucgcggcgucgcacugaaacuuuucguccaacuucugggcuguucucgcuucggaggagccgugguccgcgcgggggaagccgagccgagcggagccgcgagaagugcuagcucgggccgggaggagccgcagccggaggagggggaggaggaagaagagaaggaagaggagagggggccgcaguggcgacucggcgcucggaagccgggcucauggacgggugaggcggcggugugcgcagacagugcuccagccgcgcgcgcuccccaggcccuggcccgggccucgggccggggaggaagaguagcucgccgaggcgccgaggagagcgggccgccccacagcccgagccggagagggagcgcgagccgcgccggccccggucgggccuccgaaaccaugaacuuucugcugucuugggugcauuggagccuugccuugcugcucuaccuccaccaugccaaguggucccaggcugcacccauggcagaaggaggagggcagaaucaucacgaaguggugaaguucauggaugucuaucagcgcagcuacugccauccaaucgagacccugguggacaucuuccaggaguacccugaugagaucgaguacaucuucaagccauccugugugccccugaugcgaugcgggggcugcugcaaugacgagggccuggagugugugcccacugaggaguccaacaucaccaugcagauuaugcggaucaaaccucaccaaggccagcacauaggagagaugagcuuccuacagcacaacaaaugugaaugcagaccaaagaaagauagagcaagacaagaaaaaaaaucaguucgaggaaagggaaaggggcaaaaacgaaagcgcaagaaaucccgguauaaguccuggagcguguacguuggugcccgcugcugucuaaugcccuggagccucccuggcccccaucccugugggccuugcucagagcggagaaagcauuuguuuguacaagauccgcagacguguaaauguuccugcaaaaacacagacucgcguugcaaggcgaggcagcuugaguuaaacgaacguacuugcagaugugacaagccgaggcggugagccgggcaggaggaaggagccucccucaggguuucgggaaccagaucucucaccaggaaagacugauacagaacgaucgauacagaaaccacgcugccgccaccacaccaucaccaucgacagaacaguccuuaauccagaaaccugaaaugaaggaagaggagacucugcgcagagcacuuuggguccggagggcgagacuccggcggaagcauucccgggcgggugacccagcacggucccucuuggaauuggauucgccauuuuauuuuucuugcugcuaaaucaccgagcccggaagauuagagaguuuuauuucugggauuccuguagacacacccacccacauacauacauuuauauauauauauauuauauauauauaaaaauaaauaucucuauuuuauauauauaaaauauauauauucuuuuuuuaaauuaacagugcuaauguuauuggugucuucacuggauguauuugacugcuguggacuugaguugggaggggaauguucccacucagauccugacagggaagaggaggagaugagagacucuggcaugaucuuuuuuuugucccacuugguggggccaggguccucuccccugcccaggaaugugcaaggccagggcaugggggcaaauaugacccaguuuugggaacaccgacaaacccagcccuggcgcugagccucucuaccccaggucagacggacagaaagacagaucacagguacagggaugaggacaccggcucugaccaggaguuuggggagcuucaggacauugcugugcuuuggggauucccuccacaugcugcacgcgcaucucgcccccaggggcacugccuggaagauucaggagccugggcggccuucgcuuacucucaccugcuucugaguugcccaggagaccacuggcagaugucccggcgaagagaagagacacauuguuggaagaagcagcccaugacagcuccccuuccugggacucgcccucauccucuuccugcuccccuuccuggggugcagccuaaaaggaccuauguccucacaccauugaaaccacuaguucuguccccccaggagaccugguugugugugugugagugguugaccuuccuccauccccugguccuucccuucccuucccgaggcacagagagacagggcaggauccacgugcccauuguggaggcagagaaaagagaaaguguuuuauauacgguacuuauuuaauaucccuuuuuaauuagaaauuaaaacaguuaauuuaauuaaagaguaggguuuuuuuucaguauucuugguuaauauuuaauuucaacuauuuaugagauguaucuuuugcucucucuugcucucuuauuuguaccgguuuuuguauauaaaauucauguuuccaaucucucucucccugaucggugacagucacuagcuuaucuugaacagauauuuaauuuugcuaacacucagcucugcccuccccgauccccuggcuccccagcacacauuccuuugaaauaagguuucaauauacaucuacauacuauauauauauuuggcaacuuguauuuguguguauauauauauauauauguuuauguauauaugugauucugauaaaauagacauugcuauucuguuuuuuauauguaaaaacaaaacaagaaaaaauagagaauucuacauacuaaaucucucuccuuuuuuaauuuuaauauuuguuaucauuuauuuauuggugcuacuguuuauccguaauaauuguggggaaaagauauuaacaucacgucuuugucucuagugcaguuuuucgagauauuccguaguacauauuuauuuuuaaacaacgacaaagaaauacagauauaucuuaaaaaaaaaaaagcauuuuguauuaaagaauuuaauucugaucucaaaaaaaaaaaaaaaaaa

SEQ ID NO: 105 - protein, 412 aa, Homo sapiens
  VEGFA
MTDRQTDTAPSPSYHLLPGRRRTVDAAASRGQGPEPAPGGGVEGVGARGVALKLFVQLLGCSRFGGAVVRAGEAEPSGAARSASSGREEPQPEEGEEEEEKEEERGPQWRLGARKPGSWTGEAAVCADSAPAARAPQALARASGRGGRVARRGAEESGPPHSPSRRGSASRAGPGRASETMNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

SEQ ID NO: 106 - RNA, 10754 nt, Homo sapiens
  VEGFA isoform c
ucgcggaggcuuggggcagccggguagcucggaggucguggcgcugggggcuagcaccagcgcucugucgggaggcgcagcgguuagguggaccggucagcggacucaccggccagggcgcucggugcuggaauuugauauucauugauccggguuuuaucccucuucuuuuuucuuaaacauuuuuuuuuaaaacuguauuguuucucguuuuaauuuauuuuugcuugccauuccccacuugaaucgggccgacggcuuggggagauugcucuacuuccccaaaucacuguggauuuuggaaaccagcagaaagaggaaagagguagcaagagcuccagagagaagucgaggaagagagagacggggucagagagagcgcgcgggcgugcgagcagcgaaagcgacaggggcaaagugagugaccugcuuuugggggugaccgccggagcgcggcgugagcccucccccuugggaucccgcagcugaccagucgcgcugacggacagacagacagacaccgcccccagccccagcuaccaccuccuccccggccggcggcggacaguggacgcggcggcgagccgcgggcaggggccggagcccgcgcccggaggcgggguggagggggucggggcucgcggcgucgcacugaaacuuuucguccaacuucugggcuguucucgcuucggaggagccgugguccgcgcgggggaagccgagccgagcggagccgcgagaagugcuagcucgggccgggaggagccgcagccggaggagggggaggaggaagaagagaaggaagaggagagggggccgcaguggcgacucggcgcucggaagccgggcucauggacgggugaggcggcggugugcgcagacagugcuccagccgcgcgcgcuccccaggcccuggcccgggccucgggccggggaggaagaguagcucgccgaggcgccgaggagagcgggccgccccacagcccgagccggagagggagcgcgagccgcgccggccccggucgggccuccgaaaccaugaacuuucugcugucuugggugcauuggagccuugccuugcugcucuaccuccaccaugccaaguggucccaggcugcacccauggcagaaggaggagggcagaaucaucacgaaguggugaaguucauggaugucuaucagcgcagcuacugccauccaaucgagacccugguggacaucuuccaggaguacccugaugagaucgaguacaucuucaagccauccugugugccccugaugcgaugcgggggcugcugcaaugacgagggccuggagugugugcccacugaggaguccaacaucaccaugcagauuaugcggaucaaaccucaccaaggccagcacauaggagagaugagcuuccuacagcacaacaaaugugaaugcagaccaaagaaagauagagcaagacaagaaaaaaaaucaguucgaggaaagggaaaggggcaaaaacgaaagcgcaagaaaucccgucccugugggccuugcucagagcggagaaagcauuuguuuguacaagauccgcagacguguaaauguuccugcaaaaacacagacucgcguugcaaggcgaggcagcuugaguuaaacgaacguacuugcagaugugacaagccgaggcggugagccgggcaggaggaaggagccucccucaggguuucgggaaccagaucucucaccaggaaagacugauacagaacgaucgauacagaaaccacgcugccgccaccacaccaucaccaucgacagaacaguccuuaauccagaaaccugaaaugaaggaagaggagacucugcgcagagcacuuuggguccggagggcgagacuccggcggaagcauucccgggcgggugacccagcacggucccucuuggaauuggauucgccauuuuauuuuucuugcugcuaaaucaccgagcccggaagauuagagaguuuuauuucugggauuccuguagacacacccacccacauacauacauuuauauauauauauauuauauauauauaaaaauaaauaucucuauuuuauauauauaaaauauauauauucuuuuuuuaaauuaacagugcuaauguuauuggugucuucacuggauguauuugacugcuguggacuugaguugggaggggaauguucccacucagauccugacagggaagaggaggagaugagagacucuggcaugaucuuuuuuuugucccacuugguggggccaggguccucuccccugcccaggaaugugcaaggccagggcaugggggcaaauaugacccaguuuugggaacaccgacaaacccagcccuggcgcugagccucucuaccccaggucagacggacagaaagacagaucacagguacagggaugaggacaccggcucugaccaggaguuuggggagcuucaggacauugcugugcuuuggggauucccuccacaugcugcacgcgcaucucgcccccaggggcacugccuggaagauucaggagccugggcggccuucgcuuacucucaccugcuucugaguugcccaggagaccacuggcagaugucccggcgaagagaagagacacauuguuggaagaagcagcccaugacagcuccccuuccugggacucgcccucauccucuuccugcuccccuuccuggggugcagccuaaaaggaccuauguccucacaccauugaaaccacuaguucuguccccccaggagaccugguugugugugugugagugguugaccuuccuccauccccugguccuucccuucccuucccgaggcacagagagacagggcaggauccacgugcccauuguggaggcagagaaaagagaaaguguuuuauauacgguacuuauuuaauaucccuuuuuaauuagaaauuaaaacaguuaauuuaauuaaagaguaggguuuuuuuucaguauucuugguuaauauuuaauuucaacuauuuaugagauguaucuuuugcucucucuugcucucuuauuuguaccgguuuuuguauauaaaauucauguuuccaaucucucucucccugaucggugacagucacuagcuuaucuugaacagauauuuaauuuugcuaacacucagcucugcccuccccgauccccuggcuccccagcacacauuccuuugaaauaagguuucaauauacaucuacauacuauauauauauuuggcaacuuguauuuguguguauauauauauauauauguuuauguauauaugugauucugauaaaauagacauugcuauucuguuuuuuauauguaaaaacaaaacaagaaaaaauagagaauucuacauacuaaaucucucuccuuuuuuaauuuuaauauuuguuaucauuuauuuauuggugcuacuguuuauccguaauaauuguggggaaaagauauuaacaucacgucuuugucucuagugcaguuuuucgagauauuccguaguacauauuuauuuuuaaacaacgacaaagaaauacagauauaucuuaaaaaaaaaaaagcauuuuguauuaaagaauuuaauucugaucucaaaaaaaaaaaaaaaaaaggaggcgcagcgguuagguggaccggucagcggacucaccggccagggcgcucggugcuggaauuugauauucauugauccggguuuuaucccucuucuuuuuucuuaaacauuuuuuuuuaaaacuguauuguuucucguuuuaauuuauuuuugcuugccauuccccacuugaaucgggccgacggcuuggggagauugcucuacuuccccaaaucacuguggauuuuggaaaccagcagaaagaggaaagagguagcaagagcuccagagagaagucgaggaagagagagacggggucagagagagcgcgcgggcgugcgagcagcgaaagcgacaggggcaaagugagugaccugcuuuugggggugaccgccggagcgcggcgugagcccucccccuugggaucccgcagcugaccagucgcgcugacggacagacagacagacaccgcccccagccccagcuaccaccuccuccccggccggcggcggacaguggacgcggcggcgagccgcgggcaggggccggagcccgcgcccggaggcgggguggagggggucggggcucgcggcgucgcacugaaacuuuucguccaacuucugggcuguucucgcuucggaggagccgugguccgcgcgggggaagccgagccgagcggagccgcgagaagugcuagcucgggccgggaggagccgcagccggaggagggggaggaggaagaagagaaggaagaggagagggggccgcaguggcgacucggcgcucggaagccgggcucauggacgggugaggcggcggugugcgcagacagugcuccagccgcgcgcgcuccccaggcccuggcccgggccucgggccggggaggaagaguagcucgccgaggcgccgaggagagcgggccgccccacagcccgagccggagagggagcgcgagccgcgccggccccggucgggccuccgaaaccaugaacuuucugcugucuugggugcauuggagccuugccuugcugcucuaccuccaccaugccaaguggucccaggcugcacccauggcagaaggaggagggcagaaucaucacgaaguggugaaguucauggaugucuaucagcgcagcuacugccauccaaucgagacccugguggacaucuuccaggaguacccugaugagaucgaguacaucuucaagccauccugugugccccugaugcgaugcgggggcugcugcaaugacgagggccuggagugugugcccacugaggaguccaacaucaccaugcagauuaugcggaucaaaccucaccaaggccagcacauaggagagaugagcuuccuacagcacaacaaaugugaaugcagaccaaagaaagauagagcaagacaagaaaaaaaaucaguucgaggaaagggaaaggggcaaaaacgaaagcgcaagaaaucccgucccugugggccuugcucagagcggagaaagcauuuguuuguacaagauccgcagacguguaaauguuccugcaaaaacacagacucgcguugcaaggcgaggcagcuugaguuaaacgaacguacuugcagaugugacaagccgaggcggugagccgggcaggaggaaggagccucccucaggguuucgggaaccagaucucucaccaggaaagacugauacagaacgaucgauacagaaaccacgcugccgccaccacaccaucaccaucgacagaacaguccuuaauccagaaaccugaaaugaaggaagaggagacucugcgcagagcacuuuggguccggagggcgagacuccggcggaagcauucccgggcgggugacccagcacggucccucuuggaauuggauucgccauuuuauuuuucuugcugcuaaaucaccgagcccggaagauuagagaguuuuauuucugggauuccuguagacacacccacccacauacauacauuuauauauauauauauuauauauauauaaaaauaaauaucucuauuuuauauauauaaaauauauauauucuuuuuuuaaauuaacagugcuaauguuauuggugucuucacuggauguauuugacugcuguggacuugaguugggaggggaauguucccacucagauccugacagggaagaggaggagaugagagacucuggcaugaucuuuuuuuugucccacuugguggggccaggguccucuccccugcccaggaaugugcaaggccagggcaugggggcaaauaugacccaguuuugggaacaccgacaaacccagcccuggcgcugagccucucuaccccaggucagacggacagaaagacagaucacagguacagggaugaggacaccggcucugaccaggaguuuggggagcuucaggacauugcugugcuuuggggauucccuccacaugcugcacgcgcaucucgcccccaggggcacugccuggaagauucaggagccugggcggccuucgcuuacucucaccugcuucugaguugcccaggagaccacuggcagaugucccggcgaagagaagagacacauuguuggaagaagcagcccaugacagcuccccuuccugggacucgcccucauccucuuccugcuccccuuccuggggugcagccuaaaaggaccuauguccucacaccauugaaaccacuaguucuguccccccaggagaccugguugugugugugugagugguugaccuuccuccauccccugguccuucccuucccuucccgaggcacagagagacagggcaggauccacgugcccauuguggaggcagagaaaagagaaaguguuuuauauacgguacuuauuuaauaucccuuuuuaauuagaaauuaaaacaguuaauuuaauuaaagaguaggguuuuuuuucaguauucuugguuaauauuuaauuucaacuauuuaugagauguaucuuuugcucucucuugcucucuuauuuguaccgguuuuuguauauaaaauucauguuuccaaucucucucucccugaucggugacagucacuagcuuaucuugaacagauauuuaauuuugcuaacacucagcucugcccuccccgauccccuggcuccccagcacacauuccuuugaaauaagguuucaauauacaucuacauacuauauauauauuuggcaacuuguauuuguguguauauauauauauauauguuuauguauauaugugauucugauaaaauagacauugcuauucuguuuuuuauauguaaaaacaaaacaagaaaaaauagagaauucuacauacuaaaucucucuccuuuuuuaauuuuaauauuuguuaucauuuauuuauuggugcuacuguuuauccguaauaauuguggggaaaagauauuaacaucacgucuuugucucuagugcaguuuuucgagauauuccguaguacauauuuauuuuuaaacaacgacaaagaaauacagauauaucuuaaaaaaaaaaaagcauuuuguauuaaagaauuuaauucugaucucaaaaaaaaaaaaaaaaaaucgcggaggcuuggggcagccggguagcucggaggucguggcgcugggggcuagcaccagcgcucugucgggaggcgcagcgguuagguggaccggucagcggacucaccggccagggcgcucggugcuggaauuugauauucauugauccggguuuuaucccucuucuuuuuucuuaaacauuuuuuuuuaaaacuguauuguuucucguuuuaauuuauuuuugcuugccauuccccacuugaaucgggccgacggcuuggggagauugcucuacuuccccaaaucacuguggauuuuggaaaccagcagaaagaggaaagagguagcaagagcuccagagagaagucgaggaagagagagacggggucagagagagcgcgcgggcgugcgagcagcgaaagcgacaggggcaaagugagugaccugcuuuugggggugaccgccggagcgcggcgugagcccucccccuugggaucccgcagcugaccagucgcgcugacggacagacagacagacaccgcccccagccccagcuaccaccuccuccccggccggcggcggacaguggacgcggcggcgagccgcgggcaggggccggagcccgcgcccggaggcgggguggagggggucggggcucgcggcgucgcacugaaacuuuucguccaacuucugggcuguucucgcuucggaggagccgugguccgcgcgggggaagccgagccgagcggagccgcgagaagugcuagcucgggccgggaggagccgcagccggaggagggggaggaggaagaagagaaggaagaggagagggggccgcaguggcgacucggcgcucggaagccgggcucauggacgggugaggcggcggugugcgcagacagugcuccagccgcgcgcgcuccccaggcccuggcccgggccucgggccggggaggaagaguagcucgccgaggcgccgaggagagcgggccgccccacagcccgagccggagagggagcgcgagccgcgccggccccggucgggccuccgaaaccaugaacuuucugcugucuugggugcauuggagccuugccuugcugcucuaccuccaccaugccaaguggucccaggcugcacccauggcagaaggaggagggcagaaucaucacgaaguggugaaguucauggaugucuaucagcgcagcuacugccauccaaucgagacccugguggacaucuuccaggaguacccugaugagaucgaguacaucuucaagccauccugugugccccugaugcgaugcgggggcugcugcaaugacgagggccuggagugugugcccacugaggaguccaacaucaccaugcagauuaugcggaucaaaccucaccaaggccagcacauaggagagaugagcuuccuacagcacaacaaaugugaaugcagaccaaagaaagauagagcaagacaagaaaaaaaaucaguucgaggaaagggaaaggggcaaaaacgaaagcgcaagaaaucccgucccugugggccuugcucagagcggagaaagcauuuguuuguacaagauccgcagacguguaaauguuccugcaaaaacacagacucgcguugcaaggcgaggcagcuugaguuaaacgaacguacuugcagaugugacaagccgaggcggugagccgggcaggaggaaggagccucccucaggguuucgggaaccagaucucucaccaggaaagacugauacagaacgaucgauacagaaaccacgcugccgccaccacaccaucaccaucgacagaacaguccuuaauccagaaaccugaaaugaaggaagaggagacucugcgcagagcacuuuggguccggagggcgagacuccggcggaagcauucccgggcgggugacccagcacggucccucuuggaauuggauucgccauuuuauuuuucuugcugcuaaaucaccgagcccggaagauuagagaguuuuauuucugggauuccuguagacacacccacccacauacauacauuuauauauauauauauuauauauauauaaaaauaaauaucucuauuuuauauauauaaaauauauauauucuuuuuuuaaauuaacagugcuaauguuauuggugucuucacuggauguauuugacugcuguggacuugaguugggaggggaauguucccacucagauccugacagggaagaggaggagaugagagacucuggcaugaucuuuuuuuugucccacuugguggggccaggguccucuccccugcccaggaaugugcaaggccagggcaugggggcaaauaugacccaguuuugggaacaccgacaaacccagcccuggcgcugagccucucuaccccaggucagacggacagaaagacagaucacagguacagggaugaggacaccggcucugaccaggaguuuggggagcuucaggacauugcugugcuuuggggauucccuccacaugcugcacgcgcaucucgcccccaggggcacugccuggaagauucaggagccugggcggccuucgcuuacucucaccugcuucugaguugcccaggagaccacuggcagaugucccggcgaagagaagagacacauuguuggaagaagcagcccaugacagcuccccuuccugggacucgcccucauccucuuccugcuccccuuccuggggugcagccuaaaaggaccuauguccucacaccauugaaaccacuaguucuguccccccaggagaccugguugugugugugugagugguugaccuuccuccauccccugguccuucccuucccuucccgaggcacagagagacagggcaggauccacgugcccauuguggaggcagagaaaagagaaaguguuuuauauacgguacuuauuuaauaucccuuuuuaauuagaaauuaaaacaguuaauuuaauuaaagaguaggguuuuuuuucaguauucuugguuaauauuuaauuucaacuauuuaugagauguaucuuuugcucucucuugcucucuuauuuguaccgguuuuuguauauaaaauucauguuuccaaucucucucucccugaucggugacagucacuagcuuaucuugaacagauauuuaauuuugcuaacacucagcucugcccuccccgauccccuggcuccccagcacacauuccuuugaaauaagguuucaauauacaucuacauacuauauauauauuuggcaacuuguauuuguguguauauauauauauauauguuuauguauauaugugauucugauaaaauagacauugcuauucuguuuuuuauauguaaaaacaaaacaagaaaaaauagagaauucuacauacuaaaucucucuccuuuuuuaauuuuaauauuuguuaucauuuauuuauuggugcuacuguuuauccguaauaauuguggggaaaagauauuaacaucacgucuuugucucuagugcaguuuuucgagauauuccguaguacauauuuauuuuuaaacaacgacaaagaaauacagauauaucuuaaaaaaaaaaaagcauuuuguauuaaagaauuuaauucugaucucaaaaaaaaaaaaaaaaaa

SEQ ID NO: 107 - protein, 389 aa, Homo sapiens
  VEGFA
MTDRQTDTAPSPSYHLLPGRRRTVDAAASRGQGPEPAPGGGVEGVGARGVALKLFVQLLGCSRFGGAVVRAGEAEPSGAARSASSGREEPQPEEGEEEEEKEEERGPQWRLGARKPGSWTGEAAVCADSAPAARAPQALARASGRGGRVARRGAEESGPPHSPSRRGSASRAGPGRASETMNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRPCGPCSERRKHLFVQDPQTCKCSCKNTDSRCKARQLELNERTCRCDKPRR

SEQ ID NO: 108 - RNA, 3554 nt, Homo sapiens
  VEGF isoform d
ucgcggaggcuuggggcagccggguagcucggaggucguggcgcugggggcuagcaccagcgcucugucgggaggcgcagcgguuagguggaccggucagcggacucaccggccagggcgcucggugcuggaauuugauauucauugauccggguuuuaucccucuucuuuuuucuuaaacauuuuuuuuuaaaacuguauuguuucucguuuuaauuuauuuuugcuugccauuccccacuugaaucgggccgacggcuuggggagauugcucuacuuccccaaaucacuguggauuuuggaaaccagcagaaagaggaaagagguagcaagagcuccagagagaagucgaggaagagagagacggggucagagagagcgcgcgggcgugcgagcagcgaaagcgacaggggcaaagugagugaccugcuuuugggggugaccgccggagcgcggcgugagcccucccccuugggaucccgcagcugaccagucgcgcugacggacagacagacagacaccgcccccagccccagcuaccaccuccuccccggccggcggcggacaguggacgcggcggcgagccgcgggcaggggccggagcccgcgcccggaggcgggguggagggggucggggcucgcggcgucgcacugaaacuuuucguccaacuucugggcuguucucgcuucggaggagccgugguccgcgcgggggaagccgagccgagcggagccgcgagaagugcuagcucgggccgggaggagccgcagccggaggagggggaggaggaagaagagaaggaagaggagagggggccgcaguggcgacucggcgcucggaagccgggcucauggacgggugaggcggcggugugcgcagacagugcuccagccgcgcgcgcuccccaggcccuggcccgggccucgggccggggaggaagaguagcucgccgaggcgccgaggagagcgggccgccccacagcccgagccggagagggagcgcgagccgcgccggccccggucgggccuccgaaaccaugaacuuucugcugucuugggugcauuggagccuugccuugcugcucuaccuccaccaugccaaguggucccaggcugcacccauggcagaaggaggagggcagaaucaucacgaaguggugaaguucauggaugucuaucagcgcagcuacugccauccaaucgagacccugguggacaucuuccaggaguacccugaugagaucgaguacaucuucaagccauccugugugccccugaugcgaugcgggggcugcugcaaugacgagggccuggagugugugcccacugaggaguccaacaucaccaugcagauuaugcggaucaaaccucaccaaggccagcacauaggagagaugagcuuccuacagcacaacaaaugugaaugcagaccaaagaaagauagagcaagacaagaaaaucccugugggccuugcucagagcggagaaagcauuuguuuguacaagauccgcagacguguaaauguuccugcaaaaacacagacucgcguugcaaggcgaggcagcuugaguuaaacgaacguacuugcagaugugacaagccgaggcggugagccgggcaggaggaaggagccucccucaggguuucgggaaccagaucucucaccaggaaagacugauacagaacgaucgauacagaaaccacgcugccgccaccacaccaucaccaucgacagaacaguccuuaauccagaaaccugaaaugaaggaagaggagacucugcgcagagcacuuuggguccggagggcgagacuccggcggaagcauucccgggcgggugacccagcacggucccucuuggaauuggauucgccauuuuauuuuucuugcugcuaaaucaccgagcccggaagauuagagaguuuuauuucugggauuccuguagacacacccacccacauacauacauuuauauauauauauauuauauauauauaaaaauaaauaucucuauuuuauauauauaaaauauauauauucuuuuuuuaaauuaacagugcuaauguuauuggugucuucacuggauguauuugacugcuguggacuugaguugggaggggaauguucccacucagauccugacagggaagaggaggagaugagagacucuggcaugaucuuuuuuuugucccacuugguggggccaggguccucuccccugcccaggaaugugcaaggccagggcaugggggcaaauaugacccaguuuugggaacaccgacaaacccagcccuggcgcugagccucucuaccccaggucagacggacagaaagacagaucacagguacagggaugaggacaccggcucugaccaggaguuuggggagcuucaggacauugcugugcuuuggggauucccuccacaugcugcacgcgcaucucgcccccaggggcacugccuggaagauucaggagccugggcggccuucgcuuacucucaccugcuucugaguugcccaggagaccacuggcagaugucccggcgaagagaagagacacauuguuggaagaagcagcccaugacagcuccccuuccugggacucgcccucauccucuuccugcuccccuuccuggggugcagccuaaaaggaccuauguccucacaccauugaaaccacuaguucuguccccccaggagaccugguugugugugugugagugguugaccuuccuccauccccugguccuucccuucccuucccgaggcacagagagacagggcaggauccacgugcccauuguggaggcagagaaaagagaaaguguuuuauauacgguacuuauuuaauaucccuuuuuaauuagaaauuaaaacaguuaauuuaauuaaagaguaggguuuuuuuucaguauucuugguuaauauuuaauuucaacuauuuaugagauguaucuuuugcucucucuugcucucuuauuuguaccgguuuuuguauauaaaauucauguuuccaaucucucucucccugaucggugacagucacuagcuuaucuugaacagauauuuaauuuugcuaacacucagcucugcccuccccgauccccuggcuccccagcacacauuccuuugaaauaagguuucaauauacaucuacauacuauauauauauuuggcaacuuguauuuguguguauauauauauauauauguuuauguauauaugugauucugauaaaauagacauugcuauucuguuuuuuauauguaaaaacaaaacaagaaaaaauagagaauucuacauacuaaaucucucuccuuuuuuaauuuuaauauuuguuaucauuuauuuauuggugcuacuguuuauccguaauaauuguggggaaaagauauuaacaucacgucuuugucucuagugcaguuuuucgagauauuccguaguacauauuuauuuuuaaacaacgacaaagaaauacagauauaucuuaaaaaaaaaaaagcauuuuguauuaaagaauuuaauucugaucucaaaaaaaaaaaaaaaaaa

SEQ ID NO: 109 - protein, 371 aa, Homo sapiens
  VEGFA
MTDRQTDTAPSPSYHLLPGRRRTVDAAASRGQGPEPAPGGGVEGVGARGVALKLFVQLLGCSRFGGAVVRAGEAEPSGAARSASSGREEPQPEEGEEEEEKEEERGPQWRLGARKPGSWTGEAAVCADSAPAARAPQALARASGRGGRVARRGAEESGPPHSPSRRGSASRAGPGRASETMNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRCDKPRR

SEQ ID NO: 110 - RNA, 3519 nt, Homo sapiens
  VEGFA isoform e
ucgcggaggcuuggggcagccggguagcucggaggucguggcgcugggggcuagcaccagcgcucugucgggaggcgcagcgguuagguggaccggucagcggacucaccggccagggcgcucggugcuggaauuugauauucauugauccggguuuuaucccucuucuuuuuucuuaaacauuuuuuuuuaaaacuguauuguuucucguuuuaauuuauuuuugcuugccauuccccacuugaaucgggccgacggcuuggggagauugcucuacuuccccaaaucacuguggauuuuggaaaccagcagaaagaggaaagagguagcaagagcuccagagagaagucgaggaagagagagacggggucagagagagcgcgcgggcgugcgagcagcgaaagcgacaggggcaaagugagugaccugcuuuugggggugaccgccggagcgcggcgugagcccucccccuugggaucccgcagcugaccagucgcgcugacggacagacagacagacaccgcccccagccccagcuaccaccuccuccccggccggcggcggacaguggacgcggcggcgagccgcgggcaggggccggagcccgcgcccggaggcgggguggagggggucggggcucgcggcgucgcacugaaacuuuucguccaacuucugggcuguucucgcuucggaggagccgugguccgcgcgggggaagccgagccgagcggagccgcgagaagugcuagcucgggccgggaggagccgcagccggaggagggggaggaggaagaagagaaggaagaggagagggggccgcaguggcgacucggcgcucggaagccgggcucauggacgggugaggcggcggugugcgcagacagugcuccagccgcgcgcgcuccccaggcccuggcccgggccucgggccggggaggaagaguagcucgccgaggcgccgaggagagcgggccgccccacagcccgagccggagagggagcgcgagccgcgccggccccggucgggccuccgaaaccaugaacuuucugcugucuugggugcauuggagccuugccuugcugcucuaccuccaccaugccaaguggucccaggcugcacccauggcagaaggaggagggcagaaucaucacgaaguggugaaguucauggaugucuaucagcgcagcuacugccauccaaucgagacccugguggacaucuuccaggaguacccugaugagaucgaguacaucuucaagccauccugugugccccugaugcgaugcgggggcugcugcaaugacgagggccuggagugugugcccacugaggaguccaacaucaccaugcagauuaugcggaucaaaccucaccaaggccagcacauaggagagaugagcuuccuacagcacaacaaaugugaaugcagaccaaagaaagauagagcaagacaagaaaaucccugugggccuugcucagagcggagaaagcauuuguuuguacaagauccgcagacguguaaauguuccugcaaaaacacagacucgcguugcaagaugugacaagccgaggcggugagccgggcaggaggaaggagccucccucaggguuucgggaaccagaucucucaccaggaaagacugauacagaacgaucgauacagaaaccacgcugccgccaccacaccaucaccaucgacagaacaguccuuaauccagaaaccugaaaugaaggaagaggagacucugcgcagagcacuuuggguccggagggcgagacuccggcggaagcauucccgggcgggugacccagcacggucccucuuggaauuggauucgccauuuuauuuuucuugcugcuaaaucaccgagcccggaagauuagagaguuuuauuucugggauuccuguagacacacccacccacauacauacauuuauauauauauauauuauauauauauaaaaauaaauaucucuauuuuauauauauaaaauauauauauucuuuuuuuaaauuaacagugcuaauguuauuggugucuucacuggauguauuugacugcuguggacuugaguugggaggggaauguucccacucagauccugacagggaagaggaggagaugagagacucuggcaugaucuuuuuuuugucccacuugguggggccaggguccucuccccugcccaggaaugugcaaggccagggcaugggggcaaauaugacccaguuuugggaacaccgacaaacccagcccuggcgcugagccucucuaccccaggucagacggacagaaagacagaucacagguacagggaugaggacaccggcucugaccaggaguuuggggagcuucaggacauugcugugcuuuggggauucccuccacaugcugcacgcgcaucucgcccccaggggcacugccuggaagauucaggagccugggcggccuucgcuuacucucaccugcuucugaguugcccaggagaccacuggcagaugucccggcgaagagaagagacacauuguuggaagaagcagcccaugacagcuccccuuccugggacucgcccucauccucuuccugcuccccuuccuggggugcagccuaaaaggaccuauguccucacaccauugaaaccacuaguucuguccccccaggagaccugguugugugugugugagugguugaccuuccuccauccccugguccuucccuucccuucccgaggcacagagagacagggcaggauccacgugcccauuguggaggcagagaaaagagaaaguguuuuauauacgguacuuauuuaauaucccuuuuuaauuagaaauuaaaacaguuaauuuaauuaaagaguaggguuuuuuuucaguauucuugguuaauauuuaauuucaacuauuuaugagauguaucuuuugcucucucuugcucucuuauuuguaccgguuuuuguauauaaaauucauguuuccaaucucucucucccugaucggugacagucacuagcuuaucuugaacagauauuuaauuuugcuaacacucagcucugcccuccccgauccccuggcuccccagcacacauuccuuugaaauaagguuucaauauacaucuacauacuauauauauauuuggcaacuuguauuuguguguauauauauauauauauguuuauguauauaugugauucugauaaaauagacauugcuauucuguuuuuuauauguaaaaacaaaacaagaaaaaauagagaauucuacauacuaaaucucucuccuuuuuuaauuuuaauauuuguuaucauuuauuuauuggugcuacuguuuauccguaauaauuguggggaaaagauauuaacaucacgucuuugucucuagugcaguuuuucgagauauuccguaguacauauuuauuuuuaaacaacgacaaagaaauacagauauaucuuaaaaaaaaaaaagcauuuuguauuaaagaauuuaauucugaucucaaaaaaaaaaaaaaaaaa

SEQ ID NO: 111 - protein, 354 aa, Homo sapiens
  VEGFA
MTDRQTDTAPSPSYHLLPGRRRTVDAAASRGQGPEPAPGGGVEGVGARGVALKLFVQLLGCSRFGGAVVRAGEAEPSGAARSASSGREEPQPEEGEEEEEKEEERGPQWRLGARKPGSWTGEAAVCADSAPAARAPQALARASGRGGRVARRGAEESGPPHSPSRRGSASRAGPGRASETMNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKM

SEQ ID NO: 112 - RNA, 3422 nt, Homo sapiens
  VEGFA isoform f
ucgcggaggcuuggggcagccggguagcucggaggucguggcgcugggggcuagcaccagcgcucugucgggaggcgcagcgguuagguggaccggucagcggacucaccggccagggcgcucggugcuggaauuugauauucauugauccggguuuuaucccucuucuuuuuucuuaaacauuuuuuuuuaaaacuguauuguuucucguuuuaauuuauuuuugcuugccauuccccacuugaaucgggccgacggcuuggggagauugcucuacuuccccaaaucacuguggauuuuggaaaccagcagaaagaggaaagagguagcaagagcuccagagagaagucgaggaagagagagacggggucagagagagcgcgcgggcgugcgagcagcgaaagcgacaggggcaaagugagugaccugcuuuugggggugaccgccggagcgcggcgugagcccucccccuugggaucccgcagcugaccagucgcgcugacggacagacagacagacaccgcccccagccccagcuaccaccuccuccccggccggcggcggacaguggacgcggcggcgagccgcgggcaggggccggagcccgcgcccggaggcgggguggagggggucggggcucgcggcgucgcacugaaacuuuucguccaacuucugggcuguucucgcuucggaggagccgugguccgcgcgggggaagccgagccgagcggagccgcgagaagugcuagcucgggccgggaggagccgcagccggaggagggggaggaggaagaagagaaggaagaggagagggggccgcaguggcgacucggcgcucggaagccgggcucauggacgggugaggcggcggugugcgcagacagugcuccagccgcgcgcgcuccccaggcccuggcccgggccucgggccggggaggaagaguagcucgccgaggcgccgaggagagcgggccgccccacagcccgagccggagagggagcgcgagccgcgccggccccggucgggccuccgaaaccaugaacuuucugcugucuugggugcauuggagccuugccuugcugcucuaccuccaccaugccaaguggucccaggcugcacccauggcagaaggaggagggcagaaucaucacgaaguggugaaguucauggaugucuaucagcgcagcuacugccauccaaucgagacccugguggacaucuuccaggaguacccugaugagaucgaguacaucuucaagccauccugugugccccugaugcgaugcgggggcugcugcaaugacgagggccuggagugugugcccacugaggaguccaacaucaccaugcagauuaugcggaucaaaccucaccaaggccagcacauaggagagaugagcuuccuacagcacaacaaaugugaaugcagaccaaagaaagauagagcaagacaagaaaaaugugacaagccgaggcggugagccgggcaggaggaaggagccucccucaggguuucgggaaccagaucucucaccaggaaagacugauacagaacgaucgauacagaaaccacgcugccgccaccacaccaucaccaucgacagaacaguccuuaauccagaaaccugaaaugaaggaagaggagacucugcgcagagcacuuuggguccggagggcgagacuccggcggaagcauucccgggcgggugacccagcacggucccucuuggaauuggauucgccauuuuauuuuucuugcugcuaaaucaccgagcccggaagauuagagaguuuuauuucugggauuccuguagacacacccacccacauacauacauuuauauauauauauauuauauauauauaaaaauaaauaucucuauuuuauauauauaaaauauauauauucuuuuuuuaaauuaacagugcuaauguuauuggugucuucacuggauguauuugacugcuguggacuugaguugggaggggaauguucccacucagauccugacagggaagaggaggagaugagagacucuggcaugaucuuuuuuuugucccacuugguggggccaggguccucuccccugcccaggaaugugcaaggccagggcaugggggcaaauaugacccaguuuugggaacaccgacaaacccagcccuggcgcugagccucucuaccccaggucagacggacagaaagacagaucacagguacagggaugaggacaccggcucugaccaggaguuuggggagcuucaggacauugcugugcuuuggggauucccuccacaugcugcacgcgcaucucgcccccaggggcacugccuggaagauucaggagccugggcggccuucgcuuacucucaccugcuucugaguugcccaggagaccacuggcagaugucccggcgaagagaagagacacauuguuggaagaagcagcccaugacagcuccccuuccugggacucgcccucauccucuuccugcuccccuuccuggggugcagccuaaaaggaccuauguccucacaccauugaaaccacuaguucuguccccccaggagaccugguugugugugugugagugguugaccuuccuccauccccugguccuucccuucccuucccgaggcacagagagacagggcaggauccacgugcccauuguggaggcagagaaaagagaaaguguuuuauauacgguacuuauuuaauaucccuuuuuaauuagaaauuaaaacaguuaauuuaauuaaagaguaggguuuuuuuucaguauucuugguuaauauuuaauuucaacuauuuaugagauguaucuuuugcucucucuugcucucuuauuuguaccgguuuuuguauauaaaauucauguuuccaaucucucucucccugaucggugacagucacuagcuuaucuugaacagauauuuaauuuugcuaacacucagcucugcccuccccgauccccuggcuccccagcacacauuccuuugaaauaagguuucaauauacaucuacauacuauauauauauuuggcaacuuguauuuguguguauauauauauauauauguuuauguauauaugugauucugauaaaauagacauugcuauucuguuuuuuauauguaaaaacaaaacaagaaaaaauagagaauucuacauacuaaaucucucuccuuuuuuaauuuuaauauuuguuaucauuuauuuauuggugcuacuguuuauccguaauaauuguggggaaaagauauuaacaucacgucuuugucucuagugcaguuuuucgagauauuccguaguacauauuuauuuuuaaacaacgacaaagaaauacagauauaucuuaaaaaaaaaaaagcauuuuguauuaaagaauuuaauucugaucucaaaaaaaaaaaaaaaaaa

SEQ ID NO: 113 - protein, 327 aa, Homo sapiens
  VEGFA
MTDRQTDTAPSPSYHLLPGRRRTVDAAASRGQGPEPAPGGGVEGVGARGVALKLFVQLLGCSRFGGAVVRAGEAEPSGAARSASSGREEPQPEEGEEEEEKEEERGPQWRLGARKPGSWTGEAAVCADSAPAARAPQALARASGRGGRVARRGAEESGPPHSPSRRGSASRAGPGRASETMNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKCDKPRR

SEQ ID NO: 114 - RNA, 3488 nt, Homo sapiens
  VEGFA isoform g
ucgcggaggcuuggggcagccggguagcucggaggucguggcgcugggggcuagcaccagcgcucugucgggaggcgcagcgguuagguggaccggucagcggacucaccggccagggcgcucggugcuggaauuugauauucauugauccggguuuuaucccucuucuuuuuucuuaaacauuuuuuuuuaaaacuguauuguuucucguuuuaauuuauuuuugcuugccauuccccacuugaaucgggccgacggcuuggggagauugcucuacuuccccaaaucacuguggauuuuggaaaccagcagaaagaggaaagagguagcaagagcuccagagagaagucgaggaagagagagacggggucagagagagcgcgcgggcgugcgagcagcgaaagcgacaggggcaaagugagugaccugcuuuugggggugaccgccggagcgcggcgugagcccucccccuugggaucccgcagcugaccagucgcgcugacggacagacagacagacaccgcccccagccccagcuaccaccuccuccccggccggcggcggacaguggacgcggcggcgagccgcgggcaggggccggagcccgcgcccggaggcgggguggagggggucggggcucgcggcgucgcacugaaacuuuucguccaacuucugggcuguucucgcuucggaggagccgugguccgcgcgggggaagccgagccgagcggagccgcgagaagugcuagcucgggccgggaggagccgcagccggaggagggggaggaggaagaagagaaggaagaggagagggggccgcaguggcgacucggcgcucggaagccgggcucauggacgggugaggcggcggugugcgcagacagugcuccagccgcgcgcgcuccccaggcccuggcccgggccucgggccggggaggaagaguagcucgccgaggcgccgaggagagcgggccgccccacagcccgagccggagagggagcgcgagccgcgccggccccggucgggccuccgaaaccaugaacuuucugcugucuugggugcauuggagccuugccuugcugcucuaccuccaccaugccaaguggucccaggcugcacccauggcagaaggaggagggcagaaucaucacgaaguggugaaguucauggaugucuaucagcgcagcuacugccauccaaucgagacccugguggacaucuuccaggaguacccugaugagaucgaguacaucuucaagccauccugugugccccugaugcgaugcgggggcugcugcaaugacgagggccuggagugugugcccacugaggaguccaacaucaccaugcagauuaugcggaucaaaccucaccaaggccagcacauaggagagaugagcuuccuacagcacaacaaaugugaaugcagaccaaagaaagauagagcaagacaagaaaaucccugugggccuugcucagagcggagaaagcauuuguuuguacaagauccgcagacguguaaauguuccugcaaaaacacagacucgcguugcaaggcgaggcagcuugaguuaaacgaacguacuugcagaucucucaccaggaaagacugauacagaacgaucgauacagaaaccacgcugccgccaccacaccaucaccaucgacagaacaguccuuaauccagaaaccugaaaugaaggaagaggagacucugcgcagagcacuuuggguccggagggcgagacuccggcggaagcauucccgggcgggugacccagcacggucccucuuggaauuggauucgccauuuuauuuuucuugcugcuaaaucaccgagcccggaagauuagagaguuuuauuucugggauuccuguagacacacccacccacauacauacauuuauauauauauauauuauauauauauaaaaauaaauaucucuauuuuauauauauaaaauauauauauucuuuuuuuaaauuaacagugcuaauguuauuggugucuucacuggauguauuugacugcuguggacuugaguugggaggggaauguucccacucagauccugacagggaagaggaggagaugagagacucuggcaugaucuuuuuuuugucccacuugguggggccaggguccucuccccugcccaggaaugugcaaggccagggcaugggggcaaauaugacccaguuuugggaacaccgacaaacccagcccuggcgcugagccucucuaccccaggucagacggacagaaagacagaucacagguacagggaugaggacaccggcucugaccaggaguuuggggagcuucaggacauugcugugcuuuggggauucccuccacaugcugcacgcgcaucucgcccccaggggcacugccuggaagauucaggagccugggcggccuucgcuuacucucaccugcuucugaguugcccaggagaccacuggcagaugucccggcgaagagaagagacacauuguuggaagaagcagcccaugacagcuccccuuccugggacucgcccucauccucuuccugcuccccuuccuggggugcagccuaaaaggaccuauguccucacaccauugaaaccacuaguucuguccccccaggagaccugguugugugugugugagugguugaccuuccuccauccccugguccuucccuucccuucccgaggcacagagagacagggcaggauccacgugcccauuguggaggcagagaaaagagaaaguguuuuauauacgguacuuauuuaauaucccuuuuuaauuagaaauuaaaacaguuaauuuaauuaaagaguaggguuuuuuuucaguauucuugguuaauauuuaauuucaacuauuuaugagauguaucuuuugcucucucuugcucucuuauuuguaccgguuuuuguauauaaaauucauguuuccaaucucucucucccugaucggugacagucacuagcuuaucuugaacagauauuuaauuuugcuaacacucagcucugcccuccccgauccccuggcuccccagcacacauuccuuugaaauaagguuucaauauacaucuacauacuauauauauauuuggcaacuuguauuuguguguauauauauauauauauguuuauguauauaugugauucugauaaaauagacauugcuauucuguuuuuuauauguaaaaacaaaacaagaaaaaauagagaauucuacauacuaaaucucucuccuuuuuuaauuuuaauauuuguuaucauuuauuuauuggugcuacuguuuauccguaauaauuguggggaaaagauauuaacaucacgucuuugucucuagugcaguuuuucgagauauuccguaguacauauuuauuuuuaaacaacgacaaagaaauacagauauaucuuaaaaaaaaaaaagcauuuuguauuaaagaauuuaauucugaucucaaaaaaaaaaaaaaaaaa

SEQ ID NO: 115 - protein, 371 aa, Homo sapiens
  VEGFA
MTDRQTDTAPSPSYHLLPGRRRTVDAAASRGQGPEPAPGGGVEGVGARGVALKLFVQLLGCSRFGGAVVRAGEAEPSGAARSASSGREEPQPEEGEEEEEKEEERGPQWRLGARKPGSWTGEAAVCADSAPAARAPQALARASGRGGRVARRGAEESGPPHSPSRRGSASRAGPGRASETMNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRSLTRKD

SEQ ID NO: 116 - RNA, 3392 nt, Homo sapiens
  VEGFA isoform h
ucgcggaggcuuggggcagccggguagcucggaggucguggcgcugggggcuagcaccagcgcucugucgggaggcgcagcgguuagguggaccggucagcggacucaccggccagggcgcucggugcuggaauuugauauucauugauccggguuuuaucccucuucuuuuuucuuaaacauuuuuuuuuaaaacuguauuguuucucguuuuaauuuauuuuugcuugccauuccccacuugaaucgggccgacggcuuggggagauugcucuacuuccccaaaucacuguggauuuuggaaaccagcagaaagaggaaagagguagcaagagcuccagagagaagucgaggaagagagagacggggucagagagagcgcgcgggcgugcgagcagcgaaagcgacaggggcaaagugagugaccugcuuuugggggugaccgccggagcgcggcgugagcccucccccuugggaucccgcagcugaccagucgcgcugacggacagacagacagacaccgcccccagccccagcuaccaccuccuccccggccggcggcggacaguggacgcggcggcgagccgcgggcaggggccggagcccgcgcccggaggcgggguggagggggucggggcucgcggcgucgcacugaaacuuuucguccaacuucugggcuguucucgcuucggaggagccgugguccgcgcgggggaagccgagccgagcggagccgcgagaagugcuagcucgggccgggaggagccgcagccggaggagggggaggaggaagaagagaaggaagaggagagggggccgcaguggcgacucggcgcucggaagccgggcucauggacgggugaggcggcggugugcgcagacagugcuccagccgcgcgcgcuccccaggcccuggcccgggccucgggccggggaggaagaguagcucgccgaggcgccgaggagagcgggccgccccacagcccgagccggagagggagcgcgagccgcgccggccccggucgggccuccgaaaccaugaacuuucugcugucuugggugcauuggagccuugccuugcugcucuaccuccaccaugccaaguggucccaggcugcacccauggcagaaggaggagggcagaaucaucacgaaguggugaaguucauggaugucuaucagcgcagcuacugccauccaaucgagacccugguggacaucuuccaggaguacccugaugagaucgaguacaucuucaagccauccugugugccccugaugcgaugcgggggcugcugcaaugacgagggccuggagugugugcccacugaggaguccaacaucaccaugcagauuaugcggaucaaaccucaccaaggccagcacauaggagagaugagcuuccuacagcacaacaaaugugaaugcagaugugacaagccgaggcggugagccgggcaggaggaaggagccucccucaggguuucgggaaccagaucucucaccaggaaagacugauacagaacgaucgauacagaaaccacgcugccgccaccacaccaucaccaucgacagaacaguccuuaauccagaaaccugaaaugaaggaagaggagacucugcgcagagcacuuuggguccggagggcgagacuccggcggaagcauucccgggcgggugacccagcacggucccucuuggaauuggauucgccauuuuauuuuucuugcugcuaaaucaccgagcccggaagauuagagaguuuuauuucugggauuccuguagacacacccacccacauacauacauuuauauauauauauauuauauauauauaaaaauaaauaucucuauuuuauauauauaaaauauauauauucuuuuuuuaaauuaacagugcuaauguuauuggugucuucacuggauguauuugacugcuguggacuugaguugggaggggaauguucccacucagauccugacagggaagaggaggagaugagagacucuggcaugaucuuuuuuuugucccacuugguggggccaggguccucuccccugcccaggaaugugcaaggccagggcaugggggcaaauaugacccaguuuugggaacaccgacaaacccagcccuggcgcugagccucucuaccccaggucagacggacagaaagacagaucacagguacagggaugaggacaccggcucugaccaggaguuuggggagcuucaggacauugcugugcuuuggggauucccuccacaugcugcacgcgcaucucgcccccaggggcacugccuggaagauucaggagccugggcggccuucgcuuacucucaccugcuucugaguugcccaggagaccacuggcagaugucccggcgaagagaagagacacauuguuggaagaagcagcccaugacagcuccccuuccugggacucgcccucauccucuuccugcuccccuuccuggggugcagccuaaaaggaccuauguccucacaccauugaaaccacuaguucuguccccccaggagaccugguugugugugugugagugguugaccuuccuccauccccugguccuucccuucccuucccgaggcacagagagacagggcaggauccacgugcccauuguggaggcagagaaaagagaaaguguuuuauauacgguacuuauuuaauaucccuuuuuaauuagaaauuaaaacaguuaauuuaauuaaagaguaggguuuuuuuucaguauucuugguuaauauuuaauuucaacuauuuaugagauguaucuuuugcucucucuugcucucuuauuuguaccgguuuuuguauauaaaauucauguuuccaaucucucucucccugaucggugacagucacuagcuuaucuugaacagauauuuaauuuugcuaacacucagcucugcccuccccgauccccuggcuccccagcacacauuccuuugaaauaagguuucaauauacaucuacauacuauauauauauuuggcaacuuguauuuguguguauauauauauauauauguuuauguauauaugugauucugauaaaauagacauugcuauucuguuuuuuauauguaaaaacaaaacaagaaaaaauagagaauucuacauacuaaaucucucuccuuuuuuaauuuuaauauuuguuaucauuuauuuauuggugcuacuguuuauccguaauaauuguggggaaaagauauuaacaucacgucuuugucucuagugcaguuuuucgagauauuccguaguacauauuuauuuuuaaacaacgacaaagaaauacagauauaucuuaaaaaaaaaaaagcauuuuguauuaaagaauuuaauucugaucucaaaaaaaaaaaaaaaaaa

SEQ ID NO: 117 - protein, 317 aa, Homo sapiens
  VEGFA
MTDRQTDTAPSPSYHLLPGRRRTVDAAASRGQGPEPAPGGGVEGVGARGVALKLFVQLLGCSRFGGAVVRAGEAEPSGAARSASSGREEPQPEEGEEEEEKEEERGPQWRLGARKPGSWTGEAAVCADSAPAARAPQALARASGRGGRVARRGAEESGPPHSPSRRGSASRAGPGRASETMNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRCDKPRR

SEQ ID NO: 118 - RNA, 3677 nt, Homo sapiens; identical to SEQ ID NO: 104
  VEGFA isofrom i precursor

SEQ ID NO: 119 - protein, 232 aa, Homo sapiens
  VEGFA
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

SEQ ID NO: 120 - RNA, 3626 nt, Homo sapiens
  VEGFA isoform j precursor
ucgcggaggcuuggggcagccggguagcucggaggucguggcgcugggggcuagcaccagcgcucugucgggaggcgcagcgguuagguggaccggucagcggacucaccggccagggcgcucggugcuggaauuugauauucauugauccggguuuuaucccucuucuuuuuucuuaaacauuuuuuuuuaaaacuguauuguuucucguuuuaauuuauuuuugcuugccauuccccacuugaaucgggccgacggcuuggggagauugcucuacuuccccaaaucacuguggauuuuggaaaccagcagaaagaggaaagagguagcaagagcuccagagagaagucgaggaagagagagacggggucagagagagcgcgcgggcgugcgagcagcgaaagcgacaggggcaaagugagugaccugcuuuugggggugaccgccggagcgcggcgugagcccucccccuugggaucccgcagcugaccagucgcgcugacggacagacagacagacaccgcccccagccccagcuaccaccuccuccccggccggcggcggacaguggacgcggcggcgagccgcgggcaggggccggagcccgcgcccggaggcgggguggagggggucggggcucgcggcgucgcacugaaacuuuucguccaacuucugggcuguucucgcuucggaggagccgugguccgcgcgggggaagccgagccgagcggagccgcgagaagugcuagcucgggccgggaggagccgcagccggaggagggggaggaggaagaagagaaggaagaggagagggggccgcaguggcgacucggcgcucggaagccgggcucauggacgggugaggcggcggugugcgcagacagugcuccagccgcgcgcgcuccccaggcccuggcccgggccucgggccggggaggaagaguagcucgccgaggcgccgaggagagcgggccgccccacagcccgagccggagagggagcgcgagccgcgccggccccggucgggccuccgaaaccaugaacuuucugcugucuugggugcauuggagccuugccuugcugcucuaccuccaccaugccaaguggucccaggcugcacccauggcagaaggaggagggcagaaucaucacgaaguggugaaguucauggaugucuaucagcgcagcuacugccauccaaucgagacccugguggacaucuuccaggaguacccugaugagaucgaguacaucuucaagccauccugugugccccugaugcgaugcgggggcugcugcaaugacgagggccuggagugugugcccacugaggaguccaacaucaccaugcagauuaugcggaucaaaccucaccaaggccagcacauaggagagaugagcuuccuacagcacaacaaaugugaaugcagaccaaagaaagauagagcaagacaagaaaaaaaaucaguucgaggaaagggaaaggggcaaaaacgaaagcgcaagaaaucccgguauaaguccuggagcguucccugugggccuugcucagagcggagaaagcauuuguuuguacaagauccgcagacguguaaauguuccugcaaaaacacagacucgcguugcaaggcgaggcagcuugaguuaaacgaacguacuugcagaugugacaagccgaggcggugagccgggcaggaggaaggagccucccucaggguuucgggaaccagaucucucaccaggaaagacugauacagaacgaucgauacagaaaccacgcugccgccaccacaccaucaccaucgacagaacaguccuuaauccagaaaccugaaaugaaggaagaggagacucugcgcagagcacuuuggguccggagggcgagacuccggcggaagcauucccgggcgggugacccagcacggucccucuuggaauuggauucgccauuuuauuuuucuugcugcuaaaucaccgagcccggaagauuagagaguuuuauuucugggauuccuguagacacacccacccacauacauacauuuauauauauauauauuauauauauauaaaaauaaauaucucuauuuuauauauauaaaauauauauauucuuuuuuuaaauuaacagugcuaauguuauuggugucuucacuggauguauuugacugcuguggacuugaguugggaggggaauguucccacucagauccugacagggaagaggaggagaugagagacucuggcaugaucuuuuuuuugucccacuugguggggccaggguccucuccccugcccaggaaugugcaaggccagggcaugggggcaaauaugacccaguuuugggaacaccgacaaacccagcccuggcgcugagccucucuaccccaggucagacggacagaaagacagaucacagguacagggaugaggacaccggcucugaccaggaguuuggggagcuucaggacauugcugugcuuuggggauucccuccacaugcugcacgcgcaucucgcccccaggggcacugccuggaagauucaggagccugggcggccuucgcuuacucucaccugcuucugaguugcccaggagaccacuggcagaugucccggcgaagagaagagacacauuguuggaagaagcagcccaugacagcuccccuuccugggacucgcccucauccucuuccugcuccccuuccuggggugcagccuaaaaggaccuauguccucacaccauugaaaccacuaguucuguccccccaggagaccugguugugugugugugagugguugaccuuccuccauccccugguccuucccuucccuucccgaggcacagagagacagggcaggauccacgugcccauuguggaggcagagaaaagagaaaguguuuuauauacgguacuuauuuaauaucccuuuuuaauuagaaauuaaaacaguuaauuuaauuaaagaguaggguuuuuuuucaguauucuugguuaauauuuaauuucaacuauuuaugagauguaucuuuugcucucucuugcucucuuauuuguaccgguuuuuguauauaaaauucauguuuccaaucucucucucccugaucggugacagucacuagcuuaucuugaacagauauuuaauuuugcuaacacucagcucugcccuccccgauccccuggcuccccagcacacauuccuuugaaauaagguuucaauauacaucuacauacuauauauauauuuggcaacuuguauuuguguguauauauauauauauauguuuauguauauaugugauucugauaaaauagacauugcuauucuguuuuuuauauguaaaaacaaaacaagaaaaaauagagaauucuacauacuaaaucucucuccuuuuuuaauuuuaauauuuguuaucauuuauuuauuggugcuacuguuuauccguaauaauuguggggaaaagauauuaacaucacgucuuugucucuagugcaguuuuucgagauauuccguaguacauauuuauuuuuaaacaacgacaaagaaauacagauauaucuuaaaaaaaaaaaagcauuuuguauuaaagaauuuaauucugaucucaaaaaaaaaaaaaaaaaa

SEQ ID NO: 121 - protein, 215 aa, Homo sapiens
  VEGFA
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVPCGPCSERRKHLFVQDPQTCKCSCKNTDSRCKARQLELNERTCRCDKPRR

SEQ ID NO: 122 - RNA, 3608 nt, Homo sapiens
  VEGFA isoform k precursor
ucgcggaggcuuggggcagccggguagcucggaggucguggcgcugggggcuagcaccagcgcucugucgggaggcgcagcgguuagguggaccggucagcggacucaccggccagggcgcucggugcuggaauuugauauucauugauccggguuuuaucccucuucuuuuuucuuaaacauuuuuuuuuaaaacuguauuguuucucguuuuaauuuauuuuugcuugccauuccccacuugaaucgggccgacggcuuggggagauugcucuacuuccccaaaucacuguggauuuuggaaaccagcagaaagaggaaagagguagcaagagcuccagagagaagucgaggaagagagagacggggucagagagagcgcgcgggcgugcgagcagcgaaagcgacaggggcaaagugagugaccugcuuuugggggugaccgccggagcgcggcgugagcccucccccuugggaucccgcagcugaccagucgcgcugacggacagacagacagacaccgcccccagccccagcuaccaccuccuccccggccggcggcggacaguggacgcggcggcgagccgcgggcaggggccggagcccgcgcccggaggcgggguggagggggucggggcucgcggcgucgcacugaaacuuuucguccaacuucugggcuguucucgcuucggaggagccgugguccgcgcgggggaagccgagccgagcggagccgcgagaagugcuagcucgggccgggaggagccgcagccggaggagggggaggaggaagaagagaaggaagaggagagggggccgcaguggcgacucggcgcucggaagccgggcucauggacgggugaggcggcggugugcgcagacagugcuccagccgcgcgcgcuccccaggcccuggcccgggccucgggccggggaggaagaguagcucgccgaggcgccgaggagagcgggccgccccacagcccgagccggagagggagcgcgagccgcgccggccccggucgggccuccgaaaccaugaacuuucugcugucuugggugcauuggagccuugccuugcugcucuaccuccaccaugccaaguggucccaggcugcacccauggcagaaggaggagggcagaaucaucacgaaguggugaaguucauggaugucuaucagcgcagcuacugccauccaaucgagacccugguggacaucuuccaggaguacccugaugagaucgaguacaucuucaagccauccugugugccccugaugcgaugcgggggcugcugcaaugacgagggccuggagugugugcccacugaggaguccaacaucaccaugcagauuaugcggaucaaaccucaccaaggccagcacauaggagagaugagcuuccuacagcacaacaaaugugaaugcagaccaaagaaagauagagcaagacaagaaaaaaaaucaguucgaggaaagggaaaggggcaaaaacgaaagcgcaagaaaucccgucccugugggccuugcucagagcggagaaagcauuuguuuguacaagauccgcagacguguaaauguuccugcaaaaacacagacucgcguugcaaggcgaggcagcuugaguuaaacgaacguacuugcagaugugacaagccgaggcggugagccgggcaggaggaaggagccucccucaggguuucgggaaccagaucucucaccaggaaagacugauacagaacgaucgauacagaaaccacgcugccgccaccacaccaucaccaucgacagaacaguccuuaauccagaaaccugaaaugaaggaagaggagacucugcgcagagcacuuuggguccggagggcgagacuccggcggaagcauucccgggcgggugacccagcacggucccucuuggaauuggauucgccauuuuauuuuucuugcugcuaaaucaccgagcccggaagauuagagaguuuuauuucugggauuccuguagacacacccacccacauacauacauuuauauauauauauauuauauauauauaaaaauaaauaucucuauuuuauauauauaaaauauauauauucuuuuuuuaaauuaacagugcuaauguuauuggugucuucacuggauguauuugacugcuguggacuugaguugggaggggaauguucccacucagauccugacagggaagaggaggagaugagagacucuggcaugaucuuuuuuuugucccacuugguggggccaggguccucuccccugcccaggaaugugcaaggccagggcaugggggcaaauaugacccaguuuugggaacaccgacaaacccagcccuggcgcugagccucucuaccccaggucagacggacagaaagacagaucacagguacagggaugaggacaccggcucugaccaggaguuuggggagcuucaggacauugcugugcuuuggggauucccuccacaugcugcacgcgcaucucgcccccaggggcacugccuggaagauucaggagccugggcggccuucgcuuacucucaccugcuucugaguugcccaggagaccacuggcagaugucccggcgaagagaagagacacauuguuggaagaagcagcccaugacagcuccccuuccugggacucgcccucauccucuuccugcuccccuuccuggggugcagccuaaaaggaccuauguccucacaccauugaaaccacuaguucuguccccccaggagaccugguugugugugugugagugguugaccuuccuccauccccugguccuucccuucccuucccgaggcacagagagacagggcaggauccacgugcccauuguggaggcagagaaaagagaaaguguuuuauauacgguacuuauuuaauaucccuuuuuaauuagaaauuaaaacaguuaauuuaauuaaagaguaggguuuuuuuucaguauucuugguuaauauuuaauuucaacuauuuaugagauguaucuuuugcucucucuugcucucuuauuuguaccgguuuuuguauauaaaauucauguuuccaaucucucucucccugaucggugacagucacuagcuuaucuugaacagauauuuaauuuugcuaacacucagcucugcccuccccgauccccuggcuccccagcacacauuccuuugaaauaagguuucaauauacaucuacauacuauauauauauuuggcaacuuguauuuguguguauauauauauauauauguuuauguauauaugugauucugauaaaauagacauugcuauucuguuuuuuauauguaaaaacaaaacaagaaaaaauagagaauucuacauacuaaaucucucuccuuuuuuaauuuuaauauuuguuaucauuuauuuauuggugcuacuguuuauccguaauaauuguggggaaaagauauuaacaucacgucuuugucucuagugcaguuuuucgagauauuccguaguacauauuuauuuuuaaacaacgacaaagaaauacagauauaucuuaaaaaaaaaaaagcauuuuguauuaaagaauuuaauucugaucucaaaaaaaaaaaaaaaaaa

SEQ ID NO: 123 - protein, 209 aa, Homo sapiens
  VEGFA
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRPCGPCSERRKHLFVQDPQTCKCSCKNTDSRCKARQLELNERTCRCDKPRR

SEQ ID NO: 124 - RNA, 3554 nt, Homo sapiens; identical to SEQ ID NO: 108
  VEGFA isoform l precursor

SEQ ID NO: 125 - protein, 191 aa, Homo sapiens
  VEGFA
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRCDKPRR

SEQ ID NO: 126 - RNA, 3519 nt, Homo sapiens; identical to SEQ ID NO: 110
  VEGFA isoform m precursor

SEQ ID NO: 127 - protein, 174 aa, Homo sapiens
  VEGFA
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKM

SEQ ID NO: 128 - RNA, 3422 nt, Homo sapiens; identical to SEQ ID NO: 112
  VEGFA isoform n precursor

SEQ ID NO: 129 - protein, 147 aa, Homo sapiens
  VEGFA
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKCDKPRR

SEQ ID NO: 130 - RNA, 3488 nt, Homo sapiens; identical to SEQ ID NO: 114
  VEGFA isoform o precursor

SEQ ID NO: 131 - protein, 191 aa, Homo sapiens
  VEGFA
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRSLTRKD

SEQ ID NO: 132 - RNA, 3392 nt, Homo sapiens; identical to SEQ ID NO: 116
  VEGFA isoform p precursor

SEQ ID NO: 133 - protein, 137 aa, Homo sapiens
  VEGFA
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRCDKPRR

SEQ ID NO: 134 - RNA, 3494 nt, Homo sapiens
  VEGFA isoform q precursor
ucgcggaggcuuggggcagccggguagcucggaggucguggcgcugggggcuagcaccagcgcucugucgggaggcgcagcgguuagguggaccggucagcggacucaccggccagggcgcucggugcuggaauuugauauucauugauccggguuuuaucccucuucuuuuuucuuaaacauuuuuuuuuaaaacuguauuguuucucguuuuaauuuauuuuugcuugccauuccccacuugaaucgggccgacggcuuggggagauugcucuacuuccccaaaucacuguggauuuuggaaaccagcagaaagaggaaagagguagcaagagcuccagagagaagucgaggaagagagagacggggucagagagagcgcgcgggcgugcgagcagcgaaagcgacaggggcaaagugagugaccugcuuuugggggugaccgccggagcgcggcgugagcccucccccuugggaucccgcagcugaccagucgcgcugacggacagacagacagacaccgcccccagccccagcuaccaccuccuccccggccggcggcggacaguggacgcggcggcgagccgcgggcaggggccggagcccgcgcccggaggcgggguggagggggucggggcucgcggcgucgcacugaaacuuuucguccaacuucugggcuguucucgcuucggaggagccgugguccgcgcgggggaagccgagccgagcggagccgcgagaagugcuagcucgggccgggaggagccgcagccggaggagggggaggaggaagaagagaaggaagaggagagggggccgcaguggcgacucggcgcucggaagccgggcucauggacgggugaggcggcggugugcgcagacagugcuccagccgcgcgcgcuccccaggcccuggcccgggccucgggccggggaggaagaguagcucgccgaggcgccgaggagagcgggccgccccacagcccgagccggagagggagcgcgagccgcgccggccccggucgggccuccgaaaccaugaacuuucugcugucuugggugcauuggagccuugccuugcugcucuaccuccaccaugccaaguggucccaggcugcacccauggcagaaggaggagggcagaaucaucacgaaguggugaaguucauggaugucuaucagcgcagcuacugccauccaaucgagacccugguggacaucuuccaggaguacccugaugagaucgaguacaucuucaagccauccugugugccccugaugcgaugcgggggcugcugcaaugacgagggccuggagugugugcccacugaggaguccaacaucaccaugcagauuaugcggaucaaaccucaccaaggccagcacauaggagagaugagcuuccuacagcacaacaaaugugaaugcagaccaaagaaagauagagcaagacaagaaaaaaaaucaguucgaggaaagggaaaggggcaaaaacgaaagcgcaagaaaucccgguauaaguccuggagcguaugugacaagccgaggcggugagccgggcaggaggaaggagccucccucaggguuucgggaaccagaucucucaccaggaaagacugauacagaacgaucgauacagaaaccacgcugccgccaccacaccaucaccaucgacagaacaguccuuaauccagaaaccugaaaugaaggaagaggagacucugcgcagagcacuuuggguccggagggcgagacuccggcggaagcauucccgggcgggugacccagcacggucccucuuggaauuggauucgccauuuuauuuuucuugcugcuaaaucaccgagcccggaagauuagagaguuuuauuucugggauuccuguagacacacccacccacauacauacauuuauauauauauauauuauauauauauaaaaauaaauaucucuauuuuauauauauaaaauauauauauucuuuuuuuaaauuaacagugcuaauguuauuggugucuucacuggauguauuugacugcuguggacuugaguugggaggggaauguucccacucagauccugacagggaagaggaggagaugagagacucuggcaugaucuuuuuuuugucccacuugguggggccaggguccucuccccugcccaggaaugugcaaggccagggcaugggggcaaauaugacccaguuuugggaacaccgacaaacccagcccuggcgcugagccucucuaccccaggucagacggacagaaagacagaucacagguacagggaugaggacaccggcucugaccaggaguuuggggagcuucaggacauugcugugcuuuggggauucccuccacaugcugcacgcgcaucucgcccccaggggcacugccuggaagauucaggagccugggcggccuucgcuuacucucaccugcuucugaguugcccaggagaccacuggcagaugucccggcgaagagaagagacacauuguuggaagaagcagcccaugacagcuccccuuccugggacucgcccucauccucuuccugcuccccuuccuggggugcagccuaaaaggaccuauguccucacaccauugaaaccacuaguucuguccccccaggagaccugguugugugugugugagugguugaccuuccuccauccccugguccuucccuucccuucccgaggcacagagagacagggcaggauccacgugcccauuguggaggcagagaaaagagaaaguguuuuauauacgguacuuauuuaauaucccuuuuuaauuagaaauuaaaacaguuaauuuaauuaaagaguaggguuuuuuuucaguauucuugguuaauauuuaauuucaacuauuuaugagauguaucuuuugcucucucuugcucucuuauuuguaccgguuuuuguauauaaaauucauguuuccaaucucucucucccugaucggugacagucacuagcuuaucuugaacagauauuuaauuuugcuaacacucagcucugcccuccccgauccccuggcuccccagcacacauuccuuugaaauaagguuucaauauacaucuacauacuauauauauauuuggcaacuuguauuuguguguauauauauauauauauguuuauguauauaugugauucugauaaaauagacauugcuauucuguuuuuuauauguaaaaacaaaacaagaaaaaauagagaauucuacauacuaaaucucucuccuuuuuuaauuuuaauauuuguuaucauuuauuuauuggugcuacuguuuauccguaauaauuguggggaaaagauauuaacaucacgucuuugucucuagugcaguuuuucgagauauuccguaguacauauuuauuuuuaaacaacgacaaagaaauacagauauaucuuaaaaaaaaaaaagcauuuuguauuaaagaauuuaauucugaucucaaaaaaaaaaaaaaaaaa

SEQ ID NO: 135 - protein, 171 aa, Homo sapiens
  VEGFA
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVCDKPRR

SEQ ID NO: 136 - RNA, 3494 nt, Homo sapiens; identical to SEQ ID NO: 134
  VEGFA isoform r

SEQ ID NO: 137 - protein, 351 aa, Homo sapiens
  VEGFA
MTDRQTDTAPSPSYHLLPGRRRTVDAAASRGQGPEPAPGGGVEGVGARGVALKLFVQLLGCSRFGGAVVRAGEAEPSGAARSASSGREEPQPEEGEEEEEKEEERGPQWRLGARKPGSWTGEAAVCADSAPAARAPQALARASGRGGRVARRGAEESGPPHSPSRRGSASRAGPGRASETMNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVCDKPRR

SEQ ID NO: 138 - RNA, 2569 nt, Homo sapiens
  VEGFA isoform s
agcccgggccuggccggccgcguguucccggagccucggcugcccgaauggggagcccagaguggcgagcggcaccccuccccccgccagcccuccgcgggaaggugaccucucgaguggucccaggcugcacccauggcagaaggaggagggcagaaucaucacgaaguggugaaguucauggaugucuaucagcgcagcuacugccauccaaucgagacccugguggacaucuuccaggaguacccugaugagaucgaguacaucuucaagccauccugugugccccugaugcgaugcgggggcugcugcaaugacgagggccuggagugugugcccacugaggaguccaacaucaccaugcagauuaugcggaucaaaccucaccaaggccagcacauaggagagaugagcuuccuacagcacaacaaaugugaaugcagaccaaagaaagauagagcaagacaagaaaaucccugugggccuugcucagagcggagaaagcauuuguuuguacaagauccgcagacguguaaauguuccugcaaaaacacagacucgcguugcaaggcgaggcagcuugaguuaaacgaacguacuugcagaugugacaagccgaggcggugagccgggcaggaggaaggagccucccucaggguuucgggaaccagaucucucaccaggaaagacugauacagaacgaucgauacagaaaccacgcugccgccaccacaccaucaccaucgacagaacaguccuuaauccagaaaccugaaaugaaggaagaggagacucugcgcagagcacuuuggguccggagggcgagacuccggcggaagcauucccgggcgggugacccagcacggucccucuuggaauuggauucgccauuuuauuuuucuugcugcuaaaucaccgagcccggaagauuagagaguuuuauuucugggauuccuguagacacacccacccacauacauacauuuauauauauauauauuauauauauauaaaaauaaauaucucuauuuuauauauauaaaauauauauauucuuuuuuuaaauuaacagugcuaauguuauuggugucuucacuggauguauuugacugcuguggacuugaguugggaggggaauguucccacucagauccugacagggaagaggaggagaugagagacucuggcaugaucuuuuuuuugucccacuugguggggccaggguccucuccccugcccaggaaugugcaaggccagggcaugggggcaaauaugacccaguuuugggaacaccgacaaacccagcccuggcgcugagccucucuaccccaggucagacggacagaaagacagaucacagguacagggaugaggacaccggcucugaccaggaguuuggggagcuucaggacauugcugugcuuuggggauucccuccacaugcugcacgcgcaucucgcccccaggggcacugccuggaagauucaggagccugggcggccuucgcuuacucucaccugcuucugaguugcccaggagaccacuggcagaugucccggcgaagagaagagacacauuguuggaagaagcagcccaugacagcuccccuuccugggacucgcccucauccucuuccugcuccccuuccuggggugcagccuaaaaggaccuauguccucacaccauugaaaccacuaguucuguccccccaggagaccugguugugugugugugagugguugaccuuccuccauccccugguccuucccuucccuucccgaggcacagagagacagggcaggauccacgugcccauuguggaggcagagaaaagagaaaguguuuuauauacgguacuuauuuaauaucccuuuuuaauuagaaauuaaaacaguuaauuuaauuaaagaguaggguuuuuuuucaguauucuugguuaauauuuaauuucaacuauuuaugagauguaucuuuugcucucucuugcucucuuauuuguaccgguuuuuguauauaaaauucauguuuccaaucucucucucccugaucggugacagucacuagcuuaucuugaacagauauuuaauuuugcuaacacucagcucugcccuccccgauccccuggcuccccagcacacauuccuuugaaauaagguuucaauauacaucuacauacuauauauauauuuggcaacuuguauuuguguguauauauauauauauauguuuauguauauaugugauucugauaaaauagacauugcuauucuguuuuuuauauguaaaaacaaaacaagaaaaaauagagaauucuacauacuaaaucucucuccuuuuuuaauuuuaauauuuguuaucauuuauuuauuggugcuacuguuuauccguaauaauuguggggaaaagauauuaacaucacgucuuugucucuagugcaguuuuucgagauauuccguaguacauauuuauuuuuaaacaacgacaaagaaauacagauauaucuuaaaaaaaaaaaagcauuuuguauuaaagaauuuaauucugaucucaaaaaaaaaaaaaaaaaaaa

SEQ ID NO: 139 - protein, 163 aa, Homo sapiens
  VEGFA
MAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRCDKPRR

SEQ ID NO: 140 - RNA, 3626 nt, Homo sapiens; identical to SEQ ID NO: 120
  VEGFA isoform b

SEQ ID NO: 141 - protein, 395 aa, Homo sapiens
  VEGFA
MTDRQTDTAPSPSYHLLPGRRRTVDAAASRGQGPEPAPGGGVEGVGARGVALKLFVQLLGCSRFGGAVVRAGEAEPSGAARSASSGREEPQPEEGEEEEEKEEERGPQWRLGARKPGSWTGEAAVCADSAPAARAPQALARASGRGGRVARRGAEESGPPHSPSRRGSASRAGPGRASETMNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVPCGPCSERRKHLFVQDPQTCKCSCKNTDSRCKARQLELNERTCRCDKPRR

SEQ ID NO: 142 - RNA, 1721 nt, Homo sapiens
  VEGFB isoform VEGFB-167 precursor
gccguccccgccgccgcugcccgccgccaccggccgcccgcccgcccggcuccuccggccgccuccgcugcgcugcgcugcgcugccugcacccagggcucgggagggggccgcggaggagucgccccccgcgcccggcccccgcccgccgcgcccgggcccgcgccauggggcucuggcugucgccgccccccgcgccgccgggcuagggcgaugcgggcgcccccggcgggcggccccggcgggcaccaugagcccucugcuccgccgccugcugcucgccgcacuccugcagcuggcccccgcccaggccccugucucccagccugaugccccuggccaccagaggaaaguggugucauggauagauguguauacucgcgcuaccugccagccccgggaggugguggugcccuugacuguggagcucaugggcaccguggccaaacagcuggugcccagcugcgugacugugcagcgcugugguggcugcugcccugacgauggccuggagugugugcccacugggcagcaccaaguccggaugcagauccucaugauccgguacccgagcagucagcugggggagaugucccuggaagaacacagccagugugaaugcagaccuaaaaaaaaggacagugcugugaagccagacagccccaggccccucugcccacgcugcacccagcaccaccagcgcccugacccccggaccugccgcugccgcugccgacgccgcagcuuccuccguugccaagggcggggcuuagagcucaacccagacaccugcaggugccggaagcugcgaaggugacacauggcuuuucagacucagcagggugacuugccucagaggcuauaucccagugggggaacaaagaggagccugguaaaaaacagccaagcccccaagaccucagcccaggcagaagcugcucuaggaccugggccucucagagggcucuucugccaucccuugucucccugaggccaucaucaaacaggacagaguuggaagaggagacugggaggcagcaagaggggucacauaccagcucaggggagaauggaguacugucucaguuucuaaccacucugugcaaguaagcaucuuacaacuggcucuuccuccccucacuaagaagacccaaaccucugcauaaugggauuugggcuuugguacaagaacugugacccccaacccugauaaaagagauggaaggagcugucccugccugugucacuguuugucacuguccaggcuggcugguuugggcaugaaugucugcaucacuaaauccagagcuugucuugcucccucauugugcagauggaggaaaugaggacuaaggccccacagcagaucccaggcagggccagaauuauguauucaucacuuucaaguuauugccacgcaugggagucagggauagcccagucaauacagacugccugcccuccugcucuucaccaggguucuuuucuagaaggagacagccuucuguggccagagagcuugggguaggacccagaucuacugagugaccuugcuugucacuaccccugccucucugagcagcaguuuccacaugugcacauagagggaacagaagauugcugugguuggcguccucgggccccagagaaguuugagacuaucuuuacguaauagaaaagaacacuuguucuuccugccaggcaaaaaaaaaaaaaaaaaa

SEQ ID NO: 143 - protein, 188 aa, Homo sapiens
  VEGFB
MSPLLRRLLLAALLQLAPAQAPVSQPDAPGHQRKVVSWIDVYTRATCQPREVVVPLTVELMGTVAKQLVPSCVTVQRCGGCCPDDGLECVPTGQHQVRMQILMIRYPSSQLGEMSLEEHSQCECRPKKKDSAVKPDSPRPLCPRCTQHHQRPDPRTCRCRCRRRSFLRCQGRGLELNPDTCRCRKLRR

SEQ ID NO: 144 - RNA, 1822 nt, Homo sapiens
  VEGFB isoform VEGFB-186 precursor
gccguccccgccgccgcugcccgccgccaccggccgcccgcccgcccggcuccuccggccgccuccgcugcgcugcgcugcgcugccugcacccagggcucgggagggggccgcggaggagucgccccccgcgcccggcccccgcccgccgcgcccgggcccgcgccauggggcucuggcugucgccgccccccgcgccgccgggcuagggcgaugcgggcgcccccggcgggcggccccggcgggcaccaugagcccucugcuccgccgccugcugcucgccgcacuccugcagcuggcccccgcccaggccccugucucccagccugaugccccuggccaccagaggaaaguggugucauggauagauguguauacucgcgcuaccugccagccccgggaggugguggugcccuugacuguggagcucaugggcaccguggccaaacagcuggugcccagcugcgugacugugcagcgcugugguggcugcugcccugacgauggccuggagugugugcccacugggcagcaccaaguccggaugcagauccucaugauccgguacccgagcagucagcugggggagaugucccuggaagaacacagccagugugaaugcagaccuaaaaaaaaggacagugcugugaagccagacagggcugccacuccccaccaccguccccagccccguucuguuccgggcugggacucugcccccggagcacccuccccagcugacaucacccaucccacuccagccccaggccccucugcccacgcugcacccagcaccaccagcgcccugacccccggaccugccgcugccgcugccgacgccgcagcuuccuccguugccaagggcggggcuuagagcucaacccagacaccugcaggugccggaagcugcgaaggugacacauggcuuuucagacucagcagggugacuugccucagaggcuauaucccagugggggaacaaagaggagccugguaaaaaacagccaagcccccaagaccucagcccaggcagaagcugcucuaggaccugggccucucagagggcucuucugccaucccuugucucccugaggccaucaucaaacaggacagaguuggaagaggagacugggaggcagcaagaggggucacauaccagcucaggggagaauggaguacugucucaguuucuaaccacucugugcaaguaagcaucuuacaacuggcucuuccuccccucacuaagaagacccaaaccucugcauaaugggauuugggcuuugguacaagaacugugacccccaacccugauaaaagagauggaaggagcugucccugccugugucacuguuugucacuguccaggcuggcugguuugggcaugaaugucugcaucacuaaauccagagcuugucuugcucccucauugugcagauggaggaaaugaggacuaaggccccacagcagaucccaggcagggccagaauuauguauucaucacuuucaaguuauugccacgcaugggagucagggauagcccagucaauacagacugccugcccuccugcucuucaccaggguucuuuucuagaaggagacagccuucuguggccagagagcuugggguaggacccagaucuacugagugaccuugcuugucacuaccccugccucucugagcagcaguuuccacaugugcacauagagggaacagaagauugcugugguuggcguccucgggccccagagaaguuugagacuaucuuuacguaauagaaaagaacacuuguucuuccugccaggcaaaaaaaaaaaaaaaaaa

SEQ ID NO: 145 - protein, 207 aa, Homo sapiens
  VEGFB
MSPLLRRLLLAALLQLAPAQAPVSQPDAPGHQRKVVSWIDVYTRATCQPREVVVPLTVELMGTVAKQLVPSCVTVQRCGGCCPDDGLECVPTGQHQVRMQILMIRYPSSQLGEMSLEEHSQCECRPKKKDSAVKPDRAATPHHRPQPRSVPGWDSAPGAPSPADITHPTPAPGPSAHAAPSTTSALTPGPAAAAADAAASSVAKGGA

SEQ ID NO: 146 - RNA, 2084 nt, Homo sapiens
  VEGFD preproprotein
aagacacaugcuucugcaagcuuccaugaagguugugcaaaaaaguuucaauccagaguuggguuccagcuuucuguagcuguaagcauugguggccacaccaccuccuuacaaagcaacuagaaccugcggcauacauuggagagauuuuuuuaauuuucuggacaugaaguaaauuuagagugcuuucuaauuucagguagaagacauguccaccuucugauuauuuuuggagaacauuuugauuuuuuucaucucucucuccccaccccuaagauugugcaaaaaaagcguaccuugccuaauugaaauaauuucauuggauuuugaucagaacugauuauuugguuuucugugugaaguuuugagguuucaaacuuuccuucuggagaaugccuuuugaaacaauuuucucuagcugccugaugucaacugcuuaguaaucaguggauauugaaauauucaaaauguacagagaguggguaguggugaauguuuucaugauguuguacguccagcuggugcagggcuccaguaaugaacauggaccagugaagcgaucaucucaguccacauuggaacgaucugaacagcagaucagggcugcuucuaguuuggaggaacuacuucgaauuacucacucugaggacuggaagcuguggagaugcaggcugaggcucaaaaguuuuaccaguauggacucucgcucagcaucccaucgguccacuagguuugcggcaacuuucuaugacauugaaacacuaaaaguuauagaugaagaauggcaaagaacucagugcagcccuagagaaacgugcguggagguggccagugagcuggggaagaguaccaacacauucuucaagcccccuugugugaacguguuccgaugugguggcuguugcaaugaagagagccuuaucuguaugaacaccagcaccucguacauuuccaaacagcucuuugagauaucagugccuuugacaucaguaccugaauuagugccuguuaaaguugccaaucauacagguuguaagugcuugccaacagccccccgccauccauacucaauuaucagaagauccauccagaucccugaagaagaucgcuguucccauuccaagaaacucuguccuauugacaugcuaugggauagcaacaaauguaaauguguuuugcaggaggaaaauccacuugcuggaacagaagaccacucucaucuccaggaaccagcucucugugggccacacaugauguuugacgaagaucguugcgagugugucuguaaaacaccaugucccaaagaucuaauccagcaccccaaaaacugcaguugcuuugagugcaaagaaagucuggagaccugcugccagaagcacaagcuauuucacccagacaccugcagcugugaggacagaugccccuuucauaccagaccaugugcaaguggcaaaacagcaugugcaaagcauugccgcuuuccaaaggagaaaagggcugcccaggggccccacagccgaaagaauccuugauucagcguuccaaguuccccaucccugucauuuuuaacagcaugcugcuuugccaaguugcugucacuguuuuuuucccagguguuaaaaaaaaaauccauuuuacacagcaccacagugaauccagaccaaccuuccauucacaccagcuaaggagucccugguucauugauggaugucuucuagcugcagaugccucugcgcaccaaggaauggagaggaggggacccauguaauccuuuuguuuaguuuuguuuuuguuuuuuggugaaugagaaaggugugcuggucauggaauggcaggugucauaugacugauuacucagagcagaugaggaaaacuguagucucugaguccuuugcuaaucgcaacucuugugaauuauucugauucuuuuuuaugcagaauuugauucguaugaucaguacugacuuucugauuacuguccagcuuauagucuuccaguuuaaugaacuaccaucugauguuucauauuuaaguguauuuaaagaaaauaaacaccauuauucaagccauauaaaaaaaaaaaa

SEQ ID NO: 147 - protein, 354 aa, Homo sapiens
  VEGFD
MYREWVVVNVFMMLYVQLVQGSSNEHGPVKRSSQSTLERSEQQIRAASSLEELLRITHSEDWKLWRCRLRLKSFTSMDSRSASHRSTRFAATFYDIETLKVIDEEWQRTQCSPRETCVEVASELGKSTNTFFKPPCVNVFRCGGCCNEESLICMNTSTSYISKQLFEISVPLTSVPELVPVKVANHTGCKCLPTAPRHPYSIIRRSIQIPEEDRCSHSKKLCPIDMLWDSNKCKCVLQEENPLAGTEDHSHLQEPALCGPHMMFDEDRCECVCKTPCPKDLIQHPKNCSCFECKESLETCCQKHKLFHPDTCSCEDRCPFHTRPCASGKTACAKHCRFPKEKRAAQGPHSRKNP

SEQ ID NO: 148 - RNA, 2103 nt, Homo sapiens
  VEGFC
acuucggggaaggggagggaggagggggacgagggcucuggcggguuuggaggggcugaacaucgcgggguguucuggugucccccgccccgccucuccaaaaagcuacaccgacgcggaccgcggcggcguccucccucgcccucgcuucaccucgcgggcuccgaaugcggggagcucggauguccgguuuccugugaggcuuuuaccugacacccgccgccuuuccccggcacuggcugggagggcgcccugcaaaguugggaacgcggagccccggacccgcucccgccgccuccggcucgcccagggggggucgccgggaggagcccgggggagagggaccaggaggggcccgcggccucgcaggggcgcccgcgcccccaccccugcccccgccagcggaccggucccccacccccgguccuuccaccaugcacuugcugggcuucuucucuguggcguguucucugcucgccgcugcgcugcucccggguccucgcgaggcgcccgccgccgccgccgccuucgaguccggacucgaccucucggacgcggagcccgacgcgggcgaggccacggcuuaugcaagcaaagaucuggaggagcaguuacggucuguguccaguguagaugaacucaugacuguacucuacccagaauauuggaaaauguacaagugucagcuaaggaaaggaggcuggcaacauaacagagaacaggccaaccucaacucaaggacagaagagacuauaaaauuugcugcagcacauuauaauacagagaucuugaaaaguauugauaaugaguggagaaagacucaaugcaugccacgggagguguguauagauguggggaaggaguuuggagucgcgacaaacaccuucuuuaaaccuccauguguguccgucuacagauguggggguugcugcaauagugaggggcugcagugcaugaacaccagcacgagcuaccucagcaagacguuauuugaaauuacagugccucucucucaaggccccaaaccaguaacaaucaguuuugccaaucacacuuccugccgaugcaugucuaaacuggauguuuacagacaaguucauuccauuauuagacguucccugccagcaacacuaccacagugucaggcagcgaacaagaccugccccaccaauuacauguggaauaaucacaucugcagaugccuggcucaggaagauuuuauguuuuccucggaugcuggagaugacucaacagauggauuccaugacaucuguggaccaaacaaggagcuggaugaagagaccugucagugugucugcagagcggggcuucggccugccagcuguggaccccacaaagaacuagacagaaacucaugccagugugucuguaaaaacaaacucuuccccagccaauguggggccaaccgagaauuugaugaaaacacaugccaguguguauguaaaagaaccugccccagaaaucaaccccuaaauccuggaaaaugugccugugaauguacagaaaguccacagaaaugcuuguuaaaaggaaagaaguuccaccaccaaacaugcagcuguuacagacggccauguacgaaccgccagaaggcuugugagccaggauuuucauauagugaagaagugugucguugugucccuucauauuggaaaagaccacaaaugagcuaagauuguacuguuuuccaguucaucgauuuucuauuauggaaaacuguguugccacaguagaacugucugugaacagagagacccuuguggguccaugcuaacaaagacaaaagucugucuuuccugaaccauguggauaacuuuacagaaauggacuggagcucaucugcaaaaggccucuuguaaagacugguuuucugccaaugaccaaacagccaagauuuuccucuugugauuucuuuaaaagaaugacuauauaauuuauuuccacuaaaaauauuguuucugcauucauuuuuauagcaacaacaauugguaaaacucacugugaucaauauuuuuauaucaugcaaaauauguuuaaaauaaaaugaaaauuguauuauaagcugaaaaaaaaaaaaaaaaa

SEQ ID NO: 149 - protein, 419 aa, Homo sapiens
  VEGFC
MHLLGFFSVACSLLAAALLPGPREAPAAAAAFESGLDLSDAEPDAGEATAYASKDLEEQLRSVSSVDELMTVLYPEYWKMYKCQLRKGGWQHNREQANLNSRTEETIKFAAAHYNTEILKSIDNEWRKTQCMPREVCIDVGKEFGVATNTFFKPPCVSVYRCGGCCNSEGLQCMNTSTSYLSKTLFEITVPLSQGPKPVTISFANHTSCRCMSKLDVYRQVHSIIRRSLPATLPQCQAANKTCPTNYMWNNHICRCLAQEDFMFSSDAGDDSTDGFHDICGPNKELDEETCQCVCRAGLRPASCGPHKELDRNSCQCVCKNKLFPSQCGANREFDENTCQCVCKRTCPRNQPLNPGKCACECTESPQKCLLKGKKFHHQTCSCYRRPCTNRQKACEPGFSYSEEVCRCVPSYWKRPQMS

SEQ ID NO: 150 - RNA, 1455 nt, Homo sapiens
  plasminogen acti vator, urokinase receptor (PLAUR), transcript variant 2
gccgagccagccccuucaccaccagccggccgcgccccgggaagggaaguuuguggcggaggagguucguacgggaggagggggaggcgcccacgcaucuggggcugacucgcucuuucgcaaaacgucugggaggagucccuggggccacaaaacugccuccuuccugaggccagaaggagagaagacgugcagggaccccgcgcacaggagcugcccucgcgacaugggucacccgccgcugcugccgcugcugcugcugcuccacaccugcgucccagccucuuggggccugcggugcaugcaguguaagaccaacggggauugccguguggaagagugcgcccugggacaggaccucugcaggaccacgaucgugcgcuugugggaagaaggagaagagcuggagcugguggagaaaagcuguacccacucagagaagaccaacaggacccugagcuaucggacuggcuugaagaucaccagccuuaccgagguuguguguggguuagacuugugcaaccagggcaacucuggccgggcugucaccuauucccgaagccguuaccucgaaugcauuuccuguggcucaucagacaugagcugugagaggggccggcaccagagccugcagugccgcagcccugaagaacagugccuggauguggugacccacuggauccaggaaggugaagaagggcguccaaaggaugaccgccaccuccguggcuguggcuaccuucccggcugcccgggcuccaaugguuuccacaacaacgacaccuuccacuuccugaaaugcugcaacaccaccaaaugcaacgagggcccaauccuggagcuugaaaaucugccgcagaauggccgccaguguuacagcugcaaggggaacagcacccauggaugcuccucugaagagacuuuccucauugacugccgaggccccaugaaucaaugucugguagccaccggcacucacgaacgcucacucuggggaagcugguugccauguaaaaguacuacugcccugagaccaccaugcugugaggaagcccaagcuacucauguauaaaugccauguggagauagagccccagauguuucagccaucucagcccaggcaccagacaagugggugaagaagccaccuuggacauguagccccagcagaugugauauagagaagaaacaggaaacuuggcuauauuaguuuccuagggcugccugugauaaauuauuacaaacuuuauaaacuaacacauugugugccuauaucaaaacaucauggaaggacaggcacaguggcucaugccuguaguccuagcacuuugggagggugagaaaggaagaucucuugagcucaggaguucaagaucagccugggcaacacagugagaccucaucuccacuaaaaauaaaaaaaaauuggcuggaaaaaaaaaaaaaaaaaaaaaa

SEQ ID NO: 151 - protein, 281 aa, Homo sapiens
  plasminogen activator, urokinase receptor (PLAUR), transcript variant 2
MGHPPLLPLLLLLHTCVPASWGLRCMQCKTNGDCRVEECALGQDLCRTTIVRLWEEGEELELVEKSCTHSEKTNRTLSYRTGLKITSLTEVVCGLDLCNQGNSGRAVTYSRSRYLECISCGSSDMSCERGRHQSLQCRSPEEQCLDVVTHWIQEGEEGRPKDDRHLRGCGYLPGCPGSNGFHNNDTFHFLKCCNTTKCNEGPILELENLPQNGRQCYSCKGNSTHGCSSEETFLIDCRGPMNQCLVATGTHERSLWGSWLPCKSTTALRPPCCEEAQATHV

SEQ ID NO: 152 - RNA, 1435 nt, Homo sapiens
  plasminogen activator, urokinase receptor (PLAUR), transcript variant 3
gccgagccagccccuucaccaccagccggccgcgccccgggaagggaaguuuguggcggaggagguucguacgggaggagggggaggcgcccacgcaucuggggcugacucgcucuuucgcaaaacgucugggaggagucccuggggccacaaaacugccuccuuccugaggccagaaggagagaagacgugcagggaccccgcgcacaggagcugcccucgcgacaugggucacccgccgcugcugccgcugcugcugcugcuccacaccugcgucccagccucuuggggccugcggugcaugcaguguaagaccaacggggauugccguguggaagagugcgcccugggacaggaccucugcaggaccacgaucgugcgcuugugggaagaaggagaagagcuggagcugguggagaaaagcuguacccacucagagaagaccaacaggacccugagcuaucggacuggcuugaagaucaccagccuuaccgagguuguguguggguuagacuugugcaaccagggcaacucuggccgggcugucaccuauucccgaagccguuaccucgaaugcauuuccuguggcucaucagacaugagcugugagaggggccggcaccagagccugcagugccgcagcccugaagaacagugccuggauguggugacccacuggauccaggaaggugaagaaguccuggagcuugaaaaucugccgcagaauggccgccaguguuacagcugcaaggggaacagcacccauggaugcuccucugaagagacuuuccucauugacugccgaggccccaugaaucaaugucugguagccaccggcacucacgaaccgaaaaaccaaagcuauaugguaagaggcugugcaaccgccucaaugugccaacaugcccaccugggugacgccuucagcaugaaccacauugaugucuccugcuguacuaaaaguggcuguaaccacccagaccuggauguccaguaccgcaguggggcugcuccucagccuggcccugcccaucucagccucaccaucacccugcuaaugacugccagacuguggggaggcacucuccucuggaccuaaaccugaaaucccccucucugcccuggcuggauccgggggaccccuuugcccuucccucggcucccagcccuacagacuugcugugugaccucaggccagugugccgaccucucugggccucaguuuucccagcuaugaaaacagcuaucucacaaaguugugugaagcagaagagaaaagcuggaggaaggccgugggccaaugggagagcucuuguuauuauuaauauuguugccgcuguuguguuguuguuauuaauuaauauucauauuauuuauuuuauacuuacauaaagauuuuguaccaguggacaaggccaaaaaaaaaaaaaaaaaa

SEQ ID NO: 153 - protein, 290 aa, Homo sapiens
  plasminogen activator, urokinase receptor (PLAUR), transcript variant 3
MGHPPLLPLLLLLHTCVPASWGLRCMQCKTNGDCRVEECALGQDLCRTTIVRLWEEGEELELVEKSCTHSEKTNRTLSYRTGLKITSLTEVVCGLDLCNQGNSGRAVTYSRSRYLECISCGSSDMSCERGRHQSLQCRSPEEQCLDVVTHWIQEGEEVLELENLPQNGRQCYSCKGNSTHGCSSEETFLIDCRGPMNQCLVATGTHEPKNQSYMVRGCATASMCQHAHLGDAFSMNHIDVSCCTKSGCNHPDLDVQYRSGAAPQPGPAHLSLTITLLMTARLWGGTLLWT

SEQ ID NO: 154 - RNA, 1426 nt, Homo sapiens
  plasminogen activator, urokinase receptor (PLAUR), transcript variant 4
gccgagccagccccuucaccaccagccggccgcgccccgggaagggaaguuuguggcggaggagguucguacgggaggagggggaggcgcccacgcaucuggggcugacucgcucuuucgcaaaacgucugggaggagucccuggggccacaaaacugccuccuuccugaggccagaaggagagaagacgugcagggaccccgcgcacaggagcugcccucgcgacaugggucacccgccgcugcugccgcugcugcugcugcuccacaccugcgucccagccucuuggggccugcggugcaugcaguguaagaccaacggggauugccguguggaagagugcgcccugggacaggaccucugcaggaccacgaucgugcgcuugugggaagaaggagaagagcuggagcugguggagaaaagcuguacccacucagagaagaccaacaggacccugagcuaucggacuggcuugaagaucaccagccuuaccgagguuguguguggguuagacuugugcaaccagggcaacucuggccgggcugucaccuauucccgagccguuaccucgaaugcauuuccuguggcucaucagacaugagcugugagaggggccggcaccagagccugcagugccgcagcccugaagaacagugccuggauguggugacccacuggauccaggaaggugaagaagggcguccaaaggaugaccgccaccuccguggcuguggcuaccuucccggcugcccgggcuccaaugguuuccacaacaacgacaccuuccacuuccugaaaugcugcaacaccaccaaaugcaacgagggcccaaaaccgaaaaaccaaagcuauaugguaagaggcugugcaaccgccucaaugugccaacaugcccaccugggugacgccuucagcaugaaccacauugaugucuccugcuguacuaaaaguggcuguaaccacccagaccuggauguccaguaccgcaguggggcugcuccucagccuggcccugcccaucucagccucaccaucacccugcuaaugacugccagacuguggggaggcacucuccucuggaccuaaaccugaaaucccccucucugcccuggcuggauccgggggaccccuuugcccuucccucggcucccagcccuacagacuugcugugugaccucaggccagugugccgaccucucugggccucaguuuucccagcuaugaaaacagcuaucucacaaaguugugugaagcagaagagaaaagcuggaggaaggccgugggccaaugggagagcucuuguuauuauuaauauuguugccgcuguuguguuguuguuauuaauuaauauucauauuauuuauuuuauacuuacauaaagauuuuguaccaguggacaaggccagguaaaaaaaaaaaaaaaaaa

SEQ ID NO: 155 - protein, 70 aa, Homo sapiens
  plasminogen activator, urokinase receptor (PLAUR), transcript variant 4
MGHPPLLPLLLLLHTCVPASWGLRCMQCKTNGDCRVEECALGQDLCRTTIVRLWEEGEELELVEKSCTHS

SEQ ID NO: 156 - RNA, 1570 nt, Homo sapiens
  plasmingen activator, urokinase receptor (PLAUR), transcript variant 1
gccgagccagccccuucaccaccagccggccgcgccccgggaagggaaguuuguggcggaggagguucguacgggaggagggggaggcgcccacgcaucuggggcugacucgcucuuucgcaaaacgucugggaggagucccuggggccacaaaacugccuccuuccugaggccagaaggagagaagacgugcagggaccccgcgcacaggagcugcccucgcgacaugggucacccgccgcugcugccgcugcugcugcugcuccacaccugcgucccagccucuuggggccugcggugcaugcaguguaagaccaacggggauugccguguggaagagugcgcccugggacaggaccucugcaggaccacgaucgugcgcuugugggaagaaggagaagagcuggagcugguggagaaaagcuguacccacucagagaagaccaacaggacccugagcuaucggacuggcuugaagaucaccagccuuaccgagguuguguguggguuagacuugugcaaccagggcaacucuggccgggcugucaccuauucccgaagccguuaccucgaaugcauuuccuguggcucaucagacaugagcugugagaggggccggcaccagagccugcagugccgcagcccugaagaacagugccuggauguggugacccacuggauccaggaaggugaagaagggcguccaaaggaugaccgccaccuccguggcuguggcuaccuucccggcugcccgggcuccaaugguuuccacaacaacgacaccuuccacuuccugaaaugcugcaacaccaccaaaugcaacgagggcccaauccuggagcuugaaaaucugccgcagaauggccgccaguguuacagcugcaaggggaacagcacccauggaugcuccucugaagagacuuuccucauugacugccgaggccccaugaaucaaugucugguagccaccggcacucacgaaccgaaaaaccaaagcuauaugguaagaggcugugcaaccgccucaaugugccaacaugcccaccugggugacgccuucagcaugaaccacauugaugucuccugcuguacuaaaaguggcuguaaccacccagaccuggauguccaguaccgcaguggggcugcuccucagccuggcccugcccaucucagccucaccaucacccugcuaaugacugccagacuguggggaggcacucuccucuggaccuaaaccugaaaucccccucucugcccuggcuggauccgggggaccccuuugcccuucccucggcucccagcccuacagacuugcugugugaccucaggccagugugccgaccucucugggccucaguuuucccagcuaugaaaacagcuaucucacaaaguugugugaagcagaagagaaaagcuggaggaaggccgugggccaaugggagagcucuuguuauuauuaauauuguugccgcuguuguguuguuguuauuaauuaauauucauauuauuuauuuuauacuuacauaaagauuuuguaccaguggacaaggccaaaaaaaaaaaaaaaaaa

SEQ ID NO: 157 - protein, 70 aa, Homo sapiens; identical to SEQ ID NO: 155
  plasminogen activator, urokinase receptor (PLAUR), transcript variant 1

SEQ ID NO: 158 - RNA, 1998 nt, Homo sapiens
  Homo sapiens high mobility group AT-hook 1 (HMGA1), transcript variant 2
cauuugcauggccccgcccccugagugacacggcuggcgcgggcgggcccguccccccugccccugggucgcucuuuuuaagcuccccugagccggugcugcgcuccucuaauugggacuccgagccggggcuauuucuggcgcuggcgcggcuccaagaaggcauccgcauuugcuaccagcggcggccgcggcggagccaggccgguccucagcgcccagcaccgccgcucccggcaacccggagcgcgcaccgcaggccggcggccgagcucgcgcaucccagccaucacucuuccaccugcuccuuagagaagggaagaugagugagucgagcucgaaguccagccagcccuuggccuccaagcaggaaaaggacggcacugagaagcggggccggggcaggccgcgcaagcagccuccgaaggagcccagcgaagugccaacaccuaagagaccucggggccgaccaaagggaagcaaaaacaagggugcugccaagacccggaaaaccaccacaacuccaggaaggaaaccaaggggcagacccaaaaaacuggagaaggaggaagaggagggcaucucgcaggaguccucggaggaggagcagugacccaugcgugccgccugcuccucacuggaggagcagcuuccuucugggacuggacagcuuugcuccgcucccaccgcccccaccccuuccccaggcccaccaucaccaccgccucuggccgccacccccaucuuccaccugugcccucaccaccacacuacacagcacaccagccgcugcagggcucccaugggcugaguggggagcaguuuuccccuggccucaguucccagcuccccccgcccacccacgcauacacacaugcccuccuggacaaggcuaacaucccacuuagccgcacccugcaccugcugcguccccacucccuuggugguggggacauugcucucugggcuuuugguuugggggcgcccucucugcuccuucacuguucccucuggcuucccauaguggggccugggaggguuccccuggccuuaaaaggggcccaagccccaucucauccuggcacgcccuacuccacugcccuggcagcagcagguguggccaauggaggggggugcuggcccccaggauucccccagccaaacugucuuugucaccacguggggcucacuuuucauccuuccccaacuucccuaguccccguacuagguuggacagcccccuucgguuacaggaaggcaggaggggugaguccccuacucccucuucacuguggccacagcccccuugcccuccgccugggaucugaguacauauuguggugauggagaugcagucacuuauuguccaggugaggcccaagagcccuguggccgccaccugaggugggcuggggcugcuccccuaacccuacuuugcuuccgccacucagccauuucccccuccucagauggggcaccaauaacaaggagcucacccugcccgcucccaaccccccuccugcuccucccugccccccaagguucugguuccauuuuuccucuguucacaaacuaccucuggacaguuguguuguuuuuuguucaauguuccauucuucgacauccgucauugcugcugcuaccagcgccaaauguucauccucauugccuccuguucugcccacgauccccucccccaagauacucuuuguggggaagaggggcuggggcauggcaggcugggugaccgacuaccccagucccagggaagguggggcccugccccuaggaugcugcagcagagugagcaagggggcccaaaucgaccauaaaggguguaggggccaccuccucccccuguucuguuggggagggguagccaugauuugucccagccuggggcucccucucugguuuccuauuugcaguuacuugaauaaaaaaaauauccuuuucuggaaaaaaaaaaaaaaaaaaaaaaaaaaaaaaaaaaaaaaa

SEQ ID NO: 159 - protein, 96 aa, Homo sapiens
  Homo sapiens high mobility group AT-hook 1 (HMGA1), transcript variant 2
MSESSSKSSQPLASKQEKDGTEKRGRGRPRKQPPKEPSEVPTPKRPRGRPKGSKNKGAAKTRKTTTTPGRKPRGRPKKLEKEEEEGISQESSEEEQ

SEQ ID NO: 160 - RNA, 2031 nt, Homo sapiens
  Homo sapiens high mobility group AT-hook 1 (HGMA1), transcript variant 1
cauuugcauggccccgcccccugagugacacggcuggcgcgggcgggcccguccccccugccccugggucgcucuuuuuaagcuccccugagccggugcugcgcuccucuaauugggacuccgagccggggcuauuucuggcgcuggcgcggcuccaagaaggcauccgcauuugcuaccagcggcggccgcggcggagccaggccgguccucagcgcccagcaccgccgcucccggcaacccggagcgcgcaccgcaggccggcggccgagcucgcgcaucccagccaucacucuuccaccugcuccuuagagaagggaagaugagugagucgagcucgaaguccagccagcccuuggccuccaagcaggaaaaggacggcacugagaagcggggccggggcaggccgcgcaagcagccuccggugagucccgggacagcgcugguagggagucagaaggagcccagcgaagugccaacaccuaagagaccucggggccgaccaaagggaagcaaaaacaagggugcugccaagacccggaaaaccaccacaacuccaggaaggaaaccaaggggcagacccaaaaaacuggagaaggaggaagaggagggcaucucgcaggaguccucggaggaggagcagugacccaugcgugccgccugcuccucacuggaggagcagcuuccuucugggacuggacagcuuugcuccgcucccaccgcccccaccccuuccccaggcccaccaucaccaccgccucuggccgccacccccaucuuccaccugugcccucaccaccacacuacacagcacaccagccgcugcagggcucccaugggcugaguggggagcaguuuuccccuggccucaguucccagcuccccccgcccacccacgcauacacacaugcccuccuggacaaggcuaacaucccacuuagccgcacccugcaccugcugcguccccacucccuuggugguggggacauugcucucugggcuuuugguuugggggcgcccucucugcuccuucacuguucccucuggcuucccauaguggggccugggaggguuccccuggccuuaaaaggggcccaagccccaucucauccuggcacgcccuacuccacugcccuggcagcagcagguguggccaauggaggggggugcuggcccccaggauucccccagccaaacugucuuugucaccacguggggcucacuuuucauccuuccccaacuucccuaguccccguacuagguuggacagcccccuucgguuacaggaaggcaggaggggugaguccccuacucccucuucacuguggccacagcccccuugcccuccgccugggaucugaguacauauuguggugauggagaugcagucacuuauuguccaggugaggcccaagagcccuguggccgccaccugaggugggcuggggcugcuccccuaacccuacuuugcuuccgccacucagccauuucccccuccucagauggggcaccaauaacaaggagcucacccugcccgcucccaaccccccuccugcuccucccugccccccaagguucugguuccauuuuuccucuguucacaaacuaccucuggacaguuguguuguuuuuuguucaauguuccauucuucgacauccgucauugcugcugcuaccagcgccaaauguucauccucauugccuccuguucugcccacgauccccucccccaagauacucuuuguggggaagaggggcuggggcauggcaggcugggugaccgacuaccccagucccagggaagguggggcccugccccuaggaugcugcagcagagugagcaagggggcccaaaucgaccauaaaggguguaggggccaccuccucccccuguucuguuggggagggguagccaugauuugucccagccuggggcucccucucugguuuccuauuugcaguuacuugaauaaaaaaaauauccuuuucuggaaaaaaaaaaaaaaaaaaaaaaaaaaaaaaaaaaaaaaa

SEQ ID NO: 161 - protein, 107 aa, Homo sapiens
  Homo sapiens high mobility group AT-hook 1 (HMGA1), transcript variant 1
MSESSSKSSQPLASKQEKDGTEKRGRGRPRKQPPVSPGTALVGSQKEPSEVPTPKRPRGRPKGSKNKGAAKTRKTTTTPGRKPRGRPKKLEKEEEEGISQESSEEEQ

SEQ ID NO: 162 - RNA, 2198 nt, Homo sapiens
  Homo sapiens high mobiligy group AT-hook 1 (HMGA1), transcript variant 3
cuuuuuaagcuccccugagccggugcugcgcuccucuaauugggacuccgagccggggcuauuucuggcgcuggcgcggcuccaagaaggcgugaguucgcggccgcuccgguggcuucuuuuuuuuauaucuauaauuuaauuaaauuauuuauuuauugaggccgcgcacgggccgugcccagcuuccugccccucgccauccuucgggggagggggaauauuuuuguccccccgccuggcugugacacauaaauaccccgcgggggccugggcggcgagcacgcggcggcggcggucucugagcgccucugcucucucccgguuucagauccgcauuugcuaccagcggcggccgcggcggagccaggccgguccucagcgcccagcaccgccgcucccggcaacccggagcgcgcaccgcaggccggcggccgagcucgcgcaucccagccaucacucuuccaccugcuccuuagagaagggaagaugagugagucgagcucgaaguccagccagcccuuggccuccaagcaggaaaaggacggcacugagaagcggggccggggcaggccgcgcaagcagccuccggugagucccgggacagcgcugguagggagucagaaggagcccagcgaagugccaacaccuaagagaccucggggccgaccaaagggaagcaaaaacaagggugcugccaagacccggaaaaccaccacaacuccaggaaggaaaccaaggggcagacccaaaaaacuggagaaggaggaagaggagggcaucucgcaggaguccucggaggaggagcagugacccaugcgugccgccugcuccucacuggaggagcagcuuccuucugggacuggacagcuuugcuccgcucccaccgcccccaccccuuccccaggcccaccaucaccaccgccucuggccgccacccccaucuuccaccugugcccucaccaccacacuacacagcacaccagccgcugcagggcucccaugggcugaguggggagcaguuuuccccuggccucaguucccagcuccccccgcccacccacgcauacacacaugcccuccuggacaaggcuaacaucccacuuagccgcacccugcaccugcugcguccccacucccuuggugguggggacauugcucucugggcuuuugguuugggggcgcccucucugcuccuucacuguucccucuggcuucccauaguggggccugggaggguuccccuggccuuaaaaggggcccaagccccaucucauccuggcacgcccuacuccacugcccuggcagcagcagguguggccaauggaggggggugcuggcccccaggauucccccagccaaacugucuuugucaccacguggggcucacuuuucauccuuccccaacuucccuaguccccguacuagguuggacagcccccuucgguuacaggaaggcaggaggggugaguccccuacucccucuucacuguggccacagcccccuugcccuccgccugggaucugaguacauauuguggugauggagaugcagucacuuauuguccaggugaggcccaagagcccuguggccgccaccugaggugggcuggggcugcuccccuaacccuacuuugcuuccgccacucagccauuucccccuccucagauggggcaccaauaacaaggagcucacccugcccgcucccaaccccccuccugcuccucccugccccccaagguucugguuccauuuuuccucuguucacaaacuaccucuggacaguuguguuguuuuuuguucaauguuccauucuucgacauccgucauugcugcugcuaccagcgccaaauguucauccucauugccuccuguucugcccacgauccccucccccaagauacucuuuguggggaagaggggcuggggcauggcaggcugggugaccgacuaccccagucccagggaagguggggcccugccccuaggaugcugcagcagagugagcaagggggcccaaaucgaccauaaaggguguaggggccaccuccucccccuguucuguuggggagggguagccaugauuugucccagccuggggcucccucucugguuuccuauuugcaguuacuugaauaaaaaaaauauccuuuucuggaaaaaaaaaaaaaaaaaaaaaaaaaaaaaaaaaaaaaaa

SEQ ID NO: 163 - protein, 70 aa, Homo sapiens
  Homo sapiens high mobiligy group AT-hook 1 (HMGA1), trnascript variant 3
MSESSSKSSQPLASKQEKDGTEKRGRGRPRKQPPVSPGTALVGSQKEPSEVPTPKRPRGRPKGSKNKGAA

SEQ ID NO: 164 - RNA, 2165 nt, Homo sapiens
  Homo sapiens high mobility group AT-hook 1 (HMGA1), transcript variant 4
cuuuuuaagcuccccugagccggugcugcgcuccucuaauugggacuccgagccggggcuauuucuggcgcuggcgcggcuccaagaaggcgugaguucgcggccgcuccgguggcuucuuuuuuuuauaucuauaauuuaauuaaauuauuuauuuauugaggccgcgcacgggccgugcccagcuuccugccccucgccauccuucgggggagggggaauauuuuuguccccccgccuggcugugacacauaaauaccccgcgggggccugggcggcgagcacgcggcggcggcggucucugagcgccucugcucucucccgguuucagauccgcauuugcuaccagcggcggccgcggcggagccaggccgguccucagcgcccagcaccgccgcucccggcaacccggagcgcgcaccgcaggccggcggccgagcucgcgcaucccagccaucacucuuccaccugcuccuuagagaagggaagaugagugagucgagcucgaaguccagccagcccuuggccuccaagcaggaaaaggacggcacugagaagcggggccggggcaggccgcgcaagcagccuccgaaggagcccagcgaagugccaacaccuaagagaccucggggccgaccaaagggaagcaaaaacaagggugcugccaagacccggaaaaccaccacaacuccaggaaggaaaccaaggggcagacccaaaaaacuggagaaggaggaagaggagggcaucucgcaggaguccucggaggaggagcagugacccaugcgugccgccugcuccucacuggaggagcagcuuccuucugggacuggacagcuuugcuccgcucccaccgcccccaccccuuccccaggcccaccaucaccaccgccucuggccgccacccccaucuuccaccugugcccucaccaccacacuacacagcacaccagccgcugcagggcucccaugggcugaguggggagcaguuuuccccuggccucaguucccagcuccccccgcccacccacgcauacacacaugcccuccuggacaaggcuaacaucccacuuagccgcacccugcaccugcugcguccccacucccuuggugguggggacauugcucucugggcuuuugguuugggggcgcccucucugcuccuucacuguucccucuggcuucccauaguggggccugggaggguuccccuggccuuaaaaggggcccaagccccaucucauccuggcacgcccuacuccacugcccuggcagcagcagguguggccaauggaggggggugcuggcccccaggauucccccagccaaacugucuuugucaccacguggggcucacuuuucauccuuccccaacuucccuaguccccguacuagguuggacagcccccuucgguuacaggaaggcaggaggggugaguccccuacucccucuucacuguggccacagcccccuugcccuccgccugggaucugaguacauauuguggugauggagaugcagucacuuauuguccaggugaggcccaagagcccuguggccgccaccugaggugggcuggggcugcuccccuaacccuacuuugcuuccgccacucagccauuucccccuccucagauggggcaccaauaacaaggagcucacccugcccgcucccaaccccccuccugcuccucccugccccccaagguucugguuccauuuuuccucuguucacaaacuaccucuggacaguuguguuguuuuuuguucaauguuccauucuucgacauccgucauugcugcugcuaccagcgccaaauguucauccucauugccuccuguucugcccacgauccccucccccaagauacucuuuguggggaagaggggcuggggcauggcaggcugggugaccgacuaccccagucccagggaagguggggcccugccccuaggaugcugcagcagagugagcaagggggcccaaaucgaccauaaaggguguaggggccaccuccucccccuguucuguuggggagggguagccaugauuugucccagccuggggcucccucucugguuuccuauuugcaguuacuugaauaaaaaaaauauccuuuucuggaaaaaaaaaaaaaaaaaaaaaaaaaaaaaaaaaaaaaaa

SEQ ID NO: 165 - protein, 70 aa, Homo sapiens
  Homo sapiens high mobility group AT-hook 1 (HMGA1), transcript variant 4
MSESSSKSSQPLASKQEKDGTEKRGRGRPRKQPPKEPSEVPTPKRPRGRPKGSKNKGAAKTRKTTTTPGR

SEQ ID NO: 166 - RNA, 1884 nt, Homo sapiens
  Homo sapiens high mobility group AT-hook 1 (HMGA1), transcript variant 5
cauuugcauggccccgcccccugagugacacggcuggcgcgggcgggcccguccccccugccccugggucgcucuuuuuaagcuccccugagccggugcugcgcuccucuaauugggacuccgagccggggcuauuucuggcgcuggcgcggcuccaagaaggccaucccagccaucacucuuccaccugcuccuuagagaagggaagaugagugagucgagcucgaaguccagccagcccuuggccuccaagcaggaaaaggacggcacugagaagcggggccggggcaggccgcgcaagcagccuccgaaggagcccagcgaagugccaacaccuaagagaccucggggccgaccaaagggaagcaaaaacaagggugcugccaagacccggaaaaccaccacaacuccaggaaggaaaccaaggggcagacccaaaaaacuggagaaggaggaagaggagggcaucucgcaggaguccucggaggaggagcagugacccaugcgugccgccugcuccucacuggaggagcagcuuccuucugggacuggacagcuuugcuccgcucccaccgcccccaccccuuccccaggcccaccaucaccaccgccucuggccgccacccccaucuuccaccugugcccucaccaccacacuacacagcacaccagccgcugcagggcucccaugggcugaguggggagcaguuuuccccuggccucaguucccagcuccccccgcccacccacgcauacacacaugcccuccuggacaaggcuaacaucccacuuagccgcacccugcaccugcugcguccccacucccuuggugguggggacauugcucucugggcuuuugguuugggggcgcccucucugcuccuucacuguucccucuggcuucccauaguggggccugggaggguuccccuggccuuaaaaggggcccaagccccaucucauccuggcacgcccuacuccacugcccuggcagcagcagguguggccaauggaggggggugcuggcccccaggauucccccagccaaacugucuuugucaccacguggggcucacuuuucauccuuccccaacuucccuaguccccguacuagguuggacagcccccuucgguuacaggaaggcaggaggggugaguccccuacucccucuucacuguggccacagcccccuugcccuccgccugggaucugaguacauauuguggugauggagaugcagucacuuauuguccaggugaggcccaagagcccuguggccgccaccugaggugggcuggggcugcuccccuaacccuacuuugcuuccgccacucagccauuucccccuccucagauggggcaccaauaacaaggagcucacccugcccgcucccaaccccccuccugcuccucccugccccccaagguucugguuccauuuuuccucuguucacaaacuaccucuggacaguuguguuguuuuuuguucaauguuccauucuucgacauccgucauugcugcugcuaccagcgccaaauguucauccucauugccuccuguucugcccacgauccccucccccaagauacucuuuguggggaagaggggcuggggcauggcaggcugggugaccgacuaccccagucccagggaagguggggcccugccccuaggaugcugcagcagagugagcaagggggcccaaaucgaccauaaaggguguaggggccaccuccucccccuguucuguuggggagggguagccaugauuugucccagccuggggcucccucucugguuuccuauuugcaguuacuugaauaaaaaaaauauccuuuucuggaaaaaaaaaaaaaaaaaaaaaaaaaaaaaaaaaaaaaaa

SEQ ID NO: 167 - protein, 96 aa, Homo sapiens; identical to SEQ ID NO: 159
  Homo sapiens high mobility group AT-hook 1 (HMGA1), transcript variant 5

SEQ ID NO: 168 - RNA, 1887 nt, Homo sapiens
  Homo sapiens high mobility group AT-hook 1 (HMGA1), transcript variant 7
gguccccagcagcaagagguggggggaggcaccagaugguaugagagcuuccagggagacccgccaagaucuccaggcaggccgggccaggcucucugggauuccggacuggcuucgcccaacuggaacccccucgaugagggggccccaggcagaccuuauaugagcaucccagccaucacucuuccaccugcuccuuagagaagggaagaugagugagucgagcucgaaguccagccagcccuuggccuccaagcaggaaaaggacggcacugagaagcggggccggggcaggccgcgcaagcagccuccgaaggagcccagcgaagugccaacaccuaagagaccucggggccgaccaaagggaagcaaaaacaagggugcugccaagacccggaaaaccaccacaacuccaggaaggaaaccaaggggcagacccaaaaaacuggagaaggaggaagaggagggcaucucgcaggaguccucggaggaggagcagugacccaugcgugccgccugcuccucacuggaggagcagcuuccuucugggacuggacagcuuugcuccgcucccaccgcccccaccccuuccccaggcccaccaucaccaccgccucuggccgccacccccaucuuccaccugugcccucaccaccacacuacacagcacaccagccgcugcagggcucccaugggcugaguggggagcaguuuuccccuggccucaguucccagcuccccccgcccacccacgcauacacacaugcccuccuggacaaggcuaacaucccacuuagccgcacccugcaccugcugcguccccacucccuuggugguggggacauugcucucugggcuuuugguuugggggcgcccucucugcuccuucacuguucccucuggcuucccauaguggggccugggaggguuccccuggccuuaaaaggggcccaagccccaucucauccuggcacgcccuacuccacugcccuggcagcagcagguguggccaauggaggggggugcuggcccccaggauucccccagccaaacugucuuugucaccacguggggcucacuuuucauccuuccccaacuucccuaguccccguacuagguuggacagcccccuucgguuacaggaaggcaggaggggugaguccccuacucccucuucacuguggccacagcccccuugcccuccgccugggaucugaguacauauuguggugauggagaugcagucacuuauuguccaggugaggcccaagagcccuguggccgccaccugaggugggcuggggcugcuccccuaacccuacuuugcuuccgccacucagccauuucccccuccucagauggggcaccaauaacaaggagcucacccugcccgcucccaaccccccuccugcuccucccugccccccaagguucugguuccauuuuuccucuguucacaaacuaccucuggacaguuguguuguuuuuuguucaauguuccauucuucgacauccgucauugcugcugcuaccagcgccaaauguucauccucauugccuccuguucugcccacgauccccucccccaagauacucuuuguggggaagaggggcuggggcauggcaggcugggugaccgacuaccccagucccagggaagguggggcccugccccuaggaugcugcagcagagugagcaagggggcccaaaucgaccauaaaggguguaggggccaccuccucccccuguucuguuggggagggguagccaugauuugucccagccuggggcucccucucugguuuccuauuugcaguuacuugaauaaaaaaaauauccuuuucuggaaaaaaaaaaaaaaaaaaaaaaaaaaaaaaaaaaaaaaa

SEQ ID NO: 169 - protein, 96 aa, Homo sapiens; identical to SEQ ID NO: 159
  Homo sapiens high mobility group AT-hook 1 (HMGA1), transcript variant 7

SEQ ID NO: 170 - RNA, 90 nt, Artificial Sequence
  Gata6 Em Ref
guggauggccuugacugacggcggcuggugcuugccgaagcgcuucggggccgcgggugcggacgccagcgacuccagagccuuuccagc

SEQ ID NO: 171 - DNA, 90 bp, Artificial Sequence
  Gata6 Em Ref Clone 1
ctagaaagatttgactgacggcggctggtgcctgccaaagcgtttcggggctgctgctgcggacgccggcgactccggagcctttccagc

SEQ ID NO: 172 - DNA, 90 bp, Artificial Sequence; identical to SEQ ID NO: 171
  Gata6 Em Ref Clone 2

SEQ ID NO: 173 - DNA, 88 bp, Artificial Sequence
  Gata6 Em Ref Clone 3
tcagcagatttgactgacggcggctggtgcctgccaaagcgtttcggggctgctgccgcggacgccggcgactccgagcctttccagc

SEQ ID NO: 174 - DNA, 89 bp, Artificial Sequence
  Gata6 Em Ref Clone 4
ttagcaagatttgactgacggcggctggtgcctgccaaagcgtttcggggctgctgctgcggacgccggcgactccgagcctttccagc

SEQ ID NO: 175 - DNA, 87 bp, Artificial Sequence
  Gata6 Em Ref Clone 5
tcagcagattgactgacggcggctggtgcctgccaaagcgtttcggggctgctgctgcggacgccggcgactccgagcctttccagc

SEQ ID NO: 176 - RNA, 89 nt, Artificial Sequence
  Gata6 Ad Ref
aggacccagacugcugcccccgcccuggcgucccacuuucccugggccgaguugcauuucucucuggggcucgcguucgggcuggucag

SEQ ID NO: 177 - DNA, 89 bp, Artificial Sequence
  Gata6 Ad Ref Clone 1
aggacccagactgctgcccccgccctggcgtcccactttccctgggccgagttgcatttctctctggggctcgcgttcgggctggtcag

SEQ ID NO: 178 - DNA, 89 bp, Artificial Sequence; identical to SEQ ID NO: 177
  Gata6 Ad Ref Clone 2

SEQ ID NO: 179 - DNA, 89 bp, Artificial Sequence; identical to SEQ ID NO: 177
  Gata6 Ad Ref Clone 3

SEQ ID NO: 180 - DNA, 89 bp, Artificial Sequence; identical to SEQ ID NO: 177
  Gata6 Ad Ref Clone 4

SEQ ID NO: 181 - DNA, 89 bp, Artificial Sequence
  Gata6 Ad Ref Clone 5
aggacccagactgctgcccccgccctggcgtcccactttccctgggccgagttgcatttctctctggggctcgcgttcgggctggccag

SEQ ID NO: 182 - RNA, 79 nt, Artificial Sequence
  Nkx2-1 Em Ref
cagcgaggcuucgccuucccccucucccuuuuuuuuccuccucuuccuuccuccuccagccgccgccgaaucaugucga

SEQ ID NO: 183 - DNA, 79 bp, Artificial Sequence
  Nkx2-1 Em Ref Clone 1
cagcgaggcttcgccttccccctctcccttttttttcctcctcttccttcctcctccagccgccgccgaatcatgtcga

SEQ ID NO: 184 - DNA, 80 bp, Artificial Sequence
  Nkx2-1 Em Ref Clone 2
cagtcgaggcttcgccttccccctctcccttttttttcctcctcttccttcctcctccagccgccgccgaatcatgtcga

SEQ ID NO: 185 - DNA, 79 bp, Artificial Sequence
  Nkx2-1 Em Ref Clone 3
cagcgaggcttcgccttccccctctcccttatttttcctcctcttccttcctcctccagccgccgccgaatcatgtcga

SEQ ID NO: 186 - DNA, 80 bp, Artificial Sequence
  Nkx2-1 Em Ref Clone 4
cagcgaggcttcgccttccccctctcccttaatttttcctcctcttccttcctcctccagccgccgccgaatcatgtcga

SEQ ID NO: 187 - DNA, 79 bp, Artificial Sequence; identical to SEQ ID NO: 183
  Nkx2-1 Em Ref Clone 5

SEQ ID NO: 188 - RNA, 89 nt, Artificial Sequence
  Nkx2-1 Ad Ref
uccggaggcagugggaaggcgcggggcugggaggccgcggcgggagggaggagcagccccggcaggcucagccgccgccgaaucauguc

SEQ ID NO: 189 - DNA, 89 bp, Artificial Sequence
  Nkx2-1 Ad Ref Clone 1
tccggaggcagtgggaaggcgcggggctgggaggccgcggcgggagggaggagcagccccggcaggctcagccgccgccgaatcatgtc

SEQ ID NO: 190 - DNA, 90 bp, Artificial Sequence
  Nkx2-1 Ad Ref Clone 2
tccggaggcagtggggaaggcgcggggctgggaggccgcggcgggagggaggagcagccccggcaggctcagccgccgccgaatcatgtc

SEQ ID NO: 191 - DNA, 89 bp, Artificial Sequence; identical to SEQ ID NO: 189
  Nkx2-1 Ad Ref Clone 3

SEQ ID NO: 192 - DNA, 89 bp, Artificial Sequence; identical to SEQ ID NO: 189
  Nkx2-1 Ad Ref Clone 4

SEQ ID NO: 193 - DNA, 89 bp, Artificial Sequence; identical to SEQ ID NO: 189
  Nkx2-1 Ad Ref Clone 5

SEQ ID NO: 194 - DNA, 78 bp, Artificial Sequence
  Nkx2-1 Em Ref Clone 6
cagcgagggctcgccttccccctctccctttttttcctcctcttccttcctcctccagccgccgccgaatcatgtcga

SEQ ID NO: 195 - DNA, 89 bp, Artificial Sequence; identical to SEQ ID NO: 189
  Nkx2-1 Ad Ref Clone 6

(19) United States
(12) Patent Application Publication
(10) Pub. No.: US 2018/0051344 A1
(43) Pub. Date: Feb. 22, 2018

(54) EMBRYONIC ISOFORMS OF GATA6 AND NKX2-1 FOR USE IN LUNG CANCER DIAGNOSIS

(71) Applicants: Max-Planck-Gesellschaft Zur Förderung der Wissenschaften e. V., München (DE); Justus-Liebig-Universität Giessen, Giessen (DE)

(72) Inventors: Guillermo BARRETO, Karben (DE); Aditi MEHTA, Bad Nauheim (DE); Achim TRESCH, Köln (DE); Stephanie DOBERSCH, Giessen (DE); Addi Josua ROMERO OLMEDO, Bad Nauheim (DE); Werner SEEGER, Biebertal (DE); Thomas BRAUN, Bad Nauheim (DE); Andreas GÜNTHER, Pohlheim (DE)

(21) Appl. No.: 15/523,753

(22) PCT Filed: Nov. 3, 2015
(86) PCT No.: PCT/EP2015/075615

§ 371 Date: May 2, 2017

(30) Foreign Application Priority Data
Nov. 3, 2014 (EP) 14003697.1
Nov. 26, 2014 (EP) 14195027.9

(51) Int. Cl. C12Q 1/68; G01N 33/574; G06F 19/00; C12N 15/113

(52) U.S. Cl. CPC C12Q 1/6886; G01N 33/57423; G06F 19/30; C12Q 2600/158; C12Q 2600/112; C12Q 2600/156; C12N 15/1135

(57) ABSTRACT

The present invention relates to a Statistical method of assessing whether a subject suffers from Cancer or is prone to suffering from Cancer, said method comprising the step of performing at least one Statistical algorithm for Classification and for regression on measurement data of the subject, wherein the measurement data of the subject comprises at least one of the following: a value of GATA6 Em isoform in at least one sample taken from the subject, a value NKX2-1 Em isoform in said at least one sample, a value of GATA6 Ad isoform in said at least one sample, NKX2-1 Ad isoform in said at least one sample; and wherein at least one of the following is used as at least one classifier or a component of at least one classifier in the Statistical method: GATA6 Em isoform, NKX2-1 Em isoform, GATA6 Ad isoform, NKX2-1 Ad isoform, ratio of GATA6 Em isoform/GATA6 Ad isoform, ratio of NKX2-1 Em isoform/NKX2-1 Ad isoform.

Lung cancer (LC) is the leading cause of cancer-related deaths worldwide, accounting for an estimated 1.6 million deaths out of 1.8 million cases in 2012 (Globocan 2012). The incidence pattern of LC closely parallels the mortality rate because of persistently low survival rates. There are two major classes of LC, non-small cell lung cancer (NSCLC, representing 85% of all lung cancers) and small cell lung cancer (SCLC, the remaining 15%)¹. Histologically, NSCLC is further divided into three major subtypes; squamous cell carcinoma, adenocarcinoma and large cell carcinoma. Adenocarcinoma is the most common form and has approximately 40% prevalence, followed by squamous cell and large cell carcinoma, which represent 25% and 10%, respectively². Clinical manifestations of LC are diverse and patients are mostly asymptomatic at early stages. Symptoms, even when present, are non-specific and unfortunately mimic more common benign etiologies³. Traditional diagnostic strategies for LC include imaging tests, such as chest X-ray radiography (CXR) or computed tomography (CT), cytological assessment of sputum or bronchial suctioning and histopathological evaluation of biopsies taken during bronchoscopy, mediastinoscopy, open lung surgery or from metastasis resections⁴⁻⁶. In the majority of patients, these procedures are initiated after the development of symptoms, therefore at advanced stages of the disease, when the overall condition of the patient is already impaired and prognosis is poor, as shown by the low five-year patient survival of 1-5%¹. Strikingly, patient survival is high as 52% if LC is diagnosed early, demonstrating that early diagnosis of LC is pivotal to increase the probability of successful therapy.

Accordingly, there is a need for new techniques for diagnosis of specific cancers and their subtypes as well as for further and/or alternative treatment options in cancer therapy. Thus, the technical problem underlying the present invention is the provision of reliable means and methods for the detection of cancer, in particular lung cancer and its subtypes, and for the determination of treatment options.

The solution to this technical problem is provided by the embodiments as defined herein and as characterized in the claims.

The invention provides a statistical method for assessing whether a subject suffers from cancer or is prone to suffering from cancer. The invention provides an anti-cancer agent and/or radiation therapy, said agent or radiation therapy being selected on basis of the patient group determined by the statistical method provided herein.

The object of the invention is solved with the features of the independent claims. Dependent claims refer to preferred embodiments.

The invention provides a statistical method of assessing whether a subject suffers from cancer or is prone to suffering from cancer, said method comprising the step of performing at least one statistical algorithm for classification and for regression on measurement data of the subject, wherein the measurement data of the subject comprises at least one of the following: a value of GATA6 Em isoform in at least one sample taken from the subject, a value NKX2-1 Em isoform in said at least one sample, a value of GATA6 Ad isoform in said at least one sample, NKX2-1 Ad isoform in said at least one sample; and wherein at least one of the following is used as at least one classifier or a component of at least one classifier in the statistical method: GATA6 Em isoform, NKX2-1 Em isoform, GATA6 Ad isoform, NKX2-1 Ad isoform, ratio of GATA6 Em isoform/GATA6 Ad isoform, ratio of NKX2-1 Em isoform/NKX2-1 Ad isoform.

Statistical algorithms for classification and for regression on measurement data are generally known to the skilled person. Examples of statistical algorithms can be found in the following textbooks:
“The Elements of Statistical Learning: Data Mining, Inference, and Prediction, Second Edition (Springer Series in Statistics)”, Trevor Hastie et al., Springer, 2011
“Pattern Recognition and Machine Learning”, Christopher M. Bishop, Springer, 2011. B. Schölkopf, A. Smola, Learning with Kernels—Support Vector Machines, Regularization, Optimization and Beyond, MIT Press, Cambridge, Mass., 2002.

Preferably, these algorithms are grossly partitioned into parametric approaches that explicitly model the data by one member of a parametrized family of probability distribuions (e.g., linear discriminant analysis or logit regression), and non-parametric approaches like Neural Networks or Support Vector Machines that do not rely on a distributional assumption.

According to an embodiment, said value of the GATA6 Em isoform in said at least one sample is obtained by measuring the amount of a specific transcription factor isoform in said at least one sample of said subject, wherein said specific transcription isoform is the GATA6 Em isoform comprising the nucleic acid sequence of SEQ ID No: 1 or the GATA6 Em isoform comprising a nucleic acid sequence with up to 55 additions, deletions or substitutions of SEQ ID NO: 1.

According to an embodiment, said value of the NKX2-1 Em isoform in said at least one sample is obtained by measuring the amount of a specific transcription factor isoform in said at least one sample of said subject, wherein said specific transcription isoform is the NKX2-1 Em isoform comprising the nucleic acid sequence of SEQ ID No: 2 or the NKX2-1 Em isoform comprising a nucleic acid sequence with up to 39 additions, deletions or substitutions of SEQ ID NO: 2.

According to an embodiment, said value of GATA6 Ad isoform in said at least one sample is obtained by measuring the amount of a specific transcription factor isoform in said at least one sample of said subject, wherein said specific transcription isoform is the GATA6 Ad isoform comprising the nucleic acid sequence of SEQ ID No: 5 or the GATA6 Ad isoform comprising a nucleic acid sequence with up to 55 additions, deletions or substitutions of SEQ ID NO: 5.

According to an embodiment, said value of the NKX2-1 Ad isoform in said at least one sample is obtained by measuring the amount of a specific transcription factor isoform in said at least one sample of said subject, wherein said specific transcription isoform is the NKX2-1 Ad isoform comprising the nucleic acid sequence of SEQ ID No: 6 or the NKX2-1 Ad isoform comprising a nucleic acid sequence with up to 38 additions, deletions or substitutions of SEQ ID NO: 6.

According to an embodiment, the statistical method further comprises the step of processing the measurement data, preferably normalizing, rescaling, dimension reducing, and/or noise reducing.

Preferably, the step of processing the measurement data, preferably normalizing, rescaling, dimension reducing, and/or noise reducing is performed before performing the at least one statistical algorithm for classification and for regression on measurement data of the subject.

Preferably, the normalizing of the measurement data comprises the normalizing of at least one of the following: microarray or RNA-Seq measurements.

Preferably the normalizing of the measurement comprises obtaining abundance estimates and/or detecting outlier and/or removing outlier.

Preferably, the reducing of the dimension and/or the reducing of the noise comprises transforming the measurement data into a space where discriminatory methods achieve a higher power.

Preferably, reducing the dimension and/or reducing the noise comprises at least one of the following: principal component analysis, non-linear variant principal component analysis, singular value decomposition, non-linear variant singular value decomposition, independent component analysis, non-linear independent component analysis, a kernel principal component analysis.

According to an embodiment, the statistical method further comprises the steps of cross-validation and/or bootstrapping.

According to an embodiment, the GATA6 Em isoform of said sample is set in relation to a GATA6 Em isoform of at least one control sample and then used as a classifier in the statistical method.

Preferably, set in relation comprises at least one of the following: normalizing the value of the GATA6 Em isoform of said sample with respect to the value of the GATA6 Em isoform of the control sample, subtracting the value of the GATA6 Em isoform of at least one control sample from the GATA6 Em isoform of said sample.

Preferably, said value of the GATA6 Em isoform of said at least one control sample is obtained by measuring in said sample of said subject the amount of a specific transcription factor isoform wherein said specific transcription isoform is the GATA6 Em isoform comprising the nucleic acid sequence of SEQ ID No: 1 or the GATA6 Em isoform comprising a nucleic acid sequence with up to 55 additions, deletions or substitutions of SEQ ID NO: 1.

According to an embodiment, the NKX2-1 Em isoform in said at least one sample is set in relation to a NKX2-1 Em isoform of at least one control sample and then used as a classifier in the statistical method.

Preferably, set in relation comprises at least one of the following: normalizing the value of the NKX2-1 Em isoform of said sample with respect to the value of the NKX2-1 Em isoform of the control sample, subtracting the value of the NKX2-1 Em isoform of at least one control sample from the NKX2-1 Em isoform of said sample.

Preferably, said value of the NKX2-1 Em isoform in said at least one control sample is obtained by measuring in said at least one sample of said subject the amount of a specific transcription factor isoform wherein said specific transcription isoform is the NKX2-1 Em isoform comprising the nucleic acid sequence of SEQ ID No: 2 or the NKX2-1 Em isoform comprising a nucleic acid sequence with up to 39 additions, deletions or substitutions of SEQ ID NO: 2;

According to an embodiment, a ratio of the GATA6 Em isoform and the GATA6 Ad isoform and a ratio of the NKX2-1 Em isoform and the NKX2-1 Ad isoform are used as a classifier.

According to an embodiment, the statistical method comprises a linear classifier.

Preferably, the statistical method comprises at least one of the following: a linear classifier, preferably a support vector machine and/or a linear discriminant analysis and/or decision trees, a regression method, preferably linear, logistic or probit regression, or a penalized version of the regression, preferably a penalized version of the linear, logistic or probit regression, more preferably a Lasso and/or ridge regression, or a generalized linear model, a neural network, or a regression tree, or ensemble methods built from the above algorithms in a process, preferably boosting.

Preferably, the support vector machine is a linear kernel support vector machine. Preferably, the linear kernel support vector machine is the one implemented in the following software: Evgenia Dimitriadou, Kurt Hornik, Friedrich Leisch, David Meyer and Andreas Weingessel (2010). e1071: Misc Functions of the Department of Statistics (e1071), TU Wien. R package version 1.5-24. http://CRAN.Rproject.org/package=e1071.

Preferably, the SVM, does not assume that the data from the sample groups are drawn from a Gaussian distribution. The SVM can be considered as the more robust choice in comparison to the linear discrimination analysis. Preferably, the support vector machine finds a separating hyperplane between data from normal and cancerous samples, which is expected to yield a good generalization performance when applied to new, unseen data. Preferably, the distance to this hyperplane is determined by the following function:

LC_{score}=−α·log₂(ratio of GATA6 Em isoform/GATA6 Ad isoform)−β·log₂(ratio of NKX2-1 Em isoform/NKX2-1 Ad isoform)−γ,

wherein preferably α=0.607, β=1.431, γ=1.916.

Preferably, α=−0.607, β=−1.431, γ=−1.916

Preferably, the function comprises a prefactor (−1) such that the distance to the hyperplane is determined by the following function:

LC_{score}=(−1)−(−α·log₂(ratio of GATA6Em isoform/GATA6Ad isoform)−β·log₂(ratio of NKX2-1Em isoform/NKX2-1Ad isoform)−γ),

wherein preferably α=0.607, β=1.431, γ=1.916.

The amount of said specific transcription factor isoform(s) can be measured on the mRNA level.

The appended example shows that the expression ratio remained stable for both control donor as well as LC EBC samples until 75 ng of RNA starting material. Decreasing the starting material below 75 ng resulted in suboptimal detection of the Em-isoform in the control and the Ad-isoform in the LC group, which led to distorted ratios. If the amount of the transcription factor isoform(s) is determined/measured in accordance with the present invention, it is preferred that the starting material (mRNA/RNA) contains/is more than about 75 ng of RNA.

According to an embodiment, the amount of said specific transcription factor isoform(s) is measured via a polymerase chain reaction-based method, According to an embodiment, the amount of said specific transcription factor isoform(s) is measured via a polymerase chain reaction-based method, an in situ hybridization-based method, or a microarray. According to an embodiment, the amount of said specific transcription factor isoform(s) is measured via a polymerase chain reaction-based method. According to an embodiment, said polymerase chain reaction-based method is a quantitative reverse transcriptase polymerase chain reaction.

According to an embodiment, the step of measuring in a sample of said subject the amount of a specific transcription factor comprises the contacting of the sample with primers, wherein said primers can be used for amplifying at least one of the specific transcription factor isoforms. According to an embodiment, said primers are selected from the group of primers having a nucleic acid sequence as set forth in SEQ ID NOs 9 to 40, particularly one or more primers/primer pairs having a nucleic acid sequence as set forth in SEQ ID NOs 9 to 24. For example, one or more of the following primers/primer pairs can be used in accordance with the present invention:

| | Primers | Primers for Human (5′→3′) (For |
|---|---|---|
| Gene | for Human (5′→3′) | RNA from tissue sections) |
| Gata6-Em Fwd | SEQ ID NO 9: | SEQ ID NO 10: |
| | CTCGGCTTCTCTCCGCGCCTG | TTGACTGACGGCGGCTGGTG |
| Gata6-Em Rev | SEQ ID NO 11: | SEQ ID NO 12: |
| | AGCTGAGGCGTCCCGCAGTTG | CTCCCGCGCTGGAAAGGCTC |
| Gata6-Ad Fwd | SEQ ID NO 13: | SEQ ID NO 14: |
| | GCGGTTTCGTTTTCGGGGAC | AGGACCCAGACTGCTGCCCC |
| Gata6-Ad Rev | SEQ ID NO 15: | SEQ ID NO 16: |
| | AAGGGATGCGAAGCGTAGGA | CTGACCAGCCCGAACGCGAG |
| Nkx2-1-Em Fwd | SEQ ID NO 17: | SEQ ID NO 18: |
| | AAACCTGGCGCCGGGCTAAA | CAGCGAGGCTTCGCCTTCCC |
| Nkx2-1-Em Rev | SEQ ID NO 19: | SEQ ID NO 20: |
| | GGAGAGGGGGAAGGCGAAGCC | TCGACATGATTCGGCGGCGG |
| Nkx2-1-Ad Fwd | SEQ ID NO 21: | SEQ ID NO 22: |
| | AGCGAAGCCCGATGTGGTCC | TCCGGAGGCAGTGGGAAGGC |
| Nk2-1-Ad Rev | SEQ ID NO 23: | SEQ ID NO 24: |
| | CCGCCCTCCATGCCCACTTTC | GACATGATTCGGCGGCGGCT |
| Foxa2-Var1 Fwd | SEQ ID NO 25: | SEQ ID NO 26: |
| | TGCCATGCACTCGGCTTCCAG | CAGGGAGAGGGAGGGCGAGA |
| Foxa2-Var1 Rev | SEQ ID NO 27: | SEQ ID NO 28: |
| | TCATGTTGCCCGAGCCGCTG | CCCCCACCCCCACCCTCTTT |
| Foxa2-Var2 Fwd | SEQ ID NO 29: | SEQ ID NO 30: |
| | CTGCTAGAGGGGCTGCTTGCG | CGCTTCTCCCGAGGCCGTTC |
| Foxa2-Var2 Rev | SEQ ID NO 31: | SEQ ID NO 32: |
| | ACGGCTCGTGCCCTTCCATC | TAACTCGCCCGCTGCTGCTC |
| Id2-Var1 Fwd | SEQ ID NO 33: | SEQ ID NO 34: |
| | AACCCCTGTGGACGACCCGA | TGCGGATAAAAGCCGCCCCG |
| Id2-Var1 Rev | SEQ ID NO 35 | SEQ ID NO 36: |
| | GCCCGGGTCTCTGGTGATGC | AGCTAGCTGCGCTTGGCACC |
| Id2-Var2 Fwd | SEQ ID NO 37: | SEQ ID NO 38: |
| | CTGCGGTGCTGAACTCGCCC | CCCCCTGCGGTGCTGAACTC |
| Id2-Var2 Rev | SEQ ID NO 39: | SEQ ID NO 40: |
| | GACGAGCGGGCGCTTCCATT | TAACTCGCCCGCTGCTGCTC |

According to an embodiment, the amount of said specific transcription factor isoform(s) can be measured on the polypeptide/protein level. According to an embodiment, the amount of said specific transcription factor isoform(s) is measured by an ELISA, a gel- or blot-based method, mass spectrometry, flow cytometry or FACS.

According to an embodiment, the cancer is a lung cancer. According to an embodiment, said lung cancer is non-small cell lung cancer (NSCLC) or small cell lung cancer (SCLC).

According to an embodiment, the sample comprises tumor cells. According to an embodiment, the sample is a biopsy sample, a breath condensate sample, a blood sample, a bronchoalveolar lavage fluid sample, a mucus sample or a phlegm sample. Preferably, the sample is a breath condensate sample.

According to an embodiment, the subject is a human subject. According to an embodiment, said human subject is a subject having an increased risk for developing cancer. A human subject having an increased risk for developing cancer can, for example, be a human subject that is a current or former smoker(s); and/or that was/is exposed to smoke, like environmental smoke, cooking fumes, and/or indoor smoky coal emissions; and/or that was/is exposed to asbestos, some metals (e.g. nickel, arsenic and cadmium), radon, and/or ionizing radiation. A human subject having an increased risk for developing cancer can, for example, be a human subject that has shown cancer-like lesions in a preceding computed tomography scan.

According to an embodiment, the method further comprises the detection of one or more additional markers in a sample of said subject. According to an embodiment, said one or more additional markers are one or more markers for classifying cancer. According to an embodiment, said one or more additional markers are one or more markers for classifying lung cancer into subtypes of lung cancer. According to an embodiment, said one or more markers for classifying lung cancer are differentially expressed.

According to an embodiment, said one or more markers for classifying lung cancer are one or more markers for classifying non-small cell lung cancer (NSCLC) into subtypes of NSCLC. According to an embodiment, said one or more markers for classifying NSCLC are selected from the group consisting of SFTPA1, SFTPB, NAPSA, hsa-let7-d, VEGFA, VEGFB, VEGFC, VEGFD, PLAUR, TP63, KRT5, KRT6A, KRT7, hsa-miR9, HMGA1 and CDH1. Exemplary nucleic acid sequences and amino acid sequences of these markers are provided in the present application.

The specific transcription factor isoform(s) and/or the additional markers (like SFTPA1, SFTPB, NAPSA, VEGFA, VEGFB, VEGFC, VEGFD, PLAUR, TP63, KRT5, KRT6A, KRT7, HMGA1 and/or CDH1) can be measured on the protein/polypeptide or the mRNA level. Additional markers like hsa-let7-d, hsa-miR9, can be measured on the mRNA level.

For example, the amount can be measured via a polymerase chain reaction-based method, an in situ hybridization-based method, or a microarray, or a quantitative reverse transcriptase polymerase chain reaction.

For example, the amount can be measured on the polypeptide/protein level, for example, by an ELISA, a gel- or blot-based method, mass spectrometry, flow cytometry or FACS.

For example, if the specific transcription factor isoform(s) and/or additional marker(s) is/are measured on the protein level, contacting and binding can be performed by taking advantage of immunoagglutination, immunoprecipitation (e.g. immunodiffusion, immunelectrophoresis, immune fixation), western blotting techniques (e.g. (in situ) immuno histochemistry, (in situ) immuno cytochemistry, affinitychromatography, enzyme immunoassays), and the like. These and other suitable methods of contacting proteins are well known in the art and are, for example, also described in Sambrook and Russell (2001, loc. cit.).

In case the specific transcription factor isoform(s) and/or additional marker(s) is a protein, quantification can be performed by taking advantage of the techniques referred to above, in particular Western blotting techniques. Generally, the skilled person is aware of methods for the quantitation of polypeptides. Amounts of purified polypeptide in solution can be determined by physical methods, e.g. photometry. Methods of quantifying a particular polypeptide in a mixture rely on specific binding, e.g of antibodies. Specific detection and quantitation methods exploiting the specificity of antibodies comprise for example immunohistochemistry (in situ). Western blotting combines separation of a mixture of proteins by electrophoresis and specific detection with antibodies. Electrophoresis may be multi-dimensional such as 2D electrophoresis. Usually, polypeptides are separated in 2D electrophoresis by their apparent molecular weight along one dimension and by their isoelectric point along the other direction.

For example, if the specific transcription factor isoform(s) and/or additional marker(s) is/are measured on the RNA/mRNA level, contacting and binding can be performed by taking advantage of Northern blotting techniques or PCR techniques/via a polymerase chain reaction-based method, like quantitative reverse transcriptase polymerase chain reaction or in-situ PCR, an in situ hybridization-based method, or a microarray. These and other suitable methods for binding (specific) mRNA are well known in the art and are, for example, described in Sambrook and Russell (2001, loc. cit.).

If the specific transcription factor isoform(s) and/or additional marker(s) is an mRNA, determination can be performed by taking advantage of northern blotting techniques, hybridization on microarrays or DNA chips equipped with one or more probes or probe sets specific for mRNA transcripts or PCR techniques referred to above, like, for example, quantitative PCR techniques, such as Real time PCR. A skilled person is capable of determining the amount of the component, in particular said gene products, by taking advantage of a correlation, preferably a linear correlation, between the intensity of a Raman signal and the amount of the component to be determined.

According to an embodiment, said subtype of NSCLC is classified as adenocarcinoma, if said one or more markers for classifying NSCLC into subtypes of NSCLC are one or more of SFTPA1, SFTPB and NAPSA, and

if the level of one or more of SFTPA1, SFTPB and NAPSA is increased compared to a control. Preferably the level of SFTPA1 is the mRNA level or the protein level of SFTPA1.

According to an embodiment, said subtype of NSCLC is classified as adenocarcinoma, if said marker for classifying NSCLC into subtypes of NSCLC is hsa-let7-d, and if the level of hsa-let7-d is decreased compared to a control. Preferably the level of hsa-let7-d is the RNA level of hsa-let7-d.

According to an embodiment, said subtype of NSCLC is classified as metastatic adenocarcinoma,

if said marker for classifying NSCLC into subtypes of NSCLC is VEGFA, VEGFB, VEGFC, VEGFD and/or PLAUR, and
if the level of VEGFA, VEGFB, VEGFC, VEGFD and/or PLAUR is increased compared to a control. Preferably the level of VEGFA, VEGFB, VEGFC, VEGFD and/or PLAUR is the mRNA level or the protein level of VEGFA, VEGFB, VEGFC, VEGFD and/or PLAUR.

According to an embodiment, said subtype of NSCLC is classified as squamous cell carcinoma, if said marker for classifying NSCLC into subtypes of NSCLC is one or more of TP63, KRT5, KRT6A, KRT7 and hsa-miR9, and

if the level of one or more of one or more of TP63, KRT5, KRT6A, KRT7 and hsa-miR9, is increased compared to a control. Preferably the level of TP63, KRT5, KRT6A and KRT7 is the mRNA level or the protein level of TP63, KRT5, KRT6A and KRT7. Preferably the level of hsa-miR9 is the RNA level of hsa-miR9.

According to an embodiment, said subtype of NSCLC is classified as large cell lung carcinoma, if said marker for classifying NSCLC into subtypes of NSCLC is HMGA1, and if the level of HMGA1 is increased compared to a control. Preferably the level of HMGA1 is the mRNA level or the protein level of HMGA1.

According to an embodiment, said subtype of NSCLC is classified as large cell lung carcinoma,

if said marker for classifying NSCLC into subtypes of NSCLC is CDH1, and
if the level of CDH1 is decreased compared to a control. Preferably the level of CDH1 is the mRNA level or the protein level of CDH1.

According to an embodiment, said one or more markers for classifying lung cancer are genomic alterations. A person skilled in the art knows how to determine genomic alterations, a mutation(s) or a polymorphism(s) in a gene by his common general knowledge and the teaching provided herein. Exemplary, non-limiting techniques for determining such genomic alteration(s), mutation(s) and/or polymorphism(s) are described below.

Genomic alterations, including mutations and polymorphisms, can be detected by DNA sequencing, including pyrosequencing and Sanger sequencing methods, PCR based methods including restriction fragment length polymorphisms, taqman probes and molecular beacons, or using DNA arrays. Genomic alterations including chromosomal changes, such as translocations or deletions can be identified by conventional cytogenetic stainings, fluorescent in situ hybridization, comparative genomic hybridization and array based comparative genomic hybridization, or PCR based analysis.

According to an embodiment, said one or more markers for classifying lung cancer are one or more markers for classifying non-small cell lung cancer (NSCLC) into subtypes of NSCLC.

According to an embodiment, said subtype of NSCLC is classified as adenocarcinoma,

if said marker for classifying NSCLC into subtypes of NSCLC is KRAS G12D or G12V G-->C/T transversion at codon for Exon 12, and
if said marker is present in the sample from the subject.

Preferably, the specific mutations of KRAS found in NSCLC are one or more of: G34T, G35A, G35T and G37T and G38T (the last 2 result in mutations of codon 13 which are also oncogenic)

### Ref: 21197450.

These mutations are negative predictors of response to EGFR therapy in patients.

According to an embodiment, said subtype of NSCLC is classified as metastatic adenocarcinoma,

if said marker for classifying NSCLC into subtypes of NSCLC is KRAS G12D//TP53 mutations R172H Substitution in p53 (Li-Fraumeni syndrome), and
if said marker is present in the sample from the subject.

Preferably, metastatic adenocarcinoma is characterized/classified by a combination of KRAS and TP53 as defined above.

According to an embodiment, said subtype of NSCLC is classified as adenocarcinoma in never-smokers,

if said marker for classifying NSCLC into subtypes of NSCLC is KRAS G12D G-->G-->A (G35A) transition, and
if said marker is present in the sample from the subject.

According to an embodiment, said subtype of NSCLC is classified as adenocarcinoma or squamous cell carcinoma,

if said marker for classifying NSCLC into subtypes of NSCLC is TP53 mutations, translocations, and
if said marker is present in the sample from the subject.

Preferably, the most frequent mutations in TP53 for Adenocarinoma: G:C247T:A and for Squamous cell carincoma is G:C274T:A and for SCLC is G:C96T:A.

According to an embodiment, said subtype of NSCLC is classified as drug resistant adenocarcinoma (patients relapse after tyrosine kinase inhibitors),

if said marker for classifying NSCLC into subtypes of NSCLC is EGFR T790M mutation in exon 20, codon 790, and
if said marker is present in the sample from the subject.

According to an embodiment, said subtype of lung cancer is classified as small cell lung cancer (SCLC),

if said marker for classifying lung cancer into subtypes of lung cancer is/are TP53 mutations combined with mutations in RB1, and
if said marker is present in the sample from the subject.

The above mentioned additional markers are suitable markers to classify cancer into subtypes of cancer, and in particular lung cancer into subtypes of lung cancer. This is illustrated by the references below. Accordingly, the one or more additional markers can be suitably be used in accordance with the present invention for a refined analysis using the herein provided statistical method. For example, the expression of one or more of these additional markers can be determined in exhaled breath condensates from patients that are assessed to suffer from cancer or being prone to suffering from cancer in accordance with the statistical method can, in order to classify e.g. cancer subtype (preferably the NSCLC subtype) in the patients. The terms “transition” and “transversion” are used interchangeably herein.

For example, the following one or more markers can be used to classify NSCLC into subtypes of NSCLC:

### Adenocarcinoma:

SFTPA, SFTPB and/or NAPSA: (Garber, Troyanskaya et al. 2001, Ye, Findeis-Hosey et al. 2011, Turner, Cagle et al. 2012, Whithaus, Fukuoka et al. 2012, Taguchi, Hanash et al. 2013); and/or hsa-let7-d: (Lee and Dutta 2007, Kumar, Armenteros-Monterroso et al. 2014); and/or KRAS G12D and/or G12V: (Winslow, Dayton et al. 2011); and/or
TP53 mutations and/or TP53 translocations: (Kishimoto, Murakami et al. 1992)

The term KRAS G12D or G12V (or more particularly the term “KRAS G12D or G12V G-->C/T transversion at codon for Exon 12”) refers to an amino acid substitution at position 12 of the amino acid sequence of KRAS. The substitution is due to a transversion in the coding sequence of KRAS. Particularly the term “KRAS G12D or G12V G-->C/T transversion at codon for Exon 12”) can refer to a G(35)-->C/T transversion at position 35 of the DNA sequence of KRAS within codon 12. The DNA mutation is G→C/T at position 35 of the coding sequence of KRAS, which is changing codon 12 in the amino acid sequence of KRAS. Coding sequences of KRAS can be derived from databases like NCBI. Exemplary coding sequences of KRAS to be used herein are, for example, shown in the database under accession number GI 575403058 (Transcript variant a) or under GI 575403057 (Transcript variant b).

### Metastatic Adenocarcinoma:

VEGFA, VEGFB, VEGFC, VEGFD, and/or PLAUR: (Shijubo, Uede et al. 1999, Garber, Troyanskaya et al. 2001, Su, Yang et al. 2006) (Han, Silverman et al. 2001, Stacker, Caesar et al. 2001, Li, Hu et al. 2014, Qi, Zhu et al. 2014); and/or
KRAS G12D mutations and/or TP53 mutations (such as R172H substitution in TP53 (Li-Fraumeni syndrome)): (Kishimoto, Murakami et al. 1992, Lang, Iwakuma et al. 2004)

The term “KRAS G12D//TP53 mutation(s) R172H Substitution in TP53 (Li-Fraumeni syndrome)” can refer to KRAS G12D mutation(s) and/or TP53 mutation(s) (such as R172H substitution in TP53 (Li-Fraumeni syndrome)).

The term KRAS G12D refers to an amino acid substitution at position 12 of the amino acid sequence of KRAS. The substitution is due to a transversion in the coding sequence of KRAS, like a G-->A (G35A) transition.

The term “TP53 mutation(s)” (or more particularly the term “TP53 mutation(s) R172H Substitution in TP53”) can refer to an amino acid substitution in the amino acid sequence of TP53. The substitution is due to a transition in the coding sequence of TP53. Particularly the term “TP53 mutation(s) R172H Substitution in TP53” can refer to a G to A transition at position 515 (G515A) of the sequence encoding TP53. Coding sequences of TP53 can be derived from databases like NCBI. An exemplary coding sequence of TP53 to be used herein is, for example, shown in the database under accession number GI 23491728.

### Adenocarcinoma in Never-Smokers:

KRAS G12D G-->A (G35A) transition: (Riely, Kris et al. 2008). The terms “KRAS G12D G-->G-->A (G35A) transition” and “KRAS G12D G-->A (G35A) transition” can be used interchangeably herein.

The term “KRAS G12D” or particularly the term “KRAS G12D G-->G-->A (G35A) transition”/“KRAS G12D G-->A (G35A) transition” refers to an amino acid substitution at position 12 of the amino acid sequence of KRAS. The substitution is due to a transition in the coding sequence of KRAS. The terms “KRAS G12D G-->G-->A (G35A) transition”/“KRAS G12D G-->A (G35A) transition” can refer to a KRAS G12D G-->A (G35A) transition. Particularly the term “KRAS G12D G-->G-->A (G35A) transition” refers to an amino acid substitution at position 12 of the amino acid sequence of KRAS which is due to a G-->A (G35A) transition in the coding sequence of KRAS. The amino acid change KRAS G12D results from a change at position 35 in the coding sequence of KRAS, in this case G35 to A.

Drug Resistant Adenocarcinoma (for Example Patients Relapse after Therapy with Tyrosine Kinase Inhibitors):
EGFR T790M mutation in exon 20, codon 790: (Pao, Miller et al. 2005)

The terms “EGFR T790M mutation in exon 20, codon 790” and “EGFR T790M mutation in codon 790” can be used interchangeably herein. The terms “EGFR T790M mutation in exon 20, codon 790” or “EGFR T790M mutation in codon 790” are also known as “EGFR C2369T mutation”.

The term “EGFR T790M mutation”, or particularly the term “EGFR T790M mutation in exon 20, codon 790”, refers to an amino acid substitution at position 790 of the amino acid sequence of EGFR. The amino acid substitution can be due to a transition in the coding sequence of EGFR. Particularly the terms “EGFR T790M mutation in exon 20, codon 790”/“EGFR T790M mutation in codon 790”/“EGFR C2369T mutation” can refer to a C to T transition at position 2369 (i.e. C2369T) of the sequence encoding EGFR. Coding sequences of EGFR can be derived from databases like NCBI. An exemplary coding sequence of EGFR to be used herein is, for example, shown in the database under accession number GI 41327737 (Transcript isoform a), GI 41327731 (Transcript isoform b), GI 41327733 (Transcript isoform c) or 41327735 (Transcript isoform d).

### Squamous Cell Carcinoma:

TP63, KRT5, KRT6 and/or KRT7: (Pelosi, Pasini et al. 2002, Rekhtman, Ang et al. 2011, Whithaus, Fukuoka et al. 2012); and/or
hsa-miR9: (White, Neiman et al. 2013)
TP53 mutations and/or TP53 translocations: (Kishimoto, Murakami et al. 1992)

### Large Cell Lung Cancer/Large Cell Lung Carcinoma:

HMGA1: (Hillion, Wood et al. 2009) and/or

### CDH1: (Kase, Sugio et al. 2000, Garber, Troyanskaya et al. 2001, Asnaghi, Vass et al. 2010)

For example, the following one or more markers can be used to classify lung cancer into the subtype small cell lung cancer (SCLC): TP53 mutations in combination with mutations in RB1: (Sutherland, Proost et al. 2011). Mutations in RB1 may refer to mutations in the tumor suppressor gene Retinoblastioma, RB1. The protein is a negative regulator of cell cylce.

The invention also provides a computer program product comprising one or more computer readable media having computer executable instructions for performing the steps of one of the aforementioned methods.

The present invention relates to a method of treating a subject, said method comprising

a) selecting a subject that is assessed to suffer from cancer or is assessed to be prone to suffering from cancer according to the herein provided statistical method;
b) administering to said cancer patient an effective amount of an anti-cancer agent and/or radiation therapy.

Preferably, the gene mutations can be used to distinguish patients' response to EGFR therapy as mentioned above.

The invention also provides an anti-cancer agent and/or radiation therapy for use in the treatment of a subject, wherein the subject is assessed to suffer from cancer or is assessed to be prone to suffering from cancer according to any of the statistical methods mentioned above. Preferably, the subject/patient is a human subject/patient. In other words, the invention provides an anti-cancer agent and/or radiation therapy, said agent or radiation therapy being selected on basis of the patient group determined by the statistical method provided herein.

For example, conventional chemotherapy (like cisplatin based protocols), radiotherapy (like conventional radiotherapy or radiosurgery), and/or more modern approaches employing tyrosine kinase inhibitors (TKIs), such as gefitinib, erlotinib and/or monoclonal antibodies directed against activating mutations of the tumor (ERGF, ALK or ROS1 mutations) can be used.

If the subject is assessed to suffer from non-small cell lung cancer (NSCLC) or is assessed to be prone to suffering from non-small cell lung cancer (NSCLC) according to any of the statistical methods mentioned above, the following treatment options can be used:

The treatment options for NSCLC are, for example, based on the stage of the disease. Standard treatments include surgery, platinum-based chemotherapy, radiotherapy, combined chemoradiotherapy and/or targeted therapy. The choice of the course of treatment can depend on the stage of the disease, its spread to the surrounding tissues, patient's overall medical condition, and/or especially the patient's pulmonary reserve.

If the subtype of NSCLC (like NSCLC stage I, II or III tumors/cancers) is, for example, adenocarcinoma, squamous cell carcinoma or large cell carcinoma, the following treatment options are conceivable:

For Stage I tumors, surgery is the most consistent and successful treatment for lung cancer patients. Tumors can be removed by lobectomy, segmental, wedge or sleeve resections or pneumectomy as found appropriate (Molina, Yang et al. 2008, Schuchert, Abbas et al. 2010, 2011, Cagle and Chirieac 2012). Five-year survival rate ranges between 40-67% favoring T1N0 or earlier (Martini, Bains et al. 1995). In the patients with potentially resectable tumors but who are unfit for surgery due to an unacceptably high perioperative risk or for patients with inoperable Stage I tumors, primary radiosurgery or conventional radiation therapy is suggested (Dosoretz, Katin et al. 1992, Gauden, Ramsay et al. 1995). Unfortunately, many patients develop local recurrent or second primary tumors after surgical resection. To prevent this, adjuvant chemo or radiation therapy following surgery is recommended pending on the stage prior to surgery (Martini, Bains et al. 1995).

Stage II cancers are routinely treated with surgical resections, however, prognosis is worse than that of Stage I cancers and the 5-year survival rate varies from 25-55% (Martini, Burt et al. 1992). However, patient survival is lower for squamous cell lung cancer. In some cases, neoadjuvant chemotherapy, i.e. preoperative chemotherapy is proposed to be beneficial to reduce tumor size to facilitate surgical resection and eliminate early micrometastases (Burdett, Stewart et al. 2007). In addition, post-operative adjuvant chemotherapy, for instance with cisplatin, may significantly improve prognosis and prevent local recurrences. For inoperable tumors or patients unfit for surgery, radiation therapy is recommended (Pignon, Tribodet et al. 2008).

Stage III NSCLC includes both locally and regionally advanced disease. For resectable NSCLC, surgery to remove the complete tumor and the surrounding lymph nodes is recommended, followed by post-operative chemotherapy. Further, neoadjuvant chemotherapy to shrink the tumor and eradicate micrometastases, thus facilitating surgery, is also an approach of choice (Burdett, Stewart et al. 2007). Further, similar to Stage II, patients are shown to benefit with adjuvant chemotherapy using cisplatin. For unresectable Stage III NSCLC, radiation therapy or a concurrent or sequential combination of chemo- with radiation therapy is recommended (Furuse, Fukuoka et al. 1999).

If the subtype of NSCLC (like NSCLC stage IV tumors/cancers) is, for example, metastatic NSCLC (such as forms of all NSCLC classes/subtypes, like metastatic adenocarcinoma), adenocarcinoma, squamous cell carcinoma or large cell carcinoma the following treatment options are conceivable:

For patients with metastatic NSCLC (Stage IV), treatment is usually aimed to prolong survival and for palliation of disease related symptoms. Standard treatment options include cytotoxic chemotherapy and targeted agents. However, treatment is selected based on comorbidity, performance status, histology, and molecular genetic features of the cancer. First line cytotoxic combination chemotherapy includes a combination of platinum-based chemotherapy (cisplatin or carboplatin) and paclitaxel, gemcitabine, docetaxel, vinorelbine, irinotecan, or pemetrexed (Le Chevalier, Arriagada et al. 1992, Wozniak, Crowley et al. 1998, Mok, Wu et al. 2009). Following the initial response to chemotherapy, maintenance chemotherapy using the initial combination of drugs, or continuing single-agent chemotherapy, or using a new ‘maintenance’ agent is evaluated. (Brodowicz, Krzakowski et al. 2006, Park, Kim et al. 2007, Paz-Ares, de Marinis et al. 2012). Further, based on the molecular analysis of the cancer, patients may benefit from single-agent EGFR tyrosine kinase inhibitors or EML4-ALK inhibitors, as first line treatment (if driver mutations have been encountered) or, even in absence of driver mutations, as second or third line treatment.

If the subtype of NSCLC is, for example, adenocarcinoma, the following treatment options are conceivable:

Among the currently used combinations, definite recommendations regarding drug dose, schedule or combination cannot be made. However, the exception for this is pemetrexed for lung adenocarcinoma (Scagliotti, Parikh et al. 2008). Adenocarcinoma patients, especially adenocarcinoma in never smokers/never smoker patients, benefit from using EGFR tyrosine kinase inhibitors, such as gefitinib (Mok, Wu et al. 2009).

If the subtype of NSCLC is, for example, sqamous cell carcinoma, the following treatment options are conceivable:

In contrast, in patients with squamous cell histology (like patients with squamous cell carcinoma), patient response is significantly better using a combination of cisplatin and gemcitabine versus cisplatin and pemetrexed (Scagliotti, Parikh et al. 2008).

Lastly, for patients with Stage IV NSCLC, palliative radiotherapy may be used to control vocal cord paralysis, hemoptysis, obstructive symptoms or pain related to bone metastases. Surgical intervention may also be recommended for patients with bronchial obstructions.

Standard treatment for recurrent drug resistant NSCLC includes palliative radiation therapy (Sundstrom, Bremnes et al. 2004) and/or combination chemotherapy, for patients who have previously received platinum based chemotherapy. Chemotherapy combinations include Docetaxel, Pemetrexed, Erlotinib after failure of both platinum-based and docetaxel chemotherapies, Gefitinib, Crizotinib for EML4-ALK translocations, EGFR inhibitors in patients with or without EGFR mutations, EML4-ALK inhibitors in patients with EML-ALK translocations (Hanna, Shepherd et al. 2004, Kim, Hirsh et al. 2008, Kwak, Bang et al. 2010, Shaw, Yeap et al. 2011).

If the subtype of NSCLC is, for example, large cell lung cancer/large cell carcinoma, the treatment plan depends on the stage and no definite recommendations can be made beforehand. For example, conventional therapy, like chemotherapy/radiotherapy as disclosed herein, can be contemplated.

If the subtype of lung cancer is, for example, small-cell lung cancer (SCLC), the following treatment options are conceivable:

For treatment purposes, small-cell lung cancer (SCLC) is usually staged as either limited or extensive disease. Limited stage SCLC means that the cancer is only on one side of the chest and includes the lobes and/or lymph nodes on the same side. The tumors are often confined to a small area and can be targeted by a single radiation field. On the other hand, extensive stage represents cancers that have spread to both sides of the chest and may include distant metastases to other organs.

Chemotherapy is the mainstay of treatment of SCLC. For limited stage disease, combined modality of chemotherapy and thoracic radiation therapy, called concurrent chemoradiation, is the most widely used treatment. Active drugs usually include a combination of platinum and etoposide. Based on the patient's health status, radiation therapy may not be recommended and in this case, the patients are treated with chemotherapy alone (Pignon, Arriagada et al. 1992, Warde and Payne 1992, Murray, Coy et al. 1993). Surgical resection for SCLC is limited to management of cases with very limited disease, i.e. small tumors pathologically confined to the lobe of origin. Surgery is generally followed by adjuvant chemotherapy (Osterlind, Hansen et al. 1985, Prasad, Naylor et al. 1989, Smit, Groen et al. 1994).

For patients with extensive stage disease, combination chemotherapy, including platinum and etoposide in doses that the least toxic effects is recommended (Okamoto, Watanabe et al. 2007). Further, radiation therapy to the site of distant metastases is also a standard treatment option for patients. This is especially preferred for metastases that are unlikely to be immediately palliated by chemotherapy, such as the brain and bone (Slotman, Faivre-Finn et al. 2007).

Commonly used chemotherapy combinations
include cisplatin, carboplatin, etoposide,

| | |
|---|---|
| Standard | Etoposide + cisplatin |
| treatment | Etoposide + carboplatin |
| Other | Cisplatin + irinotecan |
| regimens | Ifosfamide + cisplatin + etoposide |
| | Cyclophosphamide + doxorubicin + etoposide |
| | Cyclophosphamide + doxorubicin + etoposide + vincristine |
| | Cyclophosphamide + etoposide + vincristine |
| | Cyclophosphamide + doxorubicin + vincristine |

Response rates to chemotherapy are high for SCLC, up to 85-95% in limited disease and 75-80% in extensive disease. However, median survival still remains low, i.e. 14-20 months for limited disease and only 7-10 months for extensive disease. Long term survival is only seen in 5-10% of the patients. (Hoffman, Mauer et al. 2000).

In accordance with the present invention the methods, in particular the statistical methods, may comprise the use of FOXA2 Em isoform and/or ID2 Em isoform.

For example, the herein provided statistical method of assessing whether a subject suffers from cancer or is prone to suffering from cancer, may (further) comprise the step of

performing at least one statistical algorithm for classification and for regression on measurement data of the subject,
wherein the measurement data of the subject comprises at least one of the following: a value of FOXA2 Em isoform in at least one sample taken from the subject, a value ID2 Em isoform in said at least one sample, a value of FOXA2 Ad isoform in said at least one sample, ID2 Ad isoform in said at least one sample; and
wherein at least one of the following is used as at least one classifier or a component of at least one classifier in the statistical method: FOXA2 Em isoform, ID2 Em isoform, FOXA2 Ad isoform, ID2 Ad isoform, ratio of FOXA2 Em isoform/FOXA2 Ad isoform, ratio of ID2 Em isoform/ID2 Ad isoform.

The term “specific transcription factor Em isoform” according to the present application may relate to FOXA2 (Uniprot-ID: Q9Y261; Gene-ID: 3170) and/or ID2 (Uniprot-ID: Q02363; Gene-ID:3398). If, for example, the amount of a specific transcription factor is measured on mRNA level, the specific transcription factor can be mRNA molecules (or transcript or splice variants). In this context, the transcription factors can be defined as
i) the FOXA2 Em isoform comprising the nucleic acid sequence of SEQ ID No: 3 or the FOXA2 Em isoform comprising nucleic acid sequence with up to 68 additions, deletions or substitutions of SEQ ID NO: 3;
ii) the ID2 Em isoform comprising the nucleic acid sequence of SEQ ID No: 4 or the ID2 Em isoform comprising nucleic acid sequence with up to 34 additions, deletions or substitutions of SEQ ID NO: 4;
iii) the FOXA2 Ad isoform comprising the nucleic acid sequence of SEQ ID No: 7 or FOXA2 Ad isoform comprising the nucleic acid sequence with up to 74 additions, deletions or substitutions of SEQ ID NO: 7; or
iv) the ID2 Ad isoform consisting of the nucleic acid sequence of SEQ ID No: 8 or ID2 Ad isoform consisting of nucleic acid sequence with up to 30 additions, deletions or substitutions of SEQ ID NO: 8;

In a certain aspect, the value of the FOXA2 Em isoform in said at least one sample is obtained by measuring the amount of a specific transcription factor isoform in said at least one sample of said subject, wherein said specific transcription isoform is the FOXA2 Em isoform comprising the nucleic acid sequence of SEQ ID No: 3 or the FOXA2 Em isoform comprising nucleic acid sequence with up to 68 additions, deletions or substitutions of SEQ ID NO: 3.

In a certain aspect, the value of the ID2 Em isoform in said at least one sample is obtained by measuring the amount of a specific transcription factor isoform in said at least one sample of said subject, wherein said specific transcription isoform is the ID2 Em isoform comprising the nucleic acid sequence of SEQ ID No: 4 or the ID2 Em isoform comprising nucleic acid sequence with up to 34 additions, deletions or substitutions of SEQ ID NO: 4.

In a certain aspect, the value of the FOXA2 Ad isoform in said at least one sample is obtained by measuring the amount of a specific transcription factor isoform in said at least one sample of said subject, wherein said specific transcription isoform is the FOXA2 Ad isoform comprising the nucleic acid sequence of SEQ ID No: 7 or FOXA2 Ad isoform comprising the nucleic acid sequence with up to 74 additions, deletions or substitutions of SEQ ID NO: 7.

In a certain aspect, the value of the ID2 Ad isoform in said at least one sample is obtained by measuring the amount of a specific transcription factor isoform in said at least one sample of said subject, wherein said specific transcription isoform is the ID2 Ad isoform consisting of the nucleic acid sequence of SEQ ID No: 8 or ID2 Ad isoform consisting of nucleic acid sequence with up to 30 additions, deletions or substitutions of SEQ ID NO: 8.

In a certain aspect, the FOXA2 Em isoform of said sample is set in relation to a FOXA2 Em isoform of at least one control sample and then used as a classifier in the statistical method; and

said value of the FOXA2 Em isoform of said at least one control sample is obtained by measuring in said sample of said subject the amount of a specific transcription factor isoform, wherein said specific transcription isoform is the FOXA2 Em isoform comprising the nucleic acid sequence of SEQ ID No: 3 or the FOXA2 Em isoform comprising nucleic acid sequence with up to 68 additions, deletions or substitutions of SEQ ID NO: 3.

In a certain aspect, the FOXA2 Ad isoform of said sample is set in relation to a FOXA2 Ad isoform of at least one control sample and then used as a classifier in the statistical method; and

said value of the FOXA2 Ad isoform of said at least one control sample is obtained by measuring in said sample of said subject the amount of a specific transcription factor isoform, wherein said specific transcription isoform is the FOXA2 Ad isoform comprising the nucleic acid sequence of SEQ ID No: 7 or FOXA2 Ad isoform comprising the nucleic acid sequence with up to 74 additions, deletions or substitutions of SEQ ID NO: 7.

In a certain aspect, the ID2 Em isoform of said sample is set in relation to a ID2 Em isoform of at least one control sample and then used as a classifier in the statistical method; and

said value of the ID2 Em isoform of said at least one control sample is obtained by measuring in said sample of said subject the amount of a specific transcription factor isoform, wherein said specific transcription isoform is the ID2 Em isoform comprising the nucleic acid sequence of SEQ ID No: 4 or the ID2 Em isoform comprising nucleic acid sequence with up to 34 additions, deletions or substitutions of SEQ ID NO: 4.

In a certain aspect, the ID2 Ad isoform of said sample is set in relation to a ID2 Ad isoform of at least one control sample and then used as a classifier in the statistical method; and

said value of the ID2 Ad isoform of said at least one control sample is obtained by measuring in said sample of said subject the amount of a specific transcription factor isoform,
wherein said specific transcription isoform is the ID2 Ad isoform consisting of the nucleic acid sequence of SEQ ID No: 8 or ID2 Ad isoform consisting of nucleic acid sequence with up to 30 additions, deletions or substitutions of SEQ ID NO: 8.

In certain aspects, a ratio of the FOXA2 Em isoform and the FOXA2 Ad isoform and a ratio of the ID2 Em isoform and the ID2 Ad isoform are used as a classifier.

The present invention also contemplates the use of obtaining the value of a transcription factor isoform in a sample e.g. by measuring the amount of a transcription factor isoform on the protein level.

If, for example, the amount of a specific transcription factor is measured on protein level, the specific transcription factor can be protein molecules. For example, they can be defined as
i) the FOXA2 Em isoform comprising the polypeptide sequence of SEQ ID No: 52 or the FOXA2 Em isoform comprising polypeptide sequence with up to 43 additions, deletions or substitutions of SEQ ID NO: 52;
ii) the ID2 Em isoform comprising the polypeptide sequence of SEQ ID No: 53 or the ID2 Em isoform comprising polypeptide sequence with up to 13 additions, deletions or substitutions of SEQ ID NO: 53;
iii) the FOXA2 Ad isoform comprising the polypeptide sequence of SEQ ID No: 56 or FOXA2 Ad isoform comprising the polypeptide sequence with up to 43 additions, deletions or substitutions of SEQ ID NO: 56; or
iv) the ID2 Ad isoform consisting of the polypeptide sequence of SEQ ID No: 57 or ID2 Ad isoform consisting of polypeptide sequence with up to 13 additions, deletions or substitutions of SEQ ID NO: 57.

In a certain aspect, the value of the FOXA2 Em isoform in said at least one sample is obtained by measuring the amount of a specific transcription factor isoform in said at least one sample of said subject, wherein said specific transcription isoform is the FOXA2 Em isoform comprising the polypeptide sequence of SEQ ID No: 52 or the FOXA2 Em isoform comprising polypeptide sequence with up to 43 additions, deletions or substitutions of SEQ ID NO: 52.

In a certain aspect, the value of the ID2 Em isoform in said at least one sample is obtained by measuring the amount of a specific transcription factor isoform in said at least one sample of said subject, wherein said specific transcription isoform is the ID2 Em isoform comprising the polypeptide sequence of SEQ ID No: 53 or the ID2 Em isoform comprising polypeptide sequence with up to 13 additions, deletions or substitutions of SEQ ID NO: 53.

In a certain aspect, the value of the FOXA2 Ad isoform in said at least one sample is obtained by measuring the amount of a specific transcription factor isoform in said at least one sample of said subject, wherein said specific transcription isoform is the FOXA2 Ad isoform comprising the polypeptide sequence of SEQ ID No: 56 or FOXA2 Ad isoform comprising the polypeptide sequence with up to 43 additions, deletions or substitutions of SEQ ID NO: 56.

In a certain aspect, the value of the ID2 Ad isoform in said at least one sample is obtained by measuring the amount of a specific transcription factor isoform in said at least one sample of said subject, wherein said specific transcription isoform is the ID2 Ad isoform consisting of the polypeptide sequence of SEQ ID No: 57 or ID2 Ad isoform consisting of polypeptide sequence with up to 13 additions, deletions or substitutions of SEQ ID NO: 57.

If, for example, the amount of a specific transcription factor is measured on protein level, the specific transcription factors can be proteins molecules. For example, they can be defined as
i) the GATA6 Em isoform comprising the polypeptide sequence of SEQ ID No: 50 or the GATA6 Em isoform comprising the polypeptide sequence with up to 30 additions, deletions or substitutions of SEQ ID NO: 50;
ii) the NKX2-1 Em isoform comprising the polypeptide sequence of SEQ ID No: 51 or the NKX2-1 Em isoform comprising the polypeptide sequence with up to 14 additions, deletions or substitutions of SEQ ID NO: 51;
iii) the GATA6 Ad isoform comprising the polypeptide sequence of SEQ ID No: 54 or the GATA6 Ad isoform polypeptide sequence with up to 23 additions, deletions or substitutions of SEQ ID NO: 54;
iv) the NKX2-1 Ad isoform comprising the polypeptide sequence of SEQ ID No: 55 or the NKX2-1 Ad isoform comprising the polypeptide sequence with up to 15 additions, deletions or substitutions of SEQ ID NO: 55.

In a certain aspect, the value of the GATA6 Em isoform in said at least one sample is obtained by measuring the amount of a specific transcription factor isoform in said at least one sample of said subject, wherein said specific transcription isoform is the GATA6 Em isoform comprising the polypeptide sequence of SEQ ID No: 50 or the GATA6 Em isoform comprising the polypeptide sequence with up to 30 additions, deletions or substitutions of SEQ ID NO: 50

In a certain aspect, the value of the NKX2-1 Em isoform in said at least one sample is obtained by measuring the amount of a specific transcription factor isoform in said at least one sample of said subject, wherein said specific transcription isoform is the NKX2-1 Em isoform comprising the polypeptide sequence of SEQ ID No: 51 or the NKX2-1 Em isoform comprising the polypeptide sequence with up to 14 additions, deletions or substitutions of SEQ ID NO: 51

In a certain aspect, the value of the GATA6 Ad isoform in said at least one sample is obtained by measuring the amount of a specific transcription factor isoform in said at least one sample of said subject, wherein said specific transcription isoform is the GATA6 Ad isoform comprising the polypeptide sequence of SEQ ID No: 54 or the GATA6 Ad isoform polypeptide sequence with up to 23 additions, deletions or substitutions of SEQ ID NO: 54

In a certain aspect, the value of the NKX2-1 Ad isoform in said at least one sample is obtained by measuring the amount of a specific transcription factor isoform in said at least one sample of said subject, wherein said specific transcription isoform is the NKX2-1 Ad isoform comprising the polypeptide sequence of SEQ ID No: 55 or the NKX2-1 Ad isoform comprising the polypeptide sequence with up to 15 additions, deletions or substitutions of SEQ ID NO: 55.

Genes can contain single nucleotide polymorphisms (SNPs). The specific transcription factor Em isoform sequences of the present invention encompass (genetic) variants thereof, for example, variants having SNPs. Without deferring from the gist of the present invention, all naturally occurring sequences of the respective isoform independent of the number and nature of the SNPs in said sequence can be used herein. To relate to currently known SNPs, the transcription factor Em isoforms of the present invention are defined such that they contain up to 55 (in the case of GATA6), up to 39 (in the case of NKX2-1), up to 68 (in the case of FOXA2) or up to 34 (in the case of ID2) additions, deletions or substitutions of the nucleic acid sequences defined by SEQ ID NOs: 1, 2, 3 and 4, respectively. Thus, respective Em transcripts of carriers of different nucleotides at the respective SNPs are covered by the present application.

The FOXA2 Em isoform according to the invention is the FOXA2 Em isoform comprising the nucleic acid sequence of SEQ ID No: 3 or the FOXA2 Em isoform comprising a nucleic acid sequence with up to 68; preferably up to 67, 66, 65, 64, 63, 62, 61, 60, 59, 58, 57, 56, 55, 54, 53 52, 51, 50, 49, 48 47, 46, 45, 44, 43, 42, 41, 40, 39, 38, 37, 36, 35, 34, 33, 32, 31, 30, 29, 28, 27, 26, 25, 24, 23, 22, 21 or 20; even more preferably up to 19, 18, 17, 16, 15, 14, 13, 12, 11, 10, 9, 8, 7. 6, 5, 4, 3 or 2; or even furthermore preferably only 1 addition(s), deletion(s) or substitution(s) of SEQ ID NO: 3. The FOXA2 Em isoform can also be defined as the FOXA2 Em isoform comprising the nucleic acid sequence of SEQ ID No: 3 or the FOXA2 Em isoform comprising the nucleic acid sequence of SEQ ID No: 3 with additions, deletions or substitutions at any of positions 168; 208; 289; 361; 368; 374; 379; 383; 404; 459; 481; 483; 494; 529; 564; 577; 584; 590; 610; 623; 641; 650; 659; 674; 773; 845; 1040; 1075; 1186; 1188; 1240; 1242; 1243; 1304; 1374; 1391; 1408; 1414; 1432; 1458; 1475; 1487; 1522; 1539; 1582; 1583; 1594; 1627; 1631; 1687; 1723; 1737; 1738; 1754; 1812; 1831; 1838; 1940; 1966; 1970; 2070; 2083; 2084; 2093; 2105; 2112; 2200 and 2388. The FOXA2 Em isoform according to the invention can also be defined as the FOXA2 Em isoform comprising the nucleic acid sequence of SEQ ID No: 3 or the FOXA2 Em isoform comprising a nucleic acid sequence with at least 93% homology to SEQ ID No: 3, preferably up to 94%, 95%, 96%, 97% or 98% homology to SEQ ID No: 3; even more preferably up to 99% homology to SEQ ID No: 3.

The ID2 Em isoform according to the invention is the ID2 Em isoform comprising the nucleic acid sequence of SEQ ID No: 4 or the ID2 Em isoform comprising a nucleic acid sequence with up to 34; preferably up to 33, 32, 31, 30, 29, 28, 27, 26, 25, 24, 23, 22, 21, 20, 19, 18, 17, 16, 15, 14, 13, 12, 11, or 10; even more preferably up to 9, 8, 7, 6, 5, 4, 3, or 2; or even furthermore preferably only 1 addition(s), deletion(s) or substitution(s) of SEQ ID NO: 4. The ID2 Em isoform can also be defined as the ID2 Em isoform comprising the nucleic acid sequence of SEQ ID No: 4 or the ID2 Em isoform comprising the nucleic acid sequence of SEQ ID No: 4 with additions, deletions or substitutions at any of positions 6; 43; 53; 55; 154; 195; 209; 224; 237; 263; 286; 360; 399; 405; 485; 501; 544; 547; 605; 662; 665; 716; 757; 871; 876; 975; 1085; 1115; 1119; 1149; 1151; 1251; 1333 and 1350. The ID2 Em isoform according to the invention can also be defined as the ID2 Em isoform comprising the nucleic acid sequence of SEQ ID No: 4 or the ID2 Em isoform comprising a nucleic acid sequence with at least 51% homology to SEQ ID No: 4, preferably up to 55%, 60%, 65%, 70%, 75%, 80%, 85% or 90% homology to SEQ ID No: 4; even more preferably up to 91%, 92%, 93%, 94%, 95%, 96%, 97%, 98% or 99% homology to SEQ ID No: 4.

Preferably, the above referred “addition(s), deletion(s) or substitution(s)” of the transcription factor isoforms are substitutions.

The person skilled in the art understands that a subject which is prone to suffering from cancer is a subject which has an increased likelihood of developing cancer within the next 30 years or preferably within the next 20 or 10 years or even more preferably within the next 9, 8, 7, 6, 5, 4, 3 or 2 years or even furthermore preferably within the next year. An increased likelihood of a subject of developing cancer can be understood as that said subject has an increased likelihood of developing cancer within a given time period as if compared to the average likelihood that a subject of the same age or a subject of the same age and the same gender develops cancer.

The term “sample” according to the present invention relates to any kind of sample which can be obtained from a subject, preferably from a human subject. The sample is a biological sample. A sample according to the present invention can be for example, but is not limited to, a blood sample, a breath condensate sample, a bronchoalveolar lavage fluid sample, a mucus sample or a phlegm sample. Preferably, the sample according to the present invention is a biopsy, a blood sample or a breath condensate sample. More preferably, the sample according to the present invention is a biopsy or a breath condensate sample. Particularly preferred is (a) (a) breath condensate sample(s).

The term “breath condensate sample” as used herein refers to an “exhaled breath condensate (sample)”. The term “exhaled breath condensate (sample)” can be abbreviated as “EBC”. Accordingly, the terms “breath condensate sample”, “exhaled breath condensate”, “exhaled breath condensate sample” and “EBC” are used interchangeably herein. The use of “breath condensate sample”, in particular “exhaled breath condensate (sample)” allows the non-invasive obtaining of samples from a subject/patient and is therefore advantageous.

The herein provided diagnostic method can lead to fast medical intervention for example by means of corresponding anti-cancer therapy, like anti-cancer medication or radiation therapy. Early stage anti-cancer therapies include, but are not limited to, radiation therapy, such as external radiation therapy, photodynamic therapy (PDT) using an endoscope and surgery (i.e. wedge resection or segmental resection for carcinoma in situ and sleeve resection or lobectomy for StageI). In addition, chemotherapy is used alone or after surgery. The chemotherapy drugs may, inter alia, comprise compounds selected from the group consisting of Cisplatin, Carboplatin, Paclitaxel (Taxol®), Albumin-bound paclitaxel (nab-paclitaxel, Abraxane®), Docetaxel (Taxotere®), Gemcitabine (Gemzar®), Vinorelbine (Navelbine®), Irinotecan (Camptosar®, CPT-11), Etoposide (VP-16®), Vinblastine and Pemetrexed (Alimta®).

The herein provided methods are primarily useful in the assessment whether a subject suffers from cancer or is prone to suffering from cancer before the subject undergoes therapeutic intervention. In other words, the sample of the subject is obtained from the subject and analyzed prior to therapeutic intervention, like conventional chemotherapy. If the subject is assessed “positive” in accordance with the present invention, i.e. assessed to suffer from cancer or prone to suffering from cancer, the appropriate therapy/therapeutic intervention can be chosen. For example, a subject may be suspected of suffering from cancer and the present methods can be used to assess whether the subject suffers indeed from said cancer in addition or in the alternative to conventional diagnostic methods.

Following positive diagnosis with the herein provided inventive method, the diagnosis may be elucidated/further verified with low-dose helical computed tomography and/or Chest X-Ray, by bronchoscopy and/or histological assessment. In early stage or Grade I tumors, surgery to to remove the lobe or the section of the lung that contains the tumor would be the first choice of treatment. It is feasible to supplement the surgery with chemotherapy, known as ‘adjuvant chemotherapy’, to prevent cancer relapse (Howington J A et al. (2013) CHEST Journal 143: e278S-e313S). At later stages, surgery is no longer feasible and a combination of chemotherapy and radiation are advised. Further, for metastatic lesions, chemotherapy and radiation are suggested, mainly for palliation of the symptoms.

The term “isoform” according to the present invention encompasses transcript variants (which are mRNA molecules) as well as the corresponding polypeptide variants (which are polypeptides) of a gene. Such transcription variants result, for example, from alternative splicing or from a shifted transcription initiation. Based on the different transcript variants, different polypeptides are generated. It is possible that different transcript variants have different translation initiation sites. A person skilled in the art will appreciate that the amount of an isoform can be measured by adequate techniques for the quantification of mRNA as far as the isoform relates to a transcript variant which is an mRNA. Examples of such techniques are polymerase chain reaction-based methods, in situ hybridization-based methods, microarray-based techniques and whole transcriptome shotgun sequencing. Further, a person skilled in the art will appreciate that the amount of an isoform can be measured by adequate techniques for the quantification of polypeptides as far as the isoform relates to a polypeptide. Non-limiting examples of such techniques for the quantification of polypeptides are ELISA (Enzyme-linked Immunosorbent Assay)-based, gel-based, blot-based, mass spectrometry-based, and flow cytometry-based methods.

Genes can contain single nucleotide polymorphisms (SNPs). The specific transcription factor Em isoform sequences of the present invention encompass (genetic) variants thereof, for example, variants having SNPs. Without deferring from the gist of the present invention, all naturally occurring sequences of the respective isoform independent of the number and nature of the SNPs in said sequence can be used herein. To relate to currently known SNPs, the transcription factor Em isoforms of the present invention are defined such that they contain up to 55 (in the case of GATA6), up to 39 (in the case of NKX2-1), additions, deletions or substitutions of the nucleic acid sequences defined by SEQ ID NOs: 1 and 2 respectively. Thus, respective Em transcripts of carriers of different nucleotides at the respective SNPs are covered by the present application.

The GATA6 Em isoform according to the invention is the GATA6 Em isoform comprising the nucleic acid sequence of SEQ ID No: 1 or the GATA6 Em isoform comprising a nucleic acid sequence with up to 55; preferably up to 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21 or 20; even more preferably up to 19, 18, 17, 16, 15, 14, 13, 12, 11, 10, 9, 8, 7. 6, 5, 4, 3 or 2; or even furthermore preferably only 1 addition(s), deletion(s) or substitution(s) of SEQ ID NO: 1. The GATA6 Em isoform can also be defined as the GATA6 Em isoform comprising the nucleic acid sequence of SEQ ID No: 1 or the GATA6 Em isoform comprising the nucleic acid sequence of SEQ ID No: 1 with additions, deletions or substitutions at any of positions 163; 293; 320; 327; 339; 430; 462; 480; 759; 1128; 1256; 1304; 1589; 1597; 1627; 1651; 1652; 1803; 1844; 1849; 1879; 1882; 1911; 1940; 1949; 1982; 2000; 2002; 2008; 2026; 2031; 2106; 2137; 2142; 2163; 2294; 2390; 2391; 2627; 2691; 3036; 3102; 3240; 3265; 3266; 3290; 3358; 3366; 3578; 3632; 3646; 3670; 3690; 3708 and 3735. The GATA6 Em isoform according to the invention can also be defined as the GATA6 Em isoform comprising the nucleic acid sequence of SEQ ID No: 1 or the GATA6 Em isoform comprising a nucleic acid sequence with at least 85% homology to SEQ ID No: 1, preferably up to 86%, 87%, 88%, 89%, 90%, 91%, 92%, 93%, 94%, 95%, 96%, 97% or 98% homology to SEQ ID No: 1; even more preferably up to 99% homology to SEQ ID No: 1.

The NKX2-1 Em isoform according to the invention is the NKX2-1 Em isoform comprising the nucleic acid sequence of SEQ ID No: 2 or the NKX2-1 Em isoform comprising a nucleic acid sequence with up to 39; preferably up to 38, 37, 36, 35, 34, 33, 32, 31, 30, 29, 28, 27, 26, 25, 24, 23, 22, 21, 20, 19, 18, 17, 16, 15, 14, 13, 12, 11, or 10; even more preferably up to 9, 8, 7, 6, 5, 4, 3, or 2; or even furthermore preferably only 1 addition(s), deletion(s) or substitution(s) of SEQ ID NO: 2. The NKX2-1 Em isoform can also be defined as the NKX2-1 Em isoform comprising the nucleic acid sequence of SEQ ID No: 2 or the NKX2-1 Em isoform comprising the nucleic acid sequence of SEQ ID No: 2 with additions, deletions or substitutions at any of positions 269; 281; 305; 304; 420; 425; 439; 441; 450; 486; 781; 785; 825; 950; 1169; 1305; 1344; 1448; 1458; 1467; 1489; 1552; 1633; 1634; 1640; 1641; 1643; 1667; 1673; 1678; 1748; 1750; 1831; 1893; 1916; 1917; 1934; 2099 and 2319. The NKX2-1 Em isoform according to the invention can also be defined as the NKX2-1 Em isoform comprising the nucleic acid sequence of SEQ ID No: 2 or the NKX2-1 Em isoform comprising a nucleic acid sequence with at least 90% homology to SEQ ID No: 2, preferably up to 91%, 92%, 93%, 94%, 95%, 96%, 97% or 98% homology to SEQ ID No: 2; even more preferably up to 99% homology to SEQ ID No: 2.

Preferably, the above referred “addition(s), deletion(s) or substitution(s)” of the transcription factor isoforms are substitutions.

Tables 1, 2, 3, 4, 5, 6, 7 and 8 below provide information on different SNPs of the transcription factors of the present invention. The present invention relates to the respective isoforms independently from the various SNPs which may occur at the different positions of the mRNAs or polypeptides. The SNPs of tables 1, 2, 3, 4, 5, 6, 7 and 8 may occur in the isoforms of the present invention in any combination. For example, a (genetic) variant of the GATA6 Em isoform to be used herein may comprise a nucleic acid sequence of SEQ ID NO:1, whereby the “G” residue at position 293 of SEQ ID NO:1 is substituted by “A”. Further variants of the isoforms to be used herein are apparent from Tables 1 to 8 to the person skilled in the art. The respective SNP information has been retrieved using dbSNP (short genetic variations) database of the NCBI. The SNP information is based on Contig Label GRCh37.p5. A person skilled in the art will understand that also SNPs which are not mentioned in tables 1 to 8 are encompassed by the present invention.

TABLE 1
SNPs of the GATA6 Em isoform

| | | | Contig | Poly- | Codon | | Protein |
|---|---|---|---|---|---|---|---|
| S. No. | Region | Position | reference | morphism | Position | Function | residue |
| 1 | 5′ UTR | 163 | C | G | | | |
| 2 | CCDS | 293 | G | A | 6 | Missense | Gly-Ser |
| 3 | CCDS | 320 | G | C | 15 | Missense | Gly-Arg |
| 4 | CCDS | 327 | C | G | 17 | Missense | Ala-Gly |
| 5 | CCDS | 339 | C | G | 21 | Missense | Ala-Gly |
| 6 | CCDS | 430 | G | T | 51 | Missense | Glu-Asp |
| 7 | CCDS | 462 | — | T | 62 | Frameshift | TA-Thr |
| 8 | CCDS | 480 | A | T | 68 | Missense | Glu-Val |
| 9 | CCDS | 759 | C | T | 161 | Missense | Ala-Val |
| 10 | CCDS | 1128 | C | G | 284 | Missense | Ala-Gly |
| 11 | CCDS | 1256 | C | A | 327 | Missense | His-Asn |
| 12 | CCDS | 1304 | G | A | 343 | Missense | Ala-Thr |
| 13 | CCDS | 1589 | C | T | 438 | Missense | Arg-Trp |
| 14 | CCDS | 1597 | T | A | 440 | Synonymous | Leu-Leu |
| 15 | CCDS | 1627 | A | G | 450 | Synonymous | Thr-Thr |
| 16 | CCDS | 1651 | C | T | 458 | Synonymous | Asn-Asn |
| 17 | CCDS | 1652 | G | A | 459 | Missense | Ala-Thr |
| 18 | CCDS | 1803 | A | G | 509 | Missense | Asn-Ser |
| 19 | CCDS | 1844 | T | C | 523 | Missense | Ser-Pro |
| 20 | CCDS | 1849 | T | C | 524 | Synonymous | Asp-Asp |
| 21 | CCDS | 1879 | A | G | 534 | Synonymous | Thr-Thr |
| 22 | CCDS | 1882 | A | G | 535 | Synonymous | Gln-Gln |
| 23 | CCDS | 1911 | T | G | 545 | Missense | Val-Gly |
| 24 | CCDS | 1940 | C | G | 555 | Missense | Pro-Ala |
| 25 | CCDS | 1949 | A | G | 558 | Missense | Ser-Gly |
| 26 | CCDS | 1982 | T | C | 569 | Missense | Tyr-His |
| 27 | CCDS | 2000 | G | C | 575 | Missense | Ala-Pro |
| 28 | CCDS | 2002 | C | T | 575 | Synonymous | Ala-Ala |
| 29 | CCDS | 2008 | G | C | 577 | Synonymous | Pro-Pro |
| 30 | CCDS | 2026 | C | T | 583 | Synonymous | Ser-Ser |
| 31 | CCDS | 2031 | G | T | 585 | Missense | Arg-Leu |
| 32 | 3′UTR | 2106 | C | T | | | |
| 33 | 3′UTR | 2137 | G | A | | | |
| 34 | 3′UTR | 2142 | A | G | | | |
| 35 | 3′UTR | 2163 | C | T | | | |
| 36 | 3′UTR | 2294 | C | T | | | |
| 37 | 3′UTR | 2390 | A | G | | | |
| 38 | 3′UTR | 2391 | T | A | | | |
| 39 | 3′UTR | 2627 | A | G | | | |
| 40 | 3′UTR | 2691 | G | T | | | |
| 41 | 3′UTR | 3036 | G | T | | | |
| 42 | 3′UTR | 3102 | A | G | | | |
| 43 | 3′UTR | 3240 | C | T | | | |
| 44 | 3′UTR | 3265 | C | G | | | |
| 45 | 3′UTR | 3266 | C | T | | | |
| 46 | 3′UTR | 3290 | A | G | | | |
| 47 | 3′UTR | 3358 | C | T | | | |
| 48 | 3′UTR | 3366 | A | T | | | |
| 49 | 3′UTR | 3578 | C | T | | | |
| 50 | 3′UTR | 3632 | — | C | | | |
| 51 | 3′UTR | 3646 | C | T | | | |
| 52 | 3′UTR | 3670 | A | G | | | |
| 53 | 3′UTR | 3690 | C | T | | | |
| 54 | 3′UTR | 3708 | A | G | | | |
| 55 | 3′UTR | 3735 | A | G | | | |

TABLE 2
SNPs of the GATA6 Ad isoform

| | | | Contig | Poly- | Codon | | Protein |
|---|---|---|---|---|---|---|---|
| S. No. | Region | Position | reference | morphism | Position | Function | residue |
| 1 | 5′UTR | 138 | C | G | | | |
| 2 | 5′UTR | 228 | G | A | | | |
| 3 | 5′UTR | 255 | G | C | | | |
| 4 | 5′UTR | 262 | C | G | | | |
| 5 | 5′UTR | 274 | C | G | | | |
| 6 | 5′UTR | 365 | G | T | | | |
| 7 | 5′UTR | 397 | — | T | | | |
| 8 | 5′UTR | 415 | A | T | | | |
| 9 | CCDS | 694 | C | T | 15 | Missense | Ala-Val |
| 10 | CCDS | 1063 | C | G | 138 | Missense | Ala- |
| | | | | | | | Gly |
| 11 | CCDS | 1191 | C | A | 181 | Missense | His- |
| | | | | | | | Asn |
| 12 | CCDS | 1239 | G | A | 197 | Missense | Ala-Thr |
| 13 | CCDS | 1524 | C | T | 292 | Missense | Arg- |
| | | | | | | | Trp |
| 14 | CCDS | 1532 | T | A | 294 | Synonymous | Leu- |
| | | | | | | | Leu |
| 15 | CCDS | 1562 | A | G | 304 | Synonymous | Thr-Thr |
| 16 | CCDS | 1586 | C | T | 312 | Synonymous | Asn- |
| | | | | | | | Asn |
| 17 | CCDS | 1587 | G | A | 313 | Missense | Ala-Thr |
| 18 | CCDS | 1738 | A | G | 363 | Missense | Asn- |
| | | | | | | | Ser |
| 19 | CCDS | 1779 | T | C | 377 | Missense | Ser-Pro |
| 20 | CCDS | 1784 | T | C | 378 | Synonymous | Asp- |
| | | | | | | | Asp |
| 21 | CCDS | 1814 | A | G | 388 | Synonymous | Thr-Thr |
| 22 | CCDS | 1817 | A | G | 389 | Synonymous | Gln- |
| | | | | | | | Gln |
| 23 | CCDS | 1846 | T | G | 399 | Missense | Val- |
| | | | | | | | Gly |
| 24 | CCDS | 1875 | C | G | 409 | Missense | Pro-Ala |
| 25 | CCDS | 1884 | A | G | 412 | Missense | Ser-Gly |
| 26 | CCDS | 1917 | T | C | 423 | Missense | Tyr-His |
| 27 | CCDS | 1935 | G | C | 429 | Missense | Ala-Pro |
| 28 | CCDS | 1937 | C | T | 429 | Synonymous | Ala-Ala |
| 29 | CCDS | 1943 | G | C | 431 | Synonymous | Pro-Pro |
| 30 | CCDS | 1961 | C | T | 437 | Synonymous | Ser-Ser |
| 31 | CCDS | 1966 | G | T | 439 | Missense | Arg- |
| | | | | | | | Leu |
| 32 | 3′UTR | 2041 | C | T | | | |
| 33 | 3′UTR | 2072 | G | A | | | |
| 34 | 3′UTR | 2077 | A | G | | | |
| 35 | 3′UTR | 2098 | C | T | | | |
| 36 | 3′UTR | 2229 | C | T | | | |
| 37 | 3′UTR | 2325 | A | G | | | |
| 38 | 3′UTR | 2326 | T | A | | | |
| 39 | 3′UTR | 2562 | A | G | | | |
| 40 | 3′UTR | 2626 | G | T | | | |
| 41 | 3′UTR | 2971 | G | T | | | |
| 42 | 3′UTR | 3037 | A | G | | | |
| 43 | 3′UTR | 3175 | C | T | | | |
| 44 | 3′UTR | 3200 | C | G | | | |
| 45 | 3′UTR | 3201 | C | T | | | |
| 46 | 3′UTR | 3225 | A | G | | | |
| 47 | 3′UTR | 3293 | C | T | | | |
| 48 | 3′UTR | 3301 | A | T | | | |
| 49 | 3′UTR | 3513 | C | T | | | |
| 50 | 3′UTR | 3567 | — | C | | | |
| 51 | 3′UTR | 3581 | C | T | | | |
| 52 | 3′UTR | 3605 | A | G | | | |
| 53 | 3′UTR | 3625 | C | T | | | |
| 54 | 3′UTR | 3643 | A | G | | | |
| 55 | 3′UTR | 3670 | A | G | | | |

TABLE 3
SNPs of the NKX2-1 Em isoform

| | | | Contig | Poly- | Codon | | Protein |
|---|---|---|---|---|---|---|---|
| S. No. | Region | Position | reference | morphism | Position | Function | residue |
| 1 | 5′UTR | 269 | C | T | | | |
| 2 | 5′UTR | 281 | A | G | | | |
| 3 | 5′UTR | 305 | — | A | | | |
| 4 | 5′UTR | 304 | — | AA | | | |
| 5 | CCDS | 420 | G | A | 27 | Missense | Val-Met |
| 6 | CCDS | 425 | C | T | 28 | Synonymous | Gly-Gly |
| 7 | CCDS | 439 | G | T | 33 | Missense | Gly-Val |
| 8 | CCDS | 441 | C | A | 34 | Missense | Leu-Ile |
| 9 | CCDS | 450 | C | T | 37 | Missense | Pro-Ser |
| 10 | CCDS | 486 | C | T | 49 | Missense | Pro-Ser |
| 11 | CCDS | 781 | G | T | 147 | Missense | Gly-Val |
| 12 | CCDS | 785 | C | T | 148 | Synonymous | Asp-Asp |
| 13 | CCDS | 825 | A | C | 162 | Synonymous | Arg-Arg |
| 14 | CCDS | 950 | G | T | 203 | Synonymous | Thr-Thr |
| 15 | CCDS | 1169 | G | A | 276 | Synonymous | Ala-Ala |
| 16 | CCDS | 1305 | G | A | 322 | Missense | Gly-Ser |
| 17 | CCDS | 1344 | G | T | 335 | Missense | Ala-Ser |
| 18 | CCDS | 1448 | G | A | 369 | Synonymous | Arg-Arg |
| 19 | 3′UTR | 1458 | C | T | | | |
| 20 | 3′UTR | 1467 | C | T | | | |
| 21 | 3′UTR | 1489 | G | T | | | |
| 22 | 3′UTR | 1552 | G | T | | | |
| 23 | 3′UTR | 1633 | A | G | | | |
| 24 | 3′UTR | 1634 | A | G | | | |
| 25 | 3′UTR | 1640 | — | T | | | |
| 26 | 3′UTR | 1641 | — | GT | | | |
| 27 | 3′UTR | 1643 | — | >6 bp | | | |
| 28 | 3′UTR | 1667 | A | T | | | |
| 29 | 3′UTR | 1673 | — | T | | | |
| 30 | 3′UTR | 1678 | — | T | | | |
| 31 | 3′UTR | 1748 | — | C | | | |
| 32 | 3′UTR | 1750 | — | C | | | |
| 33 | 3′UTR | 1831 | A | T | | | |
| 34 | 3′UTR | 1893 | G | T | | | |
| 35 | 3′UTR | 1916 | — | A | | | |
| 36 | 3′UTR | 1917 | — | A | | | |
| 37 | 3′UTR | 1934 | C | G/T | | | |
| 38 | 3′UTR | 2099 | C | G | | | |
| 39 | 3′UTR | 2319 | C | G | | | |

TABLE 4
SNPs of the NKX2-1 Ad isoform

| | | | Contig | Poly- | Codon | | Protein |
|---|---|---|---|---|---|---|---|
| S. No. | Region | Position | reference | morphism | Position | Function | residue |
| 1 | 5′UTR | 12 | G | T | | | |
| 2 | CCDS | 125 | G | A | 10 | Missense | Arg-Gln |
| 3 | CCDS | 265 | G | A | 57 | Missense | Val-Met |
| 4 | CCDS | 270 | C | T | 58 | Synonymous | Gly-Gly |
| 5 | CCDS | 284 | G | T | 63 | Missense | Gly-Val |
| 6 | CCDS | 286 | C | A | 64 | Missense | Leu-Ile |
| 7 | CCDS | 295 | C | T | 67 | Missense | Pro-Ser |
| 8 | CCDS | 331 | C | T | 79 | Missense | Pro-Ser |
| 9 | CCDS | 626 | G | T | 177 | Missense | Gly-Val |
| 10 | CCDS | 630 | C | T | 178 | Synonymous | Asp-Asp |
| 11 | CCDS | 670 | A | C | 192 | Synonymous | Arg-Arg |
| 12 | CCDS | 795 | G | T | 233 | Synonymous | Thr-Thr |
| 13 | CCDS | 1014 | G | A | 306 | Synonymous | Ala-Ala |
| 14 | CCDS | 1150 | G | A | 352 | Missense | Gly-Ser |
| 15 | CCDS | 1189 | G | T | 365 | Missense | Ala-Ser |
| 16 | CCDS | 1293 | G | A | 399 | Synonymous | Arg-Arg |
| 17 | 3′UTR | 1303 | C | T | | | |
| 18 | 3′UTR | 1312 | C | T | | | |
| 19 | 3′UTR | 1334 | G | T | | | |
| 20 | 3′UTR | 1397 | G | T | | | |
| 21 | 3′UTR | 1478 | A | G | | | |
| 22 | 3′UTR | 1479 | A | G | | | |
| 23 | 3′UTR | 1478 | — | >6 bp | | | |
| 24 | 3′UTR | 1485 | — | T | | | |
| 25 | 3′UTR | 1486 | — | GT | | | |
| 26 | 3′UTR | 1488 | — | >6 bp | | | |
| 27 | 3′UTR | 1512 | A | T | | | |
| 28 | 3′UTR | 1518 | — | T | | | |
| 29 | 3′UTR | 1523 | — | T | | | |
| 30 | 3′UTR | 1593 | — | C | | | |
| 31 | 3′UTR | 1595 | — | C | | | |
| 32 | 3′UTR | 1676 | A | T | | | |
| 33 | 3′UTR | 1738 | G | T | | | |
| 34 | 3′UTR | 1761 | — | A | | | |
| 35 | 3′UTR | 1762 | — | A | | | |
| 36 | 3′UTR | 1779 | C | G/T | | | |
| 37 | 3′UTR | 1944 | C | G | | | |
| 38 | 3′UTR | 2164 | C | G | | | |

TABLE 5
SNPs of the FOXA2 Em isoform

| | | | Contig | Poly- | Codon | | Protein |
|---|---|---|---|---|---|---|---|
| S. No. | Region | Position | reference | morphism | Position | Function | residue |
| 1 | 5′UTR | 168 | — | >6 bp | | | |
| 2 | CCDS | 208 | T | C | 8 | Missense | Leu-Pro |
| 3 | CCDS | 289 | G | A | 35 | Missense | Ser-Asn |
| 4 | CCDS | 361 | G | A | 59 | Missense | Ser-Asn |
| 5 | CCDS | 368 | G | A | 61 | Synonymous | Ser-Ser |
| 6 | CCDS | 374 | C | T | 63 | Synonymous | Asn-Asn |
| 7 | CCDS | 379 | G | A | 65 | Missense | Ser-Asn |
| 8 | CCDS | 383 | G | A | 66 | Synonymous | Ala-Ala |
| 9 | CCDS | 404 | G | T | 73 | Synonymous | Ser-Ser |
| 10 | CCDS | 459 | G | A | 92 | Missense | Ala-Thr |
| 11 | CCDS | 481 | C | T | 99 | Missense | Ser-Leu |
| 12 | CCDS | 483 | G | C | 100 | Missense | Ala-Pro |
| 13 | CCDS | 494 | C | T | 103 | Synonymous | Ala-Ala |
| 14 | CCDS | 529 | G | A | 115 | Missense | Ser-Asn |
| 15 | CCDS | 564 | A | G | 127 | Missense | Met-Val |
| 16 | CCDS | 577 | C | G | 131 | Missense | Ala-Gly |
| 17 | CCDS | 584 | C | T | 133 | Synonymous | Tyr-Tyr |
| 18 | CCDS | 590 | C | A | 135 | Missense | Asn-Lys |
| 19 | CCDS | 610 | T | C | 142 | Missense | Met-Thr |
| 20 | CCDS | 623 | G | C | 146 | Synonymous | Ala-Ala |
| 21 | CCDS | 641 | C | T | 152 | Synonymous | Arg-Arg |
| 22 | CCDS | 650 | G | A | 155 | Synonymous | Lys-Lys |
| 23 | CCDS | 659 | G | T | 158 | Missense | Arg-Ser |
| 24 | CCDS | 674 | C | T | 163 | Synonymous | His-His |
| 25 | CCDS | 773 | G | T | 196 | Missense | Met-Ile |
| 26 | CCDS | 845 | C | T | 220 | Synonymous | Asn-Asn |
| 27 | CCDS | 1040 | A | G | 285 | Synonymous | Gly-Gly |
| 28 | CCDS | 1075 | C | T | 297 | Missense | Ala-Val |
| 29 | CCDS | 1186 | C | T | 334 | Missense | Ala-Val |
| 30 | CCDS | 1188 | G | C | 335 | Missense | Ala-Pro |
| 31 | CCDS | 1240 | C | T | 352 | Missense | Ala-Val |
| 32 | CCDS | 1242 | G | A | 353 | Missense | Ala-Thr |
| 33 | CCDS | 1243 | C | G | 353 | Missense | Ala-Gly |
| 34 | CCDS | 1304 | A | C | 373 | Missense | Glu-Asp |
| 35 | CCDS | 1374 | AG | — | 397 | Frameshift | Ser-Pro |
| 36 | CCDS | 1391 | A | G | 402 | Synonymous | Gln-Gln |
| 37 | CCDS | 1408 | T | C | 408 | Missense | Leu-Pro |
| 38 | CCDS | 1414 | C | T | 410 | Missense | Ala-Val |
| 39 | CCDS | 1432 | A | C | 416 | Missense | His-Pro |
| 40 | CCDS | 1458 | C | A | 425 | Missense | Pro-Thr |
| 41 | CCDS | 1475 | G | A | 430 | Missense | Met-Ile |
| 42 | CCDS | 1487 | G | C | 434 | Synonymous | Thr-Thr |
| 43 | CCDS | 1522 | C | G | 446 | Missense | Ala-Gly |
| 44 | CCDS | 1539 | C | G | 452 | Missense | Gln-Glu |
| 45 | 3′UTR | 1582 | G | T | | | |
| 46 | 3′UTR | 1583 | A | G | | | |
| 47 | 3′UTR | 1594 | C | T | | | |
| 48 | 3′UTR | 1627 | A | G | | | |
| 49 | 3′UTR | 1631 | A | G | | | |
| 50 | 3′UTR | 1687 | A | G | | | |
| 51 | 3′UTR | 1723 | A | C | | | |
| 52 | 3′UTR | 1737 | — | G | | | |
| 53 | 3′UTR | 1738 | — | G | | | |
| 54 | 3′UTR | 1754 | A | G | | | |
| 55 | 3′UTR | 1812 | A | G | | | |
| 56 | 3′UTR | 1831 | A | T | | | |
| 57 | 3′UTR | 1838 | — | T | | | |
| 58 | 3′UTR | 1940 | A | C | | | |
| 59 | 3′UTR | 1966 | — | G/T | | | |
| 60 | 3′UTR | 1970 | — | A | | | |
| 61 | 3′UTR | 2070 | A | T | | | |
| 62 | 3′UTR | 2083 | A | G | | | |
| 63 | 3′UTR | 2084 | — | T | | | |
| 64 | 3′UTR | 2093 | — | T | | | |
| 65 | 3′UTR | 2105 | A | C | | | |
| 66 | 3′UTR | 2112 | C | T | | | |
| 67 | 3′UTR | 2200 | C | T | | | |
| 68 | 3′UTR | 2388 | A | G | | | |

TABLE 6
SNPs of the FOXA2 Em isoform

| | | | Contig | Poly- | Codon | | Protein |
|---|---|---|---|---|---|---|---|
| S. No. | Region | Position | reference | morphism | Position | Function | residue |
| 1 | 5′UTR | 5 | C | T | | | |
| 2 | 5′UTR | 37 | G | T | | | |
| 3 | 5′UTR | 65 | C | T | | | |
| 4 | 5′UTR | 68 | A | C | | | |
| 5 | 5′UTR | 70 | A | G | | | |
| 6 | 5′UTR | 88 | A | G | | | |
| 7 | 5′UTR | 128 | C | T | | | |
| 8 | CCDS | 195 | T | C | 2 | Missense | Leu-Pro |
| 9 | CCDS | 276 | G | A | 29 | Missense | Ser-Asn |
| 10 | CCDS | 348 | G | A | 53 | Missense | Ser-Asn |
| 11 | CCDS | 355 | G | A | 55 | Synonymous | Ser-Ser |
| 12 | CCDS | 361 | C | T | 57 | Synonymous | Asn-Asn |
| 13 | CCDS | 366 | G | A | 59 | Missense | Ser-Asn |
| 14 | CCDS | 370 | G | A | 60 | Synonymous | Ala-Ala |
| 15 | CCDS | 391 | G | T | 67 | Synonymous | Ser-Ser |
| 16 | CCDS | 446 | G | A | 86 | Missense | Ala-Thr |
| 17 | CCDS | 468 | C | T | 93 | Missense | Ser-Leu |
| 18 | CCDS | 470 | G | C | 94 | Missense | Ala-Pro |
| 19 | CCDS | 481 | C | T | 97 | Synonymous | Ala-Ala |
| 20 | CCDS | 516 | G | A | 109 | Missense | Ser-Asn |
| 21 | CCDS | 551 | A | G | 121 | Missense | Met-Val |
| 22 | CCDS | 564 | C | G | 125 | Missense | Ala-Gly |
| 23 | CCDS | 571 | C | T | 127 | Synonymous | Tyr-Tyr |
| 24 | CCDS | 577 | C | A | 129 | Missense | Asn-Lys |
| 25 | CCDS | 597 | T | C | 136 | Missense | Met-Thr |
| 26 | CCDS | 610 | G | C | 140 | Synonymous | Ala-Ala |
| 27 | CCDS | 628 | C | T | 146 | Synonymous | Arg-Arg |
| 28 | CCDS | 637 | G | A | 149 | Synonymous | Lys-Lys |
| 29 | CCDS | 646 | G | T | 152 | Missense | Arg-Ser |
| 30 | CCDS | 661 | C | T | 157 | Synonymous | His-His |
| 31 | CCDS | 760 | G | T | 190 | Missense | Met-Ile |
| 32 | CCDS | 832 | C | T | 214 | Synonymous | Asn-Asn |
| 33 | CCDS | 1027 | A | G | 279 | Synonymous | Gly-Gly |
| 34 | CCDS | 1062 | C | T | 291 | Missense | Ala-Val |
| 35 | CCDS | 1173 | C | T | 328 | Missense | Ala-Val |
| 36 | CCDS | 1175 | G | C | 329 | Missense | Ala-Pro |
| 37 | CCDS | 1227 | C | T | 346 | Missense | Ala-Val |
| 38 | CCDS | 1229 | G | A | 347 | Missense | Ala-Thr |
| 39 | CCDS | 1230 | C | G | 347 | Missense | Ala-Gly |
| 40 | CCDS | 1291 | A | C | 367 | Missense | Gly-Glu |
| 41 | CCDS | 1361 | AG | — | 391 | Frameshift | Ser-Pro |
| 42 | CCDS | 1378 | A | G | 396 | Synonymous | Gln-Gln |
| 43 | CCDS | 1395 | T | C | 402 | Missense | Leu-Pro |
| 44 | CCDS | 1401 | C | T | 404 | Missense | Ala-Val |
| 45 | CCDS | 1419 | A | C | 410 | Missense | His-Pro |
| 46 | CCDS | 1445 | C | A | 419 | Missense | Pro-Thr |
| 47 | CCDS | 1462 | G | A | 424 | Missense | Met-Ile |
| 48 | CCDS | 1474 | G | C | 428 | Synonymous | Thr-Thr |
| 49 | CCDS | 1509 | C | G | 440 | Missense | Ala-Gly |
| 50 | CCDS | 1526 | C | G | 446 | Missense | Gln-Glu |
| 51 | 3′UTR | 1569 | G | T | | | |
| 52 | 3′UTR | 1570 | A | G | | | |
| 53 | 3′UTR | 1581 | C | T | | | |
| 54 | 3′UTR | 1614 | A | G | | | |
| 55 | 3′UTR | 1618 | A | G | | | |
| 56 | 3′UTR | 1674 | A | G | | | |
| 57 | 3′UTR | 1710 | A | C | | | |
| 58 | 3′UTR | 1724 | — | G | | | |
| 59 | 3′UTR | 1725 | — | G | | | |
| 60 | 3′UTR | 1741 | A | G | | | |
| 61 | 3′UTR | 1799 | A | G | | | |
| 62 | 3′UTR | 1818 | A | T | | | |
| 63 | 3′UTR | 1825 | — | T | | | |
| 64 | 3′UTR | 1927 | A | C | | | |
| 65 | 3′UTR | 1953 | — | G/T | | | |
| 66 | 3′UTR | 1957 | — | A | | | |
| 67 | 3′UTR | 2057 | A | T | | | |
| 68 | 3′UTR | 2070 | A | G | | | |
| 69 | 3′UTR | 2071 | — | T | | | |
| 70 | 3′UTR | 2080 | — | T | | | |
| 71 | 3′UTR | 2092 | A | C | | | |
| 72 | 3′UTR | 2099 | C | T | | | |
| 73 | 3′UTR | 2187 | C | T | | | |
| 74 | 3′UTR | 2375 | A | G | | | |

TABLE 7
SNPs of the ID2 Em isoform

| | | | Contig | Poly- | Codon | | Protein |
|---|---|---|---|---|---|---|---|
| S. No. | Region | Position | reference | morphism | Position | Function | residue |
| 1 | 5′UTR | 6 | C | T | | | |
| 2 | 5′UTR | 43 | A | G | | | |
| 3 | 5′UTR | 53 | A | G | | | |
| 4 | 5′UTR | 55 | C | G | | | |
| 5 | 5′UTR | 154 | C | G/T | | | |
| 6 | CCDS | 195 | C | T | 4 | Missense | Phe-Phe |
| 7 | CCDS | 209 | C | T | 9 | Missense | Ser-Phe |
| 8 | CCDS | 224 | G | A | 14 | Missense | Ser-Asn |
| 9 | CCDS | 237 | C | T | 18 | Synonymous | His-His |
| 10 | CCDS | 263 | C | A | 27 | Missense | Thr-Asn |
| 11 | CCDS | 286 | C | T | 35 | Synonymous | Leu-Leu |
| 12 | CCDS | 360 | G | A | 59 | Synonymous | Val-Val |
| 13 | CCDS | 399 | C | T | 72 | Synonymous | Ile-Ile |
| 14 | CCDS | 405 | C | T | 74 | Synonymous | Asp-Asp |
| 15 | CCDS | 485 | C | T | 101 | Missense | Thr-Met |
| 16 | CCDS | 501 | C | G/T | 106 | Synonymous | Leu-Leu |
| 17 | CCDS | 544 | C | T | 121 | Missense | Pro-Ser |
| 18 | CCDS | 547 | T | A | 122 | Missense | Ser-Thr |
| 19 | 3′UTR | 605 | A | G | | | |
| 20 | 3′UTR | 662 | C | G | | | |
| 21 | 3′UTR | 665 | G | T | | | |
| 22 | 3′UTR | 716 | A | T | | | |
| 23 | 3′UTR | 757 | C | T | | | |
| 24 | 3′UTR | 871 | A | G | | | |
| 25 | 3′UTR | 876 | A | G | | | |
| 26 | 3′UTR | 975 | — | >6 bp | | | |
| 27 | 3′UTR | 1085 | — | >6 bp | | | |
| 28 | 3′UTR | 1115 | A | G | | | |
| 29 | 3′UTR | 1119 | — | AT | | | |
| 30 | 3′UTR | 1149 | C | T | | | |
| 31 | 3′UTR | 1151 | A | T | | | |
| 32 | 3′UTR | 1251 | — | CA | | | |
| 33 | 3′UTR | 1333 | A | G | | | |
| 34 | 3′UTR | 1350 | C | G | | | |

TABLE 8
SNPs of the ID2 Ad isoform

| | | | Contig | Poly- | Codon | | Protein |
|---|---|---|---|---|---|---|---|
| S. No. | Region | Position | reference | morphism | Position | Function | residue |
| 5 | 5′UTR | 93 | C | G/T | | | |
| 6 | CCDS | 134 | C | T | 4 | Missense | Phe-Phe |
| 7 | CCDS | 148 | C | T | 9 | Missense | Ser-Phe |
| 8 | CCDS | 163 | G | A | 14 | Missense | Ser-Asn |
| 9 | CCDS | 176 | C | T | 18 | Synonymous | His-His |
| 10 | CCDS | 202 | C | A | 27 | Missense | Thr-Asn |
| 11 | CCDS | 225 | C | T | 35 | Synonymous | Leu-Leu |
| 12 | CCDS | 299 | G | A | 59 | Synonymous | Val-Val |
| 13 | CCDS | 338 | C | T | 72 | Synonymous | Ile-Ile |
| 14 | CCDS | 344 | C | T | 74 | Synonymous | Asp-Asp |
| 15 | CCDS | 424 | C | T | 101 | Missense | Thr-Met |
| 16 | CCDS | 440 | C | G/T | 106 | Synonymous | Leu-Leu |
| 17 | CCDS | 483 | C | T | 121 | Missense | Pro-Ser |
| 18 | CCDS | 486 | T | A | 122 | Missense | Ser-Thr |
| 19 | 3′UTR | 544 | A | G | | | |
| 20 | 3′UTR | 601 | C | G | | | |
| 21 | 3′UTR | 604 | G | T | | | |
| 22 | 3′UTR | 655 | A | T | | | |
| 23 | 3′UTR | 696 | C | T | | | |
| 24 | 3′UTR | 810 | A | G | | | |
| 25 | 3′UTR | 815 | A | G | | | |
| 26 | 3′UTR | 914 | — | >6 bp | | | |
| 27 | 3′UTR | 1024 | — | >6 bp | | | |
| 28 | 3′UTR | 1054 | A | G | | | |
| 29 | 3′UTR | 1058 | — | AT | | | |
| 30 | 3′UTR | 1088 | C | T | | | |
| 31 | 3′UTR | 1090 | A | T | | | |
| 32 | 3′UTR | 1190 | — | CA | | | |
| 33 | 3′UTR | 1272 | A | G | | | |
| 34 | 3′UTR | 1289 | C | G | | | |

A control sample according to the present invention is a sample from a healthy control subject. Such a sample can be obtained for example from a subject known to be a healthy subject. It is also possible to generate a control sample according to the present invention as a mixture of samples obtained from several healthy subjects, for example from a group of 10, 20, 30, 50, 100 or even up to 1000 healthy subjects. A control sample according to the present invention can be generated for example from age-matched and or gender-matched healthy control subjects. A control sample according to the present invention can also be generated for example in vitro to mimic a control sample obtained from one or several healthy subjects.

Control samples can, inter alia, be healthy tissues (i.e. biopsies) from diseased individuals/subjects. “Healthy tissue from diseased individuals/subjects” can refer to tissue that is pathologically classified as “normal” or “healthy” and/or that is distant or adjacent to a (suspected) tumor. For example, the “healthy tissue from diseased individuals/subjects” can be obtained e.g. by biopsy from adjacent healthy tissue of (suspected) cancer patients.

For example, the “healthy tissue” can be obtained from the subject(s) to be assessed in accordance with the present invention for suffering from cancer or being prone to suffering from cancer. In another example, the “healthy tissue” can be obtained from other diseased patients (e.g. patients that have already been diagnosed to suffer from cancer by conventional means and methods or patients that have a history of cancer); in that case, “healthy tissue” is not obtained from subject(s) to be assessed in accordance with the present invention for suffering from cancer or being prone to suffering from cancer.

Thus, also “healthy tissue from (a) diseased individual(s)” can be used as a control sample in accordance with the present invention.

Control samples can, inter alia, be EBCs from healthy individuals. The term “healthy individuals” as used herein can refer to individuals with no history of cancer, i.e. individuals that did not suffer from cancer or that do currently (i.e. at the time the control sample is obtained) not suffer from cancer. Thus, “healthy tissue/sample” (i.e. tissue (e.g. a biopsy) or another sample (e.g. EBC) obtained from a healthy individual” can be used as a control sample in accordance with the present invention.

A subject according to the present invention is preferably a human subject. The subject according to the present invention can be a human subject which has an increased likelihood of suffering from cancer. Such an increased likelihood of suffering from cancer can for example result from certain exposures to cancerogens, for example through the habit of smoking.

The “amount of said specific transcription isoform” according to the present invention can be a relative amount or an absolute amount. The relative amount can be determined relative to a control sample. To determine the “amount of said specific transcription isoform”, the absolute or relative amount of a reference gene or reference protein can be determined in the sample from the subject and in the control sample. Non-limiting examples of reference genes/proteins are TUBA1A1 (Uniprot-ID: Q71U36, Gene-ID: 7846), HPRT1 (Uniprot-ID: P00492, Gene-ID: 3251), ACTB (Uniprot-ID: P60709, Gene-ID: 60), HMBS (Uniprot-ID: P08397, Gene-ID: 3145), RPL13A (Uniprot-ID: Q9BSQ6, Gene-ID: 23521) and UBE2A (Uniprot-ID: P49459, Gene-ID: 7319).

The herein provided method can be used to stratify/assess subjects according to the tumor/cancer grade. It can be helpful to assess whether a patient is suffering from Grade I, Grade II or Grade III tumor/cancer in order to decide which therapeutic intervention is warranted.

The definition of Grade I, Grade II and Grade III tumor is based on TNM classification recommended by the American Joint Committee on Cancer (Goldstraw P. et al. (2007) J Thorac Oncol. 2(8):706-14; Beadsmoore C J and Screaton N J (2003) Eur J Radiol. 45(1):8-17; Mountain C F (1997) Chest. 111(6):1710-7.), which is incorporated herein by reference.

Herein, lung cancer is preferred, in particular non-small cell lung cancer or small cell lung cancer. Particularly preferred is non-small cell lung cancer.

It is known by the person skilled in the art that genes can contain single nucleotide polymorphisms. The specific transcription factor Em isoform sequences of the present invention encompass all naturally occurring sequences of the respective isoform independent of the number and nature of the SNPs in said sequence. To relate to currently known SNPs, the specific transcription factor Ad isoform sequences of the present invention are defined such that they contain up to 55 (in the case of GATA6) or up to 38 (in the case of NKX2-1), up to 74 (in the case of FOXA2) or up to 30 (in the case of ID2) additions, deletions or substitutions of the nucleic acid sequences defined by SEQ ID NOs: 5, 6, 7 and 8, respectively, to also cover the respective Ad transcripts of carriers of different nucleotides at the respective SNPs. The SNPs of tables 2, 4, 6 and 8 may occur in the Ad isoforms of the present invention in any combination. For example, a (genetic) variant of the GATA6 Ad isoform to be used herein may comprise a nucleic acid sequence of SEQ ID NO:5, whereby the “C” residue at position 694 of SEQ ID NO:5 is substituted by “T”. Further variants of the isoforms to be used herein are apparent from Tables 1 to 8 to the person skilled in the art.

The GATA6 Ad isoform according to the invention is the GATA6 Ad isoform comprising the nucleic acid sequence of SEQ ID NO: 5 or the GATA6 Ad isoform comprising a nucleic acid sequence with up to 55; preferably up to 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21 or 20; even more preferably up to 19, 18, 17, 16, 15, 14, 13, 12, 11, 10, 9, 8, 7. 6, 5, 4, 3 or 2; or even furthermore preferably only 1 addition(s), deletion(s) or substitution(s) of SEQ ID NO: 5. The GATA6 Ad isoform can also be defined as the GATA6 Ad isoform comprising the nucleic acid sequence of SEQ ID NO: 5 or the GATA6 Ad isoform comprising the nucleic acid sequence of SEQ ID NO: 5 with additions, deletions or substitutions at any of positions 138; 228; 255; 262; 274; 365; 397; 415; 694; 1063; 1191; 1239; 1524; 1532; 1562; 1586; 1587; 1738; 1779; 1784; 1814; 1817; 1846; 1875; 1884; 1917; 1935; 1937; 1943; 1961; 1966; 2041; 2072; 2077; 2098; 2229; 2325; 2326; 2562; 2626; 2971; 3037; 3175; 3200; 3201; 3225; 3293; 3301; 3513; 3567; 3581; 3605; 3625; 3643 or 3670. The GATA6 Ad isoform according to the invention can also be defined as the GATA6 Ad isoform comprising the nucleic acid sequence of SEQ ID No: 5 or the GATA6 Ad isoform comprising a nucleic acid sequence with at least 85% homology to SEQ ID No: 5, preferably up to 86%, 87%, 88%, 89%, 90%, 91%, 92%, 93%, 94%, 95%, 96%, 97% or 98% homology to SEQ ID No: 5; even more preferably up to 99% homology to SEQ ID No: 5.

The NKX2-1 Ad isoform according to the invention is the NKX2-1 Ad isoform comprising the nucleic acid sequence of SEQ ID NO: 6 or the NKX2-1 Ad isoform comprising a nucleic acid sequence with up to 38; preferably up to 37, 36, 35, 34, 33, 32, 31, 30, 29, 28, 27, 26, 25, 24, 23, 22, 21, 20, 19, 18, 17, 16, 15, 14, 13, 12, 11, or 10; even more preferably up to 9, 8, 7, 6, 5, 4, 3, or 2; or even furthermore preferably only 1 addition(s), deletion(s) or substitution(s) of SEQ ID NO: 6. The NKX2-1 Ad isoform can also be defined as the NKX2-1 Ad isoform comprising the nucleic acid sequence of SEQ ID NO: 6 or the Nkx2-1 isoform Ad comprising the nucleic acid sequence of SEQ ID NO: 6 with additions, deletions or substitutions at any of positions 12; 125; 265; 270; 284; 286; 295; 331; 626; 630; 670; 795; 1014; 1150; 1189; 1293; 1303; 1312; 1334; 1397; 1478; 1479; 1478; 1485; 1486; 1488; 1512; 1518; 1523; 1593; 1595; 1676; 1738; 1761; 1762; 1779; 1944 or 2164. The NKX2-1 Ad isoform according to the invention can also be defined as the NKX2-1 Ad isoform comprising the nucleic acid sequence of SEQ ID No: 6 or the NKX2-1 Ad isoform comprising a nucleic acid sequence with at least 90% homology to SEQ ID No: 6, preferably up to 91%, 92%, 93%, 94%, 95%, 96%, 97% or 98% homology to SEQ ID No: 6; even more preferably up to 99% homology to SEQ ID No: 6.

The FOXA2 Ad isoform according to the invention is the FOXA2 Ad isoform comprising the nucleic acid sequence of SEQ ID NO: 7 or the FOXA2 Ad isoform comprising a nucleic acid sequence with up to 74; preferably up to 73, 72, 71, 70, 69, 68, 67, 66, 65, 64, 63, 62, 61, 60, 59, 58, 57, 56, 55, 54, 53 52, 51, 50, 49, 48 47, 46, 45, 44, 43, 42, 41, 40, 39, 38, 37, 36, 35, 34, 33, 32, 31, 30, 29, 28, 27, 26, 25, 24, 23, 22, 21 or 20; even more preferably up to 19, 18, 17, 16, 15, 14, 13, 12, 11, 10, 9, 8, 7. 6, 5, 4, 3 or 2; or even furthermore preferably only 1 addition(s), deletion(s) or substitution(s) of SEQ ID NO: 7. The FOXA2 Ad isoform can also be defined as the FOXA2 Ad isoform comprising the nucleic acid sequence of SEQ ID NO: 7 or the FOXA2 Ad isoform comprising the nucleic acid sequence of SEQ ID NO: 7 with additions, deletions or substitutions at any of positions 5; 37; 65; 68; 70; 88; 128; 195; 276; 348; 355; 361; 366; 370; 391; 446; 468; 470; 481; 516; 551; 564; 571; 577; 597; 610; 628; 637; 646; 661; 760; 832; 1027; 1062; 1173; 1175; 1227; 1229; 1230; 1291; 1361; 1378; 1395; 1401; 1419; 1445; 1462; 1474; 1509; 1526; 1569; 1570; 1581; 1614; 1618; 1674; 1710; 1724; 1725; 1741; 1799; 1818; 1825; 1927; 1953; 1957; 2057; 2070; 2071; 2080; 2092; 2099; 2187 or 2375. The FOXA2 Ad isoform according to the invention can also be defined as the FOXA2 Ad isoform comprising the nucleic acid sequence of SEQ ID No: 7 or the FOXA2 Ad isoform comprising a nucleic acid sequence with at least 93% homology to SEQ ID No: 7, preferably up to 92%, 93%, 94%, 95%, 96%, 97% or 98% homology to SEQ ID No: 7; even more preferably up to 99% homology to SEQ ID No: 7.

The ID2 Ad isoform according to the invention is the ID2 Ad isoform consisting the nucleic acid sequence of SEQ ID NO: 8 or the ID2 Ad isoform consisting of a nucleic acid sequence with up to 30; preferably up to 29, 28, 27, 26, 25, 24, 23, 22, 21, 20, 19, 18, 17, 16, 15, 14, 13, 12, 11, or 10; even more preferably up to 9, 8, 7, 6, 5, 4, 3, or 2; or even furthermore preferably only 1 addition(s), deletion(s) or substitution(s) of SEQ ID NO: 8. The ID2 Ad isoform can also be defined as the ID2 Ad isoform consisting the nucleic acid sequence of SEQ ID NO: 8 or the ID2 Ad isoform consisting the nucleic acid sequence of SEQ ID NO: 8 with additions, deletions or substitutions at any of positions 93; 134; 148; 163; 176; 202; 225; 299; 338; 344; 424; 440; 483; 486; 544; 601; 604; 655; 696; 810; 815; 914; 1024; 1054; 1058; 1088; 1090; 1190; 1272 or 1289. The ID2 Ad isoform according to the invention can also be defined as the ID2 Ad isoform comprising the nucleic acid sequence of SEQ ID No: 8 or the ID2 Ad isoform comprising a nucleic acid sequence with at least 51% homology to SEQ ID No: 8, preferably up to 55%, 60%, 65%, 70%, 75%, 80%, 85% or 90% homology to SEQ ID No: 8; even more preferably up to 91%, 92%, 93%, 94%, 95%, 96%, 97%, 98% or 99% homology to SEQ ID No: 8.

The term “cancer patient” as used herein refers to a patient that is suspected to suffer from cancer or being prone to suffer from cancer. The cancer to be treated in accordance with the present invention can be a solid cancer or a liquid cancer. Non-limiting examples of cancers which can be treated according to the present invention are lung cancer, ovarian cancer, colorectal cancer, kidney cancer, bone cancer, bone marrow cancer, bladder cancer, prostate cancer, esophagus cancer, salivary gland cancer, pancreas cancer, liver cancer, head and neck cancer, CNS (especially brain) cancer, cervix cancer, cartilage cancer, colon cancer, genitourinary cancer, gastrointestinal tract cancer, pancreas cancer, synovium cancer, testis cancer, thymus cancer, thyroid cancer and uterine cancer.

Preferably, the cancer patient according to the present invention is a patient suffering from lung cancer, such as non-small cell lung cancer (NSCLC) or small cell lung cancer (SLC). Particularly preferably, the patient suffers non-small cell lung cancer (NSCLC). Even more preferably, the cancer patient is a patient suffering from adenocarcinoma. The patient may also suffer from a squamous cell carcinoma or a large cell carcinoma. The adenocarcinoma can be a bronchoalveolar carcinoma.

The amount of the specific transcription factor isoform according to the invention can be measured for example by a polymerase chain reaction-based method, an in situ hybridization-based method, or a microarray. If the amount of the specific transcription factor isoform according to the invention is measured via a polymerase chain reaction-based method, it is preferably measured via a quantitative reverse transcriptase polymerase chain reaction.

The method of assessing whether a subject suffers from cancer or is prone to suffering from cancer according to the invention may comprise the contacting of a sample with primers, wherein said primers can be used for amplifying the respective specific transcription factor isoforms.

Primers for the polymerase chain reaction-based measurement of the amount of the specific transcription factor isoforms according to the invention may encompass the use of primers being selected from the Table 9.

TABLE 9
Examples of primer pairs for the amplification, detection and/or
quantification of the amount of specific transcription factor isoforms

| | Primers | Primers for Human (5′→3′) (For |
|---|---|---|
| Gene | for Human (5′→3′) | RNA from tissue sections) |
| Gata6-Em Fwd | SEQ ID NO 9: | SEQ ID NO 10: |
| | CTCGGCTTCTCTCCGCGCCTG | TTGACTGACGGCGGCTGGTG |
| Gata6-Em Rev | SEQ ID NO 11: | SEQ ID NO 12: |
| | AGCTGAGGCGTCCCGCAGTTG | CTCCCGCGCTGGAAAGGCTC |
| Gata6-Ad Fwd | SEQ ID NO 13: | SEQ ID NO 14: |
| | GCGGTTTCGTTTTCGGGGAC | AGGACCCAGACTGCTGCCCC |
| Gata6-Ad Rev | SEQ ID NO 15: | SEQ ID NO 16: |
| | AAGGGATGCGAAGCGTAGGA | CTGACCAGCCCGAACGCGAG |
| Nkx2-1-Em Fwd | SEQ ID NO 17: | SEQ ID NO 18: |
| | AAACCTGGCGCCGGGCTAAA | CAGCGAGGCTTCGCCTTCCC |
| Nkx2-1-Em Rev | SEQ ID NO 19: | SEQ ID NO 20: |
| | GGAGAGGGGGAAGGCGAAGCC | TCGACATGATTCGGCGGCGG |
| Nkx2-1-Ad Fwd | SEQ ID NO 21: | SEQ ID NO 22: |
| | AGCGAAGCCCGATGTGGTCC | TCCGGAGGCAGTGGGAAGGC |
| Nk2-1-Ad Rev | SEQ ID NO 23: | SEQ ID NO 24: |
| | CCGCCCTCCATGCCCACTTTC | GACATGATTCGGCGGCGGCT |
| Foxa2-Var1 Fwd | SEQ ID NO 25: | SEQ ID NO 26: |
| | TGCCATGCACTCGGCTTCCAG | CAGGGAGAGGGAGGGCGAGA |
| Foxa2-Var1 Rev | SEQ ID NO 27: | SEQ ID NO 28: |
| | TCATGTTGCCCGAGCCGCTG | CCCCCACCCCCACCCTCTTT |
| Foxa2-Var2 Fwd | SEQ ID NO 29: | SEQ ID NO 30: |
| | CTGCTAGAGGGGCTGCTTGCG | CGCTTCTCCCGAGGCCGTTC |
| Foxa2-Var2 Rev | SEQ ID NO 31: | SEQ ID NO 32: |
| | ACGGCTCGTGCCCTTCCATC | TAACTCGCCCGCTGCTGCTC |
| Id2-Var1 Fwd | SEQ ID NO 33: | SEQ ID NO 34: |
| | AACCCCTGTGGACGACCCGA | TGCGGATAAAAGCCGCCCCG |
| Id2-Var1 Rev | SEQ ID NO 35 | SEQ ID NO 36: |
| | GCCCGGGTCTCTGGTGATGC | AGCTAGCTGCGCTTGGCACC |
| Id2-Var2 Fwd | SEQ ID NO 37: | SEQ ID NO 38: |
| | CTGCGGTGCTGAACTCGCCC | CCCCCTGCGGTGCTGAACTC |
| Id2-Var2 Rev | SEQ ID NO 39: | SEQ ID NO 40: |
| | GACGAGCGGGCGCTTCCATT | TAACTCGCCCGCTGCTGCTC |

The diagnostic methods can be used, for example, in combination with (i.e. subsequently prior to or simultaneously with) other diagnostic techniques, like CT (short for computer tomography) and CXR (short for chest radiograph, colloquially called chest X-ray (CXR)).

The herein provided methods for the diagnosis of a patient group and the therapy of this selected patient group is particularly useful for high risk subjects/patients or patient groups, such as those that have a hereditary history and/or are exposed to tobacco smoke, environmental smoke, cooking fumes, indoor smoky coal emissions, asbestos, some metals (e.g. nickel, arsenic and cadmium), radon (particularly amongst miners) and ionizing radiation. These subjects/patients may particularly profit from an early diagnosis and, hence, treatment of the cancer in accordance with the present invention.

A method of treating a patient according to the present invention may comprise
a) obtaining a sample from a patient;
b) selecting a cancer patient according to any of the above mentioned statistical methods of assessing whether a subject suffers from cancer or is prone to suffering from cancer;
c) administering to said cancer patient an effective amount of an anti-cancer agent.

The present invention also provides a method of treating a patient, said method comprising
a) selecting a cancer patient according to any of the above mentioned statistical methods of assessing whether a subject suffers from cancer or is prone to suffering from cancer
b) administering to said cancer patient an effective amount of an anti-cancer agent, wherein the cancer agent is for example selected from the group of agents comprising Oxalaplatin, Gemcitabine (Gemzar), Paclitaxel (Taxol), Vincristine (Oncovin) and a composition for use in medicine comprising an inhibitor of
   i) the GATA6 Em isoform comprising the nucleic acid sequence of SEQ ID No: 1 or the GATA6 Em isoform comprising the nucleic acid sequence with up to 55 additions, deletions or substitutions of SEQ ID NO: 1;
   ii) the NKX2-1 Em isoform comprising the nucleic acid sequence of SEQ ID No: 2 or the NKX2-1 Em isoform comprising the nucleic acid sequence with up to 39 additions, deletions or substitutions of SEQ ID NO: 2.
   iii) the FOXA2 Em isoform comprising the nucleic acid sequence of SEQ ID No: 3 or the FOXA2 Em isoform comprising nucleic acid sequence with up to 68 additions, deletions or substitutions of SEQ ID NO: 3; and/or
   iv) the ID2 Em isoform comprising the nucleic acid sequence of SEQ ID No: 4 or the ID2 Em isoform comprising nucleic acid sequence with up to 34 additions, deletions or substitutions of SEQ ID NO: 4.

The present invention relates to a pharmaceutical composition comprising an agent for the treatment or the prevention of cancer, wherein for the patient suffering from cancer has been determined by a statistical method of the present invention and wherein the method of treatment comprises the step of determining whether or not the patient suffers from cancer. Preferably, the pharmaceutical composition according to the present invention comprises an agent for the treatment or the prevention of lung cancer, wherein for the patient lung cancer has been determined by a method of the present invention and wherein the method of treatment comprises the step of determining whether or not the patient suffers from lung cancer

For example, the pharmaceutical composition to be used herein in the treatment of patients selected according to the statistical methods provide herein can an inhibitor of
i) the GATA6 Em isoform comprising the nucleic acid sequence of SEQ ID No: 1 or the GATA6 Em isoform comprising the nucleic acid sequence with up to 55 additions, deletions or substitutions of SEQ ID NO: 1;
ii) the NKX2-1 Em isoform comprising the nucleic acid sequence of SEQ ID No: 2 or the NKX2-1 Em isoform comprising the nucleic acid sequence with up to 39 additions, deletions or substitutions of SEQ ID NO: 2;
iii) the FOXA2 Em isoform comprising the nucleic acid sequence of SEQ ID No: 3 or the FOXA2 Em isoform comprising nucleic acid sequence with up to 68 additions, deletions or substitutions of SEQ ID NO: 3; and/or
iv) the ID2 Em isoform comprising the nucleic acid sequence of SEQ ID No: 4 or the ID2 Em isoform comprising nucleic acid sequence with up to 34 additions, deletions or substitutions of SEQ ID NO: 4.

It is surprisingly found that the Em isoforms of the transcription factors of the present invention have an oncogenic potential (see Examples 4, 6 and 7). Further, it is shown that their reduction leads to the prevention of the development of tumors and allows treating cancer (see example 7). Thus, the present invention relates to inhibitors of the Em isoforms of the transcription factors GATA6, NKX2-1, FOXA2 and ID2. In particular, the present invention relates to agents that allow reducing the amount of the Em isoform of the transcription factors GATA6, NKX2-1, FOXA2 and ID2. The present invention also relates to activators of the Ad isoform of the transcription factors GATA6, NKX2-1, FOXA2 and ID2. Examples of such activators are agents, which activate the promoter of the Ad isoform of the respective transcription factors.

The inhibitors of
i) the GATA6 Em isoform comprising the nucleic acid sequence of SEQ ID No: 1 or the GATA6 Em isoform comprising the nucleic acid sequence with up to 55 additions, deletions or substitutions of SEQ ID NO: 1;
ii) the NKX2-1 Em isoform comprising the nucleic acid sequence of SEQ ID No: 2 or the NKX2-1 Em isoform comprising the nucleic acid sequence with up to 39 additions, deletions or substitutions of SEQ ID NO: 2,
iii) the FOXA2 Em isoform comprising the nucleic acid sequence of SEQ ID No: 3 or the FOXA2 Em isoform comprising nucleic acid sequence with up to 68 additions, deletions or substitutions of SEQ ID NO: 3; or
iv) the ID2 Em isoform comprising the nucleic acid sequence of SEQ ID No: 4 or the ID2 Em isoform comprising nucleic acid sequence with up to 34 additions, deletions or substitutions of SEQ ID NO: 4
   according to the present invention can for example comprise siRNAs (small interfering RNAs) or shRNAs (small hairpin RNAs) targeting said specific transcription factor Em isoforms.

The person skilled in the art knows how to design siRNAs and shRNAs, which specifically target the specific transcription factor Em isoforms of the present invention. Examples of such specific siRNAs and shRNAs targeting the specific transcription factor Em isoforms of the present invention are depicted in Tables 10 and 11.

TABLE 10
Examples of siRNA sequences for the knockdown of Gata6 Em
Gata6

| Target Sequence | Sense strand siRNA | Antisense strand siRNA |
|---|---|---|
| AATCAGGAGCGCAGGCTGCAG | SEQ ID NO: 41 | SEQ ID NO: 43 |
| (SEQ ID NO. 58) | UCAGGAGCGCAGGCUGCAGtt | CUGCAGCCUGCGCUCCUGA |
| | | tt |
| AAGAGGCGCCTCCTCTCTCCT | SEQ ID NO: 42 | SEQ ID NO: 44 |
| (SEQ ID NO. 59) | GAGGCGCCUCCUCUCUCCUtt | AGGAGAGAGGAGGCGCCU |
| | | Ctt |

Foxa2

| Target Sequence | Sense strand siRNA | Antisense strand siRNA |
|---|---|---|
| AAACCGCCATGCACTCGGCTT | SEQ ID NO: 45 | SEQ ID NO: 46 |
| (SEQ ID NO. 60) | ACCGCCAUGCACUCGGCUUtt | AAGCCGAGUGCAUGGCGG |
| | | Utt |

TABLE 11
Examples of shRNA sequences for the knockdown of
Nkx2-1
Nkx2-1
shHairpin sequence (5′-3′)
SEQ ID NO: 47
CCGGCCCATGAAGAAGAAAGCAATTCTCGAGAATTGCTTTCTTCTTCAT
GGGTTTTTG
SEQ ID NO: 48
GTACCGGGGGATCATCCTTGTAGATAAACTCGAGTTTATCTACAAGGAT
GATCCCTTTTTTG
SEQ ID NO: 49
CCGGATTCGGAATCAGCTAGCAATTCTCGAGAATTGCTAGCTGATTCCG
AATTTTTTG

The amount of the specific transcription factor isoform according to the present invention can be determined on the polypeptide level.

The amount of the specific transcription factor isoforms according to the invention can be assessed on the polypeptide level using known quantitative methods for the assessment of polypeptide levels. For example, ELISA (Enzyme-linked Immunosorbent Assay)-based, gel-based, blot-based, mass spectrometry-based, or flow cytometry-based methods can be used for measuring the amount of the specific transcription factor isoforms on the polypeptide level according to the invention.

It is apparent to the person skilled in the art that the specific transcription factor isoforms of the present invention can show certain sequence varieties between different subjects of the same ancestry and in particular between subjects of different ancestry. Non-limiting examples of the polymorphisms of the cancer specific isoforms of the present invention are given in Tables 12 and 13.

TABLE 12
Examples of polymorphisms in the sequences of GATA6, Em and Ad isoforms in
dependence of the ancestry of a subject (CEU: Utah residents with Northern and Western
European ancestry from the CEPH collection; CHB: Han Chinese in Beijing, China; JPT:
Japanese in Tokyo, Japan; YRI: Yoruban in Ibadan, Nigeria)

| S. No | Region | Position in Gata6 Em | Position in Gata6 Ad | Polymorphism | Population | Frequency of T | Frequency of C |
|---|---|---|---|---|---|---|---|
| 1 | CCDS | 1982 | 1917 | T/C | CEU | 100% | 0% |
| | | | | | JPT | 100% | 0% |
| | | | | | YRI | 100% | 0% |
| S. No | Region | Position in Gata6 Em | Position in Gata6 Ad | Polymorphism | Population | Frequency of G | Frequency of A |
| 2 | 3′UTR | 2137 | 2072 | G/A | CEU | 56% | 44% |
| | | | | | CHB | 57% | 43% |
| | | | | | JPT | 65% | 35% |
| | | | | | YRI | 45% | 55% |
| S. No | Region | Position in Gata6 Em | Position in Gata6 Ad | Polymorphism | Population | Frequency of A | Frequency of G |
| 3 | 3′UTR | 2142 | 2077 | A/G | CEU | 97% | 3% |
| | | | | | CHB | 90% | 10% |
| | | | | | JPT | 100% | 0% |
| | | | | | YRI | 100% | 0% |
| S. No | Region | Position in Gata6 Em | Position in Gata6 Ad | Polymorphism | Population | Frequency of T | Frequency of A |
| 4 | 3′UTR | 2391 | 2326 | T/A | CEU | 100% | 0% |
| | | | | | CHB | 100% | 0% |
| | | | | | JPT | 100% | 0% |
| | | | | | YRI | 100% | 0% |

TABLE 13
Examples of polymorphisms in the sequences of FOXA2 variant 1 and 2 in
dependence of the ancestry of a subject (ASW: African ancestry in Southwest USA; CEU:
Utah residents with Northern and Western European ancestry from the CEPH collection; CHB:
Han Chinese in Beijing, China; CHD: Chinese in Metropolitan Denver, Colorado; GIH:
Gujarati Indians in Houston, Texas; JPT: Japanese in Tokyo, Japan; LWK: Luhya in Webuye,
Kenya; MEX: Mexican ancestry in Los Angeles, California; MKK: Maasai in Kinyawa,
Kenya; TSI: Tuscan in Italy; YRI: Yoruban in Ibadan, Nigeria)

| S. No | Region | Position in Foxa2 Em | Position in Foxa2 Ad | Polymorphism | Population | Frequency of T | Frequency of C |
|---|---|---|---|---|---|---|---|
| 1 | CCDS | 1408 | 1395 | T/C | CEU | 100% | 0% |
| | | | | | CHB | 100% | 0% |
| | | | | | JPT | 100% | 0% |
| | | | | | YRI | 100% | 0% |
| S. No | Region | Position in Foxa2 Em | Position in Foxa2 Ad | Polymorphism | Population | Frequency of A | Frequency of G |
| 1 | 3′UTR | 1627 | 1614 | A/G | ASW | 38% | 62% |
| | | | | | CEU | 96% | 4% |
| | | | | | CHB | 84% | 16% |
| | | | | | CHD | 84% | 16% |
| | | | | | JPT | 77% | 23% |
| | | | | | GIH | 89% | 11% |
| | | | | | LWK | 27% | 73% |
| | | | | | MEX | 92% | 8% |
| | | | | | MKK | 40% | 60% |
| | | | | | TSI | 91% | 9% |
| | | | | | YRI | 20% | 80% |

In certain aspects, the present invention provides a kit for use in carrying out the statistical method of the present invention. The kit of the present invention may comprise primers and further reagents necessary for a qPCR analysis. The respective primers may be selected from the list in Table 9.

While the invention has been illustrated and described in detail in the drawings and foregoing description, such illustration and description are to be considered illustrative or exemplary and not restrictive. It will be understood that changes and modifications may be made by those of ordinary skill within the scope and spirit of the following claims. In particular, the present invention covers further embodiments with any combination of features from different embodiments described above and below.

The invention also covers all further features shown in the figures individually although they may not have been described in the afore or following description. Also, single alternatives of the embodiments described in the figures and the description and single alternatives of features thereof can be disclaimed from the subject matter of the other aspect of the invention.

Furthermore, in the claims the word “comprising” does not exclude other elements or steps, and the indefinite article “a” or “an” does not exclude a plurality. A single unit may fulfill the functions of several features recited in the claims. The terms “essentially”, “about”, “approximately” and the like in connection with an attribute or a value particularly also define exactly the attribute or exactly the value, respectively. Any reference signs in the claims should not be construed as limiting the scope.

The present invention is further described by reference to the following non-limiting figures and examples. Unless otherwise indicated, established methods of recombinant gene technology were used as described, for example, in Sambrook, Russell “Molecular Cloning, A Laboratory Manual”, Cold Spring Harbor Laboratory, N.Y. (2001)) which is incorporated herein by reference in its entirety.

The Figures show:

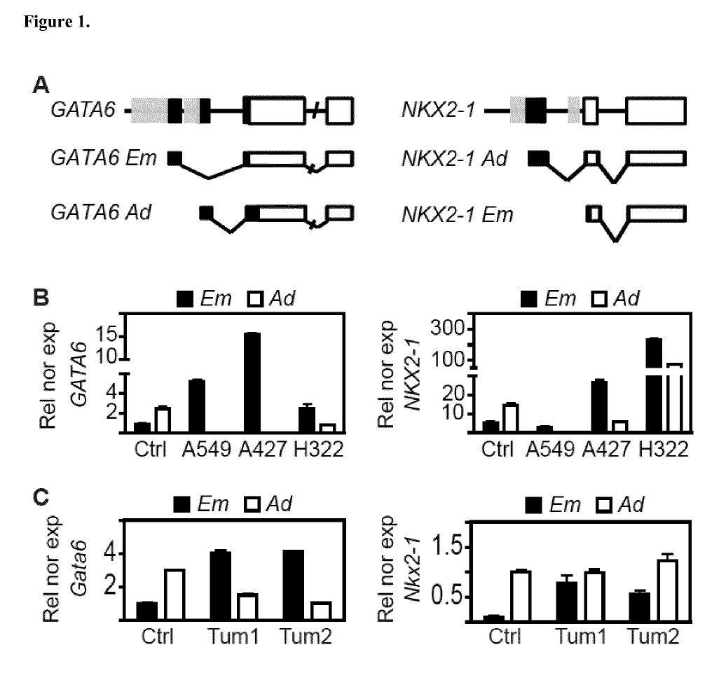

FIG. 1: Embryonic isoforms of GATA6 and NKX2-1 are highly expressed in human lung cancer cell lines and in a mouse model of experimental metastasis. (A) Schematic representation of the gene structure of human GATA6 and NKX2-1. In silico analysis of the indicated genes (top) shows an identical arrangement with two promoters (grey boxes) driving the expression of two distinct transcripts (middle and bottom; exons as black and coding region as white boxes). GATA6, GATA Binding Factor 6; NKX2-1, also known as Ttf1, Thyroid transcription factor 1; Em, Embryonic; Ad, Adult. (B) The two transcript isoforms are differentially regulated during lung cancer and show complementary expression. Isoform specific gene expression analysis was performed for both genes by q-RT PCR in control donor lung tissue (Ctrl) and lung cancer cell lines, A549, A427 (adenocarcinoma) and H322 (bronchoalveolar carcinoma). Rel nor exp, relative expression normalized to TUBA1A. Error bars, standard error of the mean (s.e.m.), n=5. (C) High expression of Em-isoform of Gata6 and Nkx2-1 in a mouse model for tumor metastasis. Isoform specific expression analysis was performed in lungs from control mice (n=3) injected with PBS (Ctrl) and lung tumors (Tum) that developed in mice (n=5) after tail vein injection of 1 million LLC1 cells. Representative are shown the results from one control and two experimental (Tuml, 2) mice. Data are represented as in B.

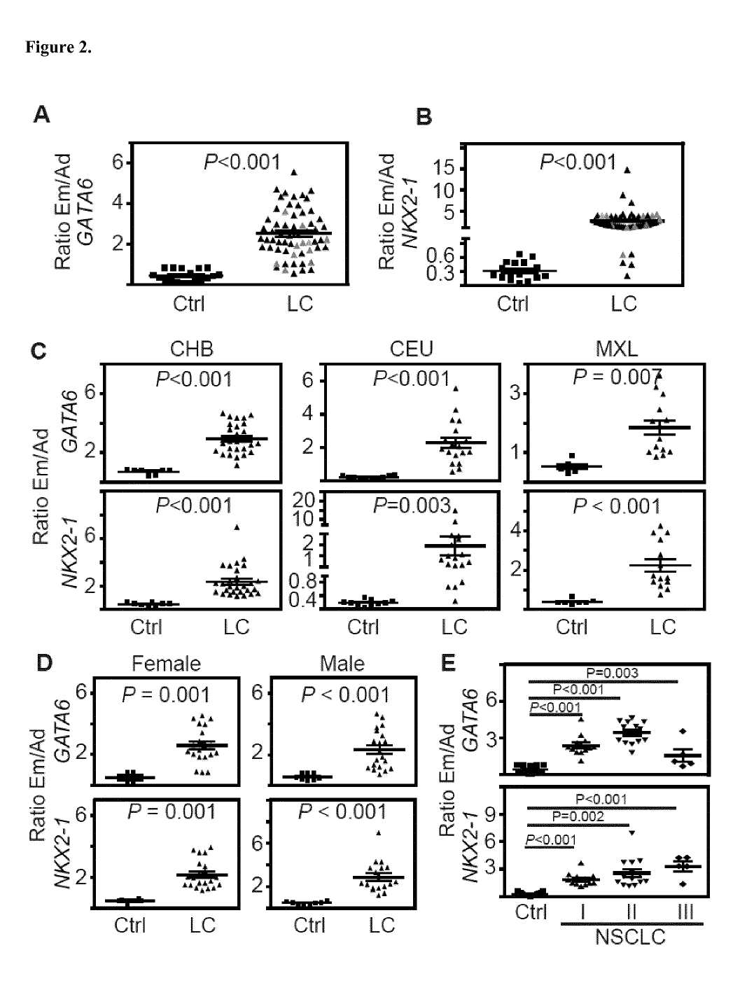

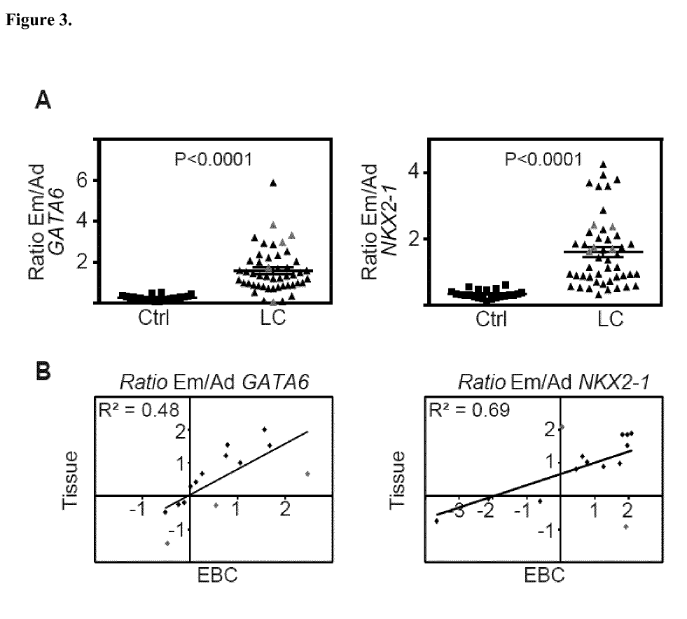

FIG. 2: Expression ratios of Em- by Ad-isoforms of GATA6 and NKX2-1 as a biomarker for lung cancer diagnosis. (A and B) Isoform specific expression of GATA6 (A) and NKX2-1 (B) was monitored by qRT-PCR after total RNA isolation from formalin fixed paraffin embedded (FFPE) lung tissue sections from control donors (Ctrl, n=34) or lung cancer (LC, n=63) patients. The Em/Ad ratio for both genes is plotted. Samples are normalized to TUB1A1 Each point represents one sample, black points represent adenocarcinoma, blue points represent squamous cell carcinoma, orange point represents adenosquamous carcinoma, red point represents large cell carcinoma, horizontal line in the middle represents the mean and the error bars represent the standard error mean (s.e.m). P values after one-way ANOVA. (C and D) High Em/Ad ratio is conserved among ethnic groups (C) and gender (D). CHB, Han Chinese in Beijing, Ctrl n=7 and LC n=32; CEU, Utah residents with ancestry from northern and western Europe, Ctrl n=19 and LC n=18; MXL, Mexican ancestry in Los Angeles, Ctrl n=8 and LC n=13; Male Ctrl n=8 and LC n=20; Female Ctrl n=4 and LC n=21. Data are represented as in A. (E) Expression of Em-isoform correlates with LC grade. Ratio of Em/Ad was monitored in lung tissue samples of control donor (Ctrl, n=7) cancer patients of Grade I (n=12), II (n=14) and III (n=5). Samples were staged according to the TNM Classification recommended by the International Union Against Cancer (UICC, 7th edition). Data are represented as in A.

FIG. 3: Detection of Em- and Ad-isoforms of GATA6 and NKX2-1 in exhaled breath condensate as non-invasive method for lung cancer diagnosis. (A) Isoform specific expression of GATA6 (left) and NKX2-1 (right) was monitored by qRT-PCR after total RNA isolation from EBCs from control donors (Ctrl, n=22) or lung cancer (LC, n=48) patients. The Em/Ad ratio for both genes is plotted. Samples are normalized to TUB1A1. Each point represents one sample, pink points represent samples of first diagnosis, horizontal line in the middle represents the mean and the error bars represent the standard error mean (s.e.m). P values after one-way ANOVA. (B) Correlation between the values obtained from lung tissue sample and EBC for each patient. The GATA6 (left) and NKX2-1 (right) Em/Ad ratio for both lung tissue (y-axis) and EBC (x-axis) samples were log 2 transformed and plotted. The linear regression was also plotted for both. Red dots, patients where the values from both sample types were significantly different.

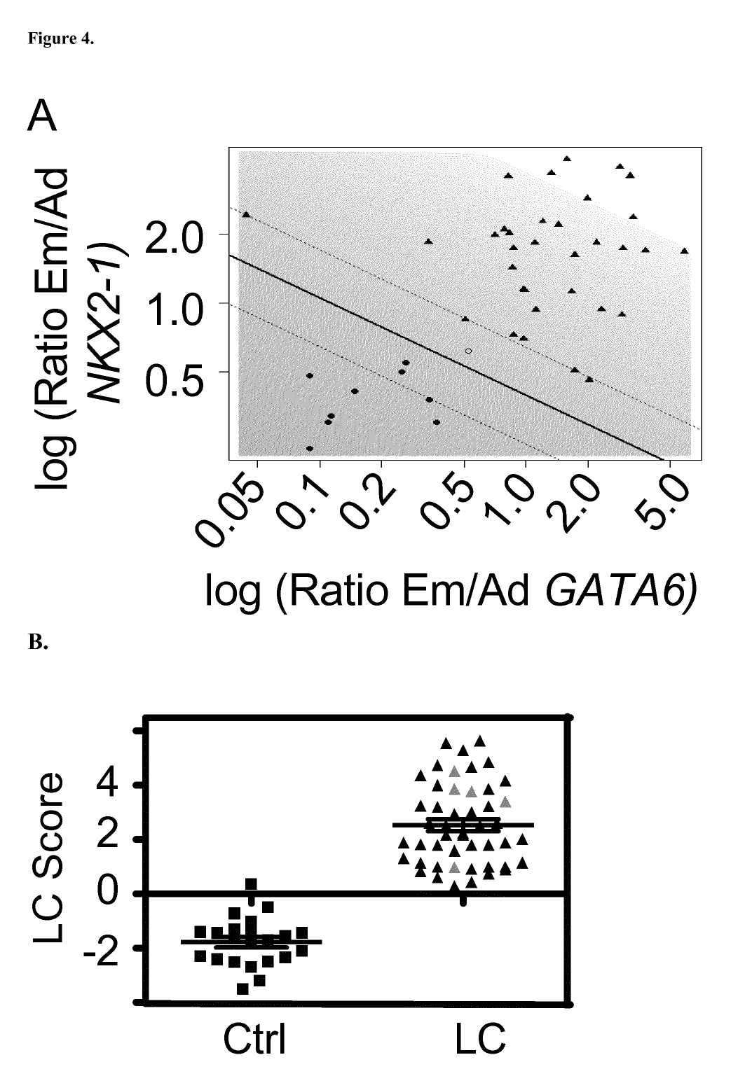

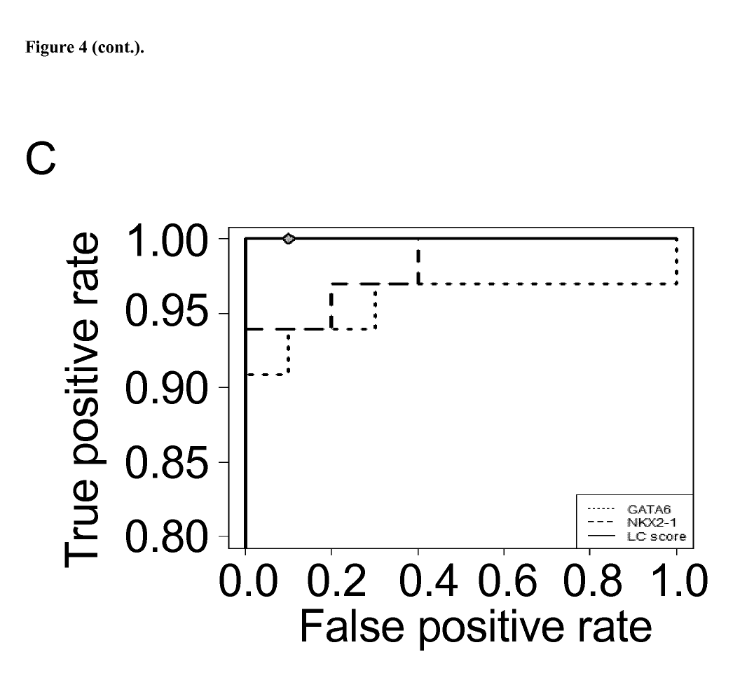

FIG. 4: Reliable diagnosis of lung cancer patients using a combination of GATA6 and NKX2-1. (A). The (log) Em/Ad ratio of GATA6 (x-axis) and NKX2-1 (y-axis) of control donors (filled and open circles) and lung cancer patients (triangles) are used to construct a linear SVM classifier, whose decision boundary is the solid line. The LC score is the distance to this boundary (dotted lines: points having LC score±1). A positive LC score indicates lung cancer (light grey shading), a negative LC score indicates a normal lung (dark grey shading). The only misclassified sample is a control sample indicated as an open circle. (B) LC score provides a clear separation of the Ctrl and LC samples. The log transformed LC score was plotted for each sample. Each point represents one sample, the horizontal line in the middle represents the mean and the error bars, standard error mean (s.e.m). The dotted line at 0 represents the decision boundary. (C) Discriminatory power of the Em/Ad ratios alone (dotted line: GATA6, dashed line: NKX2-1) and the LC score (solid line) assessed by an ROC curve. The diamond on the LC score ROC curve represents the “point of operation” (performance) of the SVM classifier³⁸.

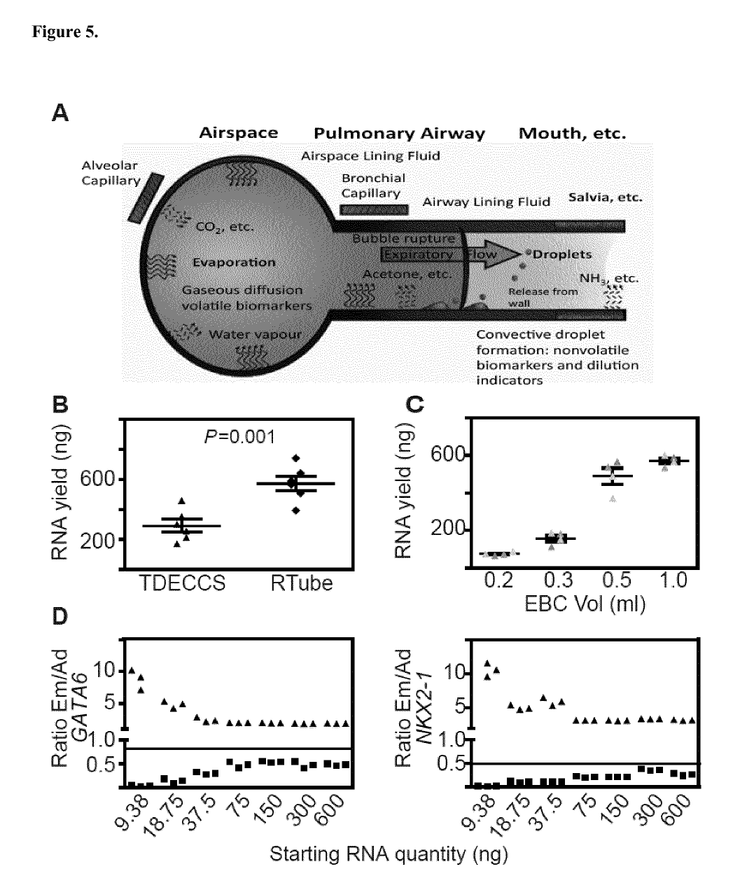

FIG. 5: Optimization of EBC based expression analysis for lung cancer diagnosis. (A) EBC as a promising source of biomarkers for lung diseases. Water vapor is rapidly diffused from the airway lining fluid (both bronchial and alveolar) into the expiratory flow. Droplet formation (nonvolatile biomarkers) takes place in the airway lining fluid, while respiratory gases (volatile biomarkers) are from both the airspaces and the airways. Modified from²⁰. (B) RTube is more suitable for RNA isolation as compared to TurboDECCS. Two main EBC collection devices were compared for the total RNA yield (y-axis, ng) obtained using the QIAGEN RNeasy Micro kit using 500 μl EBC as starting material. Data are represented as mean±s.e.m, n=6. P values after one-way ANOVA. (C) 500 μl of EBC is optimal for RNA isolation.

Total RNA isolation with the RNeasy Micro kit was compared using 200, 350, 500 and 1000 μl starting EBC volume. Data are represented as in B, n=4. (D) At least 75 ng of starting RNA is required for reliable diagnosis using EBC for isoform specific expression analysis. Different amounts of RNA (x-axis, ng) were used for cDNA synthesis by RT reaction and subsequently isoform specific expression analysis. The GATA6 (left) and NKX2-1 (right) Em/Ad ratio is plotted for both control (square) and lung cancer samples (triangle).

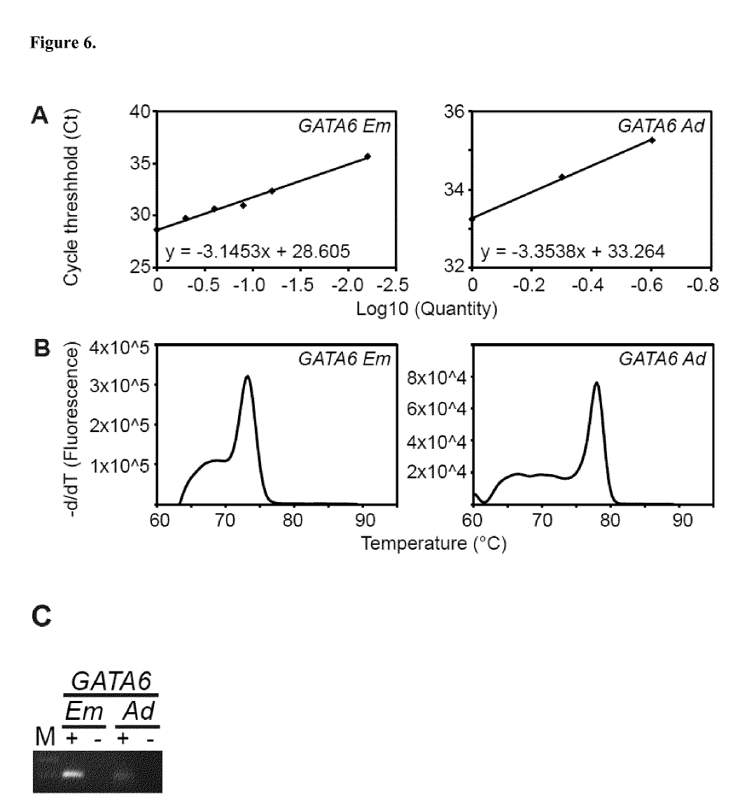

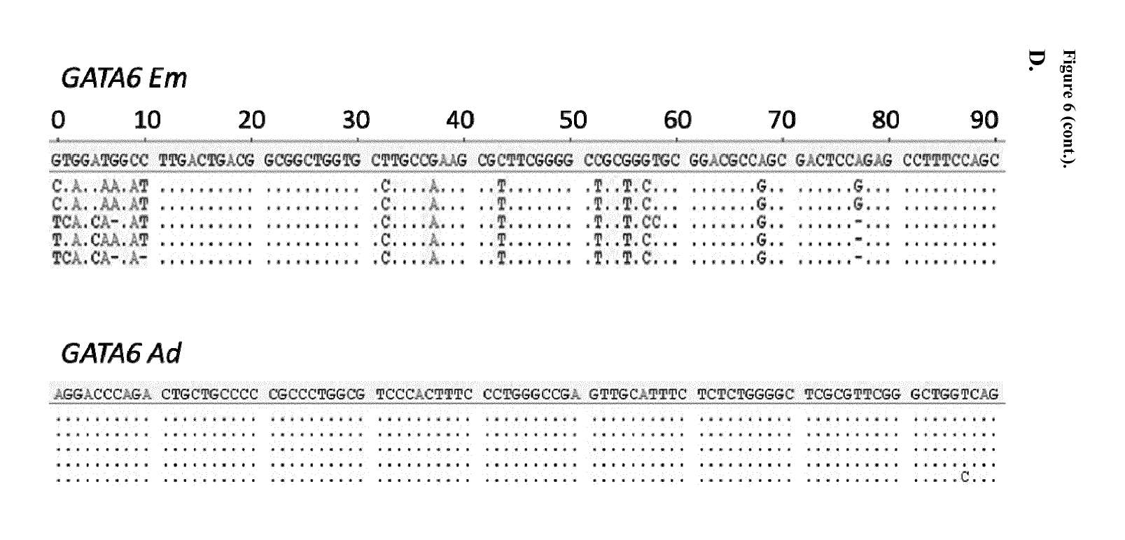

FIG. 6: Specific PCR amplification of both isoforms of GATA6. (A)

Amplification efficiency for each primer pair was calculated using serial dilutions of the cDNA template. Primer efficiency was assessed by plotting the cycle threshold values (Ct, y-axis) against the logarithm (base 10) of the fold dilution (log (Quantity), x-axis). Primer efficiency was calculated using the slope of the linear function. Data points represent mean Ct values of triplicates. (B) Dissociation curve analysis of the PCR products was performed by constantly monitoring the fluorescence with increasing temperatures from 60° C. to 95° C. Melt curves were generated by plotting the negative first derivative of the fluorescence (−d/dT (Fluorescence) 520 nm) versus temperature (degree Celsius, ° C.). (C) Specific PCR amplification was also demonstrated by agarose gel electrophoresis. PCR products after quantitative RT-PCR were analyzed by agarose gel electrophoresis. +, specific PCR reaction using EBC template; −, no RT control; M, 100 bp DNA ladder. (D) Sequencing of the PCR products of GATA6 Em and Ad demonstrates specific PCR amplification of both isoforms using EBC as template. Five clones for each primer pair (GATA6 Em and Ad) were sequenced and aligned to the reference sequence (top row, yellow highlighted). Sequence similarities are represented as dots.

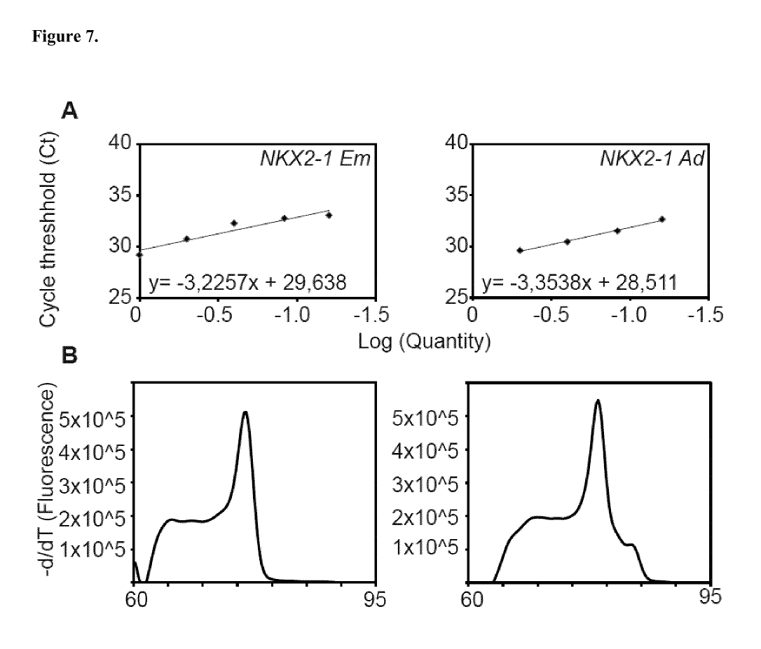

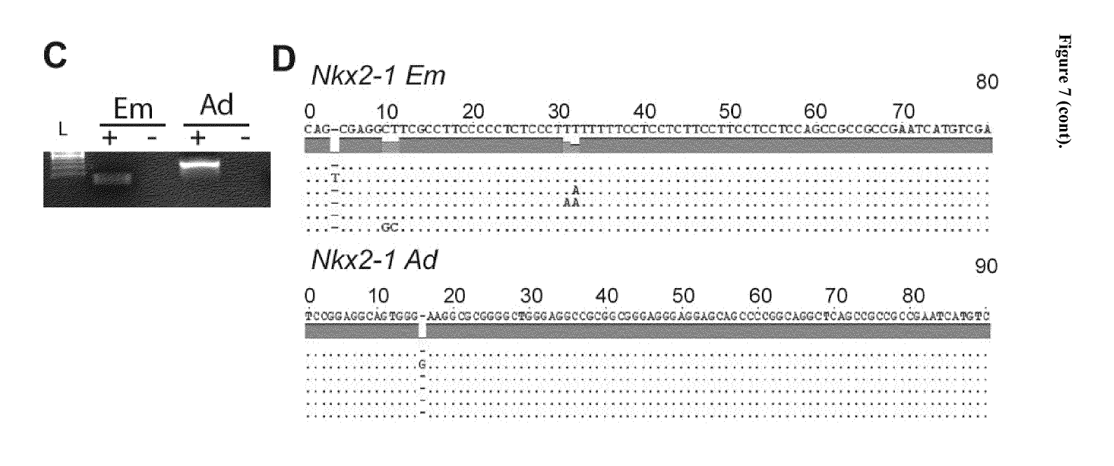

FIG. 7: Specific PCR amplification of both isoforms of NKX2-1. (A)

Amplification efficiency for each primer pair was calculated using serial dilutions of the cDNA template. Primer efficiency was assessed by plotting the cycle threshold values (Ct, y-axis) against the logarithm (base 10) of the fold dilution (log (Quantity), x-axis). Primer efficiency was calculated using the slope of the linear function. Data points represent mean Ct values of triplicates. (B) Dissociation curve analysis of the PCR products was performed by constantly monitoring the fluorescence with increasing temperatures from 60° C. to 95° C. Melt curves were generated by plotting the negative first derivative of the fluorescence (−d/dT (Fluorescence) 520 nm) versus temperature (degree Celsius, ° C.). (C) Specific PCR amplification was also demonstrated by agarose gel electrophoresis. PCR products after quantitative RT-PCR were analyzed by agarose gel electrophoresis. +, specific PCR reaction using EBC template; −, no RT control; M, 100 bp DNA ladder. (D) Sequencing of the PCR products of NKX2-1 Em and Ad demonstrates specific PCR amplification of both isoforms using EBC as template. Five clones for each primer pair (NKX2-1 Em and Ad) were sequenced and aligned to the reference sequence (top row, yellow highlighted). Sequence similarities are represented as dots.

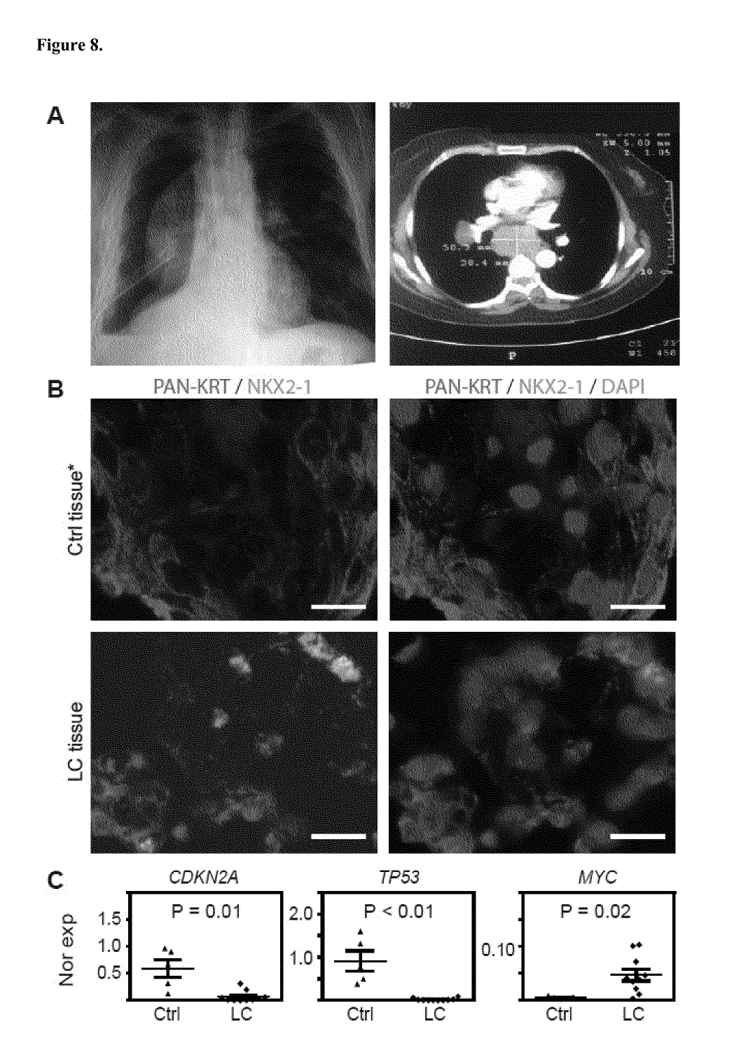

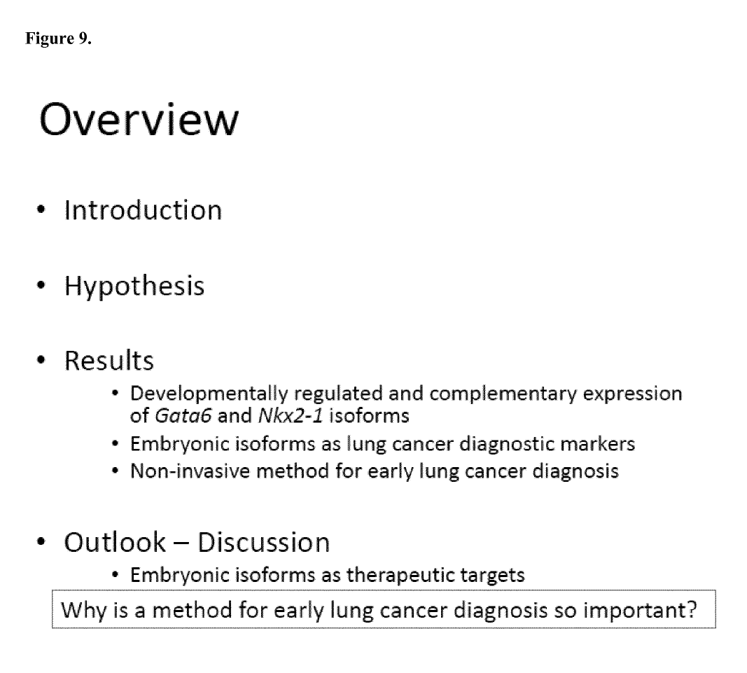

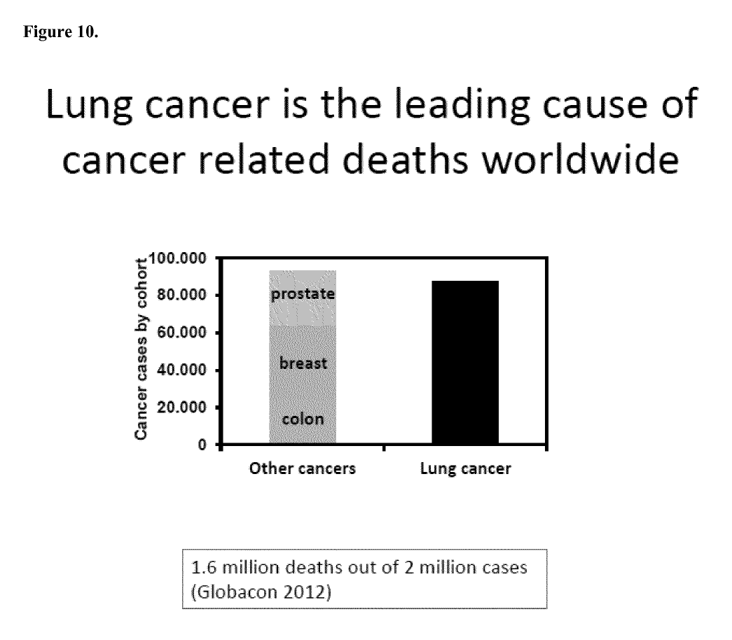

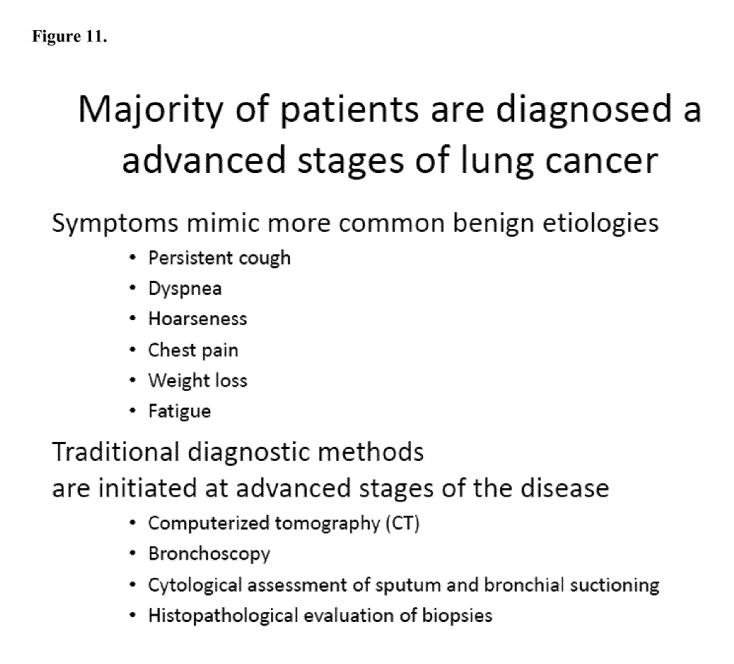

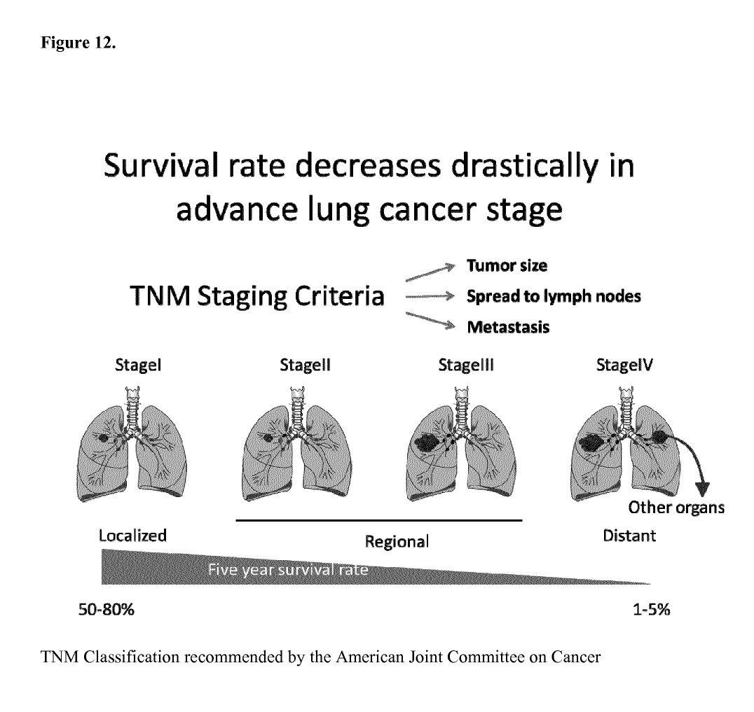

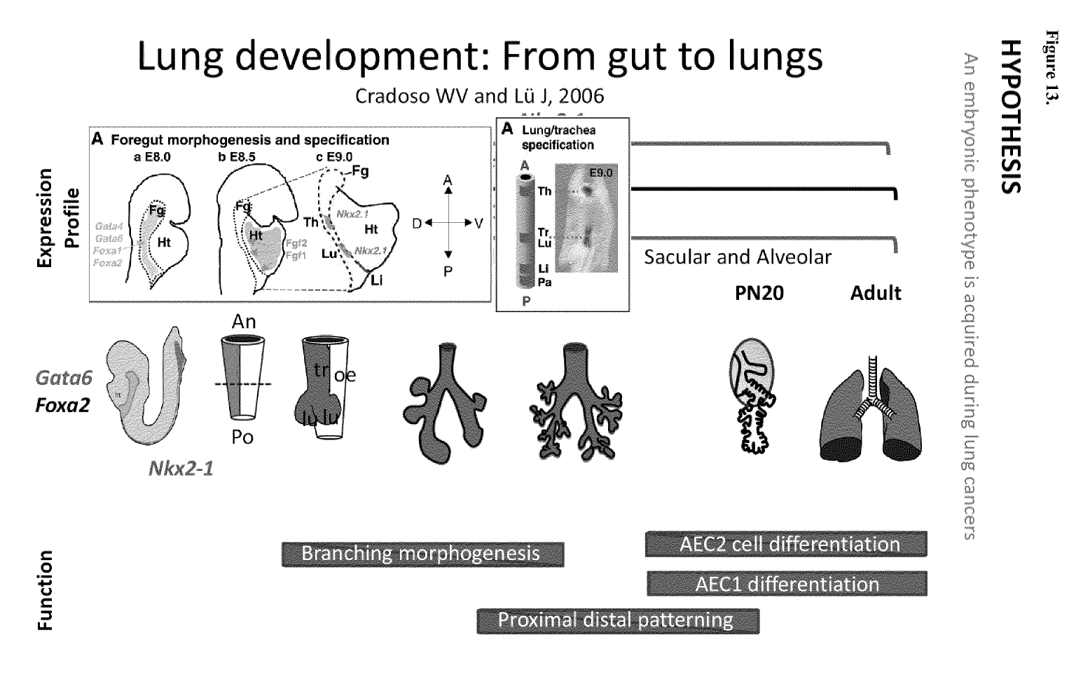

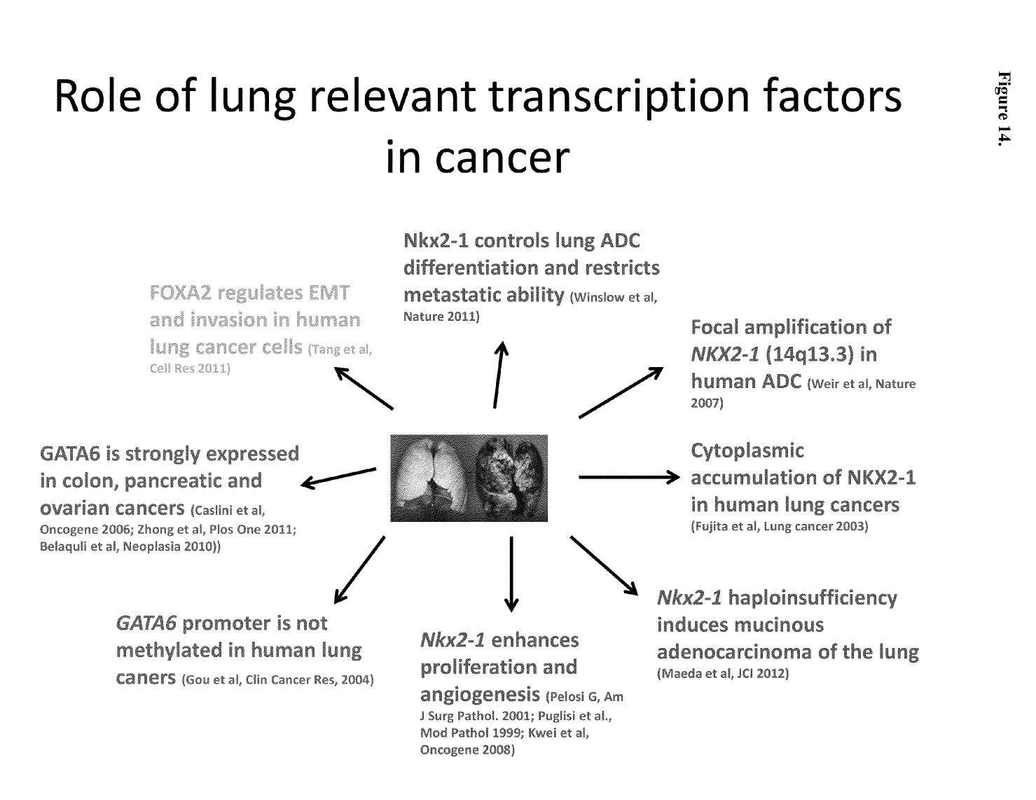

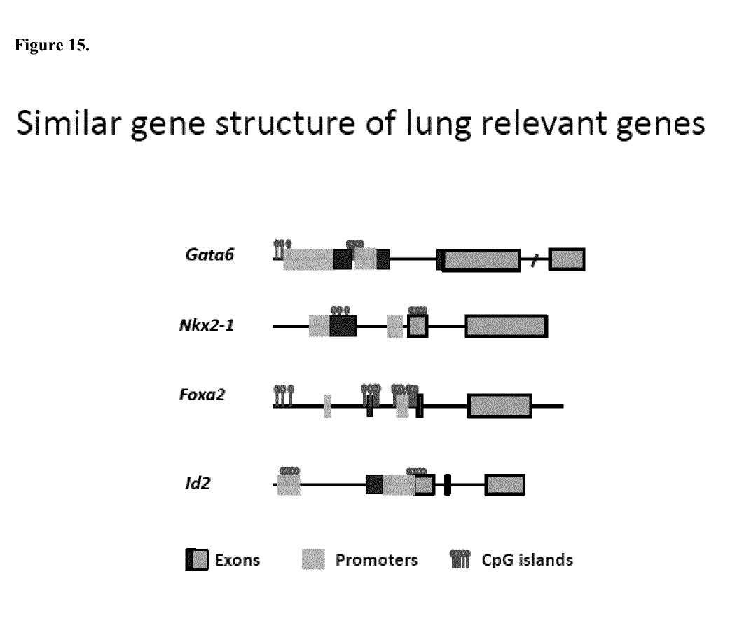

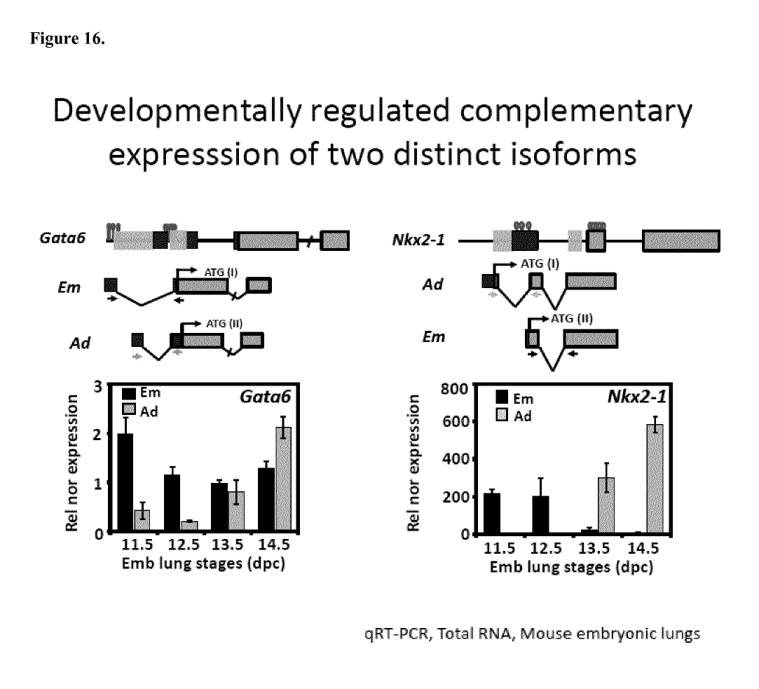

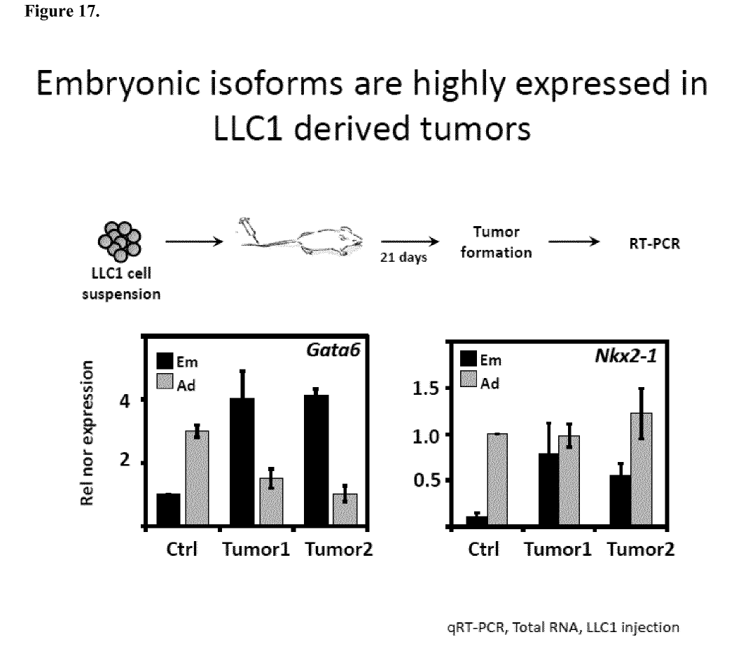

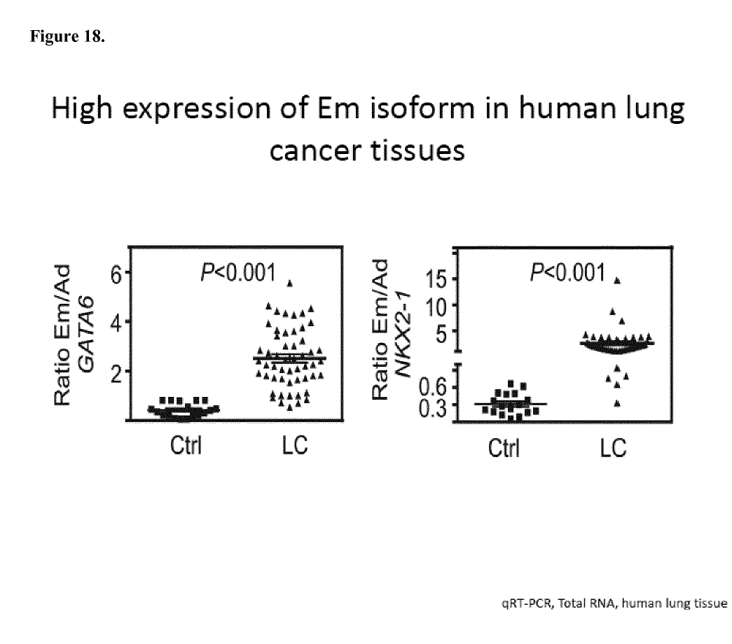

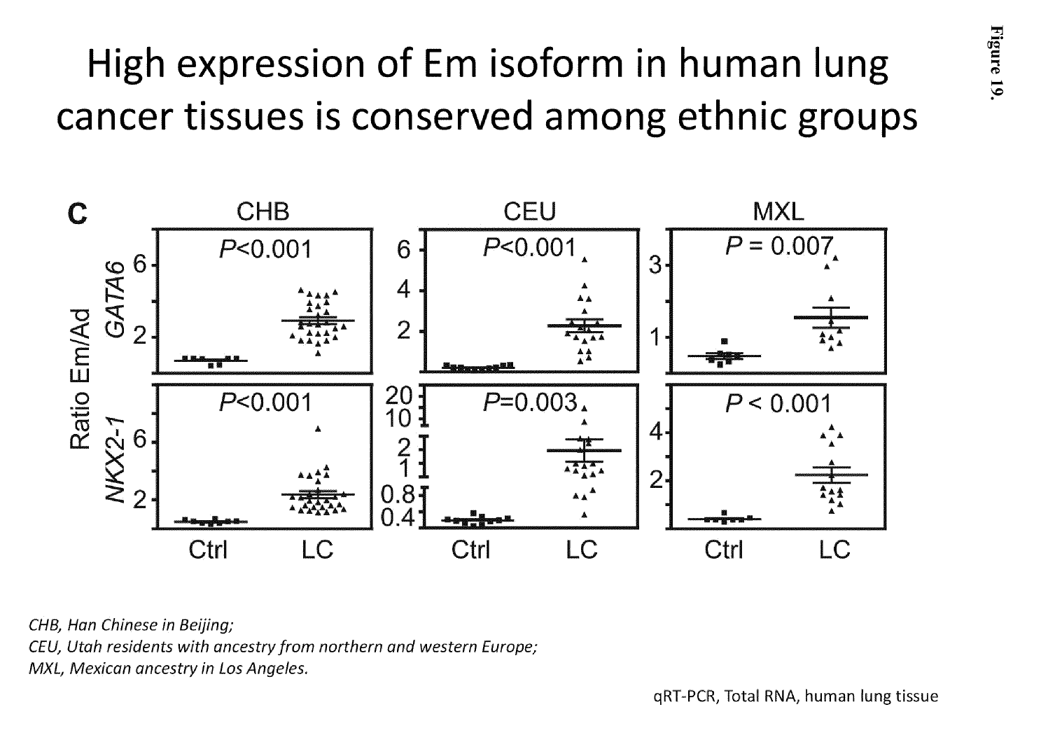

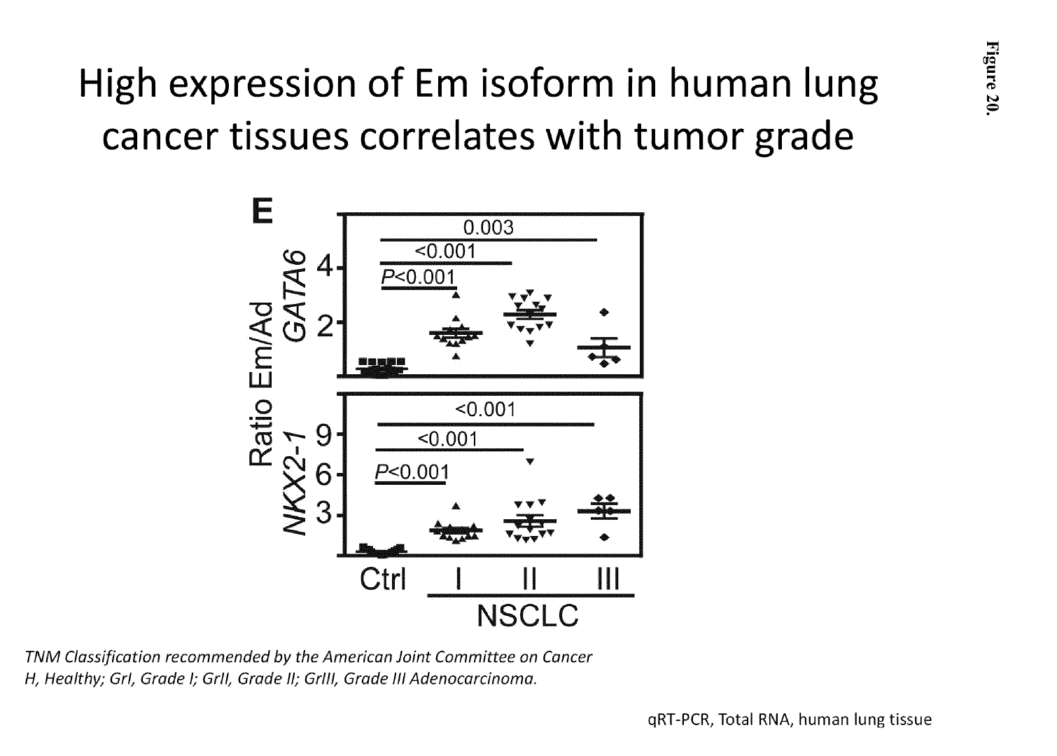

FIG. 8: EBC based lung cancer diagnosis correlates with classical methods. Representative pictures of (A) chest X-ray and (B) low-dose helical computed tomography (CT) scans from patients with lung cancer. (C) Immunohistochemistry analysis of adjacent normal (upper panel) and tumor tissue (lower panel) from a representative LC patient with the indicated antibodies. PAN-KRT, Pan Cytokeratin; NKX2-1, also known as TTF1, Thyroid transcription factor 1; DAPI, nucleus. Scale bar, 10 μm. (D) Expression analysis of known tumor suppressor and oncogenes in EBCs of healthy donors and LC patients. CDKNA2, also known as P16, cyclin-dependent kinase inhibitor 2A; TP53, tumor protein p53; MYC, v-myc avian myelocytomatosis viral oncogene homolog. Data are represented as in FIG. 2A.

### THE EXAMPLES ILLUSTRATE THE INVENTION

### Example 1: Detection of Embryonic Isoforms of GATA6 and NKX2-1 in Exhaled Breath Condensate as Non-Invasive Method for Lung Cancer Diagnosis

### Summary

BACKGROUND: Identification of reliable biomarkers and development of non-invasive detection methods for lung cancer are critical to improve prognosis of the disease.

METHODS: RNA isolation was performed from human lung tissue and exhaled breath condensates from control donors and lung cancer patients. The Em/Ad expression ratio of GATA6 and NKX2-1 was determined by qRT-PCR. Statistical analysis using R was performed to determine the separating line for the two groups of samples and to evaluate the efficiency of our diagnostic method.

RESULTS: We show that two different mRNAs are expressed from both GATA6 and NKX2-1. The expression of both transcripts from the same gene is complementary and differentially regulated during both embryonic lung development and lung cancer. One transcript is expressed during early embryonic lung development (Em-isoform), while the second transcript is expressed in later stages and in the adult lung (Ad-isoform). We detected an enrichment of the Em-isoform in lung cancer tissues, suggesting that the detection of these transcripts could be a powerful tool for early lung cancer diagnosis. The Em- to Ad-expression ratio of both GATA6 and NKX2-1 in RNA from exhaled breath condensates can be used as a non-invasive, specific and sensitive diagnostic tool. A SVM classifier was used to combine the Em/Ad ratios of GATA6 and NKX2-1 of each EBC sample to create a more powerful tool for the diagnosis of lung cancer.

CONCLUSIONS: The SVM calculates a simple linear score, LC score, that could be used as a clinical score for lung cancer detection.

### Glossary

Exhaled breath condensate: Exhaled breath condensate (EBC) is a non-invasive method of sampling the airways, allowing biomarkers of airway inflammation and oxidative stress to be measured. It is collected by cooling the exhaled breath to −20° C., resulting in condensation of the aerosol particles.

Gene expression analysis: Determination of the level of messenger RNA (mRNA) transcribed from specific genes. Different techniques can be used for this type of analysis, such as quantitative reverse transcriptase polymerase chain reaction (qRT-PCR), Northern Blot, arraybased expression analysis and, more recently, RNA sequencing. In the present manuscript we focus on qRT-PCR based expression analysis that consists of total RNA isolation, RT reaction for the synthesis of cDNA and qPCR amplification using gene specific primers.

Isoform: Different versions of mRNA from the same gene that arise by either alternative splicing or differential promoter usage.

Polymerase chain reaction: A laboratory technique used to amplify DNA sequences. Short, synthetic complementary DNA sequences called primers are used to selectively amplify the specific portion of the genome. The temperature of the sample is repeatedly raised and lowered to facilitate the copying of the target DNA sequence by a DNA-replication enzyme. Theoretically, the technique doubles the amount of target DNA molecule per cycle.

TNM staging criteria: The TNM system is one of the most widely used cancer staging systems.

It is based on the size and/or extent (reach) of the primary tumor (T), the amount of spread to nearby lymph nodes (N), and the presence of metastasis (M) or secondary tumors formed by the spread of cancer cells to other parts of the body. A number is added to each letter to indicate the size and/or extent of the primary tumor and the degree of cancer spread.

10-fold cross validation: A validation method in which the model is fitted on 90 percent of the samples and then the classification of the remaining 10 percent of the samples is predicted. The procedure is repeated 10 times such that each sample acts as a test sample once. The average error rate of all 10 parts is an estimate of the method's classification error.

### Introduction

We postulated that many of the mechanisms involved in embryonic development are recapitulated during LC initiation. To this end, two transcription factors that are key regulators of embryonic lung development, GATA6 (GATA Binding Factor 6) and NKX2-1 (NK2 homeobox 1, also known as Ttf-1, Thyroid transcription factor-1)⁷⁻¹⁰, and have been implicated in LC formation and metastasis¹¹⁻¹⁶ were analyzed. Here we show that two different mRNAs are expressed from each the GATA6 and the NKX2-1 gene. Furthermore, the expression of both transcripts from the same gene is complementary and differentially regulated during embryonic lung development as well as in LC. One transcript is expressed in early stages of embryonic lung development (Em-isoform), whereas the second transcript is expressed in later developmental stages and in the adult lung (Ad-isoform). We detected an enrichment of the Em-isoform in LC, even at early stages, making the detection of these embryonic specific transcripts a powerful tool for cancer diagnosis. Moreover, we demonstrate that isoform specific expression analysis of GATA6 and NKX2-1 in exhaled breath condensates (EBCs) can be used as a non-invasive, specific and sensitive method for both early LC diagnosis.

### Methods

### Study Population

The patients were studied according to protocols approved by the institutional review board and ethical committee of Regional Hospital of High Specialties of Oaxaca (HRAEO) which belongs to the Ministry of Health in Mexico (HRAEO—CIC-CEI 006/13), Union Hospital Hong Kong (EC003) and Medicine Faculty of the Justus Liebig University in Giessen, Germany (AZ.111/08-eurIPFreg). All cases were reviewed by an expert panel of pulmonologists and oncologists in the different cohorts according to the current diagnostic criteria for morphological features and immunophenotypes recommended by the International Union Against Cancer (UICC, 7^{th} edition).

LC tissue was obtained from 63 patients who had primary lung tumors in the last five years (Table 1). Control lung tissue was taken from macroscopically healthy adjacent regions of the lung of 15 patients. Control donor lung tissue was also obtained from 19 age-matched individuals, who have had no diagnosis or family history of LC.

EBCs were also collected from 48 LC patients that were currently undergoing diagnostic evaluation for LC (Table 1). EBC collection was performed prior to transbronchial biopsy. Further, control EBC was also collected from 22 age matched control individuals with no prior history of LC or any other lung diseases. All participants provided written informed consent.

### Cell Culture and Mouse Experiments

In this study we used human lung adenocarcinoma cell lines (A549; CCL-185 and A427; HTB-53) and a human bronchoalveolar carcinoma cell line (H322; CRL-5806). In addition, Mus musculus Lewis Lung cancer cell line (LLC1; CRL-1642) were used in a mouse model of experimental metastasis¹⁷, wherein 1 million LLC1 cells were injected into the tail vein of experimental mice (n=5) in 100 μl sterile phosphate buffer saline (PBS). Control mice (n=3) were injected with 100 μl sterile PBS.

Gene Expression Analysis by qRT-PCR

Total RNA was isolated from cell lines using the RNeasy Mini kit (Qiagen). Human lung tissue samples were obtained as formalin fixed paraffin embedded (FFPE) tissues, from which total RNA was isolated using the RecoverAll™ Total Nucleic Acid Isolation Kit for FFPE (Ambion).

Total RNA isolation from EBC was performed using 500 μl of sample with the RNeasy Micro Kit (Qiagen). Complementary DNA (cDNA) was synthetized using the High Capacity cDNA Reverse Transcription kit (Applied Biosystem) and quantitative real time PCR reactions were performed using SYBR® Green on the Step One plus Real-time PCR system (Applied Biosystems) using the primers specified in the Supplementary Table 2.

### Classifier Construction and LC Score

Log-transformed Em/Ad ratios of GATA6 and NKX2-1 were used as independent variables to predict LC. A linear kernel support vector machine (SVM)³⁹ was used to construct a linear classifier. SVM learning was done with the default parameters, without any adjustments. We preferred SVM to linear discriminant analysis (LDA), which might be the more obvious choice for low dimensional classification tasks, because the control and the LC samples did not show a Gaussian-like distribution, which is an underlying assumption of LDA. The SVM finds a robust separating line and the distance to this line is our decision score, which we call LC score. The LC score can be conveniently calculated as

$$LC Score = - 0.607 * {log}_{2} ( \frac{Em GAT A 6}{Ad GAT A 6} ) - 1.431 {log}_{2} ( \frac{Em NKX 2 - 1}{Ad NKX 2 - 1} ) - 1.916$$

or comprising a prefactor of (−1) for illustrative purposes of

$$LC Score = ( - 1 ) * ( - 0.607 * {log}_{2} ( \frac{Em GAT A 6}{Ad GAT A 6} ) - 1.431 {log}_{2} ( \frac{Em NKX 2 - 1}{Ad NKX 2 - 1} ) - 1.916 ) .$$

### Results

### Embryonic Isoforms of GATA6 and NKX2-1 are Highly Expressed in Human Lung Cancer Cell Lines and in a Mouse Model of Experimental Metastasis.

In silico analysis of GATA6 and NKX2-1 revealed a common gene structure (FIG. 1A, top). Two promoters were predicted in each of the genes, one 5′ of the first exon and the other one in the first intron. Further analysis showed that each of the predicted promoters was surrounded by CpG islands (greater than 200 bp, with more than 50% CG), suggesting that these might be epigenetically regulated, functional promoters. Indeed, expression analysis showed that each gene gave rise to two distinct transcripts (FIG. 1A, bottom) driven by different promoters. In silico analysis of the murine ortholog genes demonstrated a similar structure as in humans, which highlights that the identified gene structure was maintained during evolution and is conserved among species, reflecting its relevance. Expression analysis by qRT-PCR during mouse lung development revealed that the expression of both isoforms of the same gene was complementary and differentially regulated, with the Em-isoform being mainly expressed during early developmental stages, and the Ad-isoform being expressed at later stages and in the adult lung (data not shown). Interestingly, isoform specific expression analysis (FIG. 1B) in control donor lung tissue (Ctrl), human lung adenocarcinoma (A549, A427) and human bronchoalveolar carcinoma (H322) cell lines showed that in these cancer cell lines the expression of the Em isoforms of GATA6 and NKX2-1 was always higher than the expression of the Ad-isoforms. In control human lung tissue, we observed the opposite results, in which the Ad-isoforms were expressed at higher levels than the Em-isoforms. Moreover, in a mouse model of experimental metastasis (FIG. 1C)¹⁷, in which LLC1 cells were injected into the tail vein to induce tumor formation in the mouse lung 21 days later, we detected elevated expression of the Em-isoforms of Gata6 and Nkx2-1 in the tumors when compared to healthy lung tissue (Ctrl). Summarizing, our results suggest that the Em-isoforms of GATA6 and NKX2-1 are relevant during LC formation.

### Expression Ratios of Em- by Ad-Isoforms of GATA6 and NKX2-1 as a Biomarker for Lung Cancer Diagnosis.

To confirm that a similar increase in the expression levels of the Em-isoforms of GATA6 and NKX2-1 occurs in LC patients, we analyzed human lung tissues from control donors and LC patients (FIG. 2A-B). The pathological diagnosis of the 63 lung tissue samples was considered as the standard against which the gene expression based molecular diagnosis was compared (Table 1). Isoform specific expression analysis based on qRT-PCR showed that the Em-isoforms of GATA6 and NKX2-1 were enriched in LC tissues as compared to control donor tissue, consistent with our previous results (FIGS. 1B-C). In order to facilitate comparability, we decided to use the expression of the Ad-isoform as an internal control and calculated the Em to Ad expression ratio (Em/Ad) for each sample to minimize the effect of individual variations among the different LC specimens. In control lung tissue, Em/Ad was 0.624±0.065 (n=34) for GATA6 and 0.475±0.044 (n=34) for NKX2-1. Interestingly, Em/Ad increased in the LC tissue to 2.63±0.194 (n=63, P<0.001) for GATA6 and to 2.075±0.22 (n=63; P<0.001) for NKX2-1, supporting that an increased Em/Ad expression ratio of GATA6 and NKX2-1 could be used as marker for LC diagnosis. The diagnostic accuracy of the Em/Ad expression ratios of GATA6 and NKX2-1 was maintained after sample grouping by ethnicity (FIG. 2C) or by gender (FIG. 2D). Furthermore, sample grouping based on TNM classification recommended by the International Union Against Cancer (UICC, 7th edition) (FIG. 2E) revealed that the Em/Ad expression ratios of GATA6 and NKX2-1 increased progressively with advancing stages of LC from Grade I (2.395±0.257; P<0.001 for GATA6 and 1.878±0.129; P<0.001 for NKX2-1) through Grade II (3.436±0.243; P<0.001 for GATA6 and 2.589±0.257; P=0.002 for NKX2-1) till Grade III (1.838±0.598; P=0.003 for GATA6 and 3.787±0.392; P<0.001 for NKX2-1).

### Detection of Em- and Ad-Isoforms of GATA6 and NKX2-1 in Exhaled Breath Condensate as Non-Invasive Method for Lung Cancer Diagnosis.

EBC is a promising source of biomarkers for lung diseases since the condensed droplets contain a mixture of nonvolatile biomarkers such as adenosine, prostaglandins, leukotriene, cytokines, etc. and water soluble volatile biomarkers such as nitrogen oxides^{18-27.} We optimized different steps and parameters to establish a reliable protocol for qRT-PCR based expression analysis in EBCs (FIG. 5A-D). We also demonstrated the specificity of the different qRTPCR products detected in the EBCs (FIGS. 6A-D and 7A-D). Using the optimized conditions, we performed an isoform specific expression analysis of GATA6 and NKX2-1 in EBCs from control donors and LC patients (FIG. 3A). In control donor EBCs, the Em/Ad ratio was 0.255±0.02 (n=22) for GATA6 and 0.336±0.02 (n=22) for NKX2-1. In accordance with our previous results using lung tissues, the Em/Ad ratio increased in the EBCs of LC patients to 1.59±0.15 (n=48, P<0.0001) for GATA6 and to 1.625±0.15 (n=48; P<0.0001) for NKX2-1. Remarkably, we were able to anticipate the diagnosis of six LC patients (first diagnosis represented as pink points in the plots) measured in a blinded manner. Hence, our results support the concept that an increased Em/Ad expression ratio of GATA6 and NKX2-1 in the EBCs could be used as non-invasive technique for LC diagnosis.

To further validate our findings, EBC based expression analysis was directly compared with LC tissues from the same patient (FIG. 3B). The GATA6 (left) and NKX2-1 (right) Em/Ad ratios obtained from both types of samples of the same individuals were comparable and demonstrated a strong positive correlation. Moreover, we compared the classical methods for LC diagnosis directly with EBC based expression analysis (FIG. 8). The pathological and molecular diagnosis correlated with the increased Em/Ad of GATA6 and NKX2-1 in all cases that we tested.

### Reliable Diagnosis of Lung Cancer Patients Using a Combination of GATA6 and NKX2-1.

While the single GATA6 or NKX2-1 isoform ratios predicted LC fairly well (FIG. 3E), we combined the two ratios of each EBC sample to create a substantially improved and more powerful tool for the diagnosis of LC. A support vector machine (SVM) classifier achieved 93% accuracy in a 10-fold cross-validation, at 100% sensitivity (FIG. 4A). Further, the SVM calculates a simple linear score, which we call LC score, that can be used as a clinical score for LC detection. A sample with an LC score greater than zero is classified as a LC patient while samples with LC score less than zero are classified as control (FIG. 4B). The precision of our classification increases with the absolute value of the LC score, in the sense that no misclassifications have been made (yet) for LC scores with an absolute value larger than 1. The individual GATA6 and NKX2-1 isoform ratios, the LC score, and the SVM classification is given in Supplementary Table 3. Furthermore, receiver operating characteristic (ROC) curve analysis confirmed the superiority of the SVM classifier over the single isoforms ratios (FIG. 4C).

### Discussion

Early lung cancer diagnosis is crucial to improve patient prognosis and reduce the extremely high case-fatality-rate (95%)²⁸. Our work demonstrated that RNA isolated from EBC can be used for qRT-PCR based isoform specific expression analysis of GATA6 and NKX2-1 to determine the Em- by Ad-expression ratio as a non-invasive, specific and sensitive method for early LC diagnosis. We have analyzed 97 human lung tissue samples and 70 EBCs from three cohorts located in different continents and detected increased Em/Ad of GATA6 and NKX2-1 in NSCLC samples independent of the ethnic group, gender and NSCLC subtype. When compared to standard expression analysis, the use of isoform ratios incorporate an additional normalization step to our diagnosis method that makes it robust and reproducible by reducing variability coming from both biological and/or technical parameters.

Although the single Em/Ad ratios of GATA6 or NKX2-1 were sufficient to detect LC (FIG. 3E), the LC score, which combines the two Em/Ad ratios of each EBC, constitutes a substantially improved tool for the diagnosis of LC, as shown by the ROC analysis (FIG. 4C). Our calculation method based on a SVM classifier achieved 93% accuracy in a 10-fold crossvalidation, at 100% sensitivity (FIG. 4A). Thus, the method proposed by us may find application in the screening of high risk groups, which includes current and former smokers, individuals exposed to environmental smoke, cooking fumes, indoor smoky coal emissions, asbestos, some metals (e.g. nickel, arsenic and cadmium), radon and ionizing radiation²⁹⁻³¹.

Currently, CT and CXR are used to screen such high risk groups. CT imaging has been shown to be considerably superior to CXR in the identification of small pulmonary nodules³². However, despite the success of CT imaging for early LC diagnosis, it suffers from serious limitations, including a high detection rate of benign non calcified nodules (>90% of participants) resulting in follow-up CT scans, biopsies and frequently unnecessary resection of the benign non calcified nodules³³. Routine implementation of EBC based molecular diagnosis may improve and complement the success of CT and CXR for early LC diagnosis, and especially help to distinguish between false and true positives.

Microarray based analysis of LC samples not only led to identification of gene expression profiles that are associated with NSCLC subtypes^{34,35}, but also accurately predicted the clinical outcome^{36,37}. Although the method proposed here did not discriminate between different NSCLC subtypes, it may be superior to previous approaches of molecular and clinical LC diagnosis due to its higher sensitivity and accuracy, straightforward and fast protocol, noninvasiveness and relative low price. However, a combination of the method proposed here with the existing clinical and molecular methods of LC diagnosis will help to safely settle a LC diagnosis at an earlier, hence curable, stage of the disease. The method of LC diagnosis proposed here could be further refined to discriminate between different NSCLC subtypes by incorporating EBC based expression analysis of known markers of the different subtypes. Furthermore, it might be combined with other markers for the detection of hyper-proliferative non-cancer related diseases as idiopathic pulmonary fibrosis (IPF) or chronic obstructive pulmonary disease (COPD). Interestingly, the current method could be extended to cancer detection in other organs utilizing the expression ratio of developmentally regulated transcript isoforms of the corresponding members of the GATA and/or NKX families of transcription factors in the respective tissue. Lastly, it could be used to monitor the response of a patient to specific treatments in order to fine-tune the therapy to improve the prognosis.

Supplement TABLE 2
Primer sequences used for the analysis of GATA6 and NKX2-1.

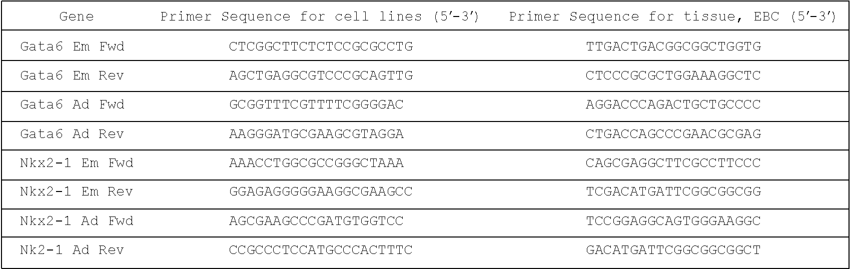

The following alternative Supplement Table 3 shows also values for the individual ratios of GATA6, NKX2-1 and the LC score, wherein the LC score has been calculated using a a prefactor of (−1) for illustrative purposes.

### Supplementary Results

FIG. 5: Optimization of EBC Based Expression Analysis for Lung Cancer Diagnosis.

EBC consists of three main components (FIG. 5A): distilled water condensed from the gas phase (>99%), droplets aerosolized from the airway lining fluid and water soluble respiratory gases (the last two make the remaining 1%)^{18,19} EBC is a promising source of biomarkers for lung diseases since the condensed droplets contain a mixture of both nonvolatile biomarkers such as adenosine, prostaglandins, leukotriene, cytokines, etc. and water soluble volatile biomarkers such as nitrogen oxides that diffuse from both airspace and airway lining fluid²⁰⁻²⁷. EBCs are typically collected through cooling devices. Here, we tested two of the most commonly used devices for EBC collection for their suitability for subsequent RNA extraction (FIG. 5B). Using the same conditions for EBC collection and RNA extraction, the RTube showed a yield of 573±48 ng RNA per 500 μl EBC (n=6), whereas the TurboDECCS showed a lower yield of 292±42 ng RNA per 500 μl EBC (n=6; P=0.001). Thus, we continued collecting the samples with the RTube and tested different EBC volumes to determine the best for RNA extraction (FIG. 5C). The RNA yield increased with the EBC volume following a sigmoid curve that reached a plateau at 573±48 ng RNA using 500 μl EBC. RNA yield did not improve further when more than 500 μl of EBC volume was used as starting material. In addition, conditions for cDNA synthesis by reverse transcription and qPCR amplification were optimized using 500 μl EBC collected with the RTube (data not shown). Further, serial dilution of the RNA template was used to determine the minimal material required for reliable diagnosis of cancer based on the Em/Ad ratio of GATA6 and NKX2-1 (FIG. 5D). The expression ratio remained stable for both control donor as well as LC EBC samples until 75 ng of RNA starting material. Decreasing the starting material below 75 ng resulted in suboptimal detection of the Em-isoform in the control and the Ad-isoform in the LC group which led to distorted ratios. Using the optimized conditions, we performed isoform specific expression analysis of GATA6 and NKX2-1 in EBCs.

### FIG. 6: Specific PCR Amplification of Both Isoforms of GATA6.

### FIG. 7: Specific PCR Amplification of Both Isoforms of NKX2-1.

The specificity of the different qRT-PCR products detected in the EBCs (FIGS. 7A-D and 8A-D) was demonstrated by dissociation curve analysis, electrophoretic gel analysis and sequencing of the different qRT-PCR products.

FIG. 8: EBC Based Lung Cancer Diagnosis Correlates with Classical Methods.

The classical methods for lung cancer diagnosis were directly compared with EBC based expression analysis. Pulmonary nodules were clearly identified by CXR (Supplementary FIG. 8A left) and low-dose helical CT (right) in the patients with elevated Em/Ad of GATA6 and NKX2-1. Furthermore, immunostaining on sections of biopsies from the same patients (FIG. 8B) using antibodies specific for the epithelial maker KRT (pan-cytokeratin) and NKX2-1 demonstrated that the nodules were primary adenocarcinomas of the lung. Lastly, to determine that markers that are used for the molecular diagnosis of cancer can be detected in EBC, we analyzed the expression of the tumor suppressor genes CDKN2A (also known as P16 or INK4A) and TP53 and the oncogene MYC in EBCs from control donors and lung cancer patients (FIG. 8C). In control donors, expression level of CDKNA2 was 0.6±0.36 (n=5) and it decreased to 0.068±0.09 (n=10; P=0.01) in lung cancer patients. Similarly, TP53 expression in control donors was 0.908±0.52 (n=5) and it decreased to 0.021±0.03 (n=10; P<0.01) in lung cancer patients. Consistently, the expression of MYC increased in lung cancer patients from 0.004±0.002 (n=5) to 0.046±0.034 (n=10; P=0.02). The pathological and molecular diagnosis correlated with the increased Em/Ad of GATA6 and NKX2-1 in all of the 10 cases from which we obtained the EBCs.

### Supplementary Methods

### Study Population:

Samples were collected in three different cohorts located in different continents (America, Asia and Europe), allowing us to investigate ethnic differences. Inclusion criteria for the present study were primary lung tumor samples including lung adenocarcinoma (Grades 1, 2, 3), lung squamous cell carcinoma (Grades 1, 2, 3), large cell carcinoma and adenosquamous carcinoma (Table 1). All tumors were graded according to the Bloom-Richardson and the TNM grading system recommended by the International Union Against Cancer (UICC, 7th edition). Secondary lung tumors and lung cancer samples older than 5 years were excluded.

In accordance with the general prevalence, the majority of the samples here represented adenocarcinoma (73.0% and 54.1% for lung cancer tissue and EBC, respectively), followed by squamous cell carcinoma (14.2% and 20.8% for lung cancer tissue and EBC, respectively) (Table 1). Correlating with the disease incidence, the majority of the patients were in the age group of 50-70 years and both male and female patients were equally represented (Supplementary Table 1). Further, the majority of the patients were in the early stage of the disease (Stage I-II) and only a very small minority (6% and 8% for tissues and EBC respectively) had a recurrent disease (Supplementary Table 1).

### Exhaled Breath Condensate Collection

EBC collection was performed using the RTube (Respiratory Research) as described online (http://www.respiratoryresearch.com/products-rtube-how.htm) with some modifications. As a precaution to avoid contaminants from the mouth, donors were asked to refrain from eating, drinking (except water) and smoking up to 3 hours before EBC collection and were asked to rinse their mouth with fresh water just prior to collection. All donors used a nose clamp to avoid nasal contaminants and breathing was only through the mouthpiece. EBCs were collected for 10 min for each donor and immediately stored at −80° C. in 500 μl aliquots. All steps during the collection and processing of EBCs were performed under RNase-free conditions, which is critical to ensure the integrity and high quality of the samples.

### Cell Culture and Mouse Experiments

Cell lines were cultured in medium and conditions recommended by the American Type Culture Collection (ATCC). Cells were used for the preparation of RNA (QIAGEN RNeasy plus mini kit) and protein extracts.

Five to 6 weeks old C57BL6 mice were used throughout this study. Animals were housed under controlled temperature and lighting [12/12-hour light/dark cycle], fed with commercial animal feed and water ad libitum. For the mouse model of experimental metastasis, LLC1 cell suspension of 1 million cells/100 μl was prepared in sterile phosphate buffer saline (PBS). Control mice (n=3) were injected with 100 μl PBS whereas experimental mice (n=5) with 100 μl of cell suspension into the tail vein of each mouse. The development of tumors was monitored 21 days post injection. Lung tissue was harvested from each mouse separately for RNA isolation and isoform specific expression analysis.

Mouse work was performed in compliance with the German Law for Welfare of Laboratory Animals. The permission to perform the experiments presented in this study was obtained from the Regional Council (Regierungspräsidium in Darmstadt, Germany). The numbers of the permissions are V54-19c20/15-B2/345; IVMr46-53r30.03.MPP04.12.02 and IVMr46-53r30.03.MPP06.12.01. Animals were killed for scientific purposes according to the law mentioned above which comply with national and international regulations.

### Statistical Analysis

Cell line and mouse experiments were performed three times. Statistical analyses were performed using Excel Solver. Samples were analyzed at least in triplicates. The data are represented as mean±Standard Error (mean±s.e.m). For human samples, each point on the graph represents an individual sample while the horizontal line represents the median±Standard Error (median±s.e.m.). One-way analysis of variance (ANOVA) was used to determine the levels of difference between the groups and P values for significance.

Gene Expression Analysis by qRT-PCR

Total RNA was isolated from cell lines using the RNeasy Mini kit (Qiagen. Human lung tissue samples were obtained as formalin fixed paraffin embedded (FFPE) tissues and 8 sections of 10 μm thickness were used for total RNA isolation using the RecoverAll™ Total Nucleic Acid Isolation Kit for FFPE (Ambion). Total RNA isolation from EBC was performed using 500 μl of sample and the RNeasy Micro Kit (Qiagen). Complementary DNA (cDNA) was synthetized using the High Capacity cDNA Reverse Transcription kit (Applied Biosystem) and 0.5-0.7m (EBC) or 1 μg (cell lines, mice and human lung cancer tissue) total RNA. Quantitative real time PCR reactions were performed using SYBR® Green on the Step One plus Real-time PCR system (Applied Biosystems) using the primers specified in the Supplementary Table 2. Briefly, 1× concentration of the SYBR green master mix, 250 nM each forward and reverse primer and 3.5 μl (EBC) or 1 μl (cell lines, mice and human lung cancer tissue) from a 6 fold diluted RT reaction were used for the gene specific qPCR reaction. The PCR results were normalized with respect to the housekeeping gene alpha 1a Tubulin (TUBA1A).

### Example 2: Further Validation of the Detection of Embryonic Isoforms of GATA6 and NKX2-1 in Exhaled Breath Condensate as Non-Invasive Method for Lung Cancer Diagnosis

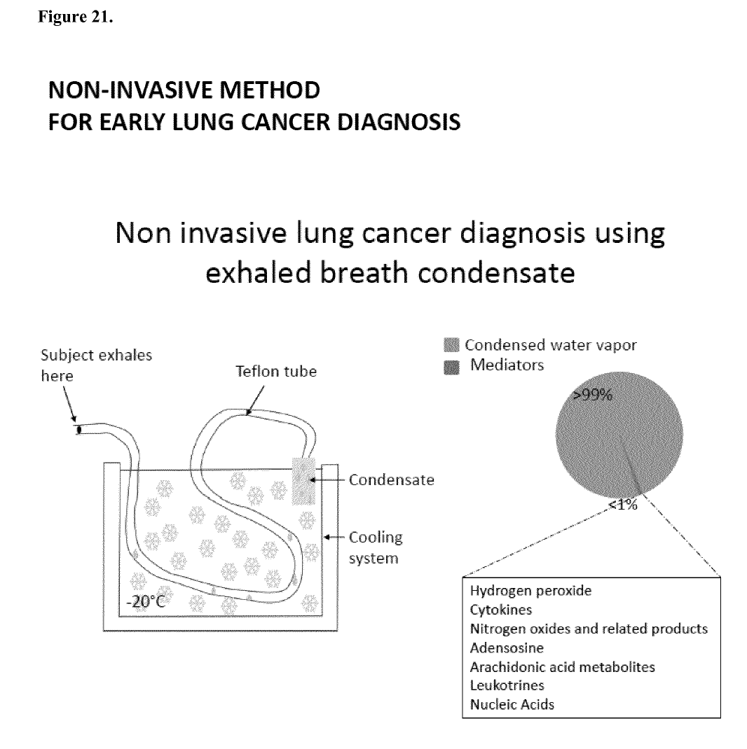

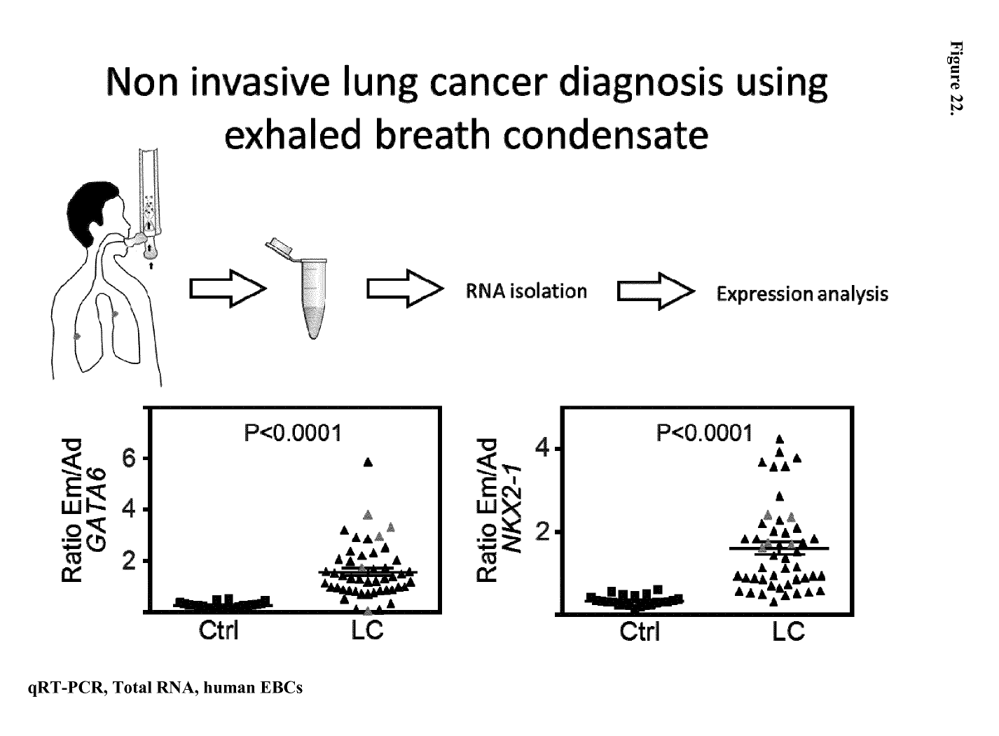

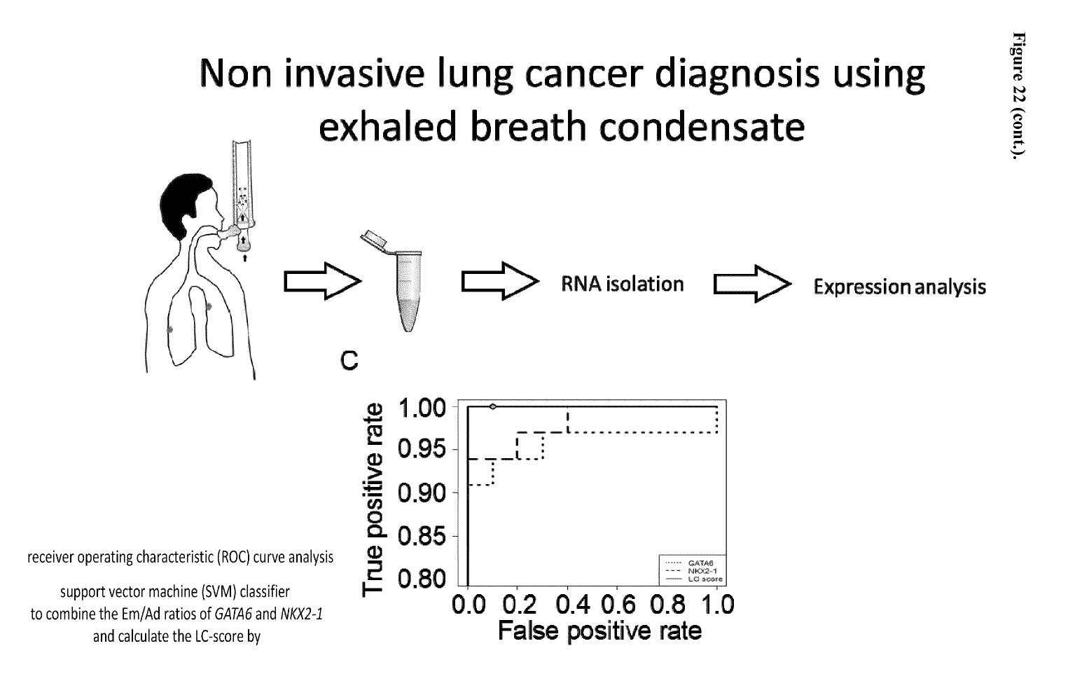

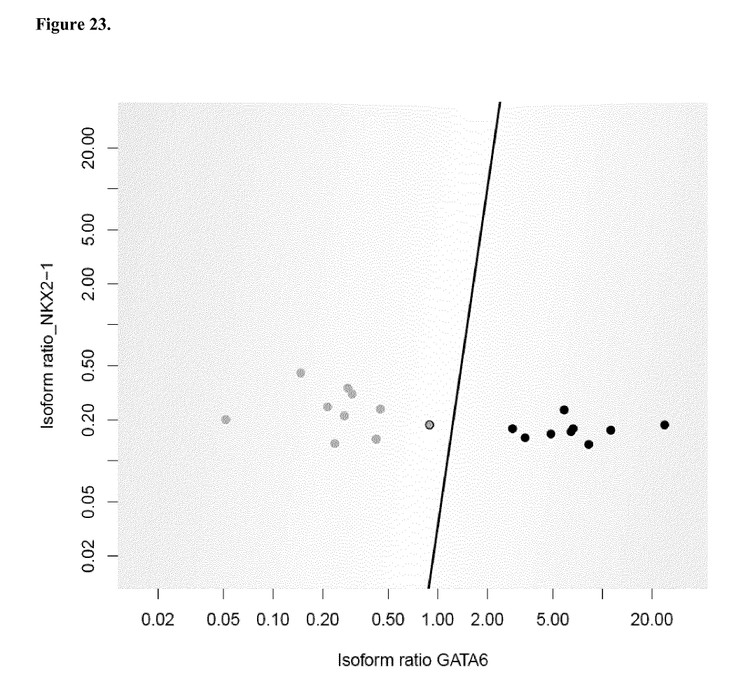

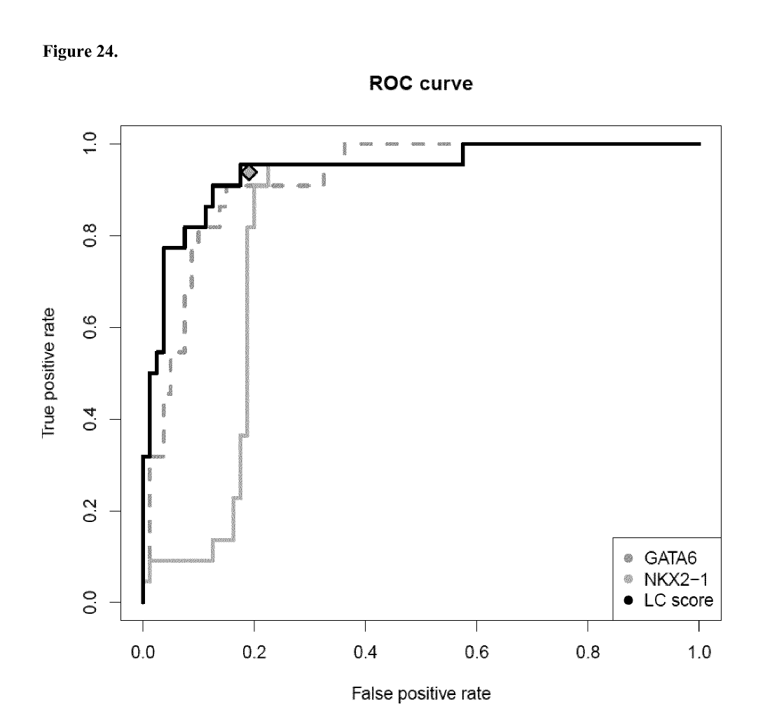

Further validation of the LC score classifier was performed on an independent set of samples (EBCs) consisting of 22 previously unseen samples (10 controls and 12 LC patient EBCs, FIG. 23). These EBCs were collected mimicking conditions of clinical use, e.g. they were collected in different centers by different operators according to optimized SOP. The protocol and algorithm were followed exactly as described in Example 1 to compute the LC Score. Performance assessment of the LC score classifier by applying it to this independently collected set of EBCs confirmed its high performance by achieving an accuracy of 91%, sensitivity of 77%, and a specificity of 95%. Receiver operating characteristic (ROC) curve analysis based on all EBCs together (training and validation FIG. 24) showed an area under the curve (AUC) of 0.8153409 for NKX2-1, 0.9204545 for GATA6 and 0.9397727 for the LC score.

### FIG. 23:

The log 2-transformed Em/Ad ratio of GATA6 (x-axis) and NKX2-1 (y-axis) of controls (light grey circles) and LC patients (black circles) for the new validation set were plotted. The solid line represents the decision boundary determined by a linear support vector machine (SVM) classifier combining the Em/Ad ratios of GATA6 and NKX2-1 of each sample. Filled circle, sample classified correctly; empty circle, sample classified wrong. LC score is the distance to the boundary.

### FIG. 24:

Discriminatory power of the Em/Ad ratios of GATA6 (grey line), NKX2-1 (grey dashed line) and the improved LC score (black line) assessed by receiver operating characteristic (ROC) curve analysis based on both sets of EBCs together (training and validation). The orange diamond represents the “point of operation” (performance) of the SVM classifier.

The present invention refers to the following nucleotide and amino acid sequences:

The sequences provided herein are available in the NCBI database and can be retrieved from www.ncbi.nlm.nih.gov/sites/entrez?db=gene; Theses sequences also relate to annotated and modified sequences. The present invention also provides techniques and methods wherein homologous sequences, and variants of the concise sequences provided herein are used. Preferably, such “variants” are genetic variants.

The following exemplary sequences relate to additional marker(s) that can be used in accordance with the present invention for classifying cancer, for example, for classifying lung cancer into subtypes of lung cancer.

The following markers are upregulated in adenocarcinoma:

SEQ ID No. 65:
Nucleotide sequence encoding Homo sapiens Surfactant protein A:
PMID 11707590

| | |
|---|---|
| gene symbol | Alias and additional info |
| SFTPA1 | Surfactant protein A |
| Accession number | Transcript variant |
| NM_001093770.2 | surfactant protein A1 (SFTPA1), |
| | transcript variant 2 |

SEQ ID No. 66:
Amino acid sequence of Homo sapiens Surfactant protein A:

| | |
|---|---|
| NP_001087239.2 | surfactant protein A1 (SFTPA1), |
| | transcript variant 2 |

SEQ ID No. 67:
Nucleotide sequence encoding Homo sapiens Surfactant protein A:

| Accession number | Transcript variant |
|---|---|
| NM_001164644.1 | surfactant protein A1 (SFTPA1), |
| | transcript variant 3 |

SEQ ID No. 68:
Amino acid sequence of Homo sapiens Surfactant protein A:

| | |
|---|---|
| NP_001158116.1 | surfactant protein A1 (SFTPA1), |
| | transcript variant 3 |

SEQ ID No. 69:
Nucleotide sequence encoding Homo sapiens Surfactant protein A:

| | |
|---|---|
| Accession number | Transcript variant |
| NM_01164645.1 | surfactant protein A1 (SFTPA1), |
| | transcript variant 5 |

SEQ ID No. 70:
Amino acid sequence of Homo sapiens Surfactant protein A:

| | |
|---|---|
| NP_001158117.1 | surfactant protein A1 (SFTPA1), |
| | transcript variant 5 |

SEQ ID No. 71:
Nucleotide sequence encoding Homo sapiens Surfactant protein A:

| | |
|---|---|
| Accession number | Transcript variant |
| NM_001164646.1 | surfactant protein A1 (SFTPA1), |
| | transcript variant 6 |

SEQ ID No. 72:
Amino acid sequence of Homo sapiens Surfactant protein A:

| | |
|---|---|
| NP_001158118.1 | surfactant protein A1 (SFTPA1), |
| | transcript variant 6 |

SEQ ID No. 73:
Nucleotide sequence encoding Homo sapiens Surfactant protein A:

| | |
|---|---|
| Accession number | Transcript variant |
| NM_001164647.1 | surfactant protein A1 (SFTPA1), |
| | transcript variant 4 |

SEQ ID No. 74:
Amino acid sequence of Homo sapiens Surfactant protein A:

| | |
|---|---|
| NP_001158119.1 | surfactant protein A1 (SFTPA1), |
| | transcript variant 4 |

SEQ ID No. 75:
Nucleotide sequence encoding Homo sapiens Surfactant protein A:

| | |
|---|---|
| Accession number | Transcript variant |
| NM_005411.4 | surfactant protein A1 (SFTPA1), |
| | transcript variant 1 |

SEQ ID No. 76:
Amino acid sequence of Homo sapiens Surfactant protein A:

| | |
|---|---|
| gene symbol | Alias and additional info |
| NP_005402.3 | surfactant protein A1 (SFTPA1), |
| | transcript variant 1 |

SEQ ID No. 77:
Nucleotide sequence encoding Homo sapiens Surfactant protein B:

| | |
|---|---|
| gene symbol | Alias and additional info |
| SFTPB | Surfactant protein B |
| Accession number | Transcript variant |
| NM_000542.3 | pulmonary surfactant-associated |
| | protein B precursor |

This variant (1) is the longer transcript. Both variants 1 and 2 encode the same protein.
SEQ ID No. 78:
Amino acid sequence of Homo sapiens Surfactant protein B:

| | |
|---|---|
| NP_000533.3 | pulmonary surfactant-associated |
| | protein B precursor |

SEQ ID No. 79:
Nucleotide sequence encoding Homo sapiens Surfactant protein B:

| | |
|---|---|
| NM_198843.2 | pulmonary surfactant-associated |
| | protein B precursor |
| | Alias and additional info |

This variant (2) lacks an internal segment in the 3' UTR, as compared to variant 1.
Both variants 1 and 2 encode the same protein
SEQ ID No. 80:
Nucleotide sequence encoding Homo sapiens napsin A aspartic peptidase:

| | | |
|---|---|---|
| NAPSA | napsin A | NM_004851.1 |
| | aspartic peptidase | |

SEQ ID No. 81:
Amino acid sequence of Homo sapiens napsin A aspartic peptidase:

| | |
|---|---|
| napsin A aspartic peptidase | NP_004842.1 |

The following markers are upregulated in Squamous cell carcinoma.
SEQ ID No. 82:
Nucleotide sequence encoding Homo sapiens tumor protein p63:

| | |
|---|---|
| PMID 21623384 | |
| gene symbol | Alias and additional info |
| TP63 | tumor protein p63 |
| Accession number | Transcript variant |
| NM_001114978.1 | tumor protein p63 (TP63), |
| | transcript variant 2 |

SEQ ID No. 83:

Amino acid sequence of Homo sapiens tumor protein p63:

| | |
|---|---|
| NP_001108450.1 | Homo sapiens tumor protein p63 (TP63), transcript variant 2 |

SEQ ID No. 84:
Nucleotide sequence encoding Homo sapiens tumor protein p63:
tumor protein p63 (TP63), transcript variant 3
NM_001114979.1
SEQ ID No. 85:
Amino acid sequence of Homo sapiens tumor protein p63:

| | |
|---|---|
| NP_001108451.1 | Homo sapiens tumor protein p63 (TP63), transcript variant 3 |

SEQ ID No. 86:
Nucleotide sequence encoding Homo sapiens tumor protein p63:

| | |
|---|---|
| NM_001114980.1 | tumor protein p63 (TP63), transcript variant 4 |

SEQ ID No. 87:
Amino acid sequence of Homo sapiens tumor protein p63:

| | |
|---|---|
| NP_001108452.1 | Homo sapiens tumor protein p63 (TP63), transcript variant 4 |

SEQ ID No. 88:
Nucleotide sequence encoding Homo sapiens tumor protein p63:

| | |
|---|---|
| NM_001114981.1 | tumor protein p63 (TP63), transcript variant 5 |

SEQ ID No. 89:
Amino acid sequence of Homo sapiens tumor protein p63:

| | |
|---|---|
| NP_001108453.1 | Homo sapiens tumor protein p63 (TP63), transcript variant 5 |

SEQ ID No. 90:
Nucleotide sequence encoding Homo sapiens tumor protein p63:

| | |
|---|---|
| NM_001114982.1 | tumor protein p63 (TP63), transcript variant 6 |

SEQ ID No. 91:
Amino acid sequence of Homo sapiens tumor protein p63:

| | |
|---|---|
| NP_001108454.1 | Homo sapiens tumor protein p63 (TP63), transcript variant 6 |

SEQ ID No. 92:
Nucleotide sequence encoding Homo sapiens tumor protein p63:

| | |
|---|---|
| NM_003722.4 | tumor protein p63 (TP63), transcript variant 1 |

SEQ ID No. 93:
Amino acid sequence of Homo sapiens tumor protein p63:

| | |
|---|---|
| NP_003713.3 | Homo sapiens tumor protein p63 (TP63), transcript variant 1 |

SEQ ID No. 94:
Nucleotide sequence encoding Homo sapiens keratin 5:

| | | |
|---|---|---|
| KRT5 | keratin 5 | NM_000424.3 |

SEQ ID No. 95:
Amino acid sequence of Homo sapiens keratin 5:
keratin 5 NP_000415.2
SEQ ID No. 96:
Nucleotide sequence encoding Homo sapiens keratin 6:

| | | |
|---|---|---|
| KRT6A | keratin6 | NM_005554.3 |

SEQ ID No. 97:
Amino acid sequence of Homo sapiens keratin 6:

| | | |
|---|---|---|
| KRT6A | keratin6 | NP_005545.1 |

SEQ ID No. 98:
Nucleotide sequence encoding Homo sapiens keratin 7:

| | | |
|---|---|---|
| KRT7 | keratin 7 | NM_005556.3 |

SEQ ID No. 99:
Amino acid sequence of Homo sapiens keratin 7:

| | | |
|---|---|---|
| KRT7 | keratin 7 | NP_005547.3 |

Nucleotide sequence of Homo sapiens hsa-miR9 and related isoforms:
SEQ ID No. 100:
PMID 23999427

| | |
|---|---|
| hsa-miR9 | micro RNA miR9 |
| NR_029691.1 | Homo sapiens microRNA |
| SEQ ID No. 101: | 9-1 (MIR9-1) |
| NR_030741.1 | Homo sapiens microRNA |
| | 9-2 (MIR9-2) |

SEQ ID No. 102:

| | |
|---|---|
| NR_029692.1 | Homo sapiens microRNA |
| | 9-3 (MIR9-3) |

The following marker is downregulated in adenocarcinoma:
SEQ ID No. 103:
Nucleotide sequence of Homo sapiens hsa-let7-d:

| ″17437991, 24305048 | | |
|---|---|---|
| ″ hsa-1et7-d | microRNA let-7d (MIRLET7D) | NR_029481.1 |

The following markers are upregulated in metastatic adenocarcinoma:

SEQ ID No. 104:
Nucleotide sequence encoding Homo sapiens VEGFA:
VEGFA
NM_001025366.2-vascular endothelial growth factor A isoform a
SEQ ID No. 105:
Amino acid sequence of Homo sapiens VEGFA:
Amino acid-NP_001020537.2
SEQ ID No. 106:
Nucleotide sequence encoding Homo sapiens VEGFA:
NM_001025367.2 vascular endothelial growth factor A isoform c
SEQ ID No. 107:
Amino acid sequence of Homo sapiens VEGFA:
Amino acid-NP_001020538.2
SEQ ID No. 108:
Nucleotide sequence encoding Homo sapiens VEGFA:
NM_001025368.2 vascular endothelial growth factor A isoform d
SEQ ID No. 109:
Amino acid sequence of Homo sapiens VEGFA:
Amino acid-NP_001020539.2
SEQ ID No. 110:
Nucleotide sequence encoding Homo sapiens VEGFA:
NM_001025369.2 vascular endothelial growth factor A isoform e
SEQ ID No. 111:
Amino acid sequence of Homo sapiens VEGFA:
Amino acid-NP_001020540.2
SEQ ID No. 112:
Nucleotide sequence encoding Homo sapiens VEGFA:
NM_001025370.2 vascular endothelial growth factor A isoform f
SEQ ID No. 113:
Amino acid sequence of Homo sapiens VEGFA:
Amino acid-NP_001020541.2
SEQ ID No. 114:
Nucleotide sequence encoding Homo sapiens VEGFA:
NM_001033756.2 vascular endothelial growth factor A isoform g
SEQ ID No. 115:
Amino acid sequence of Homo sapiens VEGFA:
Amino acid-NP_001028928.1
SEQ ID No. 116:
Nucleotide sequence encoding Homo sapiens VEGFA:
NM_001171622.1 vascular endothelial growth factor A isoform h
SEQ ID No. 117:
Amino acid sequence of Homo sapiens VEGFA:
Amino acid-NP_001165093.1
SEQ ID No. 118:
Nucleotide sequence encoding Homo sapiens VEGFA:
NM_001171623.1 vascular endothelial growth factor A isoform i precursor
SEQ ID No. 119:
Amino acid sequence of Homo sapiens VEGFA:
Amino acid-NP_001165094.1
SEQ ID No. 120:
Nucleotide sequence encoding Homo sapiens VEGFA:
NM_001171624.1 vascular endothelial growth factor A isoform j precursor
SEQ ID No. 121:
Amino acid sequence of Homo sapiens VEGFA:
Amino acid-NP_001165095.1
SEQ ID No. 122:
Nucleotide sequence encoding Homo sapiens VEGFA:
NM_001171625.1 vascular endothelial growth factor A isoform k precursor
SEQ ID No. 123:
Amino acid sequence of Homo sapiens VEGFA:
Amino acid-NP_001165096.1
SEQ ID No. 124:
Nucleotide sequence encoding Homo sapiens VEGFA:
NM_001171626.1 vascular endothelial growth factor A isoform l precursor
SEQ ID No. 125:
Amino acid sequence of Homo sapiens VEGFA:
Amino acid-NP_001165097.1
SEQ ID No. 126:
Nucleotide sequence encoding Homo sapiens VEGFA:
NM_001171627.1 vascular endothelial growth factor A isoform m precursor
SEQ ID No. 127:
Amino acid sequence of Homo sapiens VEGFA:
Amino acid-NP_001165098.1
SEQ ID No. 128:
Nucleotide sequence encoding Homo sapiens VEGFA:
NM_001171628.1 vascular endothelial growth factor A isoform n precursor
SEQ ID No. 129:
Amino acid sequence of Homo sapiens VEGFA:
Amino acid-NP_001165099.1
SEQ ID No. 130:
Nucleotide sequence encoding Homo sapiens VEGFA:
NM_001171629.1 vascular endothelial growth factor A isoform o precursor
SEQ ID No. 131:
Amino acid sequence of Homo sapiens VEGFA:
Amino acid-NP_001165100.1
SEQ ID No. 132:
Nucleotide sequence encoding Homo sapiens VEGFA:
NM_001171630.1 vascular endothelial growth factor A isoform p precursor
SEQ ID No. 133:
Amino acid sequence of Homo sapiens VEGFA:
Amino acid-NP_001165101.1
SEQ ID No. 134:
Nucleotide sequence encoding Homo sapiens VEGFA:
NM_001204384.1 vascular endothelial growth factor A isoform q precursor
SEQ ID No. 135:
Amino acid sequence of Homo sapiens VEGFA:
Amino acid-NP_001191313.1
SEQ ID No. 136:
Nucleotide sequence encoding Homo sapiens VEGFA:
NM_001204385.1 vascular endothelial growth factor A isoform r
SEQ ID No. 137:
Amino acid sequence of Homo sapiens VEGFA:
Amino acid-NP_001191314.1
SEQ ID No. 138:
Nucleotide sequence encoding Homo sapiens VEGFA:
NM_001287044.1 vascular endothelial growth factor A isoform s
SEQ ID No. 139:
Amino acid sequence of Homo sapiens VEGFA:
Amino acid-NP_001273973.1
SEQ ID No. 140:
Nucleotide sequence encoding Homo sapiens VEGFA:
NM_003376.5 vascular endothelial growth factor A isoform b
SEQ ID No. 141:
Amino acid sequence of Homo sapiens VEGFA:
Amino acid- NP_003367.4
SEQ ID No. 142:
Nucleotide sequence encoding Homo sapiens VEGFB:
VEGFB
NM_001243733.1 vascular endothelial growth factor B isoform VEGFB-167 precursor
SEQ ID No. 143:
Amino acid sequence of Homo sapiens VEGFB:
Amino acid-NP_001230662.1
SEQ ID No. 144:
Nucleotide sequence encoding Homo sapiens VEGFB:
NM_003377.4 vascular endothelial growth factor B isoform VEGFB-186 precursor
SEQ ID No. 145:
Amino acid sequence of Homo sapiens VEGFB:
Amino acid-NP_003368.1
SEQ ID No. 146:
Nucleotide sequence encoding Homo sapiens VEGFD:
VEGFD (FIGF, c-fos induced growth factor)
NM_004469.4vascular endothelial growth factor D preproprotein
SEQ ID No. 147:
Amino acid sequence of Homo sapiens VEGFD:
Amino acid-NP_004460.1
SEQ ID No. 148:
Nucleotide sequence encoding Homo sapiens VEGFC:

| | | |
|---|---|---|
| 11707590 VEGFC | Vascular endothelial growth factor C | NM_005429.4 |

SEQ ID No. 149:
Amino acid sequence of Homo sapiens VEGFC:

| | | |
|---|---|---|
| VEGFC | Vascular endothelial growth factor C | NP_005420.1 |

SEQ ID No. 150:
Nucleotide sequence encoding Homo sapiens PLAUR

| | |
|---|---|
| 11707590 PLAUR plasminogen activator urokinase receptor | NM_001005376.2 |
| plasminogen activator, urokinase receptor (PLAUR), transcript variant 2 | |
| SEQ ID No. 151: | |
| Amino acid sequence of Homo sapiens PLAUR | |

PLAUR plasminogen activator urokinase receptor NP_001005376.1 Homo sapiens
plasminogen activator, urokinase receptor (PLAUR), transcript variant 2
SEQ ID No. 152:
Nucleotide sequence encoding Homo sapiens PLAUR
11707590 PLAUR plasminogen activator urokinase receptor NM_001005377.2plasminogen
activator, urokinase receptor (PLAUR), transcript variant 3
SEQ ID No. 153:
Amino acid of Homo sapiens PLAUR
PLAUR plasminogen activator urokinase receptor Homo sapiens plasminogen activator,
urokinase receptor (PLAUR), transcript variant 3
SEQ ID No. 154:
Nucleotide sequence encoding Homo sapiens PLAUR
11707590 PLAUR plasminogen activator urokinase receptor plasminogen activator,
urokinase receptor (PLAUR), transcript variant 4
SEQ ID No. 155:
Amino acid sequence of Homo sapiens PLAUR
PLAUR plasminogen activator urokinase receptor NP_001287966.1 Homo sapiens
plasminogen activator, urokinase receptor (PLAUR), transcript variant 4
SEQ ID No. 156:
Nucleotide sequence encoding Homo sapiens PLAUR
11707590 PLAUR plasminogen activator urokinase receptor plasminogen activator,
urokinase receptor (PLAUR), transcript variant 1
SEQ ID No. 157:
Amino acid sequence of Homo sapiens PLAUR
PLAUR plasminogen activator urokinase receptor
Homo sapiens plasminogen activator, urokinase receptor (PLAUR),
NP_002650.1 transcript variant 1

The following marker is upregulated in Large cell lung cancer

SEQ ID No. 158:
Nucleotide sequence encoding Homo sapiens HMGA1
19903768 HMGA1 NM_002131.3 Homo sapiens high mobility group
AT-hook 1 (HMGA1), transcript variant 2
SEQ ID No. 159:
Amino acid sequence of Homo sapiens HMGA1
HMGA1 NP_002122.1 Homo sapiens high mobility group AT-hook 1 (HMGA1),
transcript variant 2
SEQ ID No. 160:
Nucleotide sequence encoding Homo sapiens HMGA1
Homo sapiens high mobility group AT-hook 1 (HMGA1), transcript variant 1
19903768 HMGA1 NM_145899.2 Homo sapiens high mobility group AT-hook 1
(HMGA1), transcript variant 1
SEQ ID No. 161:
Amino acid sequence of Homo sapiens HMGA1
HMGA1 NP_665906.1 Homo sapiens high mobility group AT-hook 1 (HMGA1),
transcript variant 1
SEQ ID No. 162:
Nucleotide sequence encoding Homo sapiens HMGA1
19903768 HMGA1 NM_145901.2 Homo sapiens high mobility group AT-hook 1
(HMGA1), transcript variant 3
SEQ ID No. 163:
Amino acid sequence of Homo sapiens HMGA1
HMGA1 NP_665908.1 Homo sapiens high mobility group AT-hook 1 (HMGA1),
transcript variant 3
SEQ ID No. 164:
Nucleotide sequence encoding Homo sapiens HMGA1
19903768 HMGA1
NM_145902.2 Homo sapiens high mobility group AT-hook 1 (HMGA1), transcript
variant 4
SEQ ID No. 165:
Amino acid sequence of Homo sapiens HMGA1
HMGA1 Homo sapiens high mobility group AT-hook 1 (HMGA1), transcript variant 4
SEQ ID No. 166:
19903768 HMGA1 NM_145903.2 Homo sapiens high mobility group AT-hook 1
(HMGA1), transcript variant 5
SEQ ID No. 167:
Amino acid sequence of Homo sapiens HMGA1
NP_665910.1 Homo sapiens high mobility group AT-hook 1 (HMGA1), transcript variant 5
SEQ ID No. 168:
19903768 HMGA1 NM_145905.2 Homo sapiens high mobility group
AT-hook 1 (HMGA1), transcript variant 7
SEQ ID No. 169:
Amino acid sequence of Homo sapiens HMGA1
HMGA1 NP_665912.1 Homo sapiens high mobility group AT-hook 1 (HMGA1),
transcript variant 7

### Genomic Alterations

Genomic alterations

| | | |
|---|---|---|
| PMID | | |
| | 18794081 KRAS G12D | G --> CIT transversion |
| | | at codon for Exon 12 |
| | | Adenocarcinoma |
| | 21471965 KRAS G12D// | R172H Substitution in p53 |
| | p53 mutations | (Li-Fraumeni syndrome, PMID |
| | | 15607981) |
| | | Metastatic Adenocarcinoma |
| | 18794081 KRAS G12D | G --> A transition |
| | | Adenocarcinoma in never smokers |
| | 1324794 p53 mutations, | Adenocarcinoma or Squamous |
| | translocations | cell carcinoma |
| | 15737014 EGFR T790M | mutation in exon 20, codon 790 |
| | | Drug resistant Adenocarcinoma, |
| | | patients relapse after tyrosine |
| | | kinase inhibitors |
| | 21665149 p53 mutations//Rb-/- | Small cell carcinoma |

The following table provides more detailed information in relation to genomic alterations:

| Amino acid | Genomic | Cancer | |
|---|---|---|---|
| change/Gene | Alteration | classification | Reference |
| KRAS G12D | G → C/T transversion | Adenocarcinoma | (Riely, Kris et al. 2008) |
| | G → A transition | Adenocarcinoma in | (Winslow, Dayton et al. |
| | | never smokers | 2011) |
| p53 | Mutations and | Adenocarcinoma or | (Kishimoto, |
| | translocations | Squamous cell | Murakami et al. 1992) |
| | | carcinoma | |
| P53 R172H | | Li-Fraumeni | (Lang, Iwakuma |
| Substitution in p53 | | syndrome | et al. 2004) |
| KRAS G12D//p53 | | Metastatic | |
| mutations | | Adenocarcinoma | |
| EGFR T790M | Mutations in exon 20, | Drug resistant | (Pao, Miller et al. 2005) |
| | codon 790 | Adenocarcinoma, | |
| | | patients relapse | |
| | | after tyrosine kinase | |
| | | inhibitors | |
| p53 mutations//Rb-/- | | Small cell | (Sutherland, Proost et |
| | | carcinoma | al. 2011) |

### REFERENCES

1. Herbst R S, Heymach J V, Lippman S M. Lung cancer. The New England journal of medicine 2008; 359:1367-80.
2. Hoffman P C, Mauer A M, Vokes E E. Lung cancer. Lancet 2000; 355:479-85.
3. Hyde L, Hyde C I. Clinical manifestations of lung cancer. Chest 1974; 65:299-306.
4. Strauss G M, Dominioni L. Chest X-ray screening for lung cancer: overdiagnosis, endpoints, and randomized population trials. Journal of surgical oncology 2013; 108:294-300.
5. D'Urso V, Doneddu V, Marchesi I, et al. Sputum analysis: non-invasive early lung cancer detection. Journal of cellular physiology 2013; 228:945-51.
6. Travis W D, Brambilla E, Noguchi M, et al. Diagnosis of lung cancer in small biopsies and cytology: implications of the 2011 International Association for the Study of Lung Cancer/American Thoracic Society/European Respiratory Society classification. Archives of pathology & laboratory medicine 2013; 137:668-84.
7. Keijzer R, van Tuyl M, Meijers C, et al. The transcription factor GATA6 is essential for branching morphogenesis and epithelial cell differentiation during fetal pulmonary development. Development 2001; 128:503-11.
8. Tian Y, Zhang Y, Hurd L, et al. Regulation of lung endoderm progenitor cell behavior by miR302/367. Development 2011; 138:1235-45.
9. Zhang Y, Rath N, Hannenhalli S, et al. GATA and Nkx factors synergistically regulate tissue-specific gene expression and development in vivo. Development 2007; 134:189-98.
10. Kolla V, Gonzales L W, Gonzales J, et al. Thyroid transcription factor in differentiating type II cells: regulation, isoforms, and target genes. American journal of respiratory cell and molecular biology 2007; 36:213-25.
11. Guo M, Akiyama Y, House M G, et al. Hypermethylation of the GATA genes in lung cancer. Clinical cancer research: an official journal of the American Association for Cancer Research 2004; 10:7917-24.
12. Gorshkova E V, Kaledin V I, Kobzev V F, Merkulova T I. Codon 12 region of mouse K-ras gene is the site for in vitro binding of transcription factors GATA-6 and NF-Y. Biochemistry Biokhimiia 2005; 70:1180-4.
13. Lindholm P M, Soini Y, Myllarniemi M, et al. Expression of GATA-6 transcription factor in pleural malignant mesothelioma and metastatic pulmonary adenocarcinoma. Journal of clinical pathology 2009; 62:339-44.
14. Cheung W K, Zhao M, Liu Z, et al. Control of alveolar differentiation by the lineage transcription factors GATA6 and HOPX inhibits lung adenocarcinoma metastasis. Cancer cell 2013; 23:725-38.
15. Chen P M, Wu T C, Wang Y C, et al. Activation of NF-kappaB by SOD2 promotes the aggressiveness of lung adenocarcinoma by modulating NKX2-1-mediated IKKbeta expression. Carcinogenesis 2013; 34:2655-63.
16. Winslow M M, Dayton T L, Verhaak R G, et al. Suppression of lung adenocarcinoma progression by Nkx2-1. Nature 2011; 473:101-4.
17. Elkin M, Vlodaysky I. Tail vein assay of cancer metastasis. Current protocols in cell biology/editorial board, Juan S Bonifacino [et al] 2001; Chapter 19: Unit 19 2.
18. Horvath I, Hunt J, Barnes P J, et al. Exhaled breath condensate: methodological recommendations and unresolved questions. The European respiratory journal 2005; 26:523-48.
19. Ho L P, Innes J A, Greening A P. Nitrite levels in breath condensate of patients with cystic fibrosis is elevated in contrast to exhaled nitric oxide. Thorax 1998; 53:680-4.
20. Effros R M, Casaburi R, Porszasz J, Morales E M, Rehan V. Exhaled breath condensates: analyzing the expiratory plume. American journal of respiratory and critical care medicine 2012; 185:803-4.
21. Davis M D, Montpetit A, Hunt J. Exhaled breath condensate: an overview. Immunology and allergy clinics of North America 2012; 32:363-75.
22. Shahid S K, Kharitonov S A, Wilson N M, Bush A, Barnes P J. Increased interleukin-4 and decreased interferon-gamma in exhaled breath condensate of children with asthma. American journal of respiratory and critical care medicine 2002; 165:1290-3.
23. Montuschi P, Kharitonov S A, Ciabattoni G, Barnes P J. Exhaled leukotrienes and prostaglandins in COPD. Thorax 2003; 58:585-8.
24. Kostikas K, Papatheodorou G, Psathakis K, Panagou P, Loukides S. Prostaglandin E2 in the expired breath condensate of patients with asthma. The European respiratory journal 2003; 22:743-7.
25. Huszar E, Vass G, Vizi E, et al. Adenosine in exhaled breath condensate in healthy volunteers and in patients with asthma. The European respiratory journal 2002; 20:1393-8.
26. Effros R M, Hoagland K W, Bosbous M, et al. Dilution of respiratory solutes in exhaled condensates. American journal of respiratory and critical care medicine 2002; 165:663-9.
27. Montuschi P. Analysis of exhaled breath condensate in respiratory medicine: methodological aspects and potential clinical applications. Therapeutic advances in respiratory disease 2007; 1:5-23.
28. Giangreco A, Groot K R, Janes S M. Lung cancer and lung stem cells: strange bedfellows? American journal of respiratory and critical care medicine 2007; 175:547-53.
29. National Lung Screening Trial Research T, Aberle D R, Adams A M, et al. Reduced lung-cancer mortality with low-dose computed tomographic screening. The New England journal of medicine 2011; 365:395-409.
30. Zhong L, Goldberg M S, Gao Y T, Jin F. A case-control study of lung cancer and environmental tobacco smoke among nonsmoking women living in Shanghai, China. Cancer causes & control: CCC 1999; 10:607-16.
31. Xu Z Y, Blot W J, Xiao H P, et al. Smoking, air pollution, and the high rates of lung cancer in Shenyang, China. Journal of the National Cancer Institute 1989; 81:1800-6.
32. Henschke C I, McCauley D I, Yankelevitz D F, et al. Early Lung Cancer Action Project: overall design and findings from baseline screening. Lancet 1999; 354:99-105.
33. Jett J R. Limitations of screening for lung cancer with low-dose spiral computed tomography. Clinical cancer research: an official journal of the American Association for Cancer Research 2005; 11:4988s-92s.
34. Bhattacharjee A, Richards W G, Staunton J, et al. Classification of human lung carcinomas by mRNA expression profiling reveals distinct adenocarcinoma subclasses. Proceedings of the National Academy of Sciences of the United States of America 2001; 98:13790-5.
35. Meyerson M, Carbone D. Genomic and proteomic profiling of lung cancers: lung cancer classification in the age of targeted therapy. Journal of clinical oncology: official journal of the American Society of Clinical Oncology 2005; 23:3219-26.
36. Chen H Y, Yu S L, Chen C H, et al. A five-gene signature and clinical outcome in non-small-cell lung cancer. The New England journal of medicine 2007; 356:11-20.
37. Beer D G, Kardia S L, Huang C C, et al. Gene-expression profiles predict survival of patients with lung adenocarcinoma. Nature medicine 2002; 8:816-24.
38. Sing T, Sander O, Beerenwinkel N, Lengauer T. ROCR: visualizing classifier performance in R. Bioinformatics 2005; 21:3940-1.
39 Evgenia Dimitriadou, Kurt Hornik, Friedrich Leisch, David Meyer and Andreas Weingessel (2010). e1071: Misc Functions of the Department of Statistics (e1071), T U Wien. R package version 1.5-24. http://CRAN.Rproject. org/package=e1071

### FURTHER REFERENCES

(2011). The Diagnosis and Treatment of Lung Cancer (Update). Cardiff (UK).
Asnaghi, L., W. C. Vass, R. Quadri, P. M. Day, X. Qian, R. Braverman, A. G. Papageorge and D. R. Lowy (2010). “E-cadherin negatively regulates neoplastic growth in non-small cell lung cancer: role of Rho GTPases.” Oncogene 29(19): 2760-2771.
Brodowicz, T., M. Krzakowski, M. Zwitter, V. Tzekova, R. Ramlau, N. Ghilezan, T. Ciuleanu, B. Cucevic, K. Gyurkovits, E. Ulsperger, J. Jassem, M. Grgic, P. Saip, M. Szilasi, C. Wiltschke, M. Wagnerova, N. Oskina, V. Soldatenkova, C. Zielinski, M. Wenczl and C. Central European Cooperative Oncology Group (2006). “Cisplatin and gemcitabine first-line chemotherapy followed by maintenance gemcitabine or best supportive care in advanced non-small cell lung cancer: a phase III trial.” Lung Cancer 52(2): 155-163.
Burdett, S. S., L. A. Stewart and L. Rydzewska (2007). “Chemotherapy and surgery versus surgery alone in non-small cell lung cancer.” Cochrane Database Syst Rev(3): CD006157.
Cagle, P. T. and L. R. Chirieac (2012). “Advances in treatment of lung cancer with targeted therapy.” Arch Pathol Lab Med 136(5): 504-509.
Dosoretz, D. E., M. J. Katin, P. H. Blitzer, J. H. Rubenstein, S. Salenius, M. Rashid, R. A. Dosani, G. Mestas, A. D. Siegel, T. T. Chadha and et al. (1992). “Radiation therapy in the management of medically inoperable carcinoma of the lung: results and implications for future treatment strategies.” Int J Radiat Oncol Biol Phys 24(1): 3-9.
Furuse, K., M. Fukuoka, M. Kawahara, H. Nishikawa, Y. Takada, S. Kudoh, N. Katagami and Y. Ariyoshi (1999). “Phase III study of concurrent versus sequential thoracic radiotherapy in combination with mitomycin, vindesine, and cisplatin in unresectable stage III non-small-cell lung cancer.” J Clin Oncol 17(9): 2692-2699.
Garber, M. E., O. G. Troyanskaya, K. Schluens, S. Petersen, Z. Thaesler, M. Pacyna-Gengelbach, M. van de Rijn, G. D. Rosen, C. M. Perou, R. I. Whyte, R. B. Altman, P. O. Brown, D. Botstein and I. Petersen (2001). “Diversity of gene expression in adenocarcinoma of the lung.” Proc Natl Acad Sci USA 98(24): 13784-13789.
Gauden, S., J. Ramsay and L. Tripcony (1995). “The curative treatment by radiotherapy alone of stage I non-small cell carcinoma of the lung.” Chest 108(5): 1278-1282.
Han, H., J. F. Silverman, T. S. Santucci, R. S. Macherey, T. A. d'Amato, M. Y. Tung, R. J. Weyant and R. J. Landreneau (2001). “Vascular endothelial growth factor expression in stage I non-small cell lung cancer correlates with neoangiogenesis and a poor prognosis.” Ann Surg Oncol 8(1): 72-79.
Hanna, N., F. A. Shepherd, F. V. Fossella, J. R. Pereira, F. De Marinis, J. von Pawel, U. Gatzemeier, T. C. Tsao, M. Pless, T. Muller, H. L. Lim, C. Desch, K. Szondy, R. Gervais, Shaharyar, C. Manegold, S. Paul, P. Paoletti, L. Einhorn and P. A. Bunn, Jr. (2004). “Randomized phase III trial of pemetrexed versus docetaxel in patients with non-small-cell lung cancer previously treated with chemotherapy.” J Clin Oncol 22(9): 1589-1597.
Hillion, J., L. J. Wood, M. Mukherjee, R. Bhattacharya, F. Di Cello, J. Kowalski, O. Elbahloul, J. Segal, J. Poirier, C. M. Rudin, S. Dhara, A. Belton, B. Joseph, S. Zucker and L. M. Resar (2009). “Upregulation of MMP-2 by HMGA1 promotes transformation in undifferentiated, large-cell lung cancer.” Mol Cancer Res 7(11): 1803-1812.
Hoffman, P. C., A. M. Mauer and E. E. Vokes (2000). “Lung cancer.” Lancet 355(9202): 479-485.
Kase, S., K. Sugio, K. Yamazaki, T. Okamoto, T. Yano and K. Sugimachi (2000). “Expression of E-cadherin and beta-catenin in human non-small cell lung cancer and the clinical significance.” Clin Cancer Res 6(12): 4789-4796.
Kim, E. S., V. Hirsh, T. Mok, M. A. Socinski, R. Gervais, Y. L. Wu, L. Y. Li, C. L. Watkins, M. V. Sellers, E. S. Lowe, Y. Sun, M. L. Liao, K. Osterlind, M. Reck, A. A. Armour, F. A. Shepherd, S. M. Lippman and J. Y. Douillard (2008). “Gefitinib versus docetaxel in previously treated non-small-cell lung cancer (INTEREST): a randomised phase III trial.” Lancet 372(9652): 1809-1818.
Kishimoto, Y., Y. Murakami, M. Shiraishi, K. Hayashi and T. Sekiya (1992). “Aberrations of the p53 tumor suppressor gene in human non-small cell carcinomas of the lung.” Cancer Res 52(17): 4799-4804.
Kumar, M. S., E. Armenteros-Monterroso, P. East, P. Chakravorty, N. Matthews, M. M. Winslow and J. Downward (2014). “HMGA2 functions as a competing endogenous RNA to promote lung cancer progression.” Nature 505(7482): 212-217.
Kwak, E. L., Y. J. Bang, D. R. Camidge, A. T. Shaw, B. Solomon, R. G. Maki, S. H. Ou, B. J. Dezube, P. A. Janne, D. B. Costa, M. Varella-Garcia, W. H. Kim, T. J. Lynch, P. Fidias, H. Stubbs, J. A. Engelman, L. V. Sequist, W. Tan, L. Gandhi, M. Mino-Kenudson, G. C. Wei, S. M. Shreeve, M. J. Ratain, J. Settleman, J. G. Christensen, D. A. Haber, K. Wilner, R. Salgia, G. I. Shapiro, J. W. Clark and A. J. Iafrate (2010). “Anaplastic lymphoma kinase inhibition in non-small-cell lung cancer.” N Engl J Med 363(18): 1693-1703.
Lang, G. A., T. Iwakuma, Y. A. Suh, G. Liu, V. A. Rao, J. M. Parant, Y. A. Valentin-Vega, T. Terzian, L. C. Caldwell, L. C. Strong, A. K. El-Naggar and G. Lozano (2004). “Gain of function of a p53 hot spot mutation in a mouse model of Li-Fraumeni syndrome.” Cell 119(6): 861-872.
Le Chevalier, T., R. Arriagada, M. Tarayre, M. J. Lacombe-Terrier, A. Laplanche, E. Quoix, P. Ruffle, M. Martin and J. Y. Douillard (1992). “Significant effect of adjuvant chemotherapy on survival in locally advanced non-small-cell lung carcinoma.” J Natl Cancer Inst 84(1): 58.
Lee, Y. S. and A. Dutta (2007). “The tumor suppressor microRNA let-7 represses the HMGA2 oncogene.” Genes Dev 21(9): 1025-1030.
Li, J., Y. M. Hu, Y. J. Du, L. R. Zhu, H. Qian, Y. Wu and W. L. Shi (2014). “Expressions of MUC1 and vascular endothelial growth factor mRNA in blood are biomarkers for predicting efficacy of gefitinib treatment in non-small cell lung cancer.” BMC Cancer 14(1): 848.
Martini, N., M. S. Bains, M. E. Burt, M. F. Zakowski, P. McCormack, V. W. Rusch and R. J. Ginsberg (1995). “Incidence of local recurrence and second primary tumors in resected stage I lung cancer.” J Thorac Cardiovasc Surg 109(1): 120-129.
Martini, N., M. E. Burt, M. S. Bains, P. M. McCormack, V. W. Rusch and R. J. Ginsberg (1992). “Survival after resection of stage II non-small cell lung cancer.” Ann Thorac Surg 54(3): 460-465; discussion 466.
Mok, T. S., Y. L. Wu, S. Thongprasert, C. H. Yang, D. T. Chu, N. Saijo, P. Sunpaweravong, B. Han, B. Margono, Y. Ichinose, Y. Nishiwaki, Y. Ohe, J. J. Yang, B. Chewaskulyong, H. Jiang, E. L. Duffield, C. L. Watkins, A. A. Armour and M. Fukuoka (2009). “Gefitinib or carboplatin-paclitaxel in pulmonary adenocarcinoma.” N Engl J Med 361(10): 947-957.
Molina, J. R., P. Yang, S. D. Cassivi, S. E. Schild and A. A. Adjei (2008). “Non-small cell lung cancer: epidemiology, risk factors, treatment, and survivorship.” Mayo Clin Proc 83(5): 584-594.
Murray, N., P. Coy, J. L. Pater, I. Hodson, A. Arnold, B. C. Zee, D. Payne, E. C. Kostashuk, W. K. Evans, P. Dixon and et al. (1993). “Importance of timing for thoracic irradiation in the combined modality treatment of limited-stage small-cell lung cancer. The National Cancer Institute of Canada Clinical Trials Group.” J Clin Oncol 11(2): 336-344.
Okamoto, H., K. Watanabe, H. Kunikane, A. Yokoyama, S. Kudoh, T. Asakawa, T. Shibata, H. Kunitoh, T. Tamura and N. Saijo (2007). “Randomised phase III trial of carboplatin plus etoposide vs split doses of cisplatin plus etoposide in elderly or poor-risk patients with extensive disease small-cell lung cancer: JCOG 9702.” Br J Cancer 97(2): 162-169.
Osterlind, K., M. Hansen, H. H. Hansen, P. Dombernowsky and M. Rorth (1985). “Treatment policy of surgery in small cell carcinoma of the lung: retrospective analysis of a series of 874 consecutive patients.” Thorax 40(4): 272-277.
Pao, W., V. A. Miller, K. A. Politi, G. J. Riely, R. Somwar, M. F. Zakowski, M. G. Kris and H. Varmus (2005). “Acquired resistance of lung adenocarcinomas to gefitinib or erlotinib is associated with a second mutation in the EGFR kinase domain.” PLoS Med 2(3): e73.
Park, J. O., S. W. Kim, J. S. Ahn, C. Suh, J. S. Lee, J. S. Jang, E. K. Cho, S. H. Yang, J. H. Choi, D. S. Heo, S. Y. Park, S. W. Shin, M. J. Ahn, J. S. Lee, Y. H. Yun, J. W. Lee and K. Park (2007). “Phase III trial of two versus four additional cycles in patients who are nonprogressive after two cycles of platinum-based chemotherapy in non small-cell lung cancer.” J Clin Oncol 25(33): 5233-5239.
Paz-Ares, L., F. de Marinis, M. Dediu, M. Thomas, J. L. Pujol, P. Bidoli, O. Molinier, T. P. Sahoo, E. Laack, M. Reck, J. Corral, S. Melemed, W. John, N. Chouaki, A. H. Zimmermann, C. Visseren-Grul and C. Gridelli (2012). “Maintenance therapy with pemetrexed plus best supportive care versus placebo plus best supportive care after induction therapy with pemetrexed plus cisplatin for advanced non-squamous non-small-cell lung cancer (PARAMOUNT): a double-blind, phase 3, randomised controlled trial.” Lancet Oncol 13(3): 247-255.
Pelosi, G., F. Pasini, C. Olsen Stenholm, U. Pastorino, P. Maisonneuve, A. Sonzogni, F. Maffini, G. Pruneri, F. Fraggetta, A. Cavallon, E. Roz, A. Iannucci, E. Bresaola and G. Viale (2002). “p63 immunoreactivity in lung cancer: yet another player in the development of squamous cell carcinomas?” J Pathol 198(1): 100-109.
Pignon, J. P., R. Arriagada, D. C. Ihde, D. H. Johnson, M. C. Perry, R. L. Souhami, O. Brodin, R. A. Joss, M. S. Kies, B. Lebeau and et al. (1992). “A meta-analysis of thoracic radiotherapy for small-cell lung cancer.” N Engl J Med 327(23): 1618-1624.
Pignon, J. P., H. Tribodet, G. V. Scagliotti, J. Y. Douillard, F. A. Shepherd, R. J. Stephens, A. Dunant, V. Torri, R. Rosell, L. Seymour, S. G. Spiro, E. Rolland, R. Fossati, D. Aubert, K. Ding, D. Waller, T. Le Chevalier and L. C. Group (2008). “Lung adjuvant cisplatin evaluation: a pooled analysis by the LACE Collaborative Group.” J Clin Oncol 26(21): 3552-3559.
Prasad, U. S., A. R. Naylor, W. S. Walker, D. Lamb, E. W. Cameron and P. R. Walbaum (1989). “Long term survival after pulmonary resection for small cell carcinoma of the lung.” Thorax 44(10): 784-787.
Qi, L., F. Zhu, S. H. Li, L. B. Si, L. K. Hu and H. Tian (2014). “Retinoblastoma binding protein 2 (RBP2) promotes HIF-lalpha-VEGF-induced angiogenesis of non-small cell lung cancer via the Akt pathway.” PLoS One 9(8): e106032.
Rekhtman, N., D. C. Ang, C. S. Sima, W. D. Travis and A. L. Moreira (2011). “Immunohistochemical algorithm for differentiation of lung adenocarcinoma and squamous cell carcinoma based on large series of whole-tissue sections with validation in small specimens.” Mod Pathol 24(10): 1348-1359.
Riely, G. J., M. G. Kris, D. Rosenbaum, J. Marks, A. Li, D. A. Chitale, K. Nafa, E. R. Riedel, M. Hsu, W. Pao, V. A. Miller and M. Ladanyi (2008). “Frequency and distinctive spectrum of KRAS mutations in never smokers with lung adenocarcinoma.” Clin Cancer Res 14(18): 5731-5734.
Scagliotti, G. V., P. Parikh, J. von Pawel, B. Biesma, J. Vansteenkiste, C. Manegold, P. Serwatowski, U. Gatzemeier, R. Digumarti, M. Zukin, J. S. Lee, A. Mellemgaard, K. Park, S. Patil, J. Rolski, T. Goksel, F. de Marinis, L. Simms, K. P. Sugarman and D. Gandara (2008). “Phase III study comparing cisplatin plus gemcitabine with cisplatin plus pemetrexed in chemotherapy-naive patients with advanced-stage non-small-cell lung cancer.” J Clin Oncol 26(21): 3543-3551.
Schuchert, M. J., G. Abbas, A. Pennathur, K. S. Nason, D. O. Wilson, J. D. Luketich and R. J. Landreneau (2010). “Sublobar resection for early-stage lung cancer.” Semin Thorac Cardiovasc Surg 22(1): 22-31.
Shaw, A. T., B. Y. Yeap, B. J. Solomon, G. J. Riely, J. Gainor, J. A. Engelman, G. I. Shapiro, D. B. Costa, S. H. Ou, M. Butaney, R. Salgia, R. G. Maki, M. Varella-Garcia, R. C. Doebele, Y. J. Bang, K. Kulig, P. Selaru, Y. Tang, K. D. Wilner, E. L. Kwak, J. W. Clark, A. J. Iafrate and D. R. Camidge (2011). “Effect of crizotinib on overall survival in patients with advanced non-small-cell lung cancer harbouring ALK gene rearrangement: a retrospective analysis.” Lancet Oncol 12(11): 1004-1012.
Shijubo, N., T. Uede, S. Kon, M. Maeda, T. Segawa, A. Imada, M. Hirasawa and S. Abe (1999). “Vascular endothelial growth factor and osteopontin in stage I lung adenocarcinoma.” Am J Respir Crit Care Med 160(4): 1269-1273.
Slotman, B., C. Faivre-Finn, G. Kramer, E. Rankin, M. Snee, M. Hatton, P. Postmus, L. Collette, E. Musat, S. Senan, E. R. O. Group and G. Lung Cancer (2007). “Prophylactic cranial irradiation in extensive small-cell lung cancer.” N Engl J Med 357(7): 664-672.
Smit, E. F., H. J. Groen, W. Timens, W. J. de Boer and P. E. Postmus (1994). “Surgical resection for small cell carcinoma of the lung: a retrospective study.” Thorax 49(1): 20-22.
Stacker, S. A., C. Caesar, M. E. Baldwin, G. E. Thornton, R. A. Williams, R. Prevo, D. G. Jackson, S. Nishikawa, H. Kubo and M. G. Achen (2001). “VEGF-D promotes the metastatic spread of tumor cells via the lymphatics.” Nat Med 7(2): 186-191.
Su, J. L., P. C. Yang, J. Y. Shih, C. Y. Yang, L. H. Wei, C. Y. Hsieh, C. H. Chou, Y. M. Jeng, M. Y. Wang, K. J. Chang, M. C. Hung and M. L. Kuo (2006). “The VEGF-C/Flt-4 axis promotes invasion and metastasis of cancer cells.” Cancer Cell 9(3): 209-223.
Sundstrom, S., R. Bremnes, U. Aasebo, S. Aamdal, R. Hatlevoll, P. Brunsvig, D. C. Johannessen, O. Klepp, P. M. Fayers and S. Kaasa (2004). “Hypofractionated palliative radiotherapy (17 Gy per two fractions) in advanced non-small-cell lung carcinoma is comparable to standard fractionation for symptom control and survival: a national phase III trial.” J Clin Oncol 22(5): 801-810.
Sutherland, K. D., N. Proost, I. Brouns, D. Adriaensen, J. Y. Song and A. Berns (2011). “Cell of origin of small cell lung cancer: inactivation of Trp53 and Rb1 in distinct cell types of adult mouse lung.” Cancer Cell 19(6): 754-764.
Taguchi, A., S. Hanash, A. Rundle, I. W. McKeague, D. Tang, S. Darakjy, J. M. Gaziano, H. D. Sesso and F. Perera (2013). “Circulating pro-surfactant protein B as a risk biomarker for lung cancer.” Cancer Epidemiol Biomarkers Prev 22(10): 1756-1761.
Turner, B. M., P. T. Cagle, I. M. Sainz, J. Fukuoka, S. S. Shen and J. Jagirdar (2012). “Napsin A, a new marker for lung adenocarcinoma, is complementary and more sensitive and specific than thyroid transcription factor 1 in the differential diagnosis of primary pulmonary carcinoma: evaluation of 1674 cases by tissue microarray.” Arch Pathol Lab Med 136(2): 163-171.
Warde, P. and D. Payne (1992). “Does thoracic irradiation improve survival and local control in limited-stage small-cell carcinoma of the lung? A meta-analysis.” J Clin Oncol 10(6): 890-895.
White, R. A., J. M. Neiman, A. Reddi, G. Han, S. Birlea, D. Mitra, L. Dionne, P. Fernandez, K. Murao, L. Bian, S. B. Keysar, N. B. Goldstein, N. Song, S. Bornstein, Z. Han, X. Lu, J. Wisell, F. Li, J. Song, S. L. Lu, A. Jimeno, D. R. Roop and X. J. Wang (2013). “Epithelial stem cell mutations that promote squamous cell carcinoma metastasis.” J Clin Invest 123(10): 4390-4404.
Whithaus, K., J. Fukuoka, T. J. Prihoda and J. Jagirdar (2012). “Evaluation of napsin A, cytokeratin 5/6, p63, and thyroid transcription factor 1 in adenocarcinoma versus squamous cell carcinoma of the lung.” Arch Pathol Lab Med 136(2): 155-162.
Winslow, M. M., T. L. Dayton, R. G. Verhaak, C. Kim-Kiselak, E. L. Snyder, D. M. Feldser, D. D. Hubbard, M. J. DuPage, C. A. Whittaker, S. Hoersch, S. Yoon, D. Crowley, R. T. Bronson, D. Y. Chiang, M. Meyerson and T. Jacks (2011). “Suppression of lung adeno carcinoma progression by Nkx2-1.” Nature 473(7345): 101-104.
Wozniak, A. J., J. J. Crowley, S. P. Balcerzak, G. R. Weiss, C. H. Spiridonidis, L. H. Baker, K. S. Albain, K. Kelly, S. A. Taylor, D. R. Gandara and R. B. Livingston (1998). “Randomized trial comparing cisplatin with cisplatin plus vinorelbine in the treatment of advanced non-small-cell lung cancer: a Southwest Oncology Group study.” J Clin Oncol 16(7): 2459-2465.
Ye, J., J. J. Findeis-Hosey, Q. Yang, L. A. McMahon, J. L. Yao, F. Li and H. Xu (2011). “Combination of napsin A and TTF-1 immunohistochemistry helps in differentiating primary lung adenocarcinoma from metastatic carcinoma in the lung.” Appl Immunohistochem Mol Morphol 19(4): 313-317.

All references cited herein are fully incorporated by reference. Having now fully described the invention, it will be understood by a person skilled in the art that the invention may be practiced within a wide and equivalent range of conditions, parameters and the like, without affecting the spirit or scope of the invention or any embodiment thereof.

1. A method of assessing a sample from a subject, said method comprising
a) measuring in the sample of said subject the amount of specific transcription factor isoforms wherein said specific transcription isoforms are
i) the GATA6 Em isoform comprising the nucleic acid sequence of SEQ ID No: 1 or the GATA6 Em isoform comprising a nucleic acid sequence with up to 55 additions, deletions or substitutions of SEQ ID NO: 1;
ii) the NKX2-1 Em isoform comprising the nucleic acid sequence of SEQ ID No: 2 or the NKX2-1 Em isoform comprising a nucleic acid sequence with up to 39 additions, deletions or substitutions of SEQ ID NO: 2;
iii) the GATA6 Ad isoform comprising the nucleic acid sequence of SEQ ID No: 5 or the GATA6 Ad isoform comprising a nucleic acid sequence with up to 55 additions, deletions or substitutions of SEQ ID NO: 5; and
iv) NKX2-1 Ad isoform comprising the nucleic acid sequence of SEQ ID No: 6 or the NKX2-1 Ad isoform comprising a nucleic acid sequence with up to 38 additions, deletions or substitutions of SEQ ID NO: 6;
b) determining the LC score of the sample of said subject by performing at least one statistical algorithm for classification and for regression on measurement data of the subject, said statistical algorithm comprising
$$LC Score = - 0.607 * {log}_{2} ( \frac{Em GAT A 6}{Ad GAT A 6} ) - 1.431 {log}_{2} ( \frac{Em NKX 2 - 1}{Ad NKX 2 - 1} ) - 1.916$$
and
wherein at least one of the following is used as at least one classifier or a component of at least one classifier in the statistical method: GATA6 Em isoform, NKX2-1 Em isoform, GATA6 Ad isoform, NKX2-1 Ad isoform, ratio of GATA6 Em isoform/GATA6 Ad isoform, ratio of NKX2-1 Em isoform/NKX2-1 Ad isoform.

2. (canceled)

3. The method according to claim 1,
wherein the method further comprises the step of processing the measurement data, preferably normalizing, resealing, dimension reducing, and/or noise reducing.

4. The method according to claim 1,
wherein the method further comprises the steps of cross-validation and/or bootstrapping.

5. The method according to claim 1,
wherein the classifier in the method is
a) the GATA6 Em isoform of said sample set in relation to a GATA6 Em isoform of at least one control sample and wherein said value of the GATA6 Em isoform of said at least one control sample is obtained by measuring in said sample of said subject the amount of a specific transcription factor isoform wherein said specific transcription isoform is the GATA6 Em isoform comprising the nucleic acid sequence of SEQ ID No: 1 or the GATA6 Em isoform comprising a nucleic acid sequence with up to 55 additions, deletions or substitutions of SEQ ID NO: 1;
b) the NKX2-1 Em isoform in said at least one sample set in relation to a NKX2-1 Em isoform of at least one control sample and wherein said value of the NKX2-1 Em isoform in said at least one control sample is obtained by measuring in said at least one sample of said subject the amount of a specific transcription factor isoform wherein said specific transcription isoform is the NKX2-1 Em isoform comprising the nucleic acid sequence of SEQ ID No: 2 or the NKX2-1 Em isoform comprising a nucleic acid sequence with up to 39 additions, deletions or substitutions of SEQ ID NO: 2; or
c) a ratio of the GATA6 Em isoform and the GATA6 Ad isoform and a ratio of the NKX2-1 Em isoform and the NKX2-1 Ad isoform.

6-7. (canceled)

8. The method according to claim 1,
wherein the method comprises a support vector machine.

9. The method according to claim 1, wherein the amount of said specific transcription factor isoform(s) is measured via a polymerase chain reaction-based method, an in situ hybridization-based method, or a microarray.

10. The method according to claim 9, wherein the amount of said specific transcription factor isoform(s) is measured via a polymerase chain reaction-based method.

11. The method according to claim 10, wherein said polymerase chain reaction-based method is a quantitative reverse transcriptase polymerase chain reaction.

12-13. (canceled)

14. The method according to claim 1, wherein the amount of said specific transcription factor isoform(s) is measured on the polypeptide level.

15. The method according to claim 14, wherein the amount of said specific transcription factor isoform(s) is measured by an ELISA, a gel- or blot-based method, mass spectrometry, flow cytometry or FACS.

16. The method according to claim 1, wherein the subject has a lung cancer.

17. The method according to claim 16, wherein said lung cancer is non-small cell lung cancer (NSCLC) or small cell lung cancer (SCLC).

18. The method according to claim 1, wherein said sample comprises tumor cells.

19. The method according to claim 1, wherein said sample is a biopsy sample, a breath condensate sample, a blood sample, a bronchoalveolar lavage fluid sample, a mucus sample or a phlegm sample.

20. The method according to claim 1, wherein said subject is a human subject.

21. The method according to claim 20, wherein said human subject is a subject having an increased risk for developing cancer.

22. The method according to claim 1, further comprising the detection of one or more additional markers in a sample of said subject.

23-41. (canceled)

42. A method of treating a subject, said method comprising
a) selecting a subject; by measuring in a sample of said subject the amount of specific transcription factor isoforms wherein said specific transcription isoforms are
i) the GATA6 Em isoform comprising the nucleic acid sequence of SEQ ID No: 1 or the GATA6 Em isoform comprising a nucleic acid sequence with up to 55 additions, deletions or substitutions of SEQ ID NO: 1;
ii) the NKX2-1 Em isoform comprising the nucleic acid sequence of SEQ ID No: 2 or the NKX2-1 Em isoform comprising a nucleic acid sequence with up to 39 additions, deletions or substitutions of SEQ ID NO: 2;
iii) the GATA6 Ad isoform comprising the nucleic acid sequence of SEQ ID No: 5 or the GATA6 Ad isoform comprising a nucleic acid sequence with up to 55 additions, deletions or substitutions of SEQ ID NO: 5; and
iv) NKX2-1 Ad isoform comprising the nucleic acid sequence of SEQ ID No: 6 or the NKX2-1 Ad isoform comprising a nucleic acid sequence with up to 38 additions, deletions or substitutions of SEQ ID NO: 6;
b) determining the LC score of the sample of said subject by performing at least one statistical algorithm for classification and for regression on measurement data of the subject, said statistical algorithm comprising
$$LC Score = - 0.607 * {log}_{2} ( \frac{Em GAT A 6}{Ad GAT A 6} ) - 1.431 * {log}_{2} ( \frac{Em NKX 2 - 1}{Ad NKX 2 - 1} ) - 0.916$$
wherein at least one of the following is used as at least one classifier or a component of at least one classifier in the statistical method: GATA6 Em isoform, NKX2-1 Em isoform, GATA6 Ad isoform, NKX2-1 Ad isoform, ratio of GATA6 Em isoform/GATA6 Ad isoform, ratio of NKX2-1 Em isoform/NKX2-1 Ad isoform;
b) administering to said subject an effective amount of an anti-cancer agent and/or radiation therapy.

43. (canceled)

44. A computer program product comprising one or more computer readable media having computer executable instructions for determining a LC score from user entered amounts of GATA6 Em, GATA6 Ad, NKX2 EM, and NKX2 Ad, wherein the LC score is determined by performing at least one statistical algorithm for classification and for regression on measurement data of the subject, said statistical algorithm comprising
$$LC Score = - 0.607 * {log}_{2} ( \frac{Em GAT A 6}{Ad GAT A 6} ) - 1.431 {log}_{2} ( \frac{Em NKX 2 - 1}{Ad NKX 2 - 1} ) - 1.916$$
and displaying the results in a readable format.
